(19) United States
(12) Patent Application Publication
(10) Pub. No.: US 2004/0068764 A1
(43) Pub. Date: Apr. 8, 2004

(54) Method of enhancing virus-resistance in plants and producing virus-immune plants

(76) Inventors: Paul Wing Gay Chu, Florey (AU); Ronald George Garrett, Victoria (AU); Sten Roger Kalla, Victoria (AU); Philip John Larkin, Weston (AU); German Carlos Spangenberg, Victoria (AU); Thomas Joseph Higgins, Aranda (AU)

Correspondence Address: Cooper and Dunham, 1185 Avenue of the Americas, New York, NY, 10036, US

(21) Appl. No.: 10/432,008

(22) PCT Filed: Nov. 16, 2001
(86) PCT No.: PCT/AU01/01496

(30) Foreign Application Priority Data
Nov. 17, 2000 (AU) PR1558

(51) Int. Cl. A01H 1/00; C12N 15/82

(52) U.S. Cl. 800/279; 800/280

(57) ABSTRACT

The present invention provides a method of enhancing resistance of plants to one or multiple viruses, comprising introducing to a plant cell, and preferably expressing therein, a nucleotide sequence encoding a virus-encoded polypeptide. The present invention provides a method of enhancing the proportion of virus-resistant or virus-immune lines obtained from a single transformation experiment comprising introducing to a plant cell, a nucleotide sequence encoding a virus-encoded polypeptide operably in connection with a strong promoter sequence. The present invention provides novel gene sequences encoding the coat proteins of a virus and novel dysfunctional replicase sequences as well as gene constructs comprising same, in particular binary vector constructs suitable for introducing into plants and expressing the genes therein. The present invention provides and methods using same to enhance viral resistance in plants. The present invention provides novel methods of testing transgenic plants for the presence of a transgene.

### FIELD OF THE INVENTION

This invention relates generally to a method of enhancing resistance of plants to one or multiple viruses, or conferring immunity on plants against one or multiple viruses. More specifically, the present invention provides a method of enhancing resistance of plants to one or multiple viruses selected from the group consisting of bromoviruses, potyviruses, potexviruses, and nanoviruses, comprising introducing to a plant cell in the sense orientation, and preferably expressing therein, a nucleotide sequence encoding a virus-encoded polypeptide. The present invention further provides a method of enhancing the proportion of virus-resistant or virus-immune lines obtained from a single transformation experiment comprising introducing to a plant cell in the sense orientation, and preferably expressing therein, a nucleotide sequence encoding a virus-encoded polypeptide operably in connection with a strong promoter sequence selected from the group consisting of (i) a SCSV promoter sequence; (ii) a duplicated CaMV 35S promoter sequence; and (iii) the Arabidopsis thaliana SSU promoter sequence. The present invention further provides novel gene sequences encoding the coat proteins of a virus selected from the group consisting of bromoviruses, potyviruses, potexviruses, and nanoviruses, and gene constructs comprising same, in particular binary vector constructs suitable for introducing into plants and expressing the coat protein genes therein. A further aspect of the present invention provides a method for improving the germplasm of plants to enhance their resistance to one or multiple viruses or to confer immunity to one or multiple viruses on the improved plants. The present invention further provides transformed plants produced by performance of the inventive methods described herein.

General

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps. The present invention is not to be limited in scope by the specific embodiments described herein, which are intended for the purposes of exemplification only. Functionally-equivalent products, compositions and methods are clearly within the scope of the invention, as described herein.

Bibliographic details of the publications referred to by author in this specification are collected at the end of the description. Reference herein to prior art, including any one or more prior art documents, is not to be taken as an acknowledgment, or suggestion, that said prior art is common general knowledge in Australia or forms a part of the common general knowledge in Australia.

As used herein, the term “derived from” shall be taken to indicate that a particular integer or group of integers has originated from the species specified, but has not necessarily been obtained directly from the specified source.

This specification contains nucleotide and amino acid sequence information prepared using the programme PatentIn Version 2.0, presented herein after the claims. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212>and <213>, respectively. Nucleotide and amino acid sequences referred to in the specification are defined by descriptor “SEQ ID NO:” followed by the numeric identifier. For example, SEQ ID NO: 1 refers to the information provided in the numeric indicator field designated <400>1, etc.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

The designation of amino acid residues referred to herein are also those recommended by the IUPAC-IUB Biochemical Nomenclature Commission, wherein three-letter and one-letter abbreviations for naturally-occurring amino acids are listed in Table 1. In addition to the abbreviations listed in Table 1, the three-letter symbol Asx, or the one-letter symbol B, denotes Asp or Asn; and the three-letter symbol Glx, or the one-letter symbol Z, denotes glutamic acid or glutamine or a substance, such as, for example, 4-carboxyglutamic acid (Gla) or 5-oxoproline (Glp) that yields glutamic acid upon the acid hydrolysis of a peptide.

### BACKGROUND TO THE INVENTION

Leguminous species and crops are of great ecological, agronomic and social importance, providing protein-rich sources of food and fodder of high nutritive value and often serve as a meat substitute in developing countries. Some legume species are also grown for edible oil production, fibre, timber, green manure and ornamental purposes. Legumes also fix nitrogen and improve soil fertility and increase productivity of other plants species, particularly cereals in crop rotation systems. In Australia and New Zealand, pasture legumes are the backbone of the rural industries providing improved pastures for grazing and nitrogen for cropping. White clover is the most important pasture legume to the Australian dairy industry (Mason, 1993) and is a major component of improved pastures throughout the temperate world. Subterranean clover (Trifolium subterraneum L., subclover) is the major pasture legume in Australia and is grown on more than 16 million ha of mainly acidic and infertile lands. Lucerne is a major forage legume grown worldwide and is important for improving soil fertility and stability. In Australian alone, the annual lucerne crop is worth about $2 billion. However, unreliable yields and lack of persistence are major limitations to profitability and further expansion.

Plant viruses have been estimated to cause economic losses worldwide of US $15 billion per annum (Klausner, 1987) to pasture legume crops. A widespread gradual decline in pasture yields and persistence, known as “pasture decline”, leading to a lack of feed at critical times of the year, is reducing farm profitability. Recent surveys have shown that virus diseases are major causes of reduced pasture performance, and it has been estimated that controlling the major virus pests in white clover, lucerne and subterranean clover, could increase profitability for Australian rural industries by over $860 million.

The major viruses infecting pasture legume crops are alfalfa mosaic virus (AMV), bean yellow mosaic potyvirus (BYMV), clover yellow vein virus (CYVV), white clover mosaic virus (WCMV), and subterranean clover stunt nanovirus (SCSV), particularly in Trifolium spp. and lucerne (Medicago sativa) crops (Johnstone & McLean, 1987; Helms et al., 1993; Chu et al., 1995; Jones 1994, 1996). Studies have indicated that these viruses can induce subterranean clover herbage and seed yield losses by up to 97% and 90%, respectively, and reduce the nutritional quality, nitrogen-fixing capacity and persistence of the pastures. Based on recent economic analyses of pasture improvements (Pearson et al., 1997) it is estimated that controlling these viruses in subterranean clover pastures could increase profitability for Australian rural industries by over $278 million. A recent study showed that transgenic subterranean clover with resistance to BYMV can show a significant yield improvement from 70% herbage loss in non-transgenic plants to only 20% loss in BYMV-resistant transgenic plants (Chu et al., 1999).

Additionally, various studies in Australia and overseas showed that AMV, CYVV and WCMV diseases are reducing white clover pasture production potential by up to 30% through reduced foliage yield and quality, reduced nitrogen fixing capacity and reduced persistence (Garrett, 1991, 1992; Nikandrow and Chu, 1991; Mason, 1993; Gibson et al., 1981, Campbell and Moyer, 1984; Edwardson and Christie, 1986, Latch and Skipp, 1987). For example, studies showed that AMV in white clover alone causes losses in milk production of $30 million annually (Garrett, 1991, 1992). An effective virus disease control in white clover will improve the profitability and competitiveness of the dairying industry.

Additionally, recent studies showed that alfalfa mosaic virus (AMV) is also a major factor contributing to reduced lucerne yields and persistence. Surveys in 1991-1993 showed that AMV is by far the most prevalent and serious virus of lucerne in Australia. Incidence of AMV infection in lucerne frequently reaches over 90% in Australia and overseas. Yield loss studies using four isolates of AMV and 7 cultivars of lucerne showed that AMV typically caused yield reductions of 20-40%. Other studies showed that AMV not only causes direct yield loss but also reduces forage quality, nitrogen fixing capacity and winter survival and also predispose them to infection by other pathogens, resulting in reduced plant density and rapid decline in production with age and causing an estimated annual economic loss of about $80 million in Australia alone.

AMV is an alfamovirus, CYVV is a potyvirus, and WCMV is a potexvirus. These three viruses are members of three of the largest families or groups of plant viruses, namely, the Bromoviridae (AMV), Potyviridae (CYVV), and Potexvirus (WCMV). Each of these viruses individually infect a large number of plant species causing significant production losses in many plant species, especially in pasture and grain legumes. Of the 47 different plant virus families or groups, the family Potyviridae is by far the largest, accounting for approximately 25% of all known plant viruses (Shukla et al., 1994). Currently, 198 distinct viruses world-wide have been assigned to this family and new members are being discovered and added to this list more frequently than to any other virus groups (Ward and Shukla, 1991; Shukla et al., 1994).

Most potyviruses have a wide host range infecting plants from several families, and a few members infect species in up to 30 families. They flourish in a wide range of crops and geographical regions (Hollings and Brunt, 1981a, 1981b). By 1991, the host members had increased to 2026 species, 556 genera and 81 families (Edwardson and Christie, 1991). Their relative economic importance is highlighted by the fact that in a recent survey of the ten most important filamentous viruses from each of the ten major world regions, 73% were potyviruses (Milne, 1988). It has been estimated that potyviruses account for about 20% of all losses caused by plant viruses.

Potexviruses cause mosaic or ringspots in a wide range of mono- and dicotyledonous plants. The viruses are readily transmitted in nature by mechanical contacts and have a world-wide distribution.

Members of the family Bromoviridae also have a cosmopolitan distribution. A number are important pathogens of crops and horticultural species in the plant families Graminae, Leguminoseae and Solanaceae. All of the viruses are transmissible by mechanical inoculation and in nature are transmitted by a wide variety of aphids, via pollen or through seed.

Subterranean clover stunt virus (SCSV) is the type species of a new group of plant viruses which naturally infect several major food and forage crops among their hosts (Randles et al., 2000).

The traditional means of preventing plant virus infections have been restricted to eradication of infected plants and propagating materials, vector control, selection of natural resistant plant lines and cross-protection with mild strains of the same virus (Matthews, 1991). Most of these classical methods are laborious and economically and/or environmentally unsustainable. There is no effective natural resistance to viruses in white clover or lucerne (Taylor and Gabrial, 1986; Gibson et al., 1989), and it is rare in other Trifolium species. Interspecific crosses using T. repens are difficult and require embryo rescue methods (Baker & Williams, 1987), and produce hybrids that require considerable improvement by traditional breeding methods.

There is a need for the development of alternative strategies to sustain resistance (to not select for resistance breaking strains of the virus) by using at least two or more unrelated mechanisms (eg. transgenes).

Essentially all the main biochemical processes including DNA replication, protein synthesis, active transport, and signal transduction are coupled to nucleoside triphosphate (NTP—usually ATP) hydrolysis (Gorbalenya and Koonin, 1989). Numerous, though not all, NTPases possess conserved amino acid sequences (Walker et al, 1982; Gobalenya and Koonin, 1989; Saraste et al, 1990). One such amino acid sequence is referred to as the Walker A motif, the NTP-binding motif but most commonly as the P-loop motif. The amino acid sequence of the motif is (A or G)XXXXGK(S or T) (where A is alanine, G is glycine, K is lysine, S is serine, T is Threonine and X can be any amino acid). The presence of this amino acid sequence suggests that the protein is involved in NTP binding as the sequence has been shown to be highly non-random and correlates well with demonstrated NTP binding or hydrolysis (Gorbalenya and Koonin, 1989; Saraste et al, 1990).

The P-loop motif has been mutated in the gene for the RNA-dependent RNA polymerase of potato virus X where the last three amino acids of the P-loop (GKS) were changed to AKS, GNS and GES (Davenport and Baulcombe, 1997). The changes were made to infectious clones of the virus which allowed for the testing of the effect of the mutation. Clones with the AKS mutation still infected plants whilst the GNS or GES mutations did not allow virus accumulation, either in tobacco plants or protoplasts. This is consistent with previous mutational analysis of the P-loop and the idea that the lysine residue interacts with the negatively charged phosphate group of an NTP (Logan and Knight, 1993; Story et al, 1993; Konola et al, 1994).

AMV possesses a positive-sensed single-stranded RNA genome consisting of four RNA species. The genomic RNAs1 and 2 encodes for gene products necessary for viral replication while the genomic RNA 3 encodes the movement protein required for virus spread. The coat protein gene is located on both the RNA3 and a non-replicating sub-genomic RNA 4 but is only synthesised from the latter RNA species. The proteins from AMV RNA1 and AMV RNA2, called 1a and 2a respectively, form a replication complex which replicates RNAs 1, 2 and 3 in plant cells. The replication complex requires the hydrolysis of ATP for the synthesis of new RNA molecules (Gorbalenya and Koonin, 1989) and located in the coding region of AMV RNA1 is an ATP binding motif.

In work leading up to the present invention, the inventors sought to develop methods for producing virus resistant or virus-immune lines of pasture legume crops by using genetic engineering technology. In particular, the inventors sought to develop immune or resistant lines at high frequencies, by expressing part or all of the viral genes in plants operably in connection with suitable promoter sequences. A further object of the invention was to produce immune or resistant plant lines that retained their resistance or immunity characteristics in the field. A further goal of the invention was to produce plants having immunity or resistance to multiple viruses, such as, for example, two or more viruses selected from the group consisting of: AMV, CYVV, WCMV and SCSV, particularly under field conditions. Accordingly, the inventors have introduced into elite cultivars of white clover, red clover, subterranean clover, and lucerne, the coat protein genes or replicase genes of these viruses, placed operably under the control of effective promoters which, surprisingly enhance the frequency of production of immune or resistant plants, as well as enhancing expression of the introduced viral genes. The plants generated using the procedures described herein have immunity or enhanced resistance compared to otherwise isogenic non-transformed lines, under both glasshouse and field conditions. The plants produced in accordance with the procedures described herein are particularly suitable for the development of elite germplasm having novel virus-resistance or virus-immunity characteristics.

### SUMMARY OF THE INVENTION

One aspect of the present invention provides a method of enhancing resistance of a plant to one or multiple viruses, comprising introducing to said plant a nucleotide sequence encoding one or more polypeptide(s) selected from the group consisting of virus-encoded coat proteins and dysfunctional viral replicases, wherein said virus is a plant pathogen.

Preferably the plant pathogen is an RNA virus and in particular a virus is selected from the group consisting of bromoviruses, potyviruses, potexviruses, and nanoviruses. Preferably, the virus or viruses against which immunity or resistance is conferred or enhanced, respectively, is selected from the group consisting of alfalfa mosaic virus (AMV), clover yellow vein virus (CYVV), sub-clover stunt virus (SCSV), bean yellow mosaic virus (BYMV) and white clover mosaic virus (WCMV).

Those skilled in the art will be aware that bromoviruses, potyviruses, potexviruses, and nanoviruses are pathogenic viruses of plants, and, in particular, pathogenic viruses of pasture or forage species and in particular pasture or forage legumes. Accordingly, it is preferred that the plant on which immunity is conferred or resistance is enhanced by the performance of the inventive method is a pasture species and preferably a pasture legume, more preferably a pasture legume selected from the group consisting of Trifolium spp. and Medicago spp. However, the person skilled in the art will realise that where the virus encoded polypeptide is a dysfunctional replicase, as discussed below, that the method is broadly applicable to any plant species. In a particularly preferred embodiment, immunity is conferred or resistance is enhanced in a plant selected from the group consisting of: T. repens, T. subterraneum, T. pratense, T. michelianum, T isthmocarphum, and M. sativa. Unless specifically stated otherwise, the performance of the inventive method described herein on other species of plant or pasture legumes is not excluded.

In a bioassay of such viruses on a susceptible plant host, or indicator host known to those skilled in the art, more than 50% of the plants infected with virus-containing sap derived from an infected plant will become infected with the virus and may develop symptoms of infection, such as, for example, lesions, chlorosis or necrosis of leaves, veins, or other plant organs. Similarly, following mechanical inoculation of a susceptible host plant with a virus inoculum, more than 50% of the susceptible plants will become infected.

Accordingly, the term “resistance” as used in the examples shall be taken to mean that 50%, or less, of a test sample or population of plants are capable of being infected with a virus or virus-containing plant extract, following inoculation with said virus or virus-containing, as determined by symptom recognition, infectivity, or virus bioassay data on a suitable indicator host known to those skilled in the art.

In contrast, by “enhancing resistance” or “enhanced resistance” is meant that the resistance of a non-naturally occurring plant or plant part produced in accordance with the methods described herein to a virus is made greater than the resistance of the naturally-occurring plant or plant part from which said non-naturally occurring plant or plant part is derived. It will be clear to those skilled in the art that a transformed plant or plant part, or a progeny plant or plant part derived therefrom, which comprises a nucleotide sequence encoding a virus-encoded polypeptide inserted into its genome in accordance with the inventive method, consists of a non-naturally-occurring plant or plant part. Enhanced resistance as used in this context may also be indicated by the presence of fewer viral lesions, reduced levels of infectious material, recovery or increased speed of recovery from infection or delayed or reduced spread of infection when compared to a control a test sample or population of plants. Thus enhanced resistance is a relative term and does not require that 50%, or less, of a test sample or population of plants are capable of being infected with a virus or virus-containing plant extract, following inoculation with said virus or virus-containing.

The term “immunity” shall be taken to mean that the plants of a test sample or population do not become infected with a virus or virus-containing plant extract, following inoculation with said virus or virus-containing, as determined by symptom recognition, infectivity, or virus bioassay data on a suitable indicator host known to those skilled in the art. The person skilled in the art will appreciate that the term “immunity” is not absolute, and a low level of infection in a large population will be acceptable. Preferably the level of infection is less than 20% 10%, 5% or 2% and more preferably less than 1% of the population, as determined by symptom recognition, infectivity, or virus bioassay data. Further, the plants of a test sample or population may be asymptomatic, have very low levels of infection or have only transient infection and still be immune, provided there is not substantial commercial damage to the crop.

Preferably, the inventive method results in the production of plants that have immunity against one or more viruses, or enhanced resistance against one or more viruses, under field conditions. By “field conditions” is meant that the characteristics of immunity or resistance identified in the primary regenerant (i.e. T₀ plant) are substantially stable to be exhibited by T1 or T2 progeny which also contain the introduced nucleotide sequence when grown in the field under conditions in which otherwise isogenic plants that do not contain the introduced nucleotide sequence are susceptible to the virus(es), such as, for example, by becoming infected and possibly exhibiting symptoms of infection, as determined by standard procedures of bioassay, mechanical inoculation with virus or aphid transmission tests, amongst others.

The present invention is particularly useful for conferring immunity on a plant, or enhancing the resistance of a plant, to two or more viruses, preferably three or more viruses, and even more preferably all of the viruses selected from the group consisting of alfalfa mosaic virus (AMV), clover yellow vein virus (CYVV), sub-clover stunt virus (SCSV) and white clover mosaic virus (WCMV).

As exemplified herein, immunity is conferred on pasture legumes, or resistance is enhanced in a pasture legume, against each of the viruses AMV, CYVV, SCSV, and WCMV, considered separately, by introducing the coat protein gene or a dysfunctional replicase gene of the particular virus in question into the cells of the plant.

Additionally, the inventors have herein exemplified the production of plants that have double-immunity or enhanced double-resistance against both AMV and CYVV, indicating that the approach taken is feasible and capable of application to other virus combinations. Accordingly, the present invention clearly extends to the conferring of double-immunity, or the enhancing of double-resistance, against both AMV and CYVV, or both AMV and SCSV, or both AMV and WCMV, or both CYVV and WCMV, or both CYVV and SCSV, or both WCMV and SCSV.

Triple-immunity or triple-resistance against a virus combination selected from the group consisting of (i) AMV and CYVV and WCMV; (ii) AMV and CYVV and SCSV; (iii) AMV and WCMV and SCSV; and (iv) CYVV and WCMV and SCSV; is also contemplated by the present invention.

By “isolated nucleotide sequence” is meant that the nucleotide sequence is in a non-naturally occurring form, such as, for example, contained within a gene construct, or a vector, such as, for example, a binary vector or recombinant virus vector. Accordingly, the present invention clearly does not encompass the infection of a plant with a naturally-occurring virus particle, or other introduction of a naturally-occurring virus particle to a plant. As will be apparent to those skilled in the art, once the isolated nucleic acid sequence has been introduced into the plant cell, and particularly in cases where it is subsequently integrated into the plant cell genome, it may not exist in the same form as when originally introduced. However, in so far as the nucleotide sequence encoding the virus-encoded polypeptide is present within the plant cell in a form other than that which occurs in nature (i.e. contained within the virus from which said nucleotide sequence was derived), said nucleotide sequence shall be taken to be in an isolated form. Those skilled in the art can readily determine whether a plant cell contains heterogeneous nucleic acid encoding a virus-encoded polypeptide in a form other than the native virus by standard procedures, including Southern hybridisation, northern hybridisation, or polymerase chain reaction (PCR) performed essentially as described herein. Accordingly, there is no undue burden of experimentation placed upon the skilled addressee in determining whether or not a nucleotide sequence encoding a virus-encoded polypeptide has been introduced previously into a plant cell in accordance with the procedures described herein.

Preferably, the isolated nucleotide sequence encodes one or more viral coat proteins, or a dysfunctional viral replicase polypeptide, and more preferably, one or more viral coat proteins, or a dysfunctional viral replicase polypeptide of one or more viruses selected from the Bromoviridae family and more preferably from the group consisting of bromoviruses, potyviruses, potexviruses, and nanoviruses.

For conferring multiple immunity, or enhancing multiple resistance, the isolated nucleotide sequence may comprise nucleotide sequences encoding two or more virus-encoded polypeptides, in which case multiple immunity may be conferred, or multiple resistance may be enhanced, in a single step. As exemplified herein, the binary vector pBH3 comprises the coat protein-encoding genes of both CYVV and WCMV for the purposes of conferring immunity or enhancing resistance against both viruses in plants in a single step. Alternatively, or in addition, multiple immunity may be conferred, or multiple resistance may be enhanced, in several steps, such as, for example, by sequential rounds of introducing nucleotide sequences encoding the virus-encoded polypeptides into plant cells. For example, a plant cell which carries the binary vector pBH3, or similar binary vector, may be subjected to further rounds of transformation to introduce nucleotide sequences comprising the AMV or SCSV coat-protein-encoding gene(s), thereby producing plants having immunity or enhanced resistance against three or four viruses. Similarly, two plants having immunity or enhanced resistance against one or more different viruses, wherein at least one plant has been produced by the performance of the invention, may be crossed to produce progeny plants carrying the introduced nucleotide sequences of both parents, and exhibiting multiple immunity, or multiple resistance, against the viruses to which both parents are immune or have resistance. Such procedures are exemplified herein as methods for improving the germplasm of plants.

Still more preferably, the isolated nucleotide sequence encodes one or more viral coat proteins, or a dysfunctional viral replicase polypeptide of one or more viruses selected form the group consisting of: alfalfa mosaic virus (AMV), clover yellow vein virus (CYVV), sub-clover stunt virus (SCSV) and white clover mosaic virus (WCMV). In the case of conferring single immunity or enhancing resistance against a single virus, all combinations of viral coat protein genes and/or viral replicase polypeptides of that virus are contemplated herein. In the case of conferring multiple immunity or enhancing resistance against more than one virus, all combinations of viral coat protein genes and/or viral replicase polypeptides derived from those multiple viruses are contemplated herein.

Even more preferably, the isolated nucleotide sequence comprises a sequence selected from the group consisting of:

1. an alfalfa mosaic virus coat protein-encoding sequence selected from the group consisting of: SEQ ID Nos: 1, 3, 5, 7, 9, 11, 13, 15, and 17;

2. the clover yellow vein virus coat protein-encoding sequence set forth in SEQ ID NO: 25;

3. a white clover mosaic virus coat protein-encoding sequence selected from the group consisting of SEQ ID Nos: 30, 32, and 34; and

4. a nucleotide sequence that is degenerate to any one of the sequences of (1), (2) or (3).

For the purposes of nomenclature, the nucleotide and amino acid sequences set forth in SEQ ID Nos: 1-10 relate to Type I AMV isolates. In particular, the nucleotide sequence set forth in SEQ ID NO: 1 consists of the coat protein-encoding open reading frame of AMV isolate H1, and the corresponding amino acid sequence encoded therefor is presented herein as SEQ ID NO: 2. The nucleotide sequence set forth in SEQ ID NO: 3 consists of the coat protein-encoding open reading frame of AMV isolate WC3, and the corresponding amino acid sequence encoded therefor is presented herein as SEQ ID NO: 4. The nucleotide sequence set forth in SEQ ID NO: 5 consists of the coat protein-encoding open reading frame of AMV isolate 425S, and the corresponding amino acid sequence encoded therefor is presented herein as SEQ ID NO: 6. The nucleotide sequence set forth in SEQ ID NO: 7 consists of the coat protein-encoding open reading frame of AMV isolate 425M, and the corresponding amino acid sequence encoded therefor is presented herein as SEQ ID NO: 8. The nucleotide sequence set forth in SEQ ID NO: 9 consists of the coat protein-encoding open reading frame of AMV isolate 425L, and the corresponding amino acid sequence encoded therefor is presented herein as SEQ ID NO: 10.

The nucleotide and amino acid sequences set forth in SEQ ID Nos: 11-18 relate to Type II AMV isolates. In particular, the nucleotide sequence set forth in SEQ ID NO: 11 consists of the coat protein-encoding open reading frame of AMV isolate YSMV, and the corresponding amino acid sequence encoded therefor is presented herein as SEQ ID NO: 12. The nucleotide sequence set forth in SEQ ID NO: 13 consists of the coat protein-encoding open reading frame of AMV isolate AMU12509, and the corresponding amino acid sequence encoded therefor is presented herein as SEQ ID NO: 14. The nucleotide sequence set forth in SEQ ID NO: 15 consists of the coat protein-encoding open reading frame of AMV isolate AMU12510, and the corresponding amino acid sequence encoded therefor is presented herein as SEQ ID NO: 16. The nucleotide sequence set forth in SEQ ID NO: 17 consists of the coat protein-encoding open reading frame of AMV isolate YD3.2, and the corresponding amino acid sequence encoded therefor is presented herein as SEQ ID NO: 18.

The nucleotide sequence set forth in SEQ ID NO: 25 consists of the coat protein-encoding open reading frame of CYVV isolate 300, and the corresponding amino acid sequence encoded therefor is presented herein as SEQ ID NO: 26.

The nucleotide and amino acid sequences set forth in SEQ ID Nos: 30-35 relate to different isolates of WCMV. In particular, the nucleotide sequence set forth in SEQ ID NO: 30 consists of the coat protein-encoding open reading frame of the Bundoora isolate of WCMV (syn. “WCMV B”), and the corresponding amino acid sequence encoded therefor is presented herein as SEQ ID NO: 31. The nucleotide sequence set forth in SEQ ID NO: 32 consists of the coat protein-encoding open reading frame of the WCMV isolate M, and the corresponding amino acid sequence encoded therefor is presented herein as SEQ ID NO: 33. The nucleotide sequence set forth in SEQ ID NO: 34 consists of the coat protein-encoding open reading frame of WCMV isolate O, and the corresponding amino acid sequence encoded therefor is presented herein as SEQ ID NO: 35.

In the present context, the term “virus-encoded polypeptide” shall be taken to mean a polypeptide that is normally expressed by the genome of a plant virus or a sub-genomic fragment of said genome.

Preferably, the virus-encoded polypeptide is a viral coat protein, or viral replicase. In a particularly preferred embodiment, the viral coat protein comprises an amino acid sequence selected from the group consisting of SEQ ID Nos: 2, 4, 6, 8, 10, 12, 14, 16, 18, 26, 31, 33, and 35.

In another aspect of the invention, resistance in plants to virus is conferred by the expression of dysfunctional replicase gene which is preferably dysfunctional in that it forms a complex which is unable to replicate genomic viral RNAs and thereby inhibits or slows infection by the virus. Preferably the gene is mutated so that the expressed protein can no longer undertake hydrolysis of ATP. In a preferred embodiment the gene is mutated by modifying a NTP binding motif, preferably a ATP binding motif.

In a specific aspect the invention is a method of enhandng resistance to a plant virus by the expression of a replicase gene with a mutated NTP binding (P-loop) motif. The resistance was shown in tobacco as a model plant system and in white clover as a commercial plant species with high susceptibility to AMV infection. Given the highly conserved nature of the ATP binding motif, the same mechanism will be effective for conferring resistance to other plant viruses Gorbalenya and Koonin, 1989 discuss the highly conserved nature of NTP-binding domains in dissimilar RNA viruses and exemplify a number of consensus sequences which can be utilised in the present invention. This citations is incorporated in its entirety herein by reference.

In a preferred embodiment, the defective gene is a modified AMV RNA 1 gene which expresses a dysfunctional AMV 1a protein. Without being bound by theory, the inventors suggest that the modified protein forms a complex with the AMV RNA 2a protein which is then unable to replicate the genomic viral RNAs and thereby inhibit or slow infection by the virus. By genetically engineering plants to express a mutant of the AMV 1a protein that does not bind ATP (by altering the ATP binding site), the replication of the virus is blocked.

The ATP binding motif has been positively identified only in the 1a protein of all tripartite viruses and is considered a highly non-random sequence, which has not been previously used. The sequence of the 1a protein is likely to be similar between strains and so will be widely applicable to all AMV strains.

A second protein motif identified with association to NTP-binding is referred to as the Walker B motif or Mg⁺⁺ binding site. The Walker B motif is hhhD(D or E) (where h is a bulky hydrophobic amino acid, D is aspartic acid and E is glutamic acid) (Gorbalenya and Koonin, 1989; Koonin, 1997). In all proteins where both the P-loop and Walker B motifs are present, the P-loop is on the amino-terminal side relative to the Walker B usually between 30 and 130 amino acids apart (Yoshida and Amano, 1995).

A motif search of all AMV protein sequences published revealed that two P-loop motifs exist in the genome. The first is in the putative helicase domain of the 1a protein (Table A) and the second in the 2a protein (Table B). The P-loop motif was only found in the 1a protein of all other Bromoviridae viruses (Table A). No P-loop motif was found in the 3a protein among all Bromoviridae viruses (Table C). On the basis that the P-loop motif in the 1a protein is in the putative helicase domain and is conservatively located in a similar position in closely related viruses, it is highly probable to be involved in ATP binding and hydrolysis. Furthermore, for all Bromoviridae viruses, including AMV, a Walker B motif was identified in their 1a protein at approximately 60 amino acids from the putative P-loop motif on the carboxyl-terminal side.

TABLE A

ATP binding motifs identified in the 1a protein of viruses belonging to the Bromoviridae family

| | | | | ATP Binding motif (underlined) |
|---|---|---|---|---|
| | | | GenBank Locus | and location of the first amino |
| Genera | Virus | Strain | or Accession # | acid of the motif in the protein |
| Alfamovirus | Alfalfa Mosaic Virus | 425-L | MAACG1Z | 838: V T I B D G V A G C G K T T N I K Q |
| | Alfalfa Mosaic Virus | Q | MAARNA13 | Only 3′sequence |
| Bromovirus | Broad Bean Mottle Virus | BBMIAP | 690: V V M V D G V A G C G K T T A I K E | |
| | Cowpea Chlorotic Mottle Virus | MCCP1A | 682: I S L C D G V A G C G K T T A I K S | |
| | Brome Mosaic Virus | MBRCG1Z | 685: I S M V D G V A G C G K T T A I K D | |
| | | | BRBMV1 | 685: I S M V D G V A G C G K T T A I K D |
| | | | BMV1APROT | 685: I S M V D G V A G C G K T T A I K D |
| Cucumovirus Cucumber Mosaic Virus | Y | D12537 | 715: I S Q V D G V A G C G K T M P I K S | |
| | | Q | CURNA1Q | 713: I S Q V D G V A G C G K T M P I K S |
| | | Fny | MCVFRNA1 | 714: I S Q V D G V A G C G K T M P I K S |
| | | | CMU20220 | 714: I S Q V D G V A G C G K T M P I K S |
| | | | MCVR1PB* | 99: I S Q V D G V A G C G K T M P I K S |
| | | Iizuka | MCVL1 | 714: I S Q V D G V A G C G K T M P I K S |
| | Tomato Aspermy Virus | V | TOAVRNA1 | 714: I S Q V D G V A G C G K T M P I K S |
| | Peanut Stunt Virus | J | PSVJ1A | 722: I S Q V D G V A G C G K T M P I K S |
| Ilarvirus | Tobacco Streak Virus | | AAB48983 | 806: I T I V D G V A G C G K T T H L K K |
| | Citrus leaf rugose virus | | CLU23715 | 765: V I I E D G V A G C G K T T S L L K |
| | Elm mottle virus | | SLU57047 | 774: V V I E D G V A G C G K T T S L L K |
| | Spinach latent virus | | PMOVRNA1 | 775: I V I E D G V A G C G K T T S L L K |
| | Prune dwarf virus | | PDU57648 | 770: I T I M D G V A G C G K T T K I K S |
| Oleavirus | Olive latent virus 2 | | OLV21APRT | 631: K T W I D G V A G C G K T Y E I V H |

*NOTE:
The sequence of the Cucumber Mosaic Virus entry MCVR1PB was only the 3′ end of the 1a gene.

TABLE B

ATP binding motifs identified in the 2a protein of viruses belonging to the Bromoviridae family

| | | | | |
|---|---|---|---|---|
| | | | | ATP Binding motif (underlined) |
| | | | GenBank Locus | and location of the fist amino |
| Genera | Virus | Strain | or Accession # | acid of the motif in the protein |
| Alfamovirus | Alfalfa Mosaic Virus | 425-L | MAACG2Z | 747: A L E S L G K I F A G K T L C K E C |
| | | | A1MVRNA2 | 747: A L E S L G K I F A G K T L C K E C |
| Bromovirus | Broad Bean Mottle Virus | | BBMRNA2Q | no ATP binding motif |
| | | | BBU24495 | no ATP binding motif |
| | | Mo | BBU24496 | no ATP binding motif |
| | Cowpea Chlorotic Mottle Virus | MCCRNAA2 | no ATP binding motif | |
| | Brome Mosaic Virus | | MBRCG2Z | no ATP binding motif |
| | | | BRBMV2 | no ATP binding motif |
| | | | BMV2APROT | no ATP binding motif |
| Cucumovirus | Cucumber Mosaic Virus | Fny | MCVRN2 | no ATP binding motif |
| | | Y | D12538 | no ATP binding motif |
| | | Q-CMV | CVRNA02 | no ATP binding motif |
| | | NT9 | MCV2A2 | no coding region defined |
| | | | MCVORNA2 | no ATP binding motif |
| | | | MCVL2 | no ATP binding motif |
| | Tomato Aspermy Virus | V | TOAVRNA2 | no ATP binding motif |
| | Peanut Stunt Virus | J | PSVJ2A | no ATP binding motif |
| Ilarvirus | Tobacco Streak Virus | | TSU75538 | no ATP binding |
| | Citrus leaf rugose virus | | CLU17726 | no ATP binding |
| | Elm mottle virus | | SOU34050 | no ATP binding |
| | Spinach latent virus | | PMOVRNA2 | no ATP binding |
| | Prune dwarf virus | | AF277662 | no ATP binding |
| Oleavirus | Olive latent virus 2 | | OLV22APRT | no ATP binding |

TABLE C
ATP binding motifs identified in the 3a protein of viruses belonging to the Bromoviridae family

| | | | GenBank Locus | ATP Binding motif (underlined) and location of the |
|---|---|---|---|---|
| Genera | Virus | Strain | or Accession # | first amino acid of the motif in the protein |
| Alfamovirus | Alfalfa Mosaic Virus | 425-S | ALAM19 | no ATP binding motif |
| | | YSMV | MAA32KDMP | no ATP binding motif |
| | | 425-M | MAACG3Z | no ATP binding motif |
| | | 3-L | MAARNA3L | no ATP binding motif |
| Bromovirus | Broad Bean Mottle Virus | | BBM3ACT | no ATP binding motif |
| | Cowpea Chlorotic Mottle Virus | | MCCRNA3 | no ATP binding motif |
| | | | MCCRNAA3 | no ATP binding motif |
| | Brome Mosaic Virus | Russian | MBRCG3Z | no ATP binding motif |
| | | | BRBMV3 | no ATP binding motif |
| | | | BMV3APROT | no ATP binding motif |
| Cucumovirus | Cucumber Mosaic Virus | Q | MCVRNA3A | no ATP binding motif |
| | | trk 7 | MCV3APCOAT | no ATP binding motif |
| | | O | MCVO3 | no ATP binding motif |
| | | Kor | MCVRNA3KOR | no ATP binding motif |
| | | Y | MCVRNA3 | no ATP binding motif |
| | | | CMV3ACP | no ATP binding motif |
| | | WL | MCVRNA3WL | no ATP binding motif |
| | | C | MCVRNA3C | no ATP binding motif |
| | | | CMU37227 | no ATP binding motif |
| | | | MCV3APA | no ATP binding motif |
| | | | CMU20219 | no ATP binding motif |
| | | E5 | MCVR3MPCP2 | no ATP binding motif |
| | | | CMU20668 | no ATP binding motif |
| | | C7-2 | MCVST3ACP | no ATP binding motif |
| | Tomato Aspermy Virus | C | TOARNA3 | no ATP binding motif |
| | | P | TOA3APCOAT | no ATP binding motif |
| | Peanut Stunt Virus | J | PSVRNA3 | no ATP binding motif |
| Ilarvirus | Tobacco Streak Virus | | TOTSV3 | no ATP binding |
| | Citrus leaf rugose virus | | CLU17390 | no ATP binding |
| | Elm mottle virus | | SLU57048 | no ATP binding |
| | Elm mottle virus | | EMU85399 | no ATP binding |
| | Spinach latent virus | | PMOVRNA3 | no ATP binding |
| | Prune dwarf virus | | PDVMOVCAP | no ATP binding |
| | | ch 137 | PDVMOVCAP | no ATP binding |
| | Apple Mosaic Virus | | AMU15608 | no ATP binding |
| | Hydrangea mosaic virus | | HMU35145 | no ATP binding |
| Oleavirus | Olive latent virus 2 | | OLV212 | no ATP binding |

The term “sense orientation” shall be taken to mean that the nucleotide sequence encoding the virus-encoded polypeptide is introduced into a plant cell, plant part, or whole plant, in a format suitable for its expression in said plant cell, plant part, or whole plant or in a plant cell, plant part or whole plant derived therefrom by any means including regeneration following transformation.

Likewise, the term “antisense orientation”, “negative sense” or “inverted” shall be taken to mean that the nucleotide sequence encoding the virus-encoded polypeptide is introduced into a plant cell, plant part, or whole plant, in a format the inverse of that generally suitable for its expression in said plant cell, plant part, or whole plant or in a plant cell, plant part or whole plant derived therefrom.

Inhibitory RNA (iRNA) is a little understood phenomena which utilises RNA to inhibit gene expression. Recently, targeting genes for silencing and virus resistance has been successful by the expression of so called ‘hairpin RNA’ constructs (Waterhouse et al, 1998; Wang et al, 2000). These gene constructs express an RNA that forms a hairpin like shape because it contains a sense sequence and an repeat complementary sequence. These repeats are separated by a unique sequence which forms the loop for the hairpin. The ‘hairpin RNA’ constructs induce the PTGS system to degrade the target RNA (Waterhouse et al, 2000).

The term “introducing”; in the context of introducing the isolated nucleotide sequence to the plant, shall be taken to include the transformation, or transfection, of a single plant cell or plant tissue or plant organ or whole plant with said isolated nucleotide sequence. Accordingly, it will be apparent to those skilled in the art that the isolated nucleic acid encoding one or more virus-encoded polypeptides will be taken as having been introduced to the genome of a plant that has been regenerated from an individual transformed or transfected cell (i.e. the primary regenerant or “T₀” plant).

However, the term “introducing” shall extend to the transfer of the introduced nucleotide sequence from the primary regenerant to all progeny derived therefrom which also contain the introduced nucleotide sequence, whether by virtue of sexual self-fertilisation, sexual hybridisation or out-crossing, clonal propagation, or additional rounds of transformation or transfection. Accordingly, the term “introducing” clearly includes the introgression of an isolated nucleotide sequence from a primary-transformed plant, or the progeny thereof, to another plant line, such as, for example, by selective breeding. For example, the isolated nucleotide sequence may be introduced into an elite commercial cultivar from a transformed plant (i.e. the primary regenerant or the progeny thereof which also contain the introduced nucleotide sequence) by back-crossing, to produce a plant having substantially the same commercially-useful characteristics as the elite commercial cultivar parent in addition to containing the introduced nucleotide sequence. In all such circumstances, the progeny plant shall be taken to have the isolated nucleotide sequence encoding the virus-encoded polypeptide introduced into its genome, notwithstanding that it is not the immediate end-product of a recombinant approach employing transformation or transfection technology.

In a particularly preferred embodiment, the present invention provides a method of enhancing resistance of a plant to one or multiple viruses or conferring immunity against one or multiple viruses on a plant, comprising introducing an isolated nucleotide sequence encoding a virus-encoded polypeptide to said plant in the sense orientation, and wherein said isolated nucleotide sequence is introduced to the said plant by a process comprising:

(i) transforming a plant cell with said isolated nucleotide sequence to produce a transformed plant cell;

(ii) regenerating a whole plant from said transformed plant cell; and

(iii) obtaining a progeny plant from said whole plant wherein said progeny plant contains one or more gene copies of the isolated nucleotide sequence.

It will be apparent from the preceding description that the term “obtaining” extends to the use of all means known to those skilled in the art, including sexual means, asexual means, or recombinant technologies, for transferring the introduced nucleotide sequence from the primary regenerant plant across generations.

By “one or more gene copies” is meant that the progeny plant may be heterozygous or homozygous for the introduced nucleotide sequence. Additionally, the introduced nucleotide sequence may be present at different loci within the genome of both the primary regenerant and progeny plants derived therefrom.

The introduction of an isolated nucleotide sequence encoding a virus-encoded polypeptide into a plant may be facilitated by providing said nucleic acid in the form of a gene construct or vector molecule. Accordingly, the present invention clearly extends to the use of gene constructs and vectors designed to facilitate the introduction of the introduced genes.

In the present context, the term “gene construct” refers to any nucleic acid molecule that comprises one or more isolated nucleotide sequences, each of which encodes a virus-encoded polypeptide, in a form suitable for introducing into a plant cell, tissue, organ, or plant part, including a plantlet, and preferably which is capable of being integrated into the genome of a plant. In the case of case of conferring multiple immunity or enhancing resistance against more than one virus, the isolated nucleotide sequence(s) encoding the virus-encoded polypeptides of those viruses may be contained within the same gene construct, such as, for example, in a manner similar to the binary vector pBH3 exemplified herein, or alternatively, contained within separate gene constructs for introduced separately, or in concert, to the plant cell.

As used herein, the word “vector” shall be taken to refer to a linear or circular DNA sequence which includes a gene construct as hereinbefore defined, and which includes any additional nucleotide sequences to facilitate replication in a host cell and/or integration and/or maintenance of said gene construct or a part thereof in the host cell genome.

Preferred vectors include plasmids, cosmids, plant viral vectors, and the like, such as, for example, a plasmid or cosmid containing T-DNA to facilitate the integration of the foreign nucleic acid into the plant genome, such as, for example, binary transformation vectors, super-binary transformation vectors, co-integrate transformation vectors, Ri-derived transformation vectors, suitable for use in any known method of transforming a plant, in particular a pasture legume.

The term “vector” shall also be taken to include any recombinant virus particle or cell, in particular a bacterial cell or plant cell, which comprises the gene construct of the invention. For example, a recombinant plant virus, such as a gemini virus, amongst others, may be engineered to contain the isolated nucleotide sequence encoding a virus-encoded polypeptide, or alternatively, a gene construct containing the isolated nucleotide sequence encoding a virus-encoded polypeptide may be introduced into Agrobacterium tumefaciens or Agrobacterium rhizogenes, for subsequent transfer to plant cells, tissues, organs or whole plants as described herein.

In a particularly preferred embodiment, the gene construct contains the isolated nucleotide sequence encoding a virus-encoded polypeptide cloned within a binary transformation vector that is known to those skilled in the art to be suitable for Agrobacterium-mediated transformation of plant cells, tissues, or organs, by virtue of the presence of the T-DNA left border and/or T-DNA right border sequences.

The gene constructs are introduced into a plant cell, tissue, organ, or whole plant, using standard procedures, to produce a transfected or transformed cell which may be subsequently regenerated to produce a transgenic or transformed plant. In the present context, a “transgenic to plant” or “transformed plant” shall be taken to mean a plant carrying an isolated nucleotide sequence encoding a virus-encoded polypeptide, and preferably, having said nucleotide sequence introduced into its genome, by means of transfection or transformation.

Furthermore, a “transgenic plant” or “transformed plant” shall be taken to include any cell, tissue, or organ, which is derived from a whole transgenic plant or whole transformed plant, or a cell, tissue or organ which capable of clonal propagation to produce a whole transgenic plant or whole transformed plant.

By “transfection” is meant that the process of introducing a gene construct or vector or an active fragment thereof which comprises foreign nucleic acid into a cell, tissue or organ derived from a plant, without integration into the genome of the host cell.

By “transformation” is meant the process of introducing a gene construct or vector or an active fragment thereof which comprises foreign nucleic acid into a cell, tissue or organ derived from a plant, wherein said foreign nucleic acid is stably integrated into the genome of the host cell.

Means for introducing recombinant DNA into plant tissue or a plant cell are known to those skilled in the art, and include, but are not limited to, direct DNA uptake into protoplasts (Krens et al., 1982; Paszkowski et al., 1984), PEG-mediated uptake to protoplasts (Armstrong et al. 1990), microparticle bombardment, electroporation (Fromm et al., 1985), microinjection of DNA (Crossway et al. 1986), microparticle bombardment of tissue explants or cells (Christou et al., 1988; Sanford, 1988), vacuum-infiltration of tissue with nucleic acid, T-DNA-mediated transfer of Agrobacterium to the plant tissue as described essentially by An et. al. (1985), Herrera-Estrella et al.(1983a, 1983b, 1985), or as otherwise exemplified herein.

For microparticle bombardment of cells, a microparticle is propelled into a cell to produce a transformed plant cell, tissue or organ. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed. Examples of microparticles suitable for use in such systems include 1 to 5 micron gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (eg., apical meristem, axillary buds, and root meristems), and induced meristem tissue (eg., cotyledon meristem and hypocotyl meristem).

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

A particularly preferred method of producing a transgenic plant is by Agrobacterium-mediated transformation of cotyledons, and regeneration into whole plants, essentially as exemplified herein.

The regenerated transformed plants described herein may take a variety of forms, such as, for example, chimeras of transformed cells and non-transformed cells; or clonal transformants (eg., all cells transformed to contain the expression cassette). They may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

Preferably, the nucleotide sequence is expressed in the plant to produce mRNA or the polypeptide encoded by the introduced nucleotide sequence.

By “expression” is meant transcription with or without concomitant translation, or any subsequent post-translational events which modify the biological activity, cellular or sub-cellular localization, turnover or steady-state level of the polypeptide encoded by the introduced nucleotide sequence encoding the virus-encoded polypeptide, in particular the virus-encoded coat protein(s) or virus-encoded replicase(s).

Expression of the introduced nucleotide sequence may be evidenced by direct assay known to those skilled in the art, such as, for example, by northern hybridisation, RT-PCR, or other means to measure steady state levels of mRNA, or alternatively, by comparing protein levels in the cell using ELISA or other immunoassay, SDS/PAGE, or enzyme assay. For example, the level of expression of a particular nucleotide sequence may be determined by polymerase chain reaction (PCR) following reverse transcription of an mRNA template molecule, essentially as described by McPherson et al. (1991). Alternatively, the expression level of a genetic sequence may be determined by northern hybridisation analysis or dot-blot hybridisation analysis or in situ hybridisation analysis or similar technique, wherein mRNA is transferred to a membrane support and hybridised to a probe molecule which comprises a nucleotide sequence complementary to the nucleotide sequence of the mRNA transcript encoded by the gene-of-interest, and generally labelled with a suitable reporter molecule such as a radioactively-labelled dNTP (eg [α-³²P]dCTP or [α-³⁵S]dCTP) or biotinylated of fluorescent dNTP, amongst others.

Expression may then be determined by detecting the signal produced by the reporter molecule bound to the hybridised probe molecule. Alternatively, the rate of transcription of a particular gene may be determined by nuclear run-on and/or nuclear run-off experiments, wherein nuclei are isolated from a particular cell or tissue and the rate of incorporation of rNTPs into specific mRNA molecules is determined. Alternatively, expression of a particular gene may be determined by RNase protection assay, wherein a labelled RNA probe or riboprobe which is complementary to the nucleotide sequence of mRNA encoded by said gene is annealed to said mRNA for a time and under conditions sufficient for a double-stranded mRNA molecule to form, after which time the sample is subjected to digestion by RNase to remove single-stranded RNA molecules and in particular, to remove excess unhybridised riboprobe. Such approaches are described in detail by Sambrook et al. (1989) and Ausubel (1987). Those skilled in the art will also be aware of various immunological and enzymatic methods for detecting the level of expression of a particular gene at the protein level, for example using rocket immunoelectrophoresis, ELISA, radioimmunoassay and western blot immunoelectrophoresis techniques, amongst others.

To express the isolated nucleotide sequence in order to confer immunity or enhance resistance against one or more viruses in the plant cell, the introduced nucleotide sequence is preferably capable of being expressed at the protein level. In performing such an embodiment of the invention, it is particularly preferred that the introduced nucleotide sequence will have a codon usage in any protein-encoding part thereof which is suitable for translation in the plant-of-interest. For example, to express the coat protein-encoding gene of AMV in M. sativa, it is preferred for the codon usage of that gene to be compatible with the codon preferences of M. sativa. Accordingly, the present invention clearly contemplates the expression of variants of the virus-encoded nucleotide sequences exemplified herein that have been modified merely to suit the codon preferences of a pasture legume plant, such as, for example, a pasture legume selected from the group consisting of Trifolium spp. and Medicago spp., and more particularly T. repens, T. subterraneum, T. pratense, T. michelianum, T isthmocarphum, or M. sativa.

As will be known to those skilled in the art, to ectopically express the virus-encoded polypeptide, the structural gene region or open reading frame (ORF) which encodes said polypeptide is placed in the sense orientation in operable connection with a suitable promoter sequence so as to provide for transcription and translation in the cell.

Reference herein to a “promoter” is to be taken in its broadest context and includes the transcriptional regulatory sequences of a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner, the only requirement being that said promoter sequence is capable of conferring expression of the virus-encoded polypeptide in a pasture legume plant, and more particularly, in those tissues which are otherwise susceptible to virus infection, such as, for example, the leaves, or veins.

In the present context, the term “promoter” is also used to describe a synthetic or fusion molecule, or derivative which confers, activates or enhances expression of the introduced nucleotide sequence in the plant. Preferred promoters may contain additional copies of one or more specific regulatory elements, to further enhance expression and/or to alter the spatial expression and/or temporal expression of a nucleic acid molecule to which it is operably connected.

Placing the nucleotide sequence encoding the virus-encoded polypeptide under the regulatory control of a promoter sequence means positioning said molecule such that expression is controlled by the promoter sequence. The promoter is usually, but not necessarily, positioned upstream or 5′ of said nucleotide sequence. Furthermore, the regulatory elements comprising a promoter are usually positioned within 2 kb of the start site of transcription of the gene or gene fragment the expression of which it regulates. In the construction of heterologous promoter/structural gene combinations, it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control may be defined by the positioning of the element in its natural setting (i.e., the genes from which it is derived). Again, as is known in the art, some variation in this distance can also occur.

Promoters suitable for use in expressing the virus-encoded polypeptide in a pasture legume include promoters derived from the genes of viruses, yeasts, moulds, bacteria, insects, birds, mammals and plants which are capable of functioning in the green tissues of such plants. The promoter may confer expression constitutively throughout the plant, or differentially with respect to the green tissues, or differentially with respect to the developmental stage of the green tissue in which expression occurs, or in response to external stimuli such as, for example, pathogen attack.

Preferred promoters suitable for use in the inventive method are strong constitutive promoters selected from the group consisting of: (i) a SCSV promoter sequence; (ii) pea rbcS-E9 promoter sequence; (iii) a CaMV 35S promoter sequence; (iv) a duplicated CaMV 35S promoter sequence; (v) a CaMV 19S promoter sequence; and (vi) the A. thaliana SSU promoter sequence. More preferably, to achieve the maximum benefits of the invention in terms of enhancing the proportion of immune or resistant lines of plants produced, the virus-encoded polypeptide is expressed under the control of a promoter sequence selected from the group consisting of (i) the SCSV region 4 (SCSV4) promoter sequence; (ii) a duplicated CaMV 35S promoter sequence; and (iii) the A. thaliana SSU promoter sequence, and, even more preferably, under the control of a duplicated CaMV 35S promoter sequence or the A. thaliana SSU promoter sequence.

As exemplified herein, the use of a strong constitutive promoter sequences has produced the surprising effect of enhancing the proportion of immune or resistant lines obtained from a single transformation experiment (as distinct from a single transformation event), in a manner that is independent from effects attributable to mere orientation, copy number, or expression level of the introduced nucleotide sequence. This ability of a promoter sequence to influence the numbers of immune or resistant lines of plants produced in a single transformation experiment is outside the known function of a promoter to regulate the level of expression of the introduced gene to which it is operably connected.

Accordingly, a second aspect of the present invention provides a method of producing enhanced numbers of virus-resistant or virus-immune lines of plants comprising introducing to a plant cell in the sense orientation, and preferably expressing therein, a nucleotide sequence encoding a virus-encoded polypeptide operably in connection with a strong promoter sequence selected from the group consisting of (i) a SCSV promoter sequence; (ii) a duplicated CaMV 35S promoter sequence; and (iii) the A. thaliana SSU promoter sequence.

Preferably, the promoter sequence is a duplicated CaMV 35S promoter sequence or the A. thaliana SSU promoter sequence.

As used herein, the term “duplicated CaMV 35S promoter sequence” shall be taken to refer to a promoter sequence other than a standard CaMV 35S promoter sequence known to those skilled in the art which comprises a tandem linear inverted or direct repeat of said promoter sequence or a fragment thereof sufficient to confer expression on a heterologous gene in a plant cell. Preferably, the duplicated CaMV 35S promoter sequence consists of the promoter contained within the plasmid pKYLX71:35S² described herein which comprises the nucleotide sequence set forth in SEQ ID NO: 45.

Preferably, the A. thaliana SSU gene promoter is the A. thaliana SSU-1A gene promoter contained within plasmid prbcSGPG described by Tabe et al. (1995) or a fragment thereof capable of conferring strong expression in plant cells.

Other embodiments on the performance of this aspect of the invention will be apparent from the preceding description.

Another aspect of the present invention provides a nucleotide sequence encoding the coat protein of a virus selected from the group consisting of bromoviruses, potyviruses, potexviruses, and nanoviruses, wherein said nucleotide sequence is selected from the group consisting of:

1. an alfalfa mosaic virus coat protein-encoding sequence selected from the group consisting of: SEQ ID Nos: 1, 3, and 5;

2. the clover yellow vein virus coat protein-encoding sequence set forth in SEQ ID NO: 25;

3. the white clover mosaic virus coat protein-encoding sequence set forth in SEQ ID NO: 30;

4. a nucleotide sequence that is degenerate to any one of the sequences of (1), (2) or (3); and

5. a nucleotide sequence that is complementary to any one of (1), (2), (3) or (4).

Another aspect of the present invention provides a gene construct comprising a nucleotide sequence encoding the coat protein of a virus selected from the group consisting of bromoviruses, potyviruses, potexviruses, and nanoviruses, wherein said nucleotide sequence is selected from the group consisting of:

1. an alfalfa mosaic virus coat protein-encoding sequence selected from the group consisting of: SEQ ID Nos: 1, 3, 5, 7, 9, 11, 13, 15, and 17;

2. the clover yellow vein virus coat protein-encoding sequence set forth in SEQ ID NO: 25;

3. a white clover mosaic virus coat protein-encoding sequence selected from the group consisting of SEQ ID Nos: 30, 32, and 34; and

4. a nucleotide sequence that is degenerate to any one of the sequences of (1), (2) or (3).

Preferably, the nucleotide sequence is selected from the group consisting of:

1. an alfalfa mosaic virus coat protein-encoding sequence selected from the group consisting of: SEQ ID Nos: 1, 3, and 5;

2. the clover yellow vein virus coat protein-encoding sequence set forth in SEQ ID NO: 25;

3. the white clover mosaic virus coat protein-encoding sequence set forth in SEQ ID NO: 30;

4. a nucleotide sequence that is degenerate to any one of the sequences of (1), (2) or (3); and

5. a nucleotide sequence that is complementary to any one of (1), (2), (3) or (4).

In addition to the isolated nucleotide sequence encoding the coat protein, the gene construct will generally comprise a promoter sequence for regulating expression of the said nucleotide sequence, when desired, and a terminator sequence.

The term “terminator” refers to a DNA sequence at the end of a transcriptional unit which signals termination of transcription. Terminators are 3N-non-translated DNA sequences containing a polyadenylation signal, which facilitates the addition of polyadenylate sequences to the 3N-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the gene constructs of the pr sent invention include the nopaline synthase (nos) gene terminator or octopine synthase (ocs) gene terminator of A. tumefaciens, the terminator of the Cauliflower mosaic virus (CaMV) 35S gene, the tobacco SSU gene terminator, the pea Rubisco small subunit E9 (rbcS-E9) gene terminator, or a subclover stunt virus (SCSV) gene sequence terminator, amongst others. Those skilled in the art will be aware of additional terminator sequences which may readily be used without any undue experimentation.

The gene constructs of the invention may further include an origin of replication sequence which is required for replication in a specific cell type, for example a bacterial cell, when said gene construct is required to be maintained as an episomal genetic element (eg. plasmid or cosmid molecule) in said cell. Preferred origins of replication include, but are not limited to, the f1-ori and colE1 origins of replication.

The gene construct may further comprise a selectable marker gene or genes that are functional in a cell into which said gene construct is introduced.

As used herein, the term “selectable marker gene” includes any gene which confers a phenotype on a dell in which it is expressed to facilitate the identification and/or selection of cells which are transfected or transformed with a gene construct of the invention or a derivative thereof.

Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene (S. hygroscopicus bar gene or phosphinothricin phosphotransferase gene), neomycin phosphotransferase gene (nptII), hygromycin resistance gene (hygromycin phosphotransferase gene), β-glucuronidase (GUS) gene, chloramphenicol acetyltransferase (CAT) gene and luciferase gene, amongst others. Preferred selectable marker genes for use in performing the inventive methods will be apparent from the exemplification of the invention described herein.

Preferably, the gene construct of the invention is suitable for integration into the genome of a plant, in particular a pasture legume plant selected from the group consisting of Trifolium spp. and Medicago spp. In a particularly preferred embodiment, the gene construct is a binary vector construct suitable for the A. tumefaciens-mediated transformation of a plant cell.

Yet another aspect of the present invention provides a method for improving the germplasm of plants to enhance their resistance to one or multiple viruses or to confer immunity to one or multiple viruses thereon, said method comprising:

(i) crossing a first parent plant consisting of a primary regenerant having immunity or enhanced resistance to one or more viruses with a second parent plant, wherein said first parent plant has immunity or enhanced resistance by virtue of having an isolated nucleotide sequence introduced which encodes a virus-encoded polypeptide into its genome;

(ii) obtaining the hemizygous progeny (T1) plants of said cross having immunity or enhanced resistance to said one or more viruses;

(iii) intercrossing or conducting diallel crosses of the hemizygous progeny (T1) plants;

(iv) obtaining the T2 progeny plants of said intercross having immunity or enhanced resistance to said one or more viruses;

(v) identifying those T2 plants that are homozygous for the isolated nucleotide sequence and exhibit the immunity or resistance of said first parent; and

(vi) intercrossing or polycrossing said homozygous T2 plants.

Preferably the first parent plant has immunity or enhanced resistance against one or more viruses selected from the group consisting of AMV, CYVV, WCMV, and SCSV. Plants which exhibit either single immunity or resistance, or alternatively, multiple immunity or resistance, are clearly contemplated herein.

The second parent plant may be a plant that has a desired germplasm, such as, for example, by virtue of exhibiting one or more desirable characteristics of commercial utility. The second parent plant may also be one which exhibits immunity or enhanced resistance against one or multiple plant viruses, in which case the inventive method is useful for the purposes of stacking immunity or resistance characteristics of both parent plants into an elite virus-immune or virus-resistant germplasm, and/or for producing a germplasm which utilises different mechanisms of protecting plants against the same virus, such as, for example, by combining coat protein-encoding and dysfunctional replicase-encoding sequences into the same germplasm. In such circumstances, it is particularly preferred for the second parent plant to exhibit immunity or resistance against a virus selected from the group consisting of bromoviruses, potyviruses, potexviruses, and nanoviruses.

Whilst not limiting the invention, if both parent plants exhibit virus immunity or resistance, it is also preferred for the first and/or second parent plant to contain an introduced nucleotide sequence encoding a virus-encoded polypeptide introduced into its genome such that said first and/or second parent plant exhibits immunity or enhanced resistance against a virus by virtue of said introduced nucleotide sequence. Preferably, the immunity or enhanced resistance of the first and second parent plant will be different or targeted against a different virus. As will be apparent from the description provided herein, this aspect of the invention provides for the production of a plant having enhanced and more sustainable resistance or immunity against a virus by virtue of the combination of two resistance mechanisms against that virus (i.e. by pyramiding two different resistance genes against the one virus).

To identify those T2 plants that are homozygous, test crosses may be conducted wherein the T3 progeny are screened for the presence of the selectable marker gene present on the binary vector used to produce the primary regenerant parent plant, such as, for example, by using PCR, or by determining the segregation in the T3 generation, of resistance to the antibiotic or herbicide which expression of the selectable marker gene confers. Alternatively, the numbers of T3 progeny plants that are immune or resistant to the virus(es) may be scored following mechanical inoculation of T3 plants with virus, or standard bioassay for virus immunity or resistance. In all cases, those T2 plants that are homozygous will produce 100% of progeny that are immune or resistant to virus, or exhibit resistance to the selectable marker.

A further aspect of the present invention provides a transformed plant, and preferably, a transformed pasture legume, produced by performance of the inventive methods described herein.

As used herein, the term “transformed plant” shall be taken to include the primary transformed cell, and any tissue, organ or whole plant comprising said primary transformed cell. In the present context, the term “transformed plant” shall further be taken to include any derivative of the primary transformed cell, tissue, organ or whole plant that also contains the introduced nucleotide sequence encoding the virus-encoded polypeptide to which the present invention relates. Accordingly, a transformed plant within the context of the present invention includes any T0, T1, T2, T3, . . . Tn plant derived from the primary transformed cell, subject to the proviso that said plant contains nucleic acid encoding the virus-encoded polypeptide that was present in said primary transformed cell. Whilst the selectable marker gene may also be present in the transformed plant, it is not a prerequisite feature for performance of the inventive methods described herein, and, as a consequence, in not an essential feature of the transformed plant of the present invention. For example, the selectable marker gene may be removed from the progeny of the primary regenerant plant by any means known to those skilled in the art without substantial loss of virus resistance or immunity, provided that the sequence encoding the virus-encoded polypeptide is left intact in the plant, preferably in an expressible format.

Preferably, the transformed pasture legume is selected from the group consisting of white clover, red clover, Persian clover, subterranean clover, lentil and chickpea. The performance of the inventive method is other pasture legumes is not excluded.

The transformed plants will exhibit a range of resistance and immunity characteristics evident from the preceding description, including resistance or immunity against one or more viruses selected form the group consisting of: bromoviruses, potexviruses, potyviruses, and nanoviruses, and more particularly, one or more viruses selected from the group consisting of: AMV, SCSV, WCMV, and CYVV.

The art-recognised method for identifying virus-resistant or virus-immune primary transformants or the hemizygous or homozygous progeny thereof is the virus-infectivity assay. However, that assay is labour-intensive and time-consuming, taking weeks-to-months to complete. The present inventors have developed an equally-reliable assay taking only hours-to-days to complete, based upon the detection of expression of the introduced nucleic acid (i.e. the transgene encoding the virus-encoded polypeptide).

A further aspect of the invention provides a method of identifying a gene of interest in a primary transformant plant or a progeny plant thereof comprising

(a) conducting a PCR replication cycle on a sample of interest;

(b) detecting a PCR product; and

(c) analysing the presence or absence of a PCR product above background to determine whether a plant is homozygous, heterozygous or azygous for a gene of interest.

Preferably the PCR replication cycle incorporates a marker and detection of the PCR product is by detection of the marker. Preferably the number of PCR replication cycles required to detect the PCR product above background determines whether a plant is homozygous, heterozygous or azygous for a gene of interest.

Accordingly, this further aspect of the present invention provides a reliable and time-saving method of identifying a virus-resistant primary transformant plant or a progeny plant thereof, comprising contacting mRNA from said plant with a hybridisation-effective amount of a nucleic acid probe comprising at 15 nucleotides in length for a time and under conditions sufficient for hybridisation to occur, wherein said probe is complementary to a nucleotide sequence encoding the coat protein of said virus.

The preferred format for the performance of the inventive method is a nucleic acid hybridisation reaction, such as, for example, a northern hybridisation or dot blot assay, such as described by Ausubel et al. (1987) or Sambrook et al. (1989). Those skilled in the art will be aware that, in the performance of these assay formats, mRNA is isolated from the plant, and transferred to a membrane support and hybridised to a probe molecule which comprises a nucleotide sequence complementary to the nucleotide sequence of the mRNA transcript encoded by the gene-of-interest, labelled with a suitable reporter molecule such as a radioactively-labelled dNTP (eg [α-³²P]dCTP or [α-³⁵S]dCTP), fluoresecntly-labelled dNTP (eg Fam- or Tamra-labelled dNTP), or a biotinylated dNTP, amongst others. In the case of northern hybridisations, mRNA is electrophoreses on an agarose gel, generally under denaturing conditions (eg in the presence of formaldehyde) prior to transfer to the membrane support. The mRNA is detected following hybridisation, by detecting the appearance of a signal produced by the reporter molecule bound to the hybridised probe molecule.

Preferably, the method further comprises the removal of excess background in the reaction, such as, for example, by washing of membranes and/or by incubation of hybridised membranes with Rnase enzyme.

In contrast, the alternative PCR assay is not suitable for identifying primary transformants and is less reliable in identifying transgenic progenies.

Preferred probes suitable for use in the performance of the inventive method comprise at least 5 about 20 nucleotides derived from the full-length sequence encoding the virus-encoded polypeptide, more preferably at least about 25 nucleotides in length, and even more preferably at least about 30 nucleotides in length or 35 nucleotides or 50-100 nucleotides in length. In a particularly preferred embodiment, the probe used in performing the inventive method comprises a sequence which is complementary to the entire open reading frame of a nucleotide to sequence encoding the virus-encoded polypeptide. Preferably, the probe is derived from the coat protein-encoding gene of a virus selected from the group consisting of AMV, CYVV, WCMV, and SCSV. In a particularly preferred embodiment, the probe is derived from coat protein-encoding gene of AMV.

Hybridisation to the probe is generally carried out under at least low stringency conditions, more preferably under at least moderate stringency conditions and even more preferably under at least high stringency conditions. For the purposes of defining the level of stringency, those skilled in the art will be aware that a low stringency may comprise a hybridisation and/or a wash carried out in 6×SSC buffer, 0.1% (w/v) SDS at 28EC or room temperature. A moderate stringency may comprise a hybridisation and/or wash carried out in 2×SSC buffer, 0.1% (w/v) SDS at a temperature in the range 45EC to 65EC. A high stringency may comprise a hybridisation and/or wash carried out in 0.1×SSC buffer, 0.1% (w/v) SDS or Church Buffer at a temperature of at least 65EC. Variations of these conditions will be known to those skilled in the art. As will be known to those skilled in the art, very short probes of less than about 50 nucleotides in length may require a lower stringency than longer probes, and produce higher backgrounds in the hybridisation reaction.

Generally, the stringency is increased by reducing the concentration of SSC buffer, and/or increasing the concentration of SDS in the hybridisation buffer or wash buffer and/or increasing the temperature at which the hybridisation and/or wash are performed. Conditions for hybridisations and washes are well understood by one normally skilled in the art. For the purposes of clarification of parameters affecting hybridisation between nucleic acid molecules, reference can conveniently be made to pages 2.10.8 to 2.10.16. of Ausubel et al. (1987), which is herein incorporated by reference.

### BRIEF DESCRIPTION OF THE DRAWINGS

FIG. 1 is a copy of an alignment of the coat protein genes of nine different isolates of AMV, as follows:

Type I AMV isolates: H1 (SEQ ID NO: 1); WC3 (SEQ ID NO: 3); 425S (SEQ ID NO: 5); 425M(SEQ ID NO: 7); and 425L (SEQ ID NO: 9); and

Type II AMV isolates: YSMV (SEQ ID NO: 11); AMU 12509 (SEQ ID NO: 13); AMU12510 (SEQ ID NO: 15), and YD3.2 (SEQ ID NO: 17).

Numbering refers to the nucleotide position relative to the start site for translation of the coat protein mRNA. Asterisks indicate variable residues between the sequences.

FIG. 2 is a copy of an alignment of the amino acid sequences of the coat protein genes of nine different isolates of AMV, as follows:

Type I AMV isolates: H1 (SEQ ID NO: 2); WC3 (SEQ ID NO: 4); 425S (SEQ ID NO: 6); 425M (SEQ ID NO: 8); and 425L (SEQ ID NO: 10); and

Type II AMV isolates: YSMV (SEQ ID NO: 12); AMU12509 (SEQ ID NO: 14); AMU12510 (SEQ ID NO: 16), and YD3.2 (SEQ ID NO: 18).

Numbering refers to the amino acid position relative to the first methionine residue of the coat protein. Asterisks indicate variable residues between the sequences.

FIG. 3 is a copy of a schematic representation of the plasmid pGEM5Zf(−) of Promega Biotechnology.

FIG. 4 is a copy of a schematic representation of the plasmid pWM5. Plasmid pWM5 was derived from plasmid pDH51 (Pietrzak et al., 1986) by: (i) replacing the CaMV 35S promoter and 5′-UTR sequences of pDH51 with the A. thaliana SSU-1A gene promoter and 5′-UTR sequences of plasmid prbcSGPG (Tabe et al., 1995); and (ii) replacing the CaMV 35S 3′-UTR sequence of plasmid pDH51 with the 3′-UTR of the NtSS23 gene of Nicotiana tabacum (Mazur & Chiu, 1985).

FIG. 5 is a copy of a schematic representation of the binary plasmid vector pTP5.

FIG. 6 is a copy of a schematic representation of the strategy for producing plasmid pTP5. The sub-cloning step involving the transfer of the EcoRI fragment from the intermediary vector pWM5 into the binary vectors is not shown.

FIG. 7 is a copy of a schematic representation of the binary plasmid vector pT17.

FIG. 8 is a copy of a schematic representation of the pKYLX family of vectors described by Schardl et al., (1987).

FIG. 9 is a copy of a schematic representation of the binary plasmid vector pKYLX71:35S²amv4.

FIG. 10 is a schematic representation of the genome organisation of CYVV (top row) showing the positions of the various open reading frames (ORFs), and depicting translation and polyprotein processing of the transcription/translation products of these ORFs.

FIG. 11 is a copy of a schematic representation of the T-DNA region of the binary vector pBH1.

FIG. 12 is a schematic representation of the genome organisation of WCMV (top row) showing the positions of the various open reading frames (ORFs), including the coat protein ORF (CP). The amplification products CP4 (middle row) and Cp3 (bottom row) obtained using the primers WCMV4956-f (SEQ ID NO: 27), WCMV5167-f (SEQ ID NO: 28), and WCMVKpn-3′ (SEQ ID NO: 29) are also indicated. Arrows indicate the relative orientations of the amplification primers used to produce the amplification products Cp4 and Cp3 comprising the coat protein ORF.

FIG. 13 is a copy of an alignment of the coat protein genes of three different isolates of WCMV, in particular the Bundoora isolate (top row; SEQ ID NO: 30), strain M (middle row; SEQ ID NO: 32), and strain O (bottom row; SEQ ID NO: 34). Numbering refers to the nucleotide position relative to the start site for translation of the coat protein mRNA. Translation start (ATG) and stop (TM or TGA) codons are indicated in bold type

FIG. 14 is a copy of an alignment of the amino acid sequences of the coat protein genes of three different isolates of WCMV, in particular the Bundoora isolate (top row; SEQ ID NO: 31), strain M (middle row; SEQ ID NO: 33), and strain O (bottom row; SEQ ID NO: 35). Numbering refers to the amino acid position relative to the first methionine residue of the coat protein.

FIG. 15 is a copy of a schematic representation of the T-DNA region of the binary vector pKYLX71:35S²wcm4 cp.

FIG. 16 is a copy of a schematic representation of the binary vector pPZP100.

FIG. 17 is a copy of a schematic representation of the T-DNA region of the binary vector pBH3.

FIG. 18A is a copy of a schematic representation of the T-DNA region of the binary vector pTS20 containing the SCSV coat protein-encoding gene.

FIG. 18B is a copy of a schematic representation of the T-DNA region of the binary vector pBH-2

FIG. 19 is a copy of a photographic representation of an agarose gel showing confirmation of transformation of red clover with the binary vector pTP5, using the PCR assay to detect the nptII gene.

FIG. 20 is a copy of a photographic representation of a Southern blot of genomic DNA of white clover lines transformed with the AMV coat protein ORF, hybridised to DIG-labelled probes comprising the AMV coat protein ORF.

FIG. 21 is a copy of a photographic representation of a northern blot of RNA of transformed white clover lines hybridised to a [α-³²P]dCTP-labelled probe prepared from the AMV coat protein gene of the binary vector pTP5.

FIG. 22 is a copy of a photographic representation of a Southern blot of genomic DNA of red clover lines transformed with the WCMV coat protein ORF, hybridised to DIG-labelled probes comprising an internal region of the WCMV coat protein ORF.

FIG. 23 is a copy of a photographic representation of a northern blot of RNA of transformed red clover lines hybridised to an [α-³²P]dCTP-labelled probe prepared from the WCMV coat protein gene of the binary vector pKYLX71:35S²wcm4 cp.

FIG. 24A is a copy of a photographic representation of a northern blot of RNAs of different replicates of the AMV-susceptible white clover line H9 (lanes 1-2) or line 446 (lanes 3-8) hybridised to a [α-³²P]dCTP-labelled probe prepared from the AMV coat protein gene, following inoculation with virus. The mRNAs of lanes 1-6 were derived from asymptomatic plants, whilst those present in lanes 7 and 8 were from plants exhibiting symptoms of AMV infection.

FIG. 24B is a copy of a photographic representation of a northern blot of RNAs of three AMV-resistant white clover plants (plant line 451; lanes 1-3) and four AMV-immune white clover plants (plant line 447; lanes 4-7) hybridised to a [α-³²P]dCTP-labelled probe prepared from the AMV coat protein gene, following inoculation with virus. All mRNAs were derived from asymptomatic plants.

FIG. 25 is a schematic representation showing the layout of the field trial of primary transformed white clover plants carrying the recombinant AMV coat protein-encoding gene and exhibiting resistance or immunity against AMV under glasshouse conditions.

FIGS. 26A and B are a copy of photographic representations showing the results of the field trial of primary transformed white clover plants carrying the recombinant AMV coat protein-encoding gene and exhibiting resistance or immunity against AMV under glasshouse conditions.

FIG. 27A is a graphical representation of canonical variance comparing the phenotypic, characteristics of primary-transformed white clover Cv. Haifa lines D4 and D6 to non-transformed white clover Haifa (broken lines) or Irrigation (unbroken lines) lines grown in the field. Each point represents a transformed plant of the lines D4 or D6.

FIG. 27B is a graphical representation of canonical variance comparing the phenotypic characteristics of primary-transformed white clover Cv. Irrigation lines H1 and H6 to non-transformed white clover Haifa (broken lines) or Irrigation (unbroken lines) lines grown in the field. Each point represents a transformed plant of the lines H1 or H6.

FIG. 28 is a graphical representation showing the effect of AMV genotype on virus spread in field trials. FIG. 28A shows the layout of plants in each plot infected with AMV strains YD1.2, WC28, and YD3.2 as follows: non-transformed T. repens cv. Haifa (black); non-transformed T. repens cv. Irrigation (checks); the transformed T. repens cv. Haifa lines designated as line 451 (resistant; grey boxes), line 447 (immune; boxes having white dots on grey background), line D4 (immune; horizontal lines), and line D6 (immune; cross-hatched boxes); and the transformed T. repens cv. Irrigation lines designated as line H1 (immune; stippled boxes), and line H6 (immune; open boxes). FIG. 28B shows the percentage of AMV-infected plants within each plot (abscissa) following challenge with the AMV isolates indicated on the x-axis.

FIG. 29 is a graphical representation showing the percentage of the following white clover plants in AMV field trials during the 1998 growing season which became infected with AMV: non-transformed T. repens cv. Haifa (black); and the transformed T. repens cv. Haifa lines designated as line 451 (resistant; grey boxes), line 447 (immune; boxes having white dots on grey background), line D4 (immune; boxes having horizontal lines), and line D6 (immune; crosshatched boxes).

FIG. 30 is a graphical representation showing the percentage of the following white clover plants in AMV field trials during the 1998 growing season which became infected with AMV: non-transformed T. repens cv. Haifa (black); non-transformed T. repens cv. Irrigation (hatched); the transformed T. repens cv. Haifa lines D4 (immune; horizontal lines), and D6 (immune; cross-hatched boxes); and the transformed T. repens cv. Irrigation lines H1 (immune; stippled), and H6 (immune; open boxes).

FIG. 31 is a graphical representation showing the effect of proximity of a source white clover plant infected with AMV on the spread of AMV to surrounding plants in field trials. FIG. 31A shows the arrangement of plants within a single plot of 25 plants, wherein a central AMV source plant (black star) is surrounded by 8 proximal clones (grey stars) and 14 distal clones (open stars). FIG. 31B shows the percentage of plants that are proximal or distal to the AMV-source plant that become infected with AMV in a resistant line. Accordingly, the rate of infection for plants that are proximal to the AMV source plant was approximately 3- to 5-fold the infection level observed for plants distal to the AMV-source plant.

FIG. 32 is a schematic representation of a field trial layout with 24 experimental plots (numbered 1-24) in a 2 ha paddock. Each plot contained 9 non-transgenic AMV-source plants o and experimental transformed (T0 and T1) plants, as well as control (wild-type) plants.

FIG. 33 is a schematic representation showing an individual plot design including AMV-source plants (shaded) and experimental transformed (T0 and T1) plants, as well as control wild-type plants in cells numbered 1-16 (left panel). The identities of individual plants are indicated in the right panel.

FIG. 34A is a graphical representation of the field trial data assessing AMV infection in T1 field trials at Hamilton. The percentage of AMV-infected plants of each genotype is indicated on the y-axis. The growing season and plant genotype are indicated on the x-axis.

FIG. 34B is a graphical representation of the field trial data assessing AMV infection in T1 field trials at Howlong. The percentage of AMV-infected plants of each genotype is indicated on the y-axis. The growing season and plant genotype are indicated on the x-axis.

FIG. 35 is a graphical representation of the field trial data assessing AMV infection in T1 field trials at Hamilton during the 1999/2000 growing season. The percentage of AMV-infected plants of each genotype is indicated on the y-axis. The growing season and plant genotype are indicated on the x-axis.

FIG. 36 is a copy of a photographic representation showing the production of super-transformed white clover by pyramiding multiple virus resistance genes. FIG. 36A shows seed containing the AMV coat protein-encoding gene expression cassette following 3 days of co-cultivation with A. tumefaciens strain AGL1 containing the binary vector pBH3. FIGS. 36B and 36C show cotyledons transformed with the binary vector pBH3 following selection on hygromycin and cefotaxime. The majority of cotyledons appear to be dead, however closer inspection reveals some green areas on stalks. FIG. 36D shows non-transformed cotyledons incubated on media containing hygromycin, all of which became necrotic. FIG. 36E shows cotyledons which have been co-cultivated with A. tumefaciens containing pBH3 and placed on media containing hygromycin. New hygromycin-resistant shoots appear regenerating from the base of the cotyledons. FIG. 36F shows the growth of hygromycin-resistant cotyledons that have been removed from selective media after approximately 4 weeks and placed onto media containing RM73 and cefotaxime, without hygromycin selection. FIG. 36G shows pBH3transformed plantlets growing in root-inducing media. FIG. 36H shows pBH3-transformed plantlets growing in a glasshouse.

FIG. 37A is a schematic representation of the binary vector pBH3.

FIG. 37B is a copy of a photographic representation showing the detection of DNA of the binary vector pBH3 in transformed lines of white clover, using PCR. Top Left: AMV coat protein-encoding gene. Top Right: nptII selectable marker gene. Lower Left: CYVV coat protein-encoding gene. Lower Right: hph selectable marker gene. Lanes 1-4 in each panel are transformed lines; Lane CP in each panel is a positive control.

FIG. 37C is a copy of a photographic representation of a Southern blot hybridisation showing the detection of CYVV coat protein-encoding DNA (Left) or WCMV coat protein-encoding DNA in super-transformed lines of white clover. Lanes 1-3 in each panel are transformed lines; Lane CP in each panel is a positive control.

FIG. 38A is a copy of a photographic representation of DNAs from T0 transformed white clover plants carrying a single T-DNA insertion, probed with the nptII specific gene sequence. Lane 1, genotype H6; Lane 2, genotype H1; Lane 3, genotype H2; Lane 4, genotype H3; Lane C, negative control untransformed white clover; Lane P, pKYLX71:35S²amv4 plasmid DNA.

FIG. 38B is a copy of a photographic representation of DNAs from T0 transformed white clover plants carrying a single T-DNA insertion, probed with the AMV coat protein-encoding gene sequence. Lane 1, genotype H6; Lane 2, genotype H1; Lane 3, genotype H2; Lane 4, genotype H3; Lane C, negative control untransformed white clover; Lane P, pKYLX71:35S²amv4 plasmid DNA.

FIG. 38C is a copy of a photographic representation of DNAs from T0 transformed white clover plant genotype H1 and six corresponding T1 transgenic plants from crosses to wild-type white clover selected for field evaluation in the extension PR64X trial. DNAs were hybridised with the AMV4 coat protein gene. Data show the meiotic stability of the introduced gene.

FIG. 38D is a copy of a photographic representation of a northern blot hybridisation of RNAs from T0 transformed white clover plant genotype H1 and six corresponding T1 transgenic plants derived from crosses to wild-type white clover selected for field evaluation in the extension PR64/PR67 trial. RNAs were hybridised with the AMV4 coat protein gene. Data show the meiotic stability of expression of the introduced gene.

FIG. 39 is a schematic representation of the strategy for developing an elite transgenic white clover germplasm, based upon the identification of plants that are homozygous for introduced transgenes using test crosses and selective progeny screening.

FIG. 40 is a copy of a photographic representation of a northern blot hybridisation of RNAs of progeny plants from a cross between the T1 transformed white clover genotype H1 expressing the AMV4 coat protein and elite line 9. Data show the expression of the introduced gene in the progeny plants (lanes 1-12) and in a TO control plant. Lane C, negative control untransformed white clover.

FIG. 41 is a schematic representation of the strategy for developing an elite transgenic white clover germplasm, based upon the identification of plants that are homozygous for introduced transgenes using high-throughput quantitative PCR for transgene detection.

FIG. 42 is a schematic representation of the strategy for pyramiding of single virus resistance phenotypes in plants. FIG. 42A shows pyramiding of AMV and SCSV resistances using the AMV4 and SCSV coat protein encoding genes and the bar selectable marker gene, and identifying the double virus resistant progenies by basta selection and PCR screening as described in FIG. 41. FIG. 42B shows pyramiding of resistances to AMV and CYVV by crossing the AMV immune lines H1 (top) or H6 (below) with CYVV resistant lines transformed with the plasmid pBH1, followed by selection on kanamycin-containing media and PCR screening as described in FIG. 41.

FIG. 43 is a schematic representation of the strategy for pyramiding of double virus resistance phenotypes to produce triple virus-resistant plants. Double AMV and CYVV resistant lines H1 x pBH1 CYVV resistance (top) or H6 x pBH1 CYVV resistance (below), derived by crossing as described in FIG. 42B are crossed with WCMV resistant lines transformed with a binary vector containing the WCMV resistance gene, such as coat protein-encoding gene. Following selection on kanamycin, and PCR screening as described in FIG. 41, those plants containing AMV, CYVV, and WCMV coat protein-encoding genes are selected and screened to isolate those lines possessing resistance to all three viruses.

FIG. 44 is a copy of a photographic representation showing the results of the field trial of transformed white clover plants carrying the recombinant AMV coat protein-encoding gene and exhibiting resistance or immunity against AMV. Side A shows virus infected susceptible white clover plant cv. ‘Irrigation’. Side B shows virus immune transgenic white clover plant cv. ‘Irrigation’.

FIG. 45 is a copy of a printout form the TaqMan quantative PCR system showing a wide window of discrimination between transgenic individuals (left curves) from the non-transgenic ones (right curves)

FIG. 46 is a schematic representation of the strategy for Production of triple virus-resistant lines by crossing an AMV resistant white clover line with a CYVV plus WCVV double virus resistant white clover line.

FIG. 47 depicts the Cloning strategy for the development of AMV RNA 1 infectious clone mutant derivatives expressing defective ATP binding motif.

FIG. 48 is a graphical representation of the mean number of local lesions per half leaf on Cowpeas following inoculation with infectious clones for mutant AMV RNA 1 along with each of the AMV RNA 24 infectious clones.

FIG. 49 is a graphical representation of the mean number of lesions per half leaf on cowpeas following co-inoculation with various combinations of mutant AMV RNA 1 infectious clones and AMV RNA2-4 infectious clones and the amount of those AMV infectious clones.

FIG. 50 depicts the cloning strategy for the development of the binary vectors containing the wild type or mutant AMV RNA 1 gene.

FIGS. 51 and 52 are a graphical representation of the relative visual score six days after inoculation of transgenic tobacco plants containing the: 1) wild type AMV RNA 1 gene; 2) Mutant T AMV RNA 1 gene and; 3) Mutant G AMV RNA 1 gene.

FIG. 53 is a graphical representation of the relative visual score vs the relative ELISA value for transgenic tobacco expressing the three different AMV RNA 1 constructs.

FIG. 54A is a graphical representation of the relative virus concentration in susceptible and resistant transgenic tobacco plants inoculated with AMV isolate WC28 viral inocula. Sap was extracted from leaf discs taken from the 1^{st}, 2^{nd} and 3^{rd} inoculated leaves of three replicate plants for each line six days after inoculation with 1:100 w/v dilution of the AMV isolate WC28 viral inocula.

FIG. 54B is a graphical representation of the Relative virus concentration in susceptible and resistant transgenic systemic leaves of tobacco plants inoculated with AMV isolate WC28 viral inocula. Sap was extracted from leaf strips taken from the 1^{st} systemic leaf of three replicate plants for each line six days after inoculation with 1:100 w/v dilution of the AMV isolate WC28 viral inocula.

FIG. 55 is a photograph showing local and systemic symptoms on tobacco plants inoculated with a 1:50 w/v dilution of AMV isolate WC28 eight days after inoculation. A) Untransformed control line W38, B) Line W6 transformed with the wild-type AMV 1a protein gene, C) Line G9 transformed with the G mutant AMV 1a protein gene, and D) Line T12 transformed with the T mutant AMV 1a protein gene.

FIG. 56 is a graphical representation of relative dry weight herbage biomass yield of different white clover lines inoculated with AMV isolate WC28 compare to the un-inoculated plants of the same line.

FIGS. 57A and B are copies of photographic representations of Northern blots using a probe derived from a fragment of the AMV RNA1 gene in leaf RNA samples from A) tobacco and B) white clover.

FIGS. 58A and B are copies of photographic representations of RT-PCR analysis of nptII transcript in RNA from A) tobacco and B) white clover.

### DETAILED DESCRIPTION OF THE PREFERRED EMBODIMENTS

Part I of the Experimental Section:

Virus Strains, Coat Protein Genes and the Production of Gene Constructs

### EXAMPLE 1.1

### Isolation and Characterisation of Australian Isolates of AMV

Australian AMV isolates were identified by bioassay on Cowpea(Vigna unguiculata) and Chenopodium quinoa which produced typical necrotic local lesions. Isolates of AMV (Table 1) were obtained from field-grown white clover and lucerne plants showing virus-like symptoms, which plants were collected from various regions of Australia. Of the isolates presented in Table 1, AMV isolates designated H1, WC10, and WC28 are Type I AMV isolates, whilst YD1.2, YD3.2, and YD5.2 are Type II isolates. Single-lesion isolates of the viruses were confirmed by host range studies, ELISA and electron microscopy

For all experiments involving AMV, the virus was maintained in Nicotiana glutinosa or Vigna unguiculata and purified from tobacco. Quality of the virus preparations was examined by UV spectral analysis and electron microscopy. Isolates of AMV that were representative of both subgroup I and subgroup II were used in experiments involving the challenge of transgenic plants expressing AMV coat protein.

### EXAMPLE 1.2

### Nucleotide Sequences of AMV Coat Protein Genes

Local isolates of AMV (H1, YD3.2 and WC3), obtained from single lesions as described in the preceding Example 1, were used as a virus source for AMV coat protein genes.

The nucleotide and deduced amino acid sequences of the cloned coat protein gene from three Australian isolates of AMV (H1, YD3.2 and WC3) have been determined using the dideoxy chain termination method (Sanger et al., 1977) to sequence either M13 ssDNA templates (Sambrook et al., 1989) or double-stranded DNA templates prepared by the CTAB method (Del Sal et al., 1989). Sequence analysis was carried out using the University of Wisconsin Genetics Computer Group Sequence Analysis Software Package (Devereaux et al., 1984).

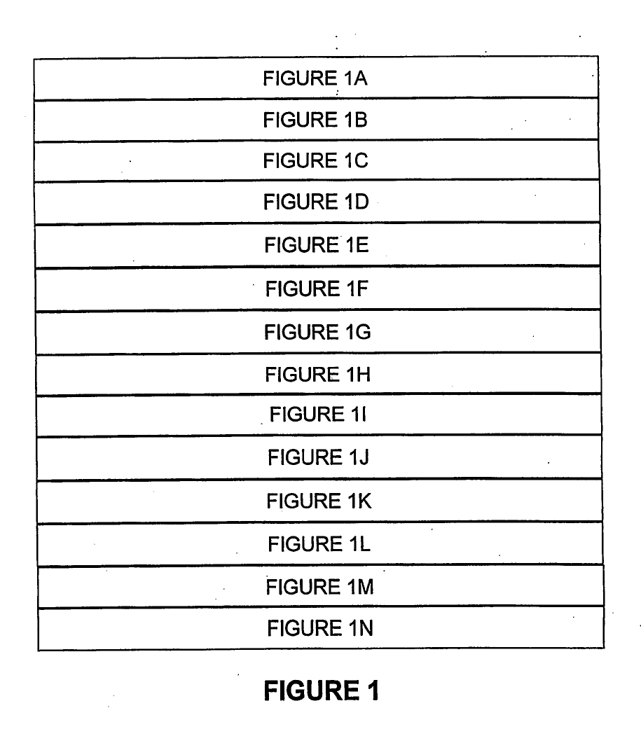

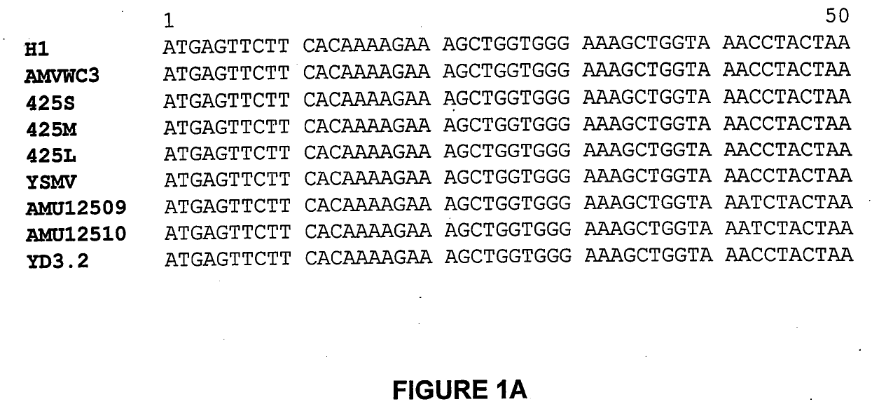

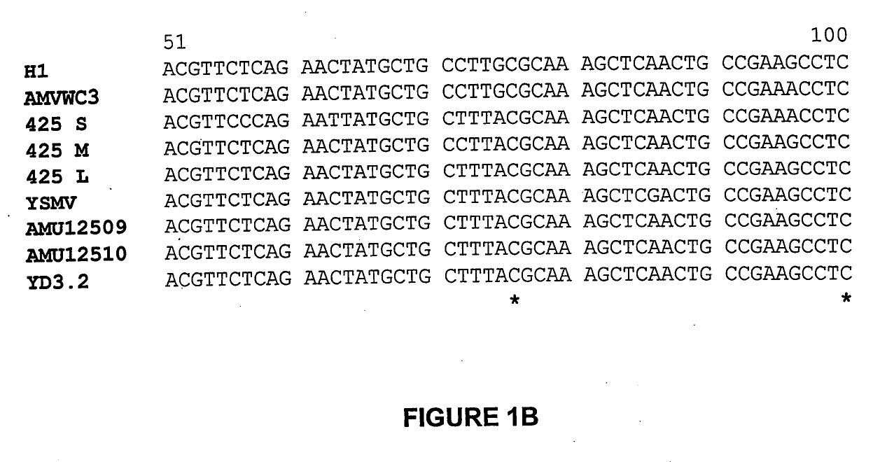

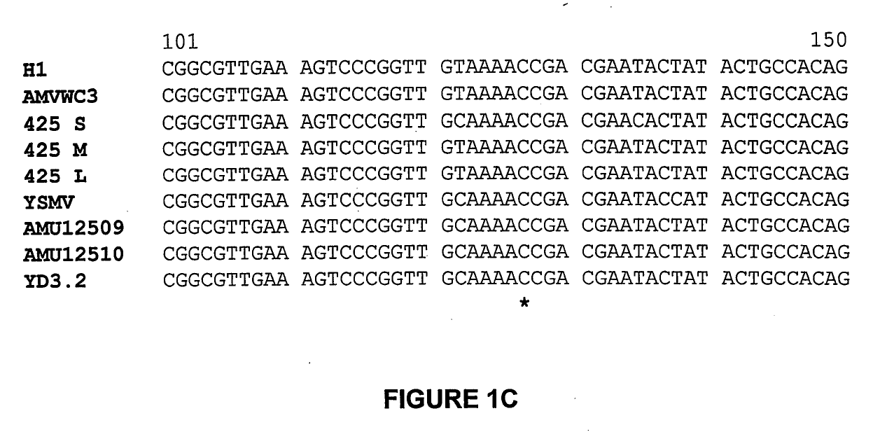

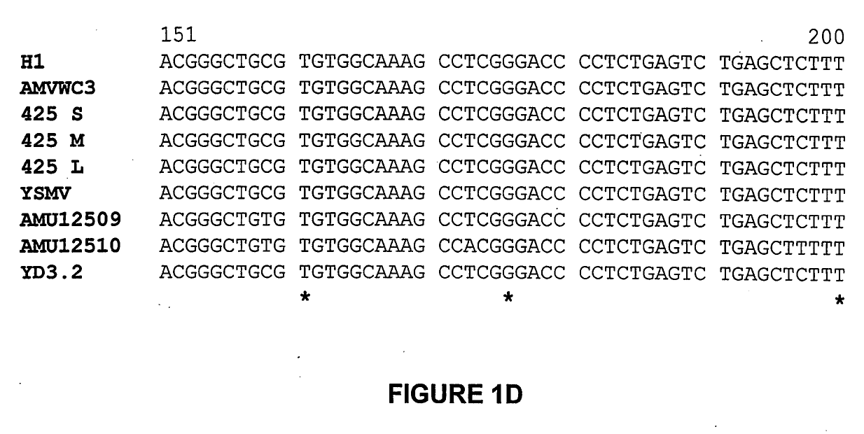

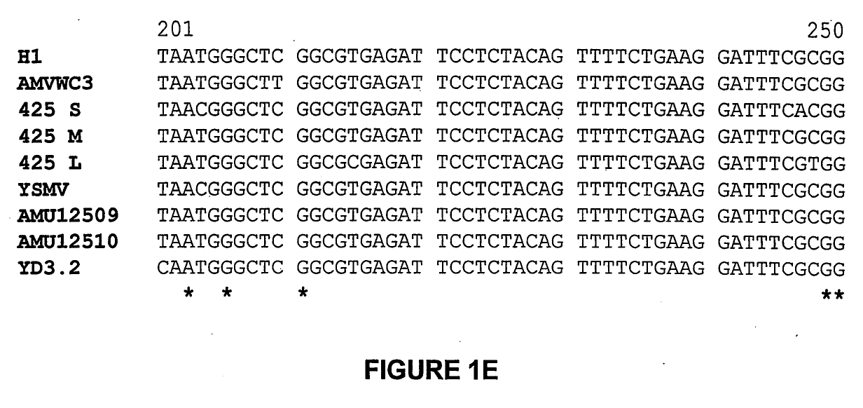

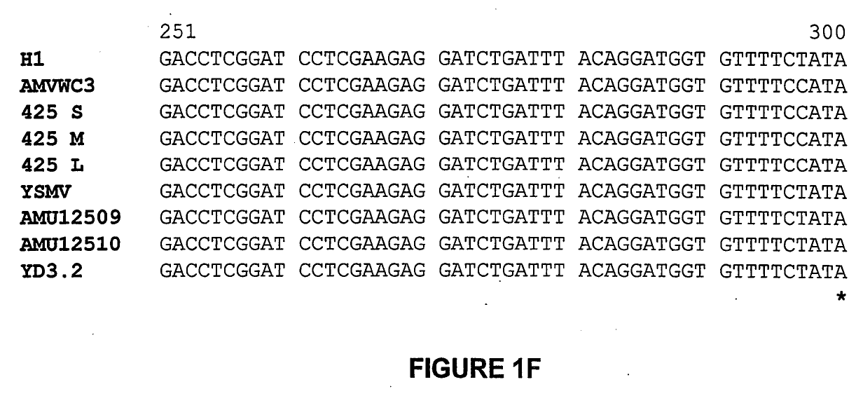

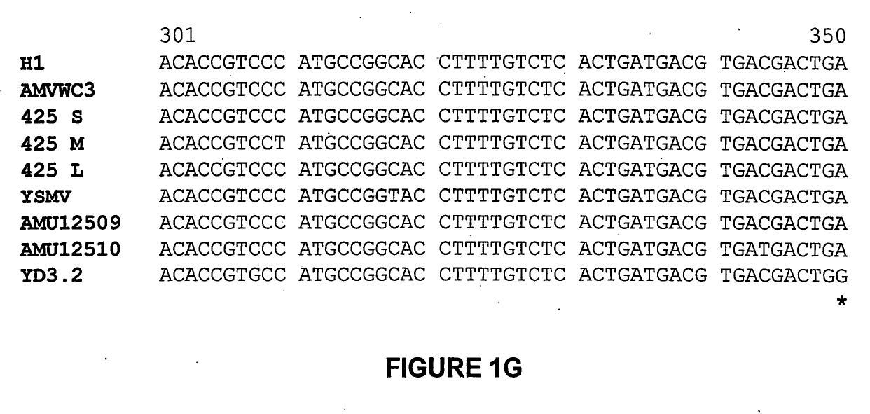

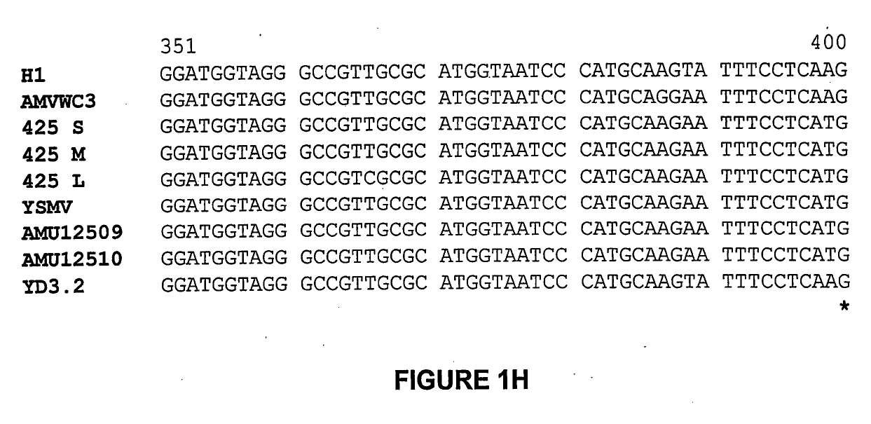

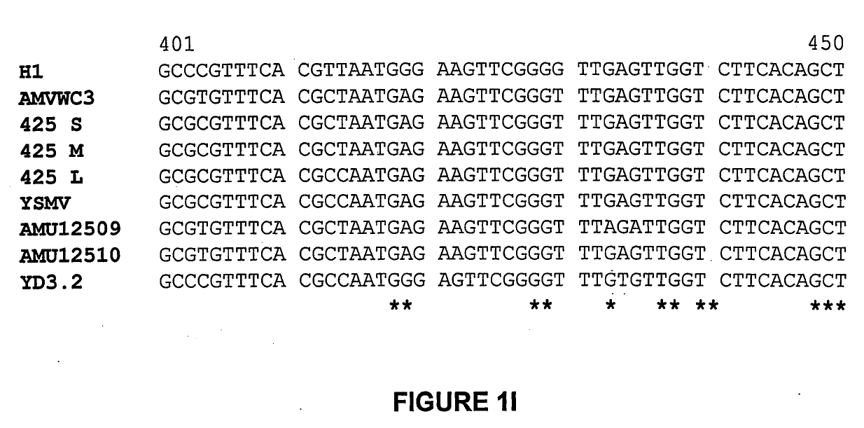

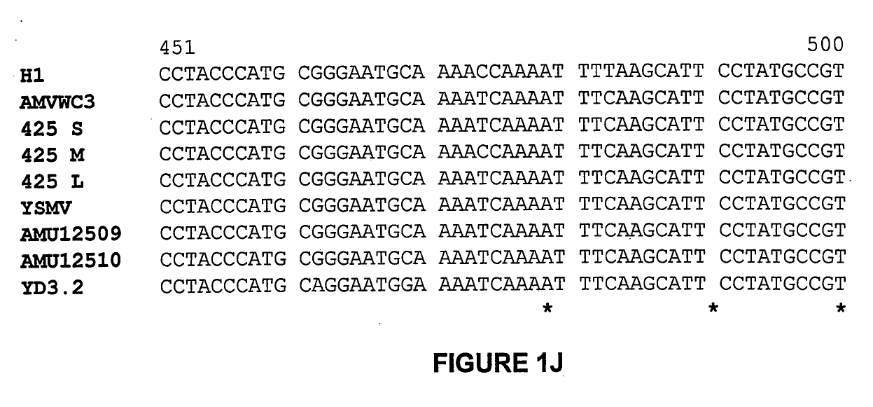

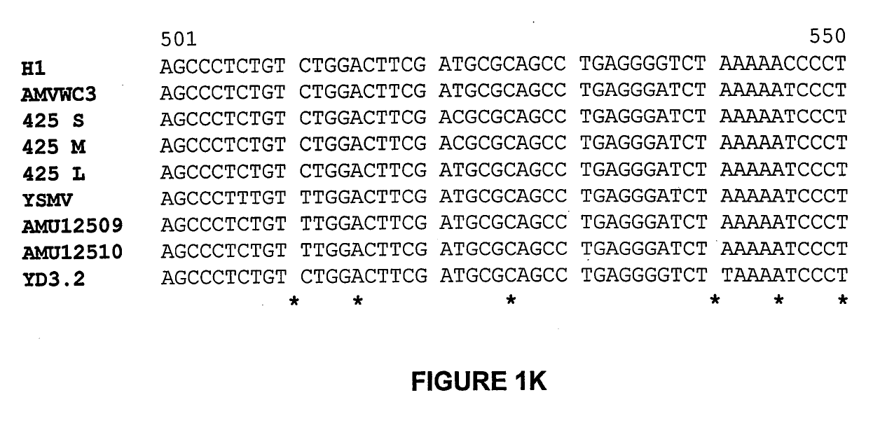

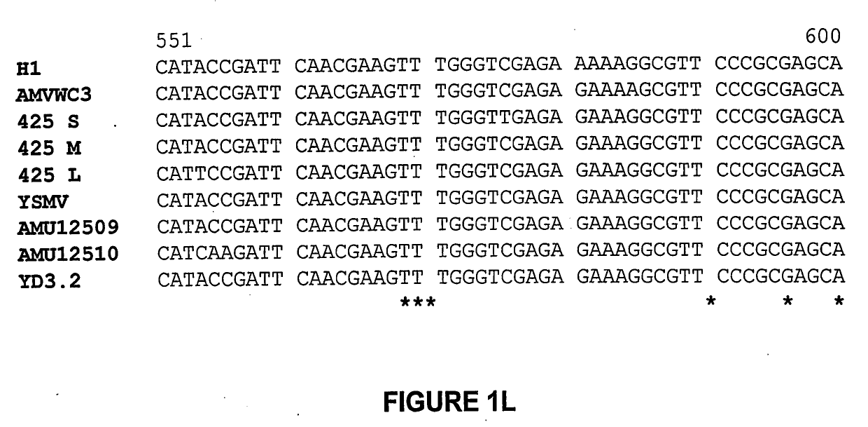

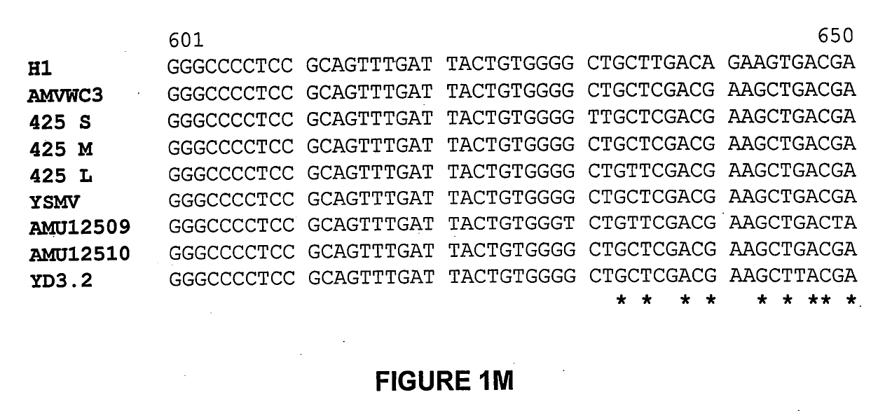

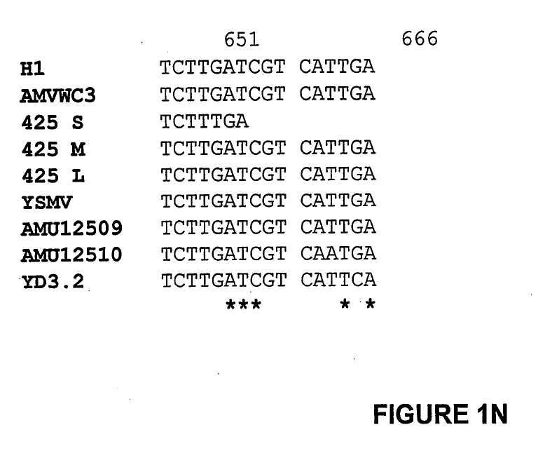

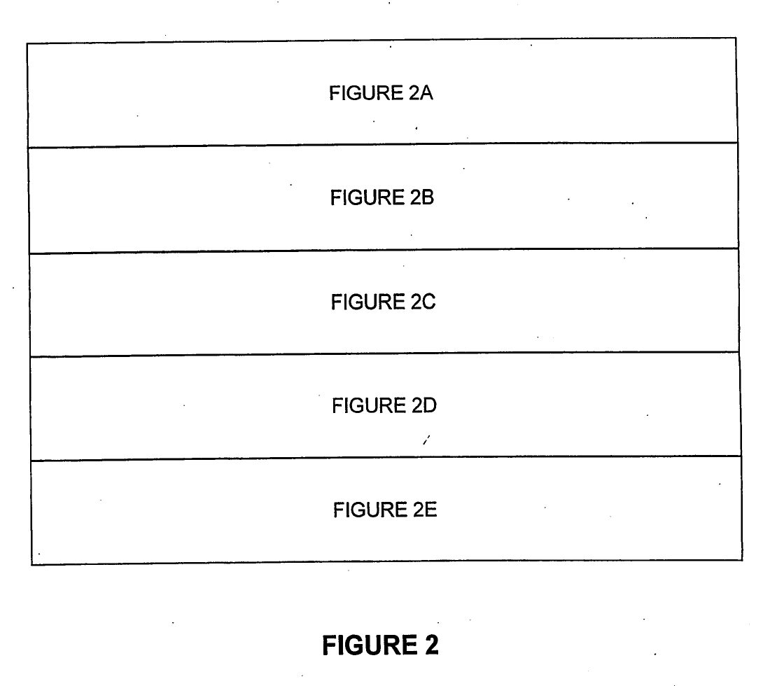

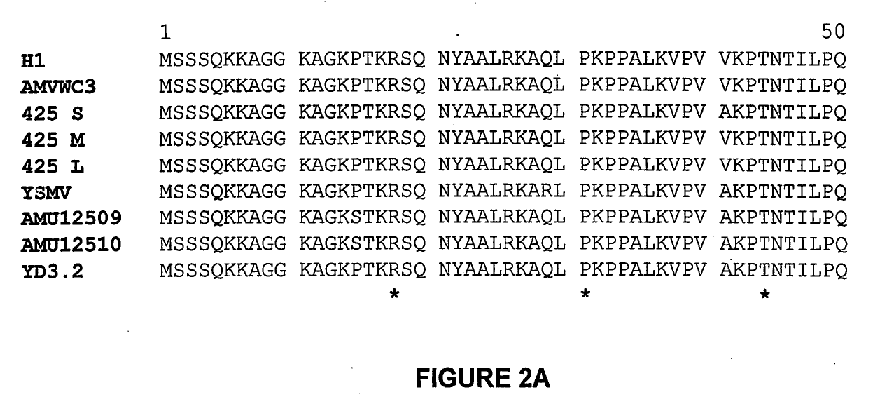

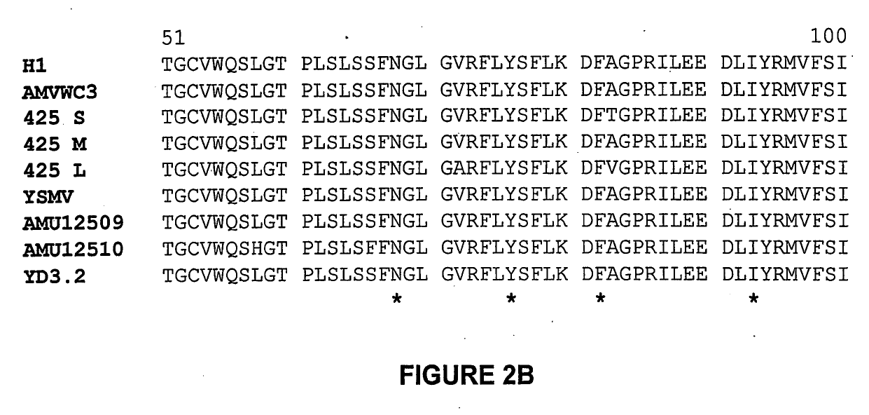

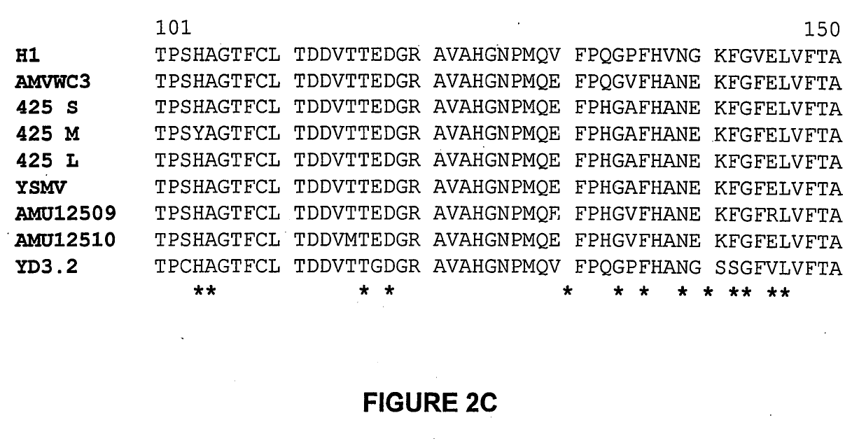

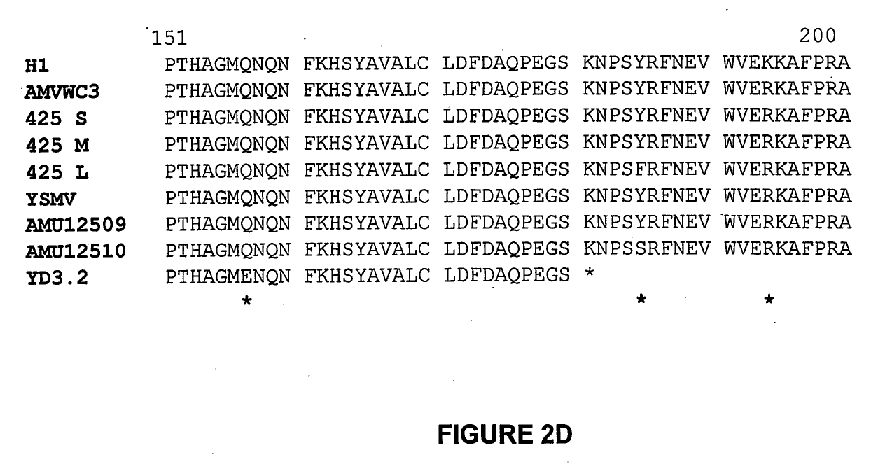

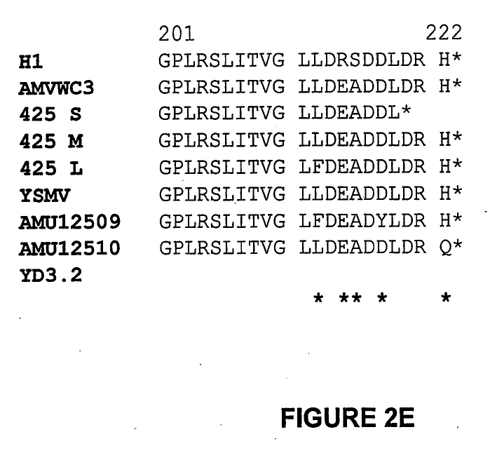

The nucleotide sequences of the coat protein genes of the Type I isolates H1 and WC3, and the Type II isolate YD3.2 are presented in FIG. 1, aligned to the sequences of the coat protein genes of other Type I isolates (i.e. isolates 425S, 425M, and 425L) and the coat protein genes of other Type 11 isolates (i.e. isolates YSMV, AMU12509, and AMU12510). Corresponding amino acid sequences are presented in FIG. 2. The nucleotide and amino acid sequences of the coat protein genes of these nine different isolates of AMV are also presented in SEQ ID Nos: 1-18. The nucleotide sequences were found to share over 92% identity with the corresponding sequences from other AMV strains while the amino acid sequence comparison revealed over 95% identity with known AMV coat protein sequences.

### EXAMPLE 1.3

### Construction of Vectors Comprising the AMV Coat Protein Gene

All cloning procedures used in the preparation of gene constructs comprising the AMV coat protein genes were as described by Sambrook et al. (1989). The cloning strategy used to create recombinant binary vectors containing the AMV coat protein gene of the H1 isolate driven by the Arabidopsis thaliana SSU promoter and containing either Basta resistance (pTW5) or kanamycin resistance (pTP5) is described below:

1. The AMV resistance gene was derived by RT-PCR amplification of the coat protein ORF from partially-purified RNA of AMV strain H1 (a Subgroup I AMV from South Australia, isolated by the late Dr Richard Francki of the Waite Agricultural Research Institute, University of Adelaide, South Australia, Australia), using primers deduced from published sequences of the AMV genome, each of which incorporates a BglII site (bold, underlined text), as follows:

Forward primer: 5′-CCAGATCTTCCATCATGAGTTC-3′ SEQ ID NO: 19; and

Reverse primer: 5′-CCAGATCTTCAATGACGATCMGATC-3′ SEQ ID NO: 20; The amplified coat protein ORF is presented herein as SEQ ID NO: 1.

2. The amplified AMV coat protein PCR fragment was blunt-ended and the resulting fragment was ligated into the EcoRV site of the vector pGEM5Zf(−) (Promega Biotechnology, USA);

3. The vector containing the amplified coat protein-encoding fragment was isolated by digestion with BglII, purified and ligated into the compatible BamHI site of the expression vector pWM5, between the Arabidopsis thaliana SSU promoter and tobacco SSU terminator of said vector, and those constructs comprising the inserted DNA in the sense orientation, capable of expressing AMV coat protein under control of the A. thaliana SSU promoter were selected; and

4. A fragment comprising the A. thaliana SSU promoter plus AMV coat protein ORF plus tobacco SSU terminator was excised from the recombinant pWM5 vector and cloned into a pGA472-based binary vector (e.g., pTAB10 with Basta resistance selectable marker gene, or a member of the pKYLX series of vectors with kanamycin resistance selectable marker) to produce a binary A. tumefaciens vector expressing the coat protein ORF under the control of the A. thaliana SSU promoter and operably connected to the tobacco SSU terminator sequence, for transformation experiments.

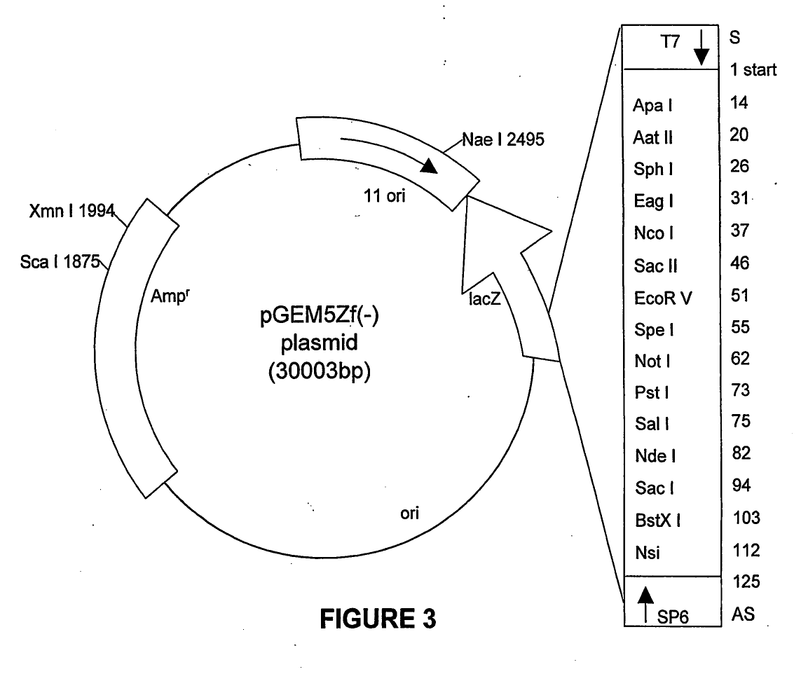

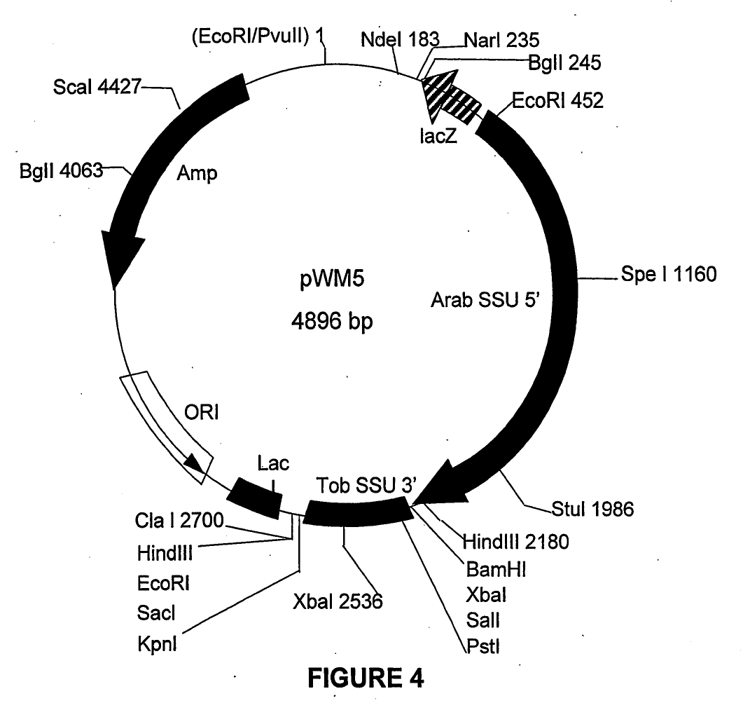

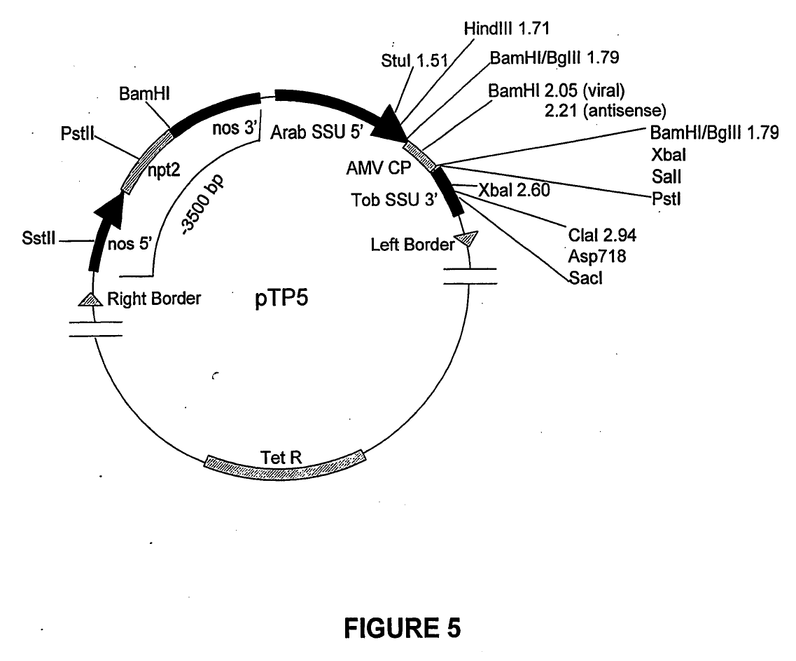

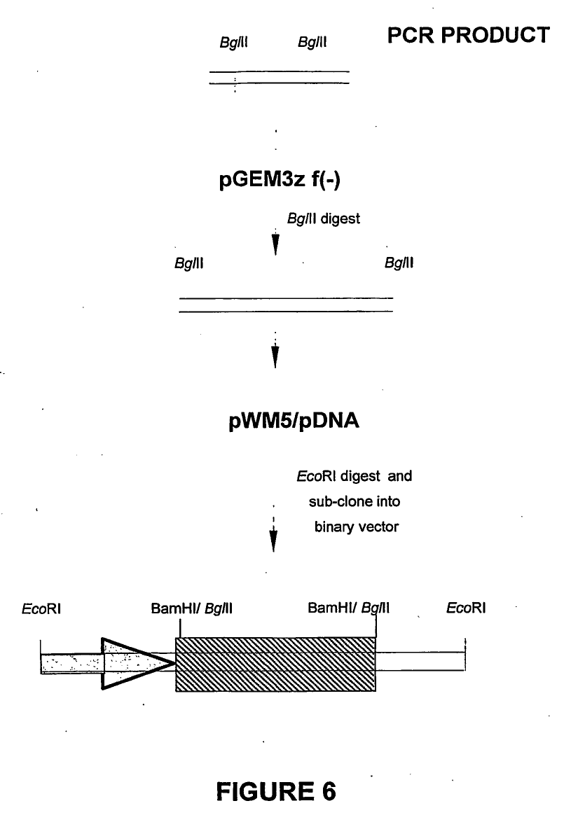

The maps of the plasmids used in constructing the recombinant binary vector pTP5 (FIG. 5) with kanamycin resistance, in particular the plasmids pGEM5Zf(−) and pWM5 are presented in FIGS. 3-4, respectively. The strategy for producing plasmid pTW5, as described herein above, is provided in FIG. 6.

TABLE 1
Summary of Alfalfa Mosaic Virus Isolates Used

| | | | | Systemic Symptoms | | | |
|---|---|---|---|---|---|---|---|
| Isolate | Plant | | | | | White clover cv. | White clover cv. |
| Name | Source | Origin | Broadbean | Cowpea | N. glutinosa | Waverley | Irrigation |
| H1 | Lucerne | Naracoorte, SA | Interv. chlorosis | No symptom | Yellow chlorosis | Mod chlorosis | Mod chlorosis |
| WC10 | White | Glen Innes, | Interv. chlorosis | No symptom | Yellow chlorosis | Mod chlorosis | Mod chlorosis |
| | clover | NSW | | | | | |
| WC28 | White | Glen Innes, | Interv. chlorosis | No symptom | Yellow chlorosis | Mod chlorosis | Mod chlorosis |
| | clover | NSW | | | | | |
| YD1.2 | Lucerne | Yanco, NSW | Interv. chlorosis | Yellow chlorosis | Mild yellow chlorosis | Mod chlorosis | Mod chlorosis |
| YD3.2 | Lucerne | Yanco, NSW | Interv. chlorosis | Yellow chlorosis | Mild yellow chlorosis | Mod chlorosis | Mod chlorosis |
| YD5.2 | Lucerne | Yanco, NSW | Interv. chlorosis | Yellow chlorosis | Mild yellow chlorosis | Mod chlorosis | Mod chlorosis |

Interv., interveinal
Mod. chlorosis, moderate chlorosis

The AMV coat protein genes were also cloned into other binary vectors containing different promoters, terminators and selectable markers, as follows:

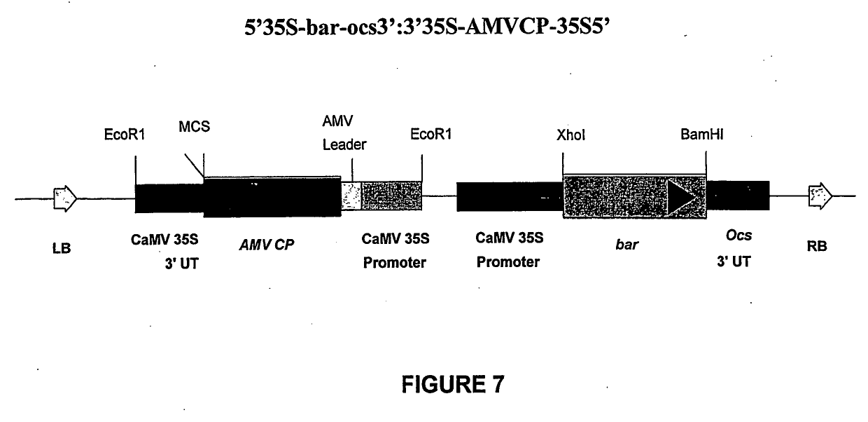

1. The plasmid pTAB10 (Khan et al., 1994; Tabe et al., 1995) containing the bar gene of Streptomyces hygroscopicus encoding phosphinothricin acetyl transferase (De Block et al., 1987; Jones et al., 1992) and conferring resistance to phosphinothricin (PPT) or the commercial herbicide preparations bialophos or Basta® In particular, plasmid pT17 (FIG. 7), a binary vector containing the coat protein gene of AMV isolate H1 was constructed essentially as described for the other AMV coat protein binary vectors, for the transformation of white clover and subterranean clover. The same primers were used for reverse transcription-PCR of the coat protein ORF which was blunt-end ligated to the pGEM5Zf(−) vector at the EcoRV site. The BglII fragment containing the AMV coat protein coding region from the recombinant pGEM vector was then ligated to the expression vector pDHA at the BamHI site. The viral sense construct was selected and cloned into pTAB10 binary vector to produce the A. tumefaciens vector pT17. The T-DNA in the pT17 binary vector thus contains the inserted AMV coat protein coding sequence between a CaMV 35S promoter and a CaMV 35S terminator, together with the bar gene placed operably under the control of a CaMV 35S promoter and an ocs gene terminator; and

2. The plasmid pKYLX71::35S², a derivative of plasmid pKYLX71 (Schardl et al., 1987) wherein the CaMV 35S promoter sequence has been duplicated to increase the level of expression of the gene-of-interest.

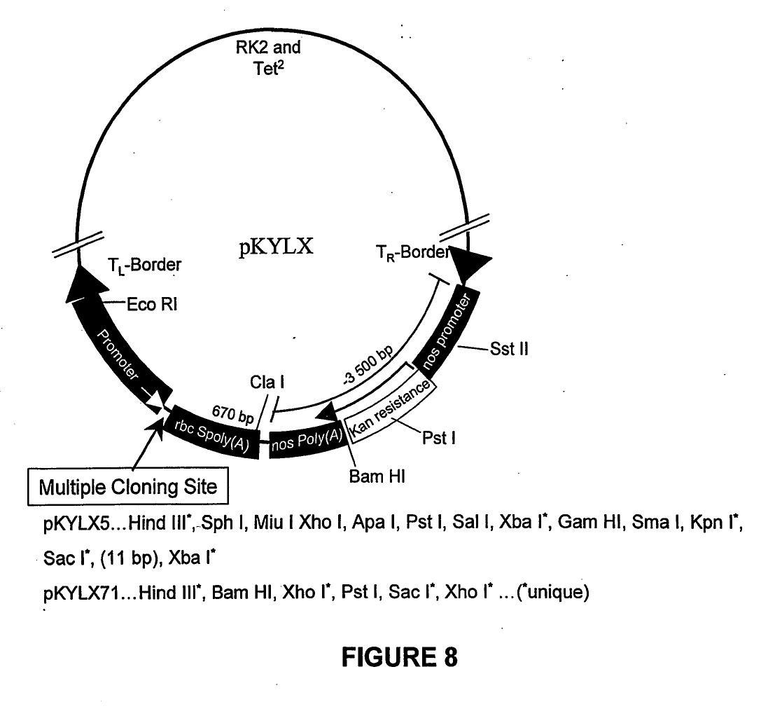

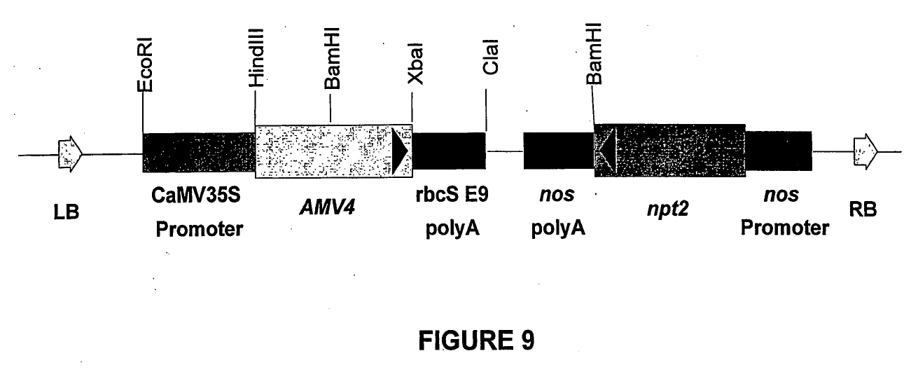

A map of the pKYLX series of plasmids, showing the general design of these vectors is presented in FIG. 8. A map of the binary vector pKYLX71:35S²AMV4, containing the AMV4 coat protein-encoding ORF cloned into the binary vector pKYLX71:35S², is presented in FIG. 9.

### EXAMPLE 1.4

### Isolation and Characterisation of Australian Isolates of CYVV

Australian isolates of CYN (summarised in Table 2) from white clover and other plants were obtained from tissues showing virus-like symptoms collected from various sites. The virus was identified by bioassay on Chenopodium quinoa which produced typical necrotic local lesions followed by local and systemic leaf necrosis and death. Single-lesion isolates of CYVV were confirmed by host range analysis, electron microscopy and ELISA, and were propagated and maintained in broadbeans and white clover, cv. Waverley. Isolates of CYVV that are infectious on all three representative non-transgenic irrigation white clover plants were used for challenging transgenic plants, and the infectivity data are presented in Table 3.

### EXAMPLE 1.5

### Nucleotide Sequences of CYVV Coat Protein Genes

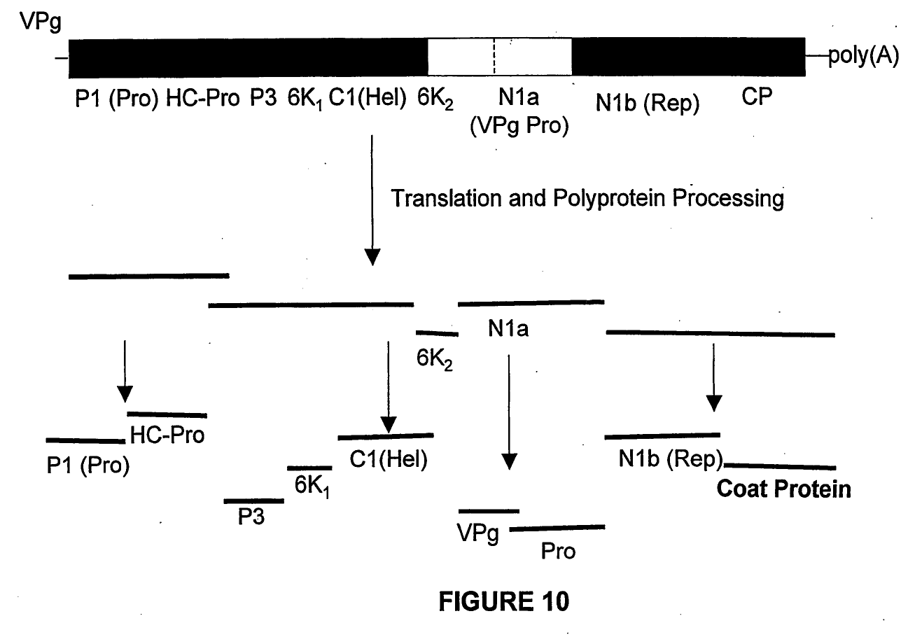

Clover Yellow Vein Virus (CYVV) belongs to the genus potyvirus in the family Potyviridae. Members of this genus have a monopartite genome consisting of a single positive-sense, single-stranded RNA of about 10 kb in length (FIG. 10). A protein (Vpg) is covalently linked to the 5′-terminal nucleotide. A poly(A) tract is present at the 3′ terminus. The genome is organised as a single open reading frame and codes for a polyprotein which is processed by co-translational and post-translational proteolytic cleavage by three virus-coded proteases to produce the mature proteins (Reichmann et al., 1992). The coat protein coding region is located in the 3′-terminus of the genome (ORF designated “Coat Protein” in FIG. 10).

A search of the gene sequence database showed that the published CYVV coat protein nucleotide sequences were highly variable. Attempts were made to amplify the coat protein coding region by RT-PCR using sets of CYVV-specific primers deduced from the various published sequences (SEQ ID Nos: 21-24). A DNA fragment having a length expected for a nucleotide sequence encoding the CYVV coat protein (SEQ ID NO: 25) was obtained from the isolate CYVV300, which isolate was obtained from Dr John Thomas of the Queensland Department of Primary Industry at Malery, Queensland, Australia.

The nucleotide and deduced amino acid sequences of the cloned coat protein gene from CYVV300 was determined using the dideoxy chain termination method (Sanger et al., 1977). Sequence analysis was carried out using the University of Wisconsin Genetics Computer Group Sequence Analysis Software Package (Devereaux et al., 1984). Sequence analysis of the protein encoded by the ORF set forth in SEQ ID NO: 25 indicated that it has about 92% identity to known CYVV coat proteins.

TABLE 2
Summary of CYVV Isolates Used

| | | | Chenopodium symptoms | | N. glutinosa | |
|---|---|---|---|---|---|---|
| Isolate | Source | Host | Local lesions | Systemic | Symptoms | Results |
| ACT 1 | ACT WC | Wav | Nec & ch spots, v. nec | V. ch | NS | CYVV |
| ACT 3 | ACT WC | Wav | Nec spots, v. nec | V. ch | NS | CYVV |
| ACT 4 | ACT WC | Wav | Nec spots, v. nec | V. ch | NS | CYVV |
| Site 6 | ACT WC | Wav | Nec spots, v. nec | V. ch | NS | CYVV |
| PC1 | Florey WC | Wav | Nec spots, v. nec | V. ch | NS | CYVV |
| PC2 | Florey WC | Wav | Nec spots, v. nec | V. ch | NS | CYVV |
| PC5 | Florey WC | Wav | Nec spots, v. nec | V. ch | NS | CYVV |
| PC9 | Florey WC | Wav | Nec spots, v. nec | V. ch | NS | CYVV |
| WC1 | Glenn Innes WC | Wav | Nec spots, v. nec | V. ch | NS | CYVV |
| WC8 | Glenn Innes WC | Wav | Nec spots, v. nec | V. ch | NS | CYVV |
| WC9 | Glenn Innes WC | Wav | Nec spots, v. nec | V. ch | NS | CYVV |
| WC14 | Glenn Innes WC | Wav | Nec spots, v. nec | V. ch | NS | CYVV |
| WC15 | Glenn Innes WC | Wav | Nec spots, v. nec | V. ch | NS | CYVV |
| WC16 | Glenn Innes WC | Wav | Nec spots, v. nec | V. ch | NS | CYVV |
| WC18 | Glenn Innes WC | Wav | Nec spots, v. nec | V. ch | NS | CYVV |
| 300 | QLD Snow pea | Wav | Nec spots, v. nec | V. ch | NS | CYVV |
| Peter | ACT Subclover | Wav | Nec spots, v. nec | V. ch | NS | CYVV |
| 10/13 | PBC WC | Wav | Nec spots, v. nec | V. ch | NS | CYVV |
| Persian | Ham Persian | BB | Ch or mild nec spots, wilt | occ ch spots | NS | CYVV(mild) |

Nec spots, Necrotic spots;
v nec, veinal necrosis;
V ch, veinal chlorosis;
wilt, wilting;
occ ch spots, occasional chlorotic spots;
NS, no symptoms

TABLE 3
Infectivity of CYVV Isolates on Non-Transgenic
White Clover cv. Irrigation Controls NT I2, NT I4 and NT I5

| | Infection Results (extent and location of chlorotic lesions) | | | | | | |
|---|---|---|---|---|---|---|---|
| | NT I2 | | NT I4 | | NT I5 | | |
| CYVV Isolate | Symptoms | Bioassay* | Symptoms | Bioassay* | Symptoms | Bioassay* | Final Results |
| Uninoculated | NS | 0 | NS | 0 | NS | 0 | NS |
| ACT 1 | FV | 4 | NS | 0 | MV | 25 | I4 Resistant |
| ACT 3 + 4 | FV | 12 | NS | 4 | FV | 26 | I4 Susceptible |
| Site 6 | FV | 10 | NS | 0 | VFV | 12 | I4 Resistant |
| PC1 + 2 | VFV | 21 | NS | 9 | VFV | 38 | I4 Susceptible |
| PC5 + 9 | FV + IS | 24 | NS | 0 | NS or VFV | 3 | I4 Resistant |
| WC1 | NS | 0 | VFV | 4 | MV + IY | 21 | I4 Susceptible |
| WC8 + 9 | FV + IS | 56 | NS | 5 | MV + IY | 32 | I4 Susceptible |
| WC14 + 15 | MV + IS | 33 | NS | 6 | FV + IS | 39 | I4 Susceptible |
| WC16 | FV + IS | 54 | VFV + IS | 11 | FV + IS | 37 | I4 Susceptible |
| WC18 | FV | 118 | NS | 23 | MV + IS | 46 | I4 Susceptible |
| 300 | NS | 1? | NS | 0 | NS or VFV | 0 | I4 Resistant |
| Peter | NS | 5 | NS | 0 | FV | 36 | I4 Resistant |
| TWC121 | VFV | 22 | NS | 0 | MV + IY | 23 | I4 Resistant |
| 10/13 | VFV | 14 | NS | 21 | MV + IY | 27 | I4 Susceptible |
| Persian | NS | 2 | NS | 0 | NS or VFV | 0 | I4 Resistant |

NS, no symptoms;
FV, faint veinal chlorosis;
VFV, very faint veinal chlorosis;
IS, interveinal chlorotic spots;
MV, moderate veinal cxhlorosis;
IY, interveinal yellowing:
*number of local lesions on Chenopodium amaranticolor.

### EXAMPLE 1.6

### Construction f Vectors Comprising the CYVV Coat Protein Gene

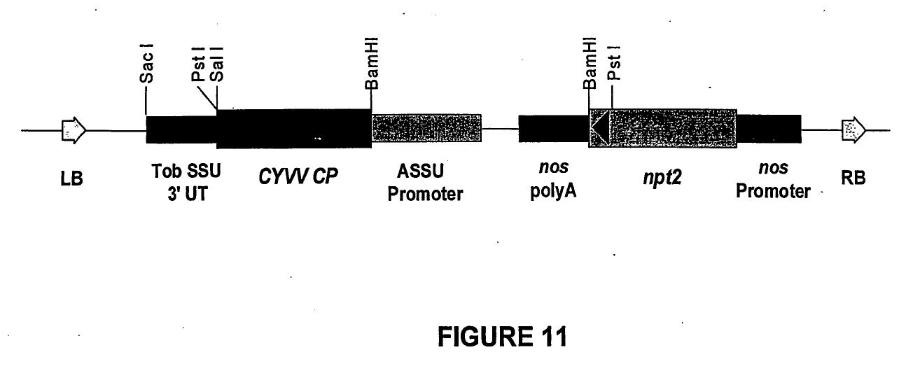

The open reading frame of the coat protein gene of CYVV300 (SEQ ID NO: 25) was cloned in operable connection with the A. thaliana SSU promoter and upstream of the tobacco SSU gene transcription terminator, in the binary vector pKYLX71, to produce the vector pBH1 (FIG. 11). Briefly, plasmid pBH1 was produced as follows:

1. The amplified CYVV coat protein ORF was cloned between the SP6 and T7 promoter sequences in the vector pGEM-T (Promega Biotechnology, USA);

2. The CYVV coat protein ORF was then sub-cloned in operable connection with the Arabidopsis thaliana SSU promoter and tobacco SSU terminator of plasmid pWM5 (FIG. 4), and those constructs comprising the inserted DNA in the sense orientation, capable of expressing CYVV coat protein under control of the A. thaliana SSU promoter were selected; and

3. A fragment comprising the A. thaliana SSU promoter plus CYVV coat protein ORF plus tobacco SSU terminator was excised from the recombinant pWM5 vector and cloned into the binary vector pKYLX71:35S2 (FIG. 8) from which the Cla I to EcoRI fragment was removed, to produce pBH1, which is a binary A. tumefaciens vector expressing the CYVV coat protein ORF under the control of the A. thaliana SSU promoter and operably connected to the tobacco SSU terminator sequence, for transformation experiments.

### EXAMPLE 1.7

### Isolation and Characterisation of Australian Isolates of WCMV

Australian isolates of White Clover Mosaic Virus (WCMV) were obtained from white clover field samples showing virus-like symptoms collected from various sites in Australia, and these isolates are summarised in Table 4.

TABLE 4
Summary of White Clover Mosaic Virus Isolates from White Clover

| | | Systemic Symptoms (extent of veinal chlorosis) | | | | |
|---|---|---|---|---|---|---|
| | | | | | White clover | |
| Isolate Name | Origin | Broadbean | Cowpea# | N. glutinosa | Waverley# | Irrigation |
| WC9 | Glen Innes | Moderate | Mild | NS | Moderate | Mild |
| WC10 | Glen Innes | Mild | Mild | NS | *Moderate | Mild |
| WC16 | Glen Innes | Mild | Mild | NS | *Moderate | Mild |
| WC18 | Glen Innes | Mild | Mild | NS | Moderate | Mild |
| Site2 | ACT | Moderate | Mild | NS | Moderate | Mild |
| PC2 | Florey, ACT | Moderate | Mild | NS | Mild | Mild |
| PC3 | Florey, ACT | Severe | Mild | NS | Moderate | Mild |
| PC4 | Florey, ACT | Severe | Mild | NS | Moderate | Mild |
| PC5 | Florey, ACT | Moderate | Mild | NS | *Moderate | Mild |
| PBC W97 | PBC/AV | Moderate | Mild | NS | Moderate | Mild |
| 4/3 S98 | PBC/AV | Mild | Mild | NS | *Moderate | Mild |
| 4/13 S98 | PBC/AV | Severe | Mild | NS | Moderate | Mild |
| 9/13 S98 | PBC/AV | Moderate | Mild | NS | Moderate | Mild |
| 10/13 S98 | PBC/AV | Severe | Mild | NS | Moderate | Mild |
| 16/13 S98 | PBC/AV | Severe | Mild | NS | Moderate | Mild |
| 22/13 S98 | PBC/AV | Moderate | Mild | NS | Moderate | Mild |
| 22/18 S98 | PBC/AV | Not Determined | Mild | NS | Moderate | Mild |

NS, No Symptoms;
*Most infectious isolates, infecting all lines and producing most distinct symptoms;
#Maintenance host

The virus was identified by bioassay on cowpea, which produced typical chlorotic local lesions followed by mild systemic leaf veinal chlorosis. Single-lesion isolates of the WCMV were confirmed by host range analysis, electron microscopy and ELISA and were propagated and maintained in cowpea and white clover, cv. Waverley. Isolates of WCMV that are infectious on all three representative non-transgenic Irrigation white clover plants were used for challenging transgenic plants.

### EXAMPLE 1.8

### Nucleotide Sequence of the WCMV Coat Protein Gene

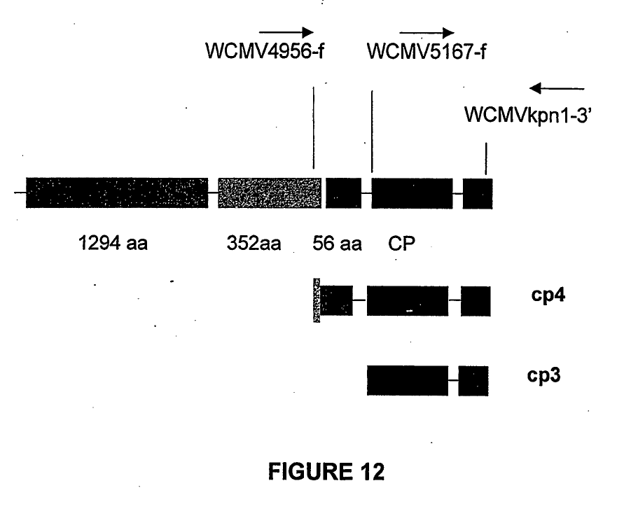

White clover mosaic virus (WCMV) is a member of the potexvirus group. The genome of WCMV (FIG. 12) consists of a positive-sense single-stranded RNA of 5.84 kb, with a 5′-terminal cap structure and a 3′-terminal poly(A) tract. The coat protein gene is in the 3′-terminus of the genome and is expressed through a subgenomic mRNA (sg RNA4) which also has a 5′ cap and a 3′ poly(A) (FIG. 12).

Briefly, fresh white clover leaf tissues exhibiting WCMV field symptoms were collected from sites in the Bundoora campus of La Trobe University, Victoria, Australia. Infection by WCMV was confirmed by electron microscopy and ELISA. This WCMV isolate is referred to hereinafter as “the Bundoora WCMV isolate”. Full-length clones of the WCMV coat protein gene were amplified from purified viral RNA by RT-PCR using WCMV-specific primers, as follows:

| | | |
|---|---|---|
| 1. WCMV4956-f: | 5′AAACTCGAGCATGGACTTCACTACTTTA-3′; | (SEQ ID NO: 27) |
| 2. WCMVKpn1-3′: | 5′-CAGGTACCCTGAAATTTTATTAAACAGAAAGCACACAC-3′; | (SEQ ID NO: 29) |

Restriction sites in the above primers are underlined.

These primers amplify a fragment designated Cp4 (FIG. 12) which consists of nucleotides 4,956 to 5,846 of the WCMV genome and containing a 5′-leader sequence upstream of the coat protein gene. The fragment was cloned into the vector pGEM-7Z (Promega) using standard procedures, to produce p7ZWCMVcp4.

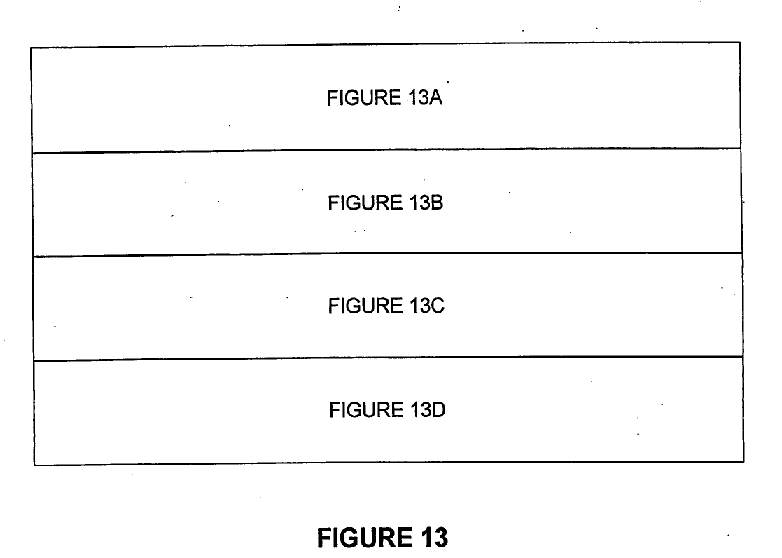

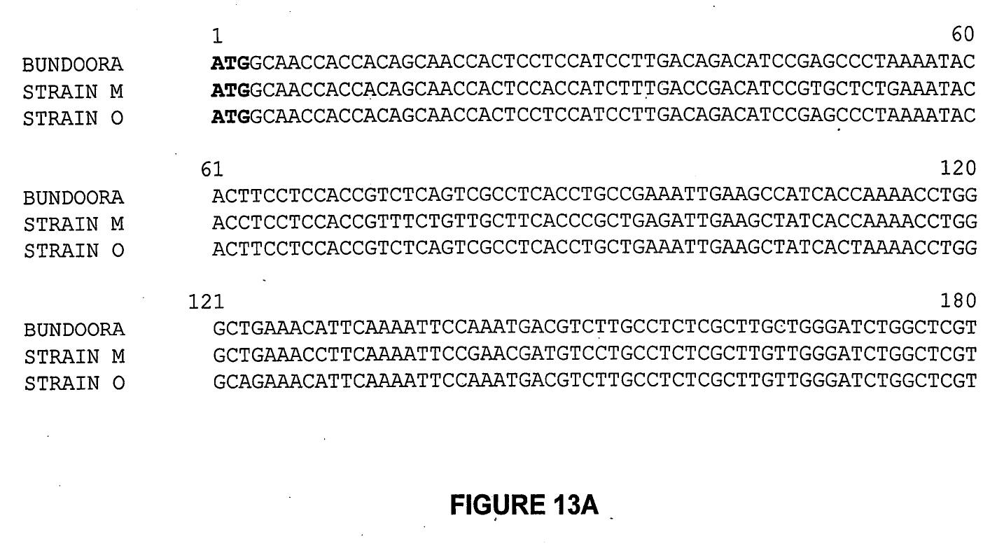

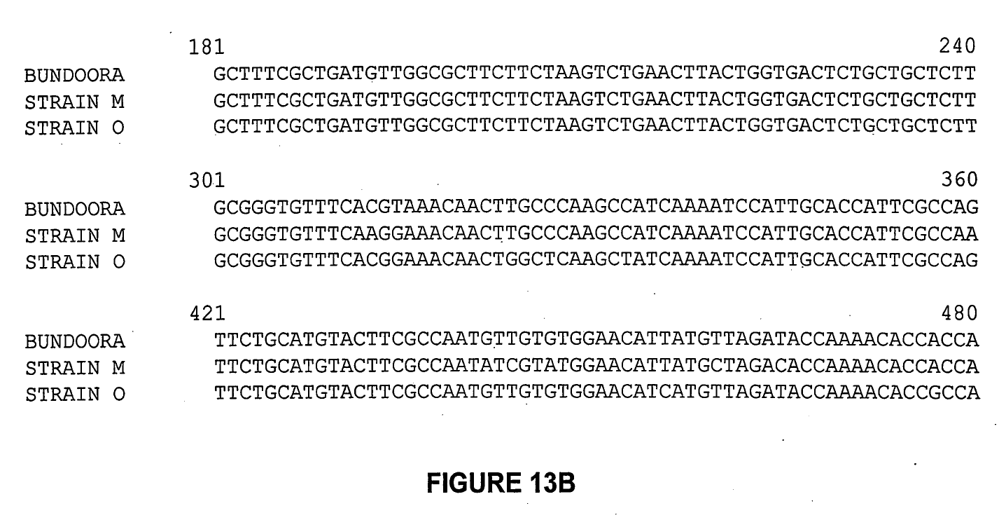

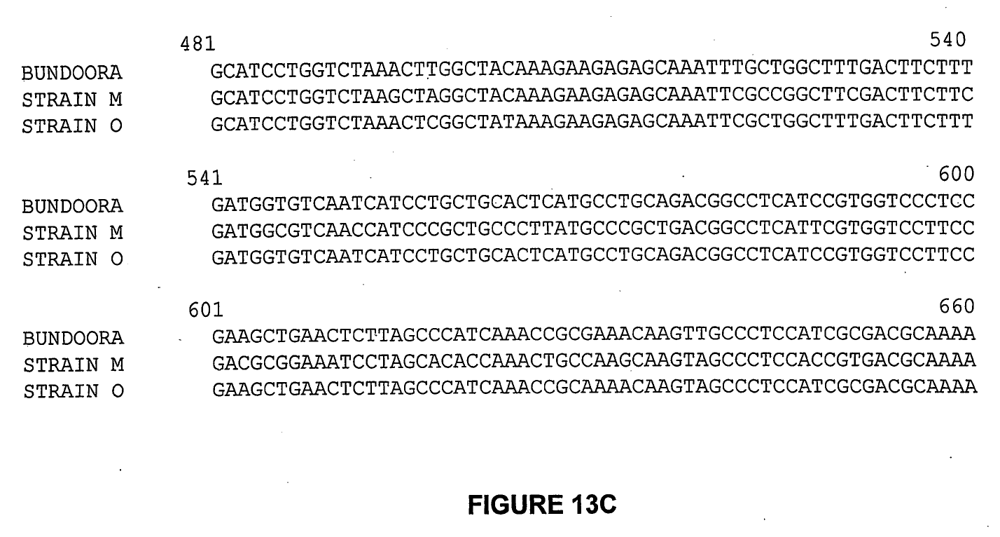

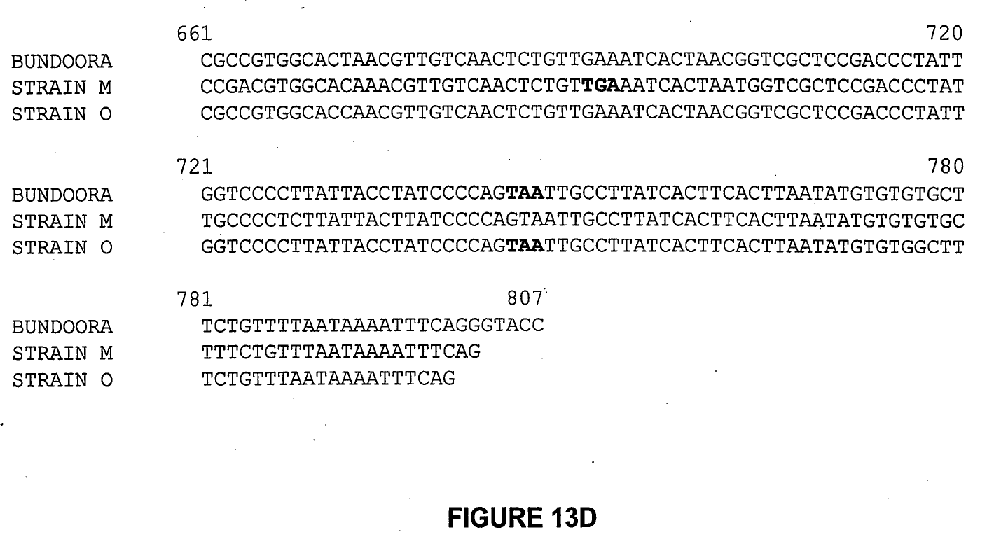

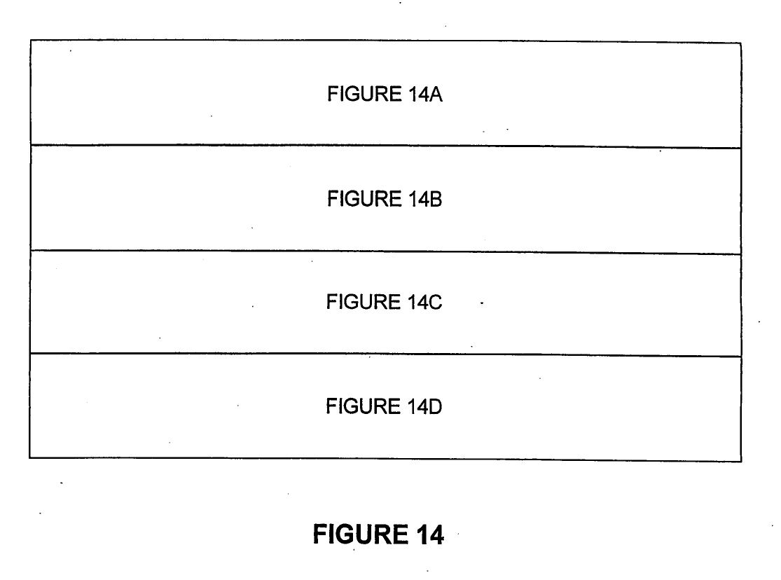

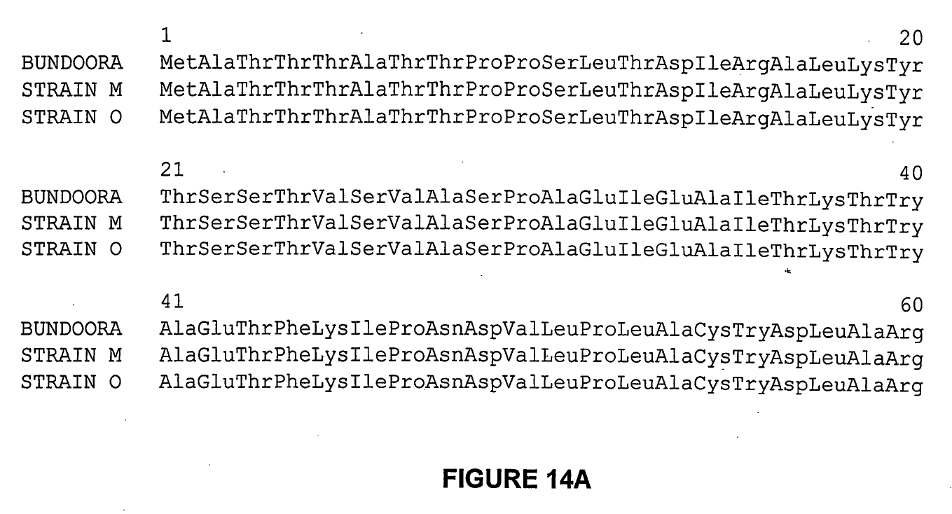

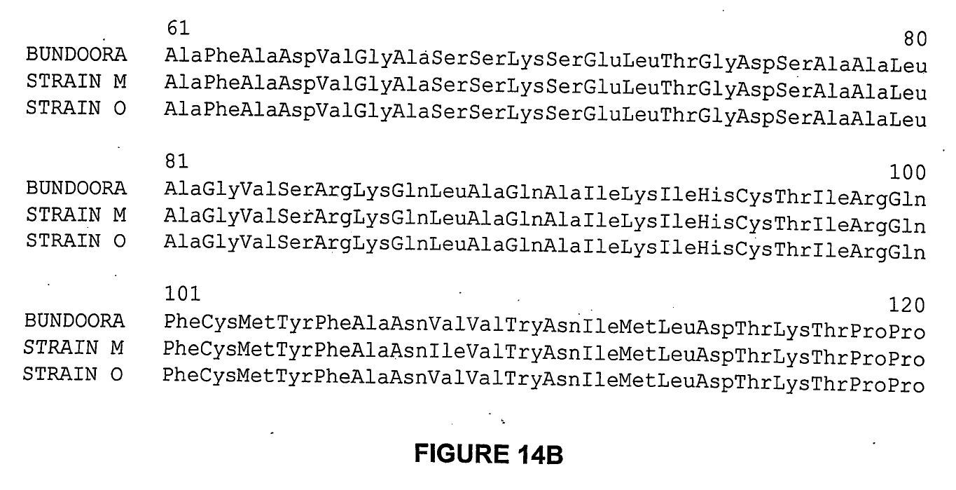

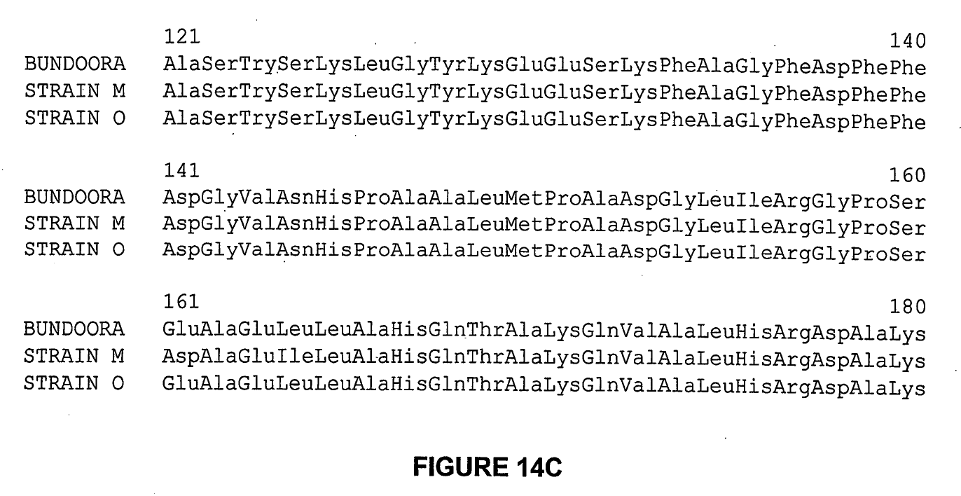

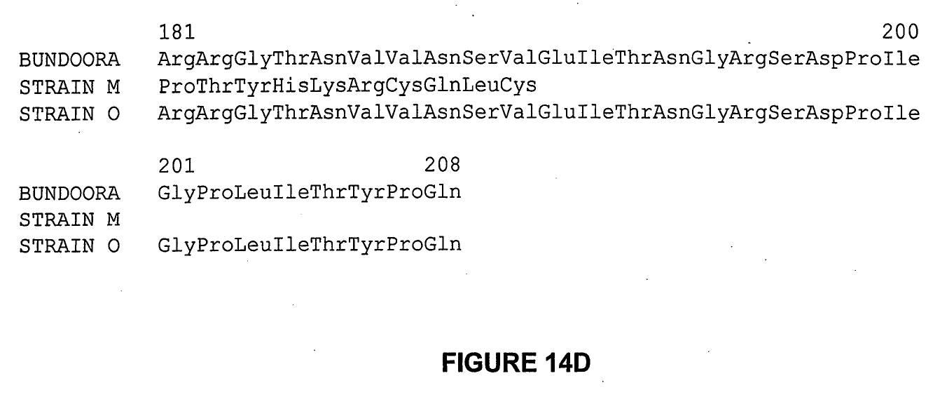

The nucleotide and deduced amino acid sequences of the cloned coat protein gene from the Bundoora WCMV isolate was determined using the dideoxy chain termination method (Sanger et al., 1977). Sequence analysis was carried out using the University of Wisconsin Genetics Computer Group Sequence Analysis Software Package (Devereaux et al., 1984). The nucleotide sequence of the coat protein gene of the Bundoora WCMV isolate is presented in SEQ ID NO: 30, and has 96% identity with the nucleotide sequence of the coat protein of WCMV strain O (SEQ ID NO: 32), and 85% identity with the nucleotide sequence of the coat protein gene of WCMV strain M (SEQ ID NO: 34). The aligned nucleotide sequences of these three coat protein genes are presented in FIG. 13. At the amino acid level, the Bundoora isolate has 100% identity with the coat protein of WCMV strain O and 89% identity with the coat protein of WCMV strain M (FIG. 14). Accordingly, the Bundoora WCMV isolate is not an a typical isolate in terms of the coat protein gene sequence.

### EXAMPLE 1.9

### Construction of Vectors Comprising the WCMV Coat Protein Gene

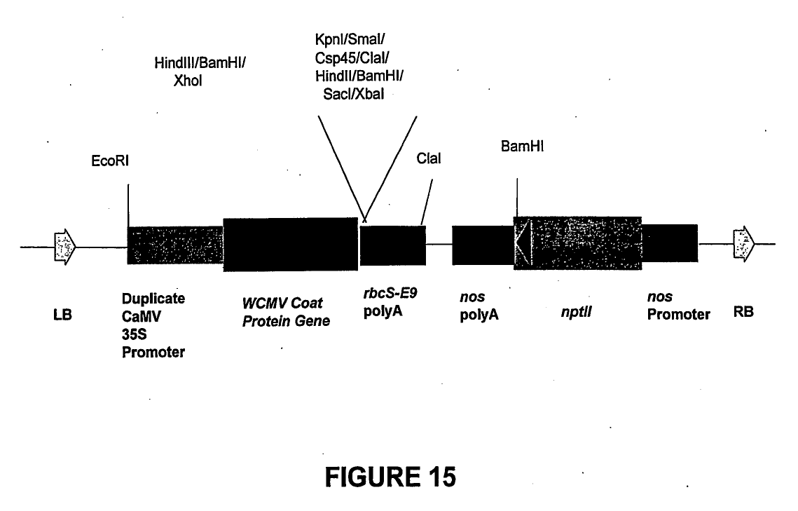

Nucleotides 4,956 to 5,846 of the WCMV genome were sub-cloned from p7ZWCMVcp4 into the binary vector pKYLX71:35S² (FIG. 8; Schardl et al., 1987), in operable connection with the duplicated CaMV 35S promoter sequence contained in that binary plasmid. The resultant plasmid was designated pKYLX71:35S²wcm4 cp. A schematic representation of the T-DNA region of map of the binary vector, pKYLX71:35S²wcm4 cp, is shown in FIG. 15.

### EXAMPLE 1.10

### Construction of Vectors Comprising the CYVV and WCMV Coat Protein Genes

A binary vector containing both the CYVV coat protein gene (Example 5; SEQ ID NO: 25) and the WCMV coat protein gene (Example 8; SEQ ID NO: 30) was constructed for producing plants having dual resistance against CYVV and WCMV, and for super-transforming AMV-resistant lines to thereby produce plants having triple resistance to AMV, CYVV, and WCMV.

The binary construct was designed such that duplication of any transgenic sequences was avoided. Additionally, this gene construct was designed to contain the hph gene, a selectable marker gene that is expressed to confer resistance to hygromycin, to facilitate the selection of transformed cells and Ussues derived from already-transformed lines containing the nptII gene, such as, for example, explants derived from plants containing the T-DNA of a binary plasmid selected from the group consisting of: pTP5 (FIG. 5); vectors based upon any one of the pKYLX vectors (FIG. 8); pBH1 (FIG. 11); and pKYLX71:35S²wcm4 cp (FIG. 15).

Briefly, intermediate plasmid vectors were produced by cloning the entire coding regions of the CYVV and WCMV coat protein genes (SEQ ID Nos: 25 and 30), as follows:

1. The CYVV coat protein ORF was cloned in the sense orientation in operable connection with the A. thaliana SSU promoter and upstream of the tobacco SSU terminator sequence, as described for the production of pTP5 herein above; and

2. The WCMV coat protein ORF was cloned in the sense orientation in operable connection the pea rbcS-E9 promoter and placed upstream of the pea rbcS-E9 terminator sequence, as described for the production of plasmid pKYLX71:35S²wcm4 cp herein above.

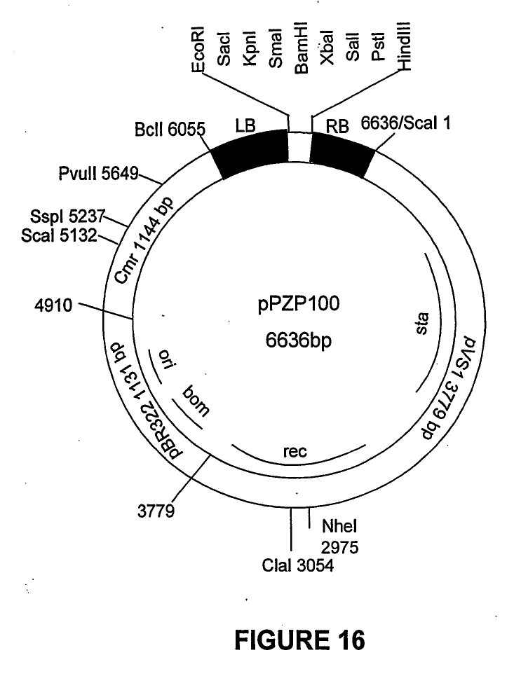

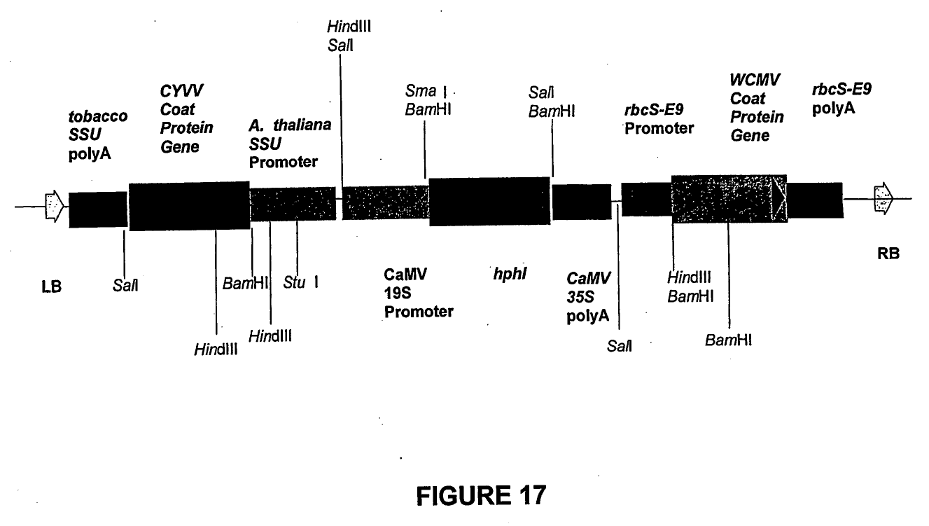

The gene cassettes from each of these intermediate vectors were sequentially cloned, in opposing orientations into the binary vector pPZP100 (FIG. 16; Hajdukiewicz et al., 1994). First, the WCMV coat protein expression cassette was cloned into the HindIII site of pPZP100, creating pPZP100:WCMV4. Then, the CYVV expression cassette was cloned into EcoRI site of pPZP100:WCMV4 creating pPZP100:WCMV4: CYVV CP. A hph gene expression cassette, comprising the hph gene placed operably in connection with the CaMV 19S promoter sequence and placed upstream of the CaMV 35S terminator sequence, was then excised from the plasmid p19Shph using XhoI and SacI, and inserted into the SmaI site of pPZP100:WCMV4: CYVV CP, to produce plasmid pBH3. The T-DNA region of plasmid pBH3 is presented in FIG. 17.

The CaMV 19S promoter in p19Shph was derived from plasmid p19SGUS (supplied by Thomas Hohn of the Friedrich Miescher Institute, Switzerland) as a EcoRV-SalI fragment, and subcloned to produce pB19S. Then p19Shph was produced by inserting the hph coding region and CaMV 35S transcription termination sequences from pGL2 (Bilang et al., 1991) in operable conneciton with the CaMV 19S promoter in pB19S.

In the preparation of plants having triple resistance, the use of the binary plasmid pBH3 for the transformation of AMV-resistant plant lines that were produced as described herein using a binary vector containing the AMV coat protein gene (SEQ ID Nos: 1, 3, 5, 7, 9, 11, 13, 15, or 17), such as, for example, pTP5 or a vector based upon any one of the pKYLX vectors, is particularly preferred.

### EXAMPLE 1.11

### Constructi n of V ct rs Comprising the SCSV Coat Protein Gene

The F isolate of SCSV (Chu and Helms, 1988; Boevink et al., 1995) was used as virus source for the coat protein gene of subterranean clover stunt virus (SCSV). The cloning strategy used to create the recombinant binary vector containing the SCSV coat protein gene is described below:

1. The SCSV resistance gene was derived by PCR of the coat protein ORF from partially purified genomic DNA of SCSV strain F using the following primers deduced from the published sequence of the viral coat protein gene and incorporating a BgIII site (in bold):

| | | | |
|---|---|---|---|
| Forward primer: | 5′-CCAGATCTCAACGAAGATGGTTGCTGTTC-3′; | (SEQ ID NO: 40) | +TL,43 |
| and | | | |
| Reverse primer: | 5′-CCAGATCTTTATACATCAATATAC-3′ 3′. | (SEQ ID NO: 41) | |

Only the coat protein coding sequence was amplified;

2. The SCSV coat protein PCR fragment was blunt-end ligated to pGEM5Zf(−) vector (Promega) at the EcoRV site;

3. The BglII fragment containing the SCSV coat protein coding region from pGEM vector was ligated to the expression vector pDHA, a derivative of pDH51 (Pietrzak et al., 1986), at the BamHI site. The viral sense construct was selected;

4. The SCSV coat protein gene driven by the cauliflower mosaic virus 35S promoter and the 35S terminator was excised with Eco R1 from the recombinant pDHA vector, and cloned into the Eco R1 site of the binary vector pTAB10 to produce the binary A. tumefaciens vector pTS20 for subterranean clover transformation; and

5. The T-DNA in the pTS20 binary vector, containing the inserted SCSV coat protein coding sequence between a CaMV 35S promoter and a CaMV 35S terminator, together with the bar gene franked by a CaMV 35S promoter and the transcription termination sequence from the octopine synthase (OCS) gene of Agrobacterium tumefaciens, were transferred into subterranean clover cells using Agrobacterium tumefaciens-mediated gene transfer with strain AGLI (Lazo et al., 1991) which carries the disarmed hypervirulence plasmid pTi Bo542 derived from strain A281.

The source of plasmids used in constructing pTS20 (pDHA and pTAB10 vectors), are as follows:

1. Plasmid PDHA

Plasmid pDHA was derived from pDH51 (Pietrzak et al., 1986) in which the EcoRV to BamH1 fragment was replaced with a fragment from AMV9 which incorporated a 45 bp 5′ UTR from the AMV (Jobling & Gehrke, 1987) fused to +1 of the 35S promoter (Tabe et al., 1995).

2. Plasmid pTAB10

Plasmid pTAB10 (Khan et al., 1994; Tabe et al., 1995) containing the bar gene from Streptomyces hygroscopicus coding for phosphinothricin acetyl transferase (De Block et al., 1987; Jones et al., 1992). The bar gene serves as a selectable marker in the plant by conferring resistance to phosphinothricin (PPT) or the commercial herbicide preparations bialophos or Basta®.

### EXAMPLE 1.12

### Construction of Binary Vector Containing Hairpin for CYVV Coat Protein

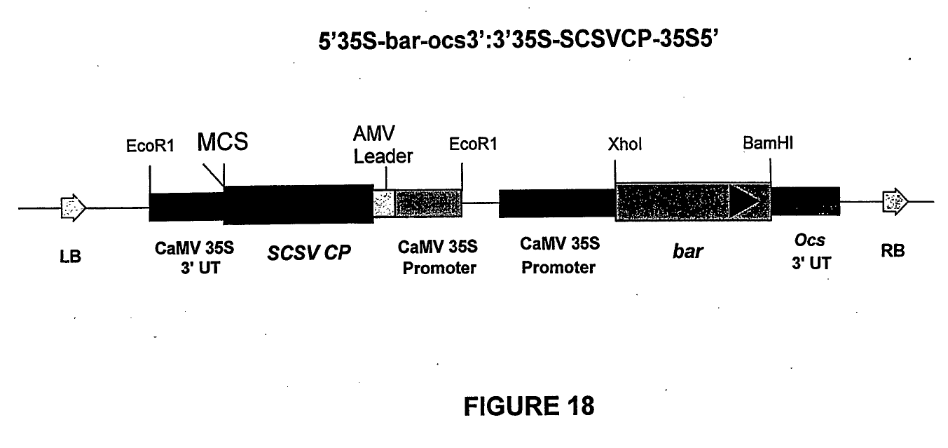

This example shows the production of construct pBH2 containing a CYVV sense/antisense inverted repeat derived from the coat protein gene (FIG. 18B). The inverted repeat is composed of the sense sequence of the CYVV coat protein gene from nucleotides 1-820 and the antisense sequence from nucleotides 1-530 ligated at the salI and EcoRI sites.

The following intermediary plasmids and steps were used in the development of the inverted repeat CYVV coat protein binary vector pBH2 comprising ASSU5′-CYN CP:AS CYVV CP-Tob3′: SC1:hph(CAT-1):SC3:

| Plasmid Name | Gene Content |
|---|---|
| pPZP100:CYVV | ASSU5′-CYVV CP-Tob3′ |
| pBS:hph | SC15′:hph(CAT-1):SC33′ |
| pPZP100:hph:CYVV | ASSU5′-CYVV CP-Tob3′: |
| | SC15′:hph(CAT-1):SC33′ |
| pGEM-7:ASSU:CYVV CP:AS | SP6:ASSU5′-CYVV |
| CYVV CP:Tob | CP:AS CYVV CP-Tob3′:T7 |
| pPZP100:hph:CYVV:AS CYVV | RNAASSU5′-CYVV |
| | CP:AS CYVV CP-Tob3′: |
| | SC1:hph(CAT-1):SC3 |

Part II of the Experimental Section:

Pr ducti n and m lecular Characterisation of Transform d Plants

### EXAMPLE 2.1

### Transformation of Trifolium Species

Our transformation system for Trifolium species, such as, for example, white clover (T. repens), red clover (T. pratense), balanse clover (T. michelianum) and T. isthmocarphum, is based on using cotyledons of imbibed mature seeds (Larkin et al., 1996). Celis of these Trifolium spp. were transformed by Agrobacterium tumefaciens-mediated gene transfer using A. tumefaciens strain AGL1 (Lazo et al., 1991) which carries the disarmed hypervirulence plasmid pTi Bo542 derived from A. tumefaciens strain A281. This strain of A. tumefaciens strain is a wide host range strain, and can be removed from the plant culture by including the antibiotic Timentin in culture media and by axenic culture of tissues until roots are formed and the plants are adapted to soil.

Binary vectors comprising the nptII selectable marker gene or the S. hygroscopicus bar selectable marker gene, and a either the AMV coat protein-encoding gene, CYVV coat protein-encoding gene, or WCMV coat protein-encoding gene were used for white clover and red clover transformation.

Briefly, transformation was achieved by co-cultivating imbibed cotyledons, freshly dissected from seeds, with A. tumefaciens strain AGL1 that had been transformed with a binary plasmid vector described in the preceding section, in particular a binary plasmid selected from the group consisting of: pT17, pTW5, pTP5 (FIG. 5); vectors based upon any one of the pKYLX vectors (FIG. 8); pBH1 (FIG. 11); pKYLX71:35S²wcm4 cp (FIG. 15) and pBH3 (FIG. 17), amongst others. The cotyledons were then grown for a further period of 3 weeks in the selection medium comprising Timentin and either 3-5 mg/L Basta for vectors containing the bar gene, such as pTW5, 25-50 μg/ml kanamycin (or 10 μg/ml geneticin) for the vectors pTP5, the pKYLX-based vectors, or pBH1, or alternatively, hygromycin for the vector pBH3, to select transformed cells. Cotyledons having green shoot initials that developed on the selection medium were grown for a further 3 weeks in fresh selection medium. For selection on kanamycin, some untransformed shoots generally developed during the initial stages, however those died following subsequent subculturing on selection medium, and we found that selection with repeated rounds of subculturing ensured that untransformed shoots were not allowed to develop. The frequency of non-transgenic, kanamycin-resistant plants produced using this procedure was low in repeated experiments. For selection on hygromycin, a clear suppression of growth of non-transformed tissues did not always occur, and some toxicity was observed in respect of putative transformed tissues. Notwithstanding these drawbacks of the hygromycin selection system as applied to white clover, transformed plants containing the hph marker gene stably integrated into the genome were obtained. Basta-resistant green shoots carrying the plasmids pT17 or pTW5, kanamycin-resistant green shoots carrying the plasmids pTP5, pKYLX-based vectors, or pBH1, and hygromycin-resistant green shoots carrying the plasmid pBH3, were then transferred to a rooting medium. Roots generally developed within 2-3 weeks, and, at this stage, plantlets were screened by PCR to confirm the presence of the gene. Plant regeneration frequencies observed by this procedure were generally high, with about 65%-90% of cotyledonary explants of different genotypes and cultivars of T. repens, T. pratense, T. michelianum and T. isthmocarphum producing plantlets in a large number of independent experiments. The plantlets were then transferred to sterilised soil and grown in a PC2 glasshouse. At least 20 independent transformed plants were produced with each binary vector construct. High transformation efficiencies, particularly for Basta and kanamycin selection, corresponding to more than 5% of cotyledons yielding transformed plants, were achieved with the white clover cultivars Irrigation, Haifa and Waverley. This transformation frequency of transformation is an improvement over other methods published for clover, such as, for example, the procedure of Voisey et al., (1994), which produced a transformation efficiency of only 1%. Whilst not limiting the invention to any mode of action, the higher transformation efficiency may be due to our use of cotyledons of imbibed seed that have not germinated completely, such that the transgenic shoots emerge from the lower portion of the cotyledon and the cotyledon stalk. Additionally, we selected only one green plantlet from each cotyledon, even in cases where multiple shoots were observed, to ensure all regenerants were derived from independent transformation events. Moreover, since white clover and red clover are obligate outbreeding species, and, as a consequence, highly heterogeneous, the high frequencies of transformation observed for the different genotypes of these species implies that there is likely to be little effective difference in transformability of other cultivars of these species.

More particular details of the transformation procedure under kanamycin selection are as follows:

To prepare the A. tumefaciens culture, MGL medium (2 ml) containing 20 mg/l rifampicin were inoculated with transformed A. tumefaciens strain AGL1 containing the binary plasmid, and incubated at an angle of about 30 ° on an orbital shaker (150 rpm), at 28° C. for 24 h. The starting inoculum (2 ml) was transferred to 25 ml MGL medium containing 20 mg/l rifampicin and incubated at 28° C. for 48 h. On the same day as commencing co-cultivation of cotyledonary explants, an aliquot 3-4 ml) of this culture was then used to inoculate a further 25 ml MGL medium containing 20 mg/l rifampicin and incubated at 28° C. until reaching OD₆₀₀=0.35 (0.2-0.4).

To prepare white clover or red clover cotyledonary explants for transformation, seeds were washed for 5 min in tap water and surface-sterilised by stirring continuously for 5 min in 30 ml of 70% (v/v) ethanol, followed by a further soaking for 45-60 min in 12.5 ml of 1.5% (w/v) sodium hypochlorite solution containing 3 drops of 0.05%(v/v) Tween 20 detergent as a wetting agent. Seeds were then rinsed 6-8 times with sterile double-distilled water. Finally, seeds were imbibed overnight in about 30 ml of sterile double-distilled water at 10-15° C. Surface sterilized seed were kept for no more than 24 hr at 10-15° C., before removing the seedcoat and endosperm, and excising the cotyledons and at least about 1.5 mm² of cotyledon stalk, into MGL medium.

The excised cotyledonary explants were then transferred to the A. tumefaciens culture prepared as described above, and incubated for 40 min on a rotary shaker at 50 rpm. Following this initial incubation, about 20 cotyledonary explants were washed in sterile RM 73 culture medium, blotted dry, and transferred to culture media plates and incubated for 3 days at 25° C. under a photoperiod comprising 16 hr light and 8 hr dark. Following this cocultivation, the explants were removed, transferred to 9 cm plates containing about 20-30 ml sterile double-distilled water, and washed by gentle shaking. This wash was repeated twice. The explants were then blotted dry and transferred to 9 cm plates containing RM 73 medium plus 250 mg/l cefotaxime or Timentin and kanamycin at a density of 25 explants per plate, by inserting the cotyledonary stalk into the medium. Plates were incubated at 25° C. under a photoperiod comprising 16 hr light and 8 hr dark. Explants were sub-cultured 2-3 times into fresh media every three weeks.

Transgenic white clover or red clover shoots which developed were excised and transferred onto root induction medium (RIM).

### EXAMPLE 2.2

### Transformation of Lucerne (M dicag sativa)

A reliable transformation and regeneration system has been developed for the commercial lucerne cultivars Siriver and Aquarius when coupled to an effective selection system based on the bar gene (Basta selection) or nptII gene (kanamycin selection).

Binary vectors comprising the nptII selectable marker gene or the S. hygroscopicus bar selectable marker gene and an AMV coat protein-encoding gene (Part I of the examples supra); were introduced into M. sativa cv. Siriver and M. sativa cv. Aquarius tissue using the A. tumefaciens strain AGL1 (Lazo et al., 1991). The transformation and regeneration protocols used were essentially as described by Schroder et al. (1991) and as modified by Tabe et al. (1995).

Briefly, transformed A. tumefaciens strain AGL1 carrying the appropriate binary vector construct was co-cultivated with leaf explant material, and the leaf explants were then grown in the presence of Timentin and a selection agent (e.g., kanamycin or Basta, as appropriate for the binary vector used), for 3 weeks. Explants that produced green shoot initials on the selection medium were grown for a further 3 weeks in fresh media comprising Timentin and the selection agent to allow for the development of green shoots that were resistant tot he selection agent. The resistant green shoots were then transferred to a rooting medium until roots developed. The plantlets were then transferred to sterilised soil and grown in a PC2 glasshouse. Thus, transgenic plants could be transferred to soil within 12 weeks of the Agrobacterium co-cultivation after two-three rounds of selection.

For lucerne, kanamycin was less satisfactory than Basta selection, initially allowing some untransformed shoots to develop, though these died in the second or third subculture on selection. The efficiency of transgenic plant recovery was consistently an order of magnitude better with PPT than with kanamycin.

### EXAMPLE 2.3

### Transformation of Subterranean Clover (Trifolium subterraneum L. subclover)

Transformation and regeneration of subterranean clover (cv Gosse) was as described in Khan et al (1994), using an A. tumefaciens-mediated gene delivery system. Developing transgenic shoots were excised and dipped for 1 min into 1 mg/ml IBA solution, before transferring onto RIM containing 3 mg/L IBA. In all cases roots generally developed within 8-20 days. Transgenic plants appearing to contain the bar gene, identified by their ability to grow in the presence of phosphinothricin (PPT, 50 mg/L) in tissue culture, were transferred to the glasshouse in autumn and acclimatised as described in Khan et al (1994) except that the day/night temperature was 23° C./16° C.

### EXAMPLE 2.4

### Procedures for Characterising Transgenic Trifolium spp. and M. sativa Lines

Putative Trifolium spp. And M. sativa transformants carrying various virus resistance genes (either the coding region or the entire sub-genomic messenger RNA (RNA4) of the coat protein genes of the various viruses) (see above), were confirmed as being true transformants by a combination of the following procedures as appropriate: (i) testing for the expression of the bar gene in the Basta resistant lines (e.g., pT17 and pTW5) by the phosphinothricin acetyl transferase (PAT) assay; (ii) testing for the expression of the bar gene in the Basta resistant lines by the leaf painting assay with Basta, (iii) assaying for NPTII enzyme activity in those lines transformed with the kanamycin resistance gene nptII (e.g., pTP5); (iv) PCR assays to detect the selectable marker genes (nptII or hph) or the virus coat protein gene; (v) Southern analyses of genomic DNA to detect both the selectable marker gene and virus coat protein genes; (vi) Northern analysis and RT-PCR to detect mRNA encoded by the introduced coat protein gene construct; and (vii) western blot for detecting virus coat protein in transgenic plants, as described below:

1. PAT Assay

The PAT assay (Spencer et al., 1990) was used to detect the expression of the bar gene in plants transformed with pTAB10-derived vectors. Prior to transplanting plantets to soil, leaves of putative transformants were ground in an equal volume (w/v) of extraction buffer (100 mM Na-phosphate, pH 7; 20 mM NaCl; 1 mM PMSF; 1 mg/ml BSA), the homogenate clarified by centrifugation, and the supernatant retained. The Bradford procedure was used to determine the protein concentration in the supernatant (Bradford, 1976). The supernatant was diluted to a protein concentration of about 1.8-2.0 mg/μl using extraction buffer. Reactions were commenced by adding 6 μl of extraction buffer containing substrate solution (6 mM phosphinothricin; 0.01 μCi/μl [¹⁴C]acetyl CoA (50-60 mCi/mmol; Amersham)) to 16 μl protein extract, and incubating the reaction mixtures at 37° C. for 30 min. Reaction mixtures (15 μl) were then spotted onto silica gel thin layer chromatography (TLC) plates (Merck plastic-backed 0.2 mm Kieselgel60) and allowed to dry for 2 hr. The TLC plates were developed for 2 hr using a solvent solution comprising 1-propanol: 28% (v/v) ammonia solution [3:2 (v/v)], and then allowed to dry for 1 h. TLC plates were then coated with enhancer and allowed to dry for a further 30 min. [¹⁴C]acetylated PPT was detected by fluorography at −80° C. for 18-20 hr.

2. Basta Leaf Painting Assay

Expression of the bar gene in plants was tested by painting duplicate, young, fully expanded leaflets with 1 g/L PPT and scoring 7d after treatment. Transgenic plants expressing the bar gene were resistant to the PPT while those of non-transgenic plants were killed by the herbicide treatment (Khan et al., 1994).

3. NPTII Enzyme Assay

The NPTII enzyme assay was employed according to standard procedures (McDonnell et al., 1987) to identify plants carrying the nptII constructs.

4 (a). PCR Assay to Detect the nptII Gene

The nptII gene present in binary vectors was amplified from transformed plant tissues by PCR, to using TaqI DNA polymerase (Promega) and nptII-specific primers, to identify those plantlets containing the introduced nptII gene constructs. The amplification primers used in this assay were as follows:

| | |
|---|---|
| Primer NPT1: | |
| 5′-GAGGCTATTCGGCTATGACTG-3′; | (SEQ ID NO: 36) |
| and | |
| Primer NPT2: | |
| 5′-ATCGGGAGCGGCGATACCGTA-3′. | (SEQ ID NO: 37) |

These primers are specific for the nptII coding region (nucleotide positions 201-222 and 879-900, respectively, in ISTN5X, AC V00618). In use, these primers produce a fragment of about 600 bp in length which is diagnostic of the introduced nptII gene.

Amplification reactions comprised 1 μg genomic DNA in a standard TaqI reaction buffer and dNTP mixture, and were performed at 95° C. for 5 min, followed by 25 cycles, each cycle comprising 1 min at 95° C., 1 min at 55° C., 1 min at 72° C., and an extension cycle of 3 min at 72° C.

Amplification products were analysed by electrophoresis in 1% (w/v) agarose gels.

4 (b). PCR Assay to Detect the hph Gene

The hph gene present in binary vectors was amplified from transformed plant tissues by PCR, using TaqI DNA polymerase (Promega) and hph-specific primers, to identify those plantlets containing the introduced hph gene construct. The amplification primers used in this assay were as follows:

| | |
|---|---|
| Primer HPH1: | |
| 5′-GCTGGGGCGTCGGTTTCCACTATCGG-3′; | (SEQ ID NO: 38) |
| and | |
| Primer HPH2: | |
| 5′-CGCATAACAGCGCTCATTGACTGGAGC-3′. | (SEQ ID NO: 39) |

These primers are specific for the hph coding region (nucleotide positions 3776-3802 and 3427-3454, respectively, in pGL2). In use, these primers produce a fragment of about 375 bp in length which is diagnostic of the introduced hph gene.

Amplification reactions comprised 1 μg genomic DNA in a standard TaqI reaction buffer and dNTP mixture, and were performed at 95° C. for 5 min, followed by 25 cycles, each cycle comprising 1 min at 95° C., 1 min at 55° C., 1 min at 72° C., and an extension cycle of 3 min at 72° C.

Amplification products were analysed by electrophoresis in 1% (w/v) agarose gels.

4 (c). PCR Assay to Detect Virus Coat Protein Genes

Coat protein genes transformed into plants were amplified using the same sets of primers used for RT-PCR of the respective virus.

Amplification products were analysed by electrophoresis in 1% (w/v) agarose gels.

5. Southern Blot Analyses

Total DNA was isolated from freeze-dried shoot leaf tissue using the CTAB method of Del Sal et al. (1989) with additional phenol/chloroform extractions following the initial chloroform extraction. RNA was removed from the DNA preparations by incubation with RNase at 37° C. for 30 min (10 μg RNaseA and 200U RNaseT1 [Ambion]). The DNA was then extracted with phenol/chloroform, precipitated in ethanol and resuspended in 0.1×TE buffer. Concentrations of the DNA preparations were assessed spectrophotometrically as well as by comparison against known concentrations of salmon sperm DNA. DNA concentrations were normalised as required before Southern analysis.

Ten μg of genomic DNA was digested with appropriate restriction enzymes which cut the introduced T-DNA fragments once, thereby yielding unique DNA fragment sizes wherein one end of each fragment comprised surrounding plant DNA sequences. Digested DNA and a preparation of DIG-labelled DNA molecular weight marker II (Boehringer) was electrophoresed in 1% (w/v) agarose gels and transferred to nylon membrane (Amersham) according to the procedure of Sambrook et al., (1989). DNA immobilised on nylon membrane was crosslinked by UV treatment according to standard procedures.

For the detection of the nptII gene, an internal 1 kb HindIII fragment of the nptII coding sequence was isolated and used to generate a randomly-primed DIG-labelled probe. DIG labelling of the DNA fragment was performed as described by the manufacturer (Boehringer Mannheim GmbH, Germany). Membranes containing DNA were prehybridised and hybridised overnight to DIG-labelled probe as described by the manufacturer. Chemiluminescence was developed using the Anti-Digoxigenin-Alkaline Phosphate conjugate and CSPD substrate solutions. The chemiluminescence signal was visualised after an exposure to X-ray film for 15 min to 1 hour at room temperature.

6 (a). Northern Blot Analyses

Total RNA was isolated from 250-300 mg young folded leaf material using a modified and enhanced Trizol preparation (GibcoBRL) comprising 1 ml of Trizol per 200 mg of frozen and ground sample (Khandjian, 1987; Higgins and Spencer, 1991). RNA recovered by this method was used for Northern analysis.

For northern blot analyses, about 3.5-5.0 μg of total RNA was separated on a 1% (w/v) denaturing formaldehyde agarose gel, transferred by capillary action to HybondN membrane (Amersham), and cross-linked to said membrane by UV exposure. An [α-³²P]dCTP-labelled probe was prepared from isolated coat protein gene derived from the binary vector using the Megaprime DNA labelling system (Amersham) according to the manufacturer's instructions.

For northern blot analyses to detect the expression of genes in T. subterraneum, total RNA was isolated from young leaves of transgenic subterranean clover plants using the method described in Khan et al (1994). Northern blot analyses were performed as outlined in Higgins & Spencer (1991). The AMV and SCSV coat protein-encoding ORFs were obtained by [³²P]-labelling of the respective PCR-generated fragment using an Amersham Megaprime DNA-labelling system according to the manufacturers instructions.

6 (b). RT-PCR Analyses of Transformed Plants

For RT-PCR analyses of transformed plants, total RNA was isolated from 0.6-1.0 gram (fresh weight) young folded leaf material using a modified and enhanced Trizol preparation (Gibco BRL) comprising 1 ml of Trizol per 200 mg of frozen and ground sample (Khandjian, 1987; Higgins and Spencer, 1991). An additional DNase treatment of total RNA was included over the procedure described previously, by incubating the RNA preparations at 37° C. for 1 hour with DNasel.

7. Western Blot Analysis

Leaf tissue (˜0.5 g) from primary transformants (i.e. T₀ plants) was homogenised in 0.5 ml of extraction buffer [100 mM TES (pH 7.8), 200 mM NaCl, 1 mM EDTA, 0.1 mM pefabloc and 2% β-mercaptoethanol], centrifuged, and an aliquot (27 μl) of the supernatant was mixed with loading buffer (Laemmli and Favre, 1973). Coat proteins were dissolved in SDS gel loading buffer to give a final concentration of 1 mg/ml and denatured at 100° C. for 3 min. Approximately 2.5 μg of each sample was electrophoresed in 12.5% polyacrylamide-SDS gels. Following electrophoresis (Laemmli and Favre, 1973), the protein was blotted onto nitrocellulose membranes(Towbin et al., 1979) and probed with anti-virus antiserum followed by peroxidase-labelled goat anti-rabbit antibodies (Bio-Rad), or biotinylated goat anti-rabbit second antibody and streptavidin-conjugated horseradish peroxidase. The reactions were developed using 4-chloronaphthol (Sigma) as instructed by the manufacturer.

### EXAMPLE 2.5

### Characterisation of Transgenic Trifolium spp. Lines Carrying the AMV Coat Protein-Encoding Gene

White clover was successfully transformed with the coding region of the AMV coat protein gene from three Australian isolates of subgroups I and II AMV, using binary vectors containing either the bar or the nptII resistance marker gene. Independent transformed plants produced with each construct were successfully identified by using the appropriate assays. For example, pTW5 transformed lines carrying the bar gene were identified with the PAT assay, followed by confirmation using Southern analyses of genomic DNA to detect for the presence virus coat protein genes (Table 5A). Northern analysis was used to demonstrate the presence of the message of the coat protein transgene (Table 5A) and western blot was used to demonstrate the presence of AMV coat protein in the transgenic plants (Table 5A). Highest expression levels were found in the transgenic lines designated 208, 148, 144, and 135. In contrast to transgenic lines, non-transformed plants did not possess any detectable signals in these assays.

TABLE 5A
Molecular analysis of white clover transformed with binary
vector pTW5 containing the AMV coat protein ORF and the
bar selectable marker gene.

| | | Relative | Relative | Relative | Southern |
|---|---|---|---|---|---|
| | | PAT | AMV | AMV | hybridisation |
| White | | Enzyme | Northern | Western | (AMV |
| Clover | | Activity | Signal | Signal | Transgene |
| Lines | Genotype | (+/−) | (+/−) | (+/−) | Copy No.) |
| Non- | H9 | − | − | − | 0 |
| transgenic | H12 | − | − | − | 0 |
| control | HNN | − | − | − | 0 |
| lines | | | | | |
| pTW5 | 208 | + | +++ | +++ | 1 |
| transformant | 148 | + | ++++ | +++ | 4 |
| lines | 144 | + | +++ | +++ | 1 |
| | 135 | + | +++ | + | 4 |
| | 109 | + | − | + | ND |
| | 130 | + | + | + | 1 |
| | 134 | + | − | + | 8 |
| | 142 | + | − | − | ND |
| | 122 | + | − | − | ND |
| | 123 | + | − | − | ND |

ND = Not Done,
− = no signal;
+ = positive signal.

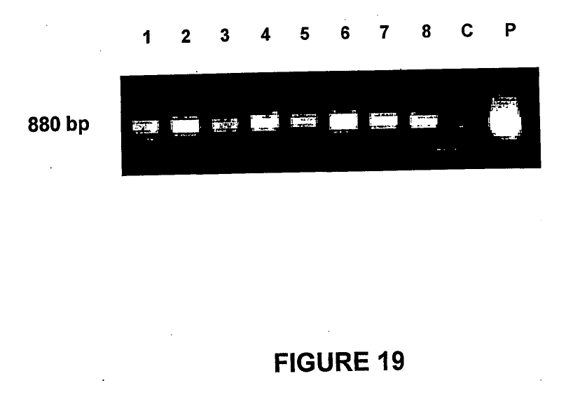

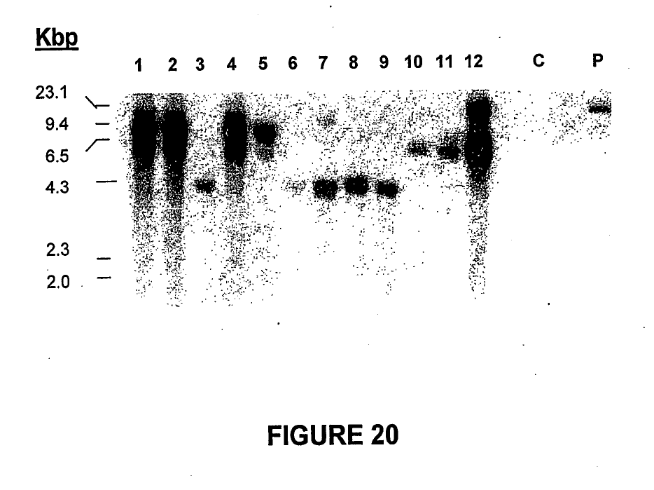

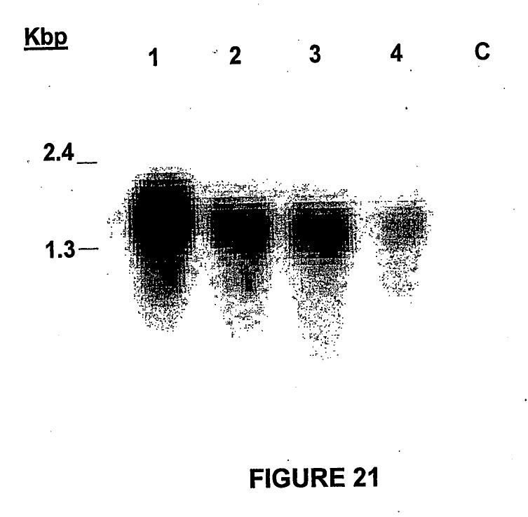

Additionally, several independent lines of red clover (Trifolium pretense) cv. Renegade were successfully transformed with the binary vector pKYLX71:35S²AMV4, (FIG. 9) carrying the AMV coat protein gene, as evidence by PCR assays to detect the introduced npt2 selectable marker gene (FIG. 19)., Southern analyses of genomic DNA to detect the AMV coat protein gene (FIG. 20). and northern analysis to detect expression of the coat protein-encoding mRNA (FIG. 21). The results of the complete molecular analysis of a total of 11 independent lines containing the AMV coat protein gene is shown in Table 5B. Moreover, transformations with same vector were performed for two other red clover cultivars namely, cv. Astred and cv. Redquin. A number of antibiotic resistant putatively transgenic red clover plantlets were screened by PCR assays to detect the introduced npt2 selectable marker gene (Table 5C). In contrast to transgenic lines, non-transformed plants did not possess any detectable signals in these assays (FIGS. 19-21).

TABLE 5B
Transgenic Tₒ Red Clover Plants Containing
the AMV Coat Protein Gene

| | T. pratense | Plant | AMV | Northern |
|---|---|---|---|---|
| Plasmid | Cultivar | Code | Copy no¹ | Analyses² |
| pKYLX71:35S²AMV4 | Renegade | RA1.2 | 4 | − |
| pKYLX71:35S²AMV4 | Renegade | RA1.4 | 4 | − |
| pKYLX71:35S²AMV4 | Renegade | RA3.1 | 4 | + |
| pKYLX71:35S²AMV4 | Renegade | RA3.2 | 1 | + |
| pKYLX71:35S²AMV4 | Renegade | RA4.2 | 1 | + |
| pKYLX71:35S²AMV4 | Renegade | RA4.3 | 1 | + |
| pKYLX71:35S²AMV4 | Renegade | RA5.2 | 1 | +++ |
| pKYLX71:35S²AMV4 | Renegade | RA7.1 | 1 | ++ |
| pKYLX71:35S²AMV4 | Renegade | RA9.1 | 1 | ++ |
| pKYLX71:35S²AMV4 | Renegade | RA9.2 | 2 | ++ |
| pKYLX71:35S²AMV4 | Renegade | RA10.1 | 2 | + |

¹Number of inserted TDNA copies carrying the AMV4 gene estimated by southern blot analysis
²Steady state levels of accumulated AMV4 mRNA transcript estimated by Northern blot analysis
+ = Northern positive
− = Northern negative

TABLE 5C
Production of transgenic Trifolium pratense plants (cultivars
Astred and Redquin) containing virus resistance

| | | Antibiotic | PCR |
|---|---|---|---|
| T. pratense | | resistant | screened |
| cultivar | Plasmid | plantlets | (npt2 +ve)¹ |
| Astred | pKYLX71:35S²AMV4 | 8 | 3 |
| Redquin | pKYLX71:35S²AMV4 | 25 | 7 |

¹PCR reactions were performed with primers detecting the npt2 selectable marker gene

Additionally, putative transgenic subterranean clover plants transformed with pT17 that grew in the presence of Basta selection were tested for expression of the bar gene by the Basta leaf painting bioassay and analysed for the presence of the AMV CP transcript by northern blot analysis. Data presented in Table 5D show that all control non-transgenic subterranean clover plants (normal plants grown from commercial seed and control non-transgenic regenerants) were susceptible to Basta and did not contain any RNA molecules that are able to hybridise to the AMV CP probe. Two transformed plant lines resistant to Basta painting with a readily visible hybridising AMV coat protein mRNA band were obtained.

TABLE 5D
Evaluation of AMV coat protein transgenic subterranean clover lines

| | Northern | Basta |
|---|---|---|
| | (AMV CP) | Resistance of |
| Subterranean Clover Line | in T₀ Parent | T₁ Progeny |
| Gosse Non-transgenic line 1 | − | 0/17 |
| Gosse Non-transgenic line 2 | − | 0/13 |
| Gosse Non-transgenic line 3 | − | 0/19 |
| Gosse Control Regenerant line 1 | − | 0/15 |
| Gosse Control Regenerant line 2 | − | 0/17 |
| Gosse Control Regenerant line 3 | − | 0/18 |
| AMV CP transgenic line 1 | +++ | 12/17 |
| AMV CP transgenic line 2 | +++ | 13/19 |

### EXAMPLE 2.6

### Characterisation of Transgenic Trifolium spp. Lines Carrying the CYVV Coat Protein Gene

Eleven independent transformed white clover lines were successfully produced, as evidenced by NPTII enzyme activity of putative transformants, PCR assays to detect the nptII selectable marker gene present in the binary plasmid vector pBH1 (FIG. 11), and the results of Southern hybridisations of plant genomic DNA to the nptII selectable marker gene. Additionally, northern blot hybridisation analysis successfully detected mRNA encoding the CYVV coat protein in these transformed lines. Data are presented in Table 6. Non-transformed plants did not possess any detectable signals in these assays (not shown).

TABLE 6
Molecular analysis of white clover transformed with binary vector pBH1
containing the CYVV coat protein ORF.

| | | | | | relative CYVV | |
|---|---|---|---|---|---|---|
| | Nptll | Nptll | Nptll | CYVV coat | coat protein | |
| Transgenic | Enzyme | PCR | Southern | protein ORF | mRNA levels | |
| Lines | Activity | assay | assay | Copy No. | (Rating 0-5) | Conclusion |
| BH1-1 | Pos | Pos | Pos | ND | Neg (0) | Transgenic |
| BH1-2 | Pos | Pos | Pos | ND | Pos (5) | Transgenic |
| BH1-3 | Pos | Pos | Pos | ND | Pos (3) | Transgenic |
| BH1-4 | Pos | Pos | Pos | 1 | Pos (1) | Transgenic |
| BH1-5 | Neg | Neg | Neg | ND | Neg (0) | Non-transgenic |
| BH1-6 | Pos | Pos | Pos | ND | Pos (5) | Transgenic |
| BH1-7 | Neg | Neg | Neg | ND | Neg (0) | Non-transgenic |
| BH1-8 | Neg | Neg | Neg | 0 | Neg (0) | Non-transgenic |
| BH1-9 | Neg | Pos | Pos | ND | Neg (0) | Transgenic |
| BH1-10 | Neg | Pos | Pos | ND | Pos (1) | Transgenic |
| BH1-11 | Pos | Pos | Pos | 2 | Pos (2) | Transgenic |
| BH1-12 | Pos | Pos | Pos | 2 | Neg (0) | Transgenic |
| BH1-13 | Pos | Pos | Pos | 3-4 | Pos (1.5) | Transgenic |
| BH1-14 | Neg | Neg | Neg | 0 | Neg (0) | Non-transgenic |
| BH1-15 | Pos | Pos | Pos | ND | Neg (0) | Transgenic |

ND = Not done
Neg = Negative
Pos = Positive

### EXAMPLE 2.7

### Characterisation of Transgenic Trifolium spp. Lines Carrying the WCMV Coat Protein Gene

Independent transformed white clover plants were successfully produced, as evidenced by the results of PCR assays to detect the nptII gene; Southern analyses of genomic DNA for the nptII selectable marker gene present in the binary vector pKYLX71:35S wcm4 cp (FIG. 15); and northern analyses to detect mRNA encoding the WCMV coat protein (Table 7A). Non-transformed plants did not possess any detectable signals in these assays.

TABLE 7A
White Clover Transformed with WCMV C at Protein
(pKYLX71:35S2WCMV4)

| | | Southern | |
|---|---|---|---|
| Plant | | (WCMV4 | Northern |
| Code | Nptll PCR | Copy #) | (WCMV mRNA) |
| NT IC1 | − | 0 | − |
| 4S1 | + | 2 | ++ |
| 4S2 | + | 1 | +++ |
| 4S3 | + | 1 | − |
| 4S4 | + | 2 | − |
| 4S7 | + | 1 | ++ |
| 4S8 | + | 1 | +++ |
| 4S9 | + | 2 | ++ |
| 4S10 | + | 1 | ++ |
| 4S11 | + | 1 | ++ |
| 4S12 | + | 3 | + |
| 4S30 | + | 2 | + |
| 4S36 | + | 2 | ++ |
| 4S55 | + | 2 | +++ |

−, Northern negative;
+, Northern positive

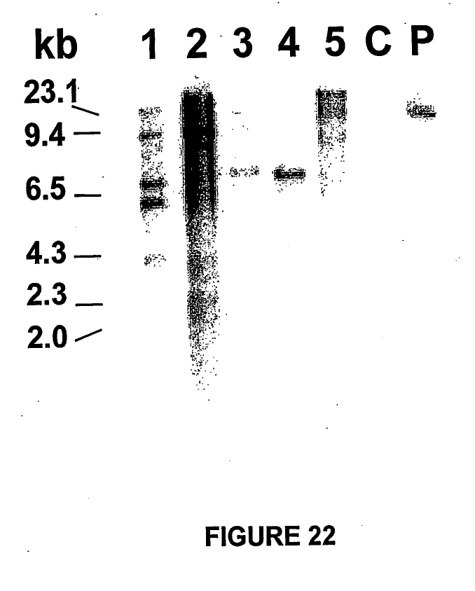

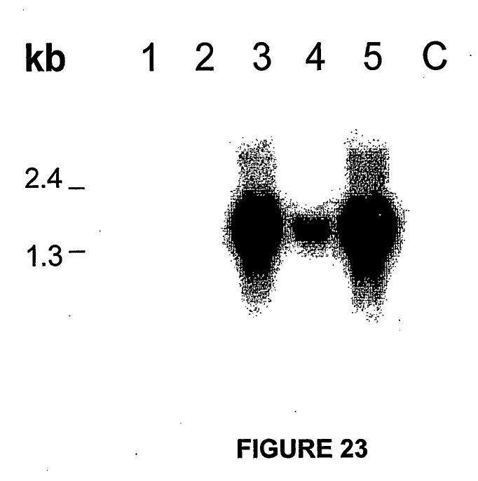

Independent transgenic red clover plants (cv. Renegade) transformed with the binary vector pKYLX71:35S²WCMV4 (FIG. 15) carrying the coat protein gene construct as evidenced by Southern analyses of genomic DNA to detect the WCMV coat protein gene, and northern to analysis to detect expression of the coat protein-encoding mRNA (FIG. 22) were also successfully produced. The results of the complete molecular analysis of a total of 20 independent lines containing the WCMV coat protein gene is shown in Table 7B. Moreover, the same vector was transformed into red clover cv. Astred and cv. Redquin, producing transgenic plantlets that were shown by PCR assays to contain the introduced npt2 selectable marker gene (Table 7C). Non-transformed plants did not possess any detectable signals in these assays (FIGS. 22 and 23).

TABLE 7B
Transgenic WCMV4-Red Clover T0 Plants

| | T. repens | Plant | WCMV4 | Northern |
|---|---|---|---|---|
| Plasmid | cultivar | Code | Copy no¹ | Analyses² |
| pKYLX71:35S²WCMV4 | Renegade | RW1.2 | 5 | − |
| pKYLX71:35S²WCMV4 | Renegade | RW4.1 | 1 | ++ |
| pKYLX71:35S²WCMV4 | Renegade | RW5.1 | 1 | ++++ |
| pKYLX71:35S²WCMV4 | Renegade | RW6.3 | 3 | − |
| pKYLX71:35S²WCMV4 | Renegade | RW7.1 | >5 | − |
| pKYLX71:35S²WCMV4 | Renegade | RW8.1 | 1 | +++ |
| pKYLX71:35S²WCMV4 | Renegade | RW9.3 | 1 | ++ |
| pKYLX71:35S²WCMV4 | Renegade | RW11.1 | 3 | + |
| pKYLX71:35S²WCMV4 | Renegade | RW11.2 | 3 | + |
| pKYLX71:35S²WCMV4 | Renegade | RW12.1 | 2 | ++ |
| pKYLX71:35S²WCMV4 | Renegade | RW12.3 | 2 | ++ |
| pKYLX71:35S²WCMV4 | Renegade | RW13.2 | 1 | +++ |
| pKYLX71:35S²WCMV4 | Renegade | RW15.1 | 3 | − |
| pKYLX71:35S²WCMV4 | Renegade | RW18.1 | 1 | + |
| pKYLX71:35S²WCMV4 | Renegade | RW26.1 | 1 | ++++ |
| pKYLX71:35S²WCMV4 | Renegade | RW35.2 | 3 | − |
| pKYLX71:35S²WCMV4 | Renegade | RW36.1 | 1 | +++ |
| pKYLX71:35S²WCMV4 | Renegade | RW38 | 1 | ++ |
| pKYLX71:35S²WCMV4 | Renegade | RW39.2 | 2 | ++ |
| pKYLX71:35S²WCMV4 | Renegade | RW40 | 1 | ++ |

¹Number of inserted TDNA copies carrying the WCMV4 gene estimated by southern blot analysis
²Steady state levels of accumulated WCMV4 mRNA transcript estimated by Northern blot analysis
+ = Northern positive
− = Northern negative

TABLE 7C
Production of transgenic Trifolium pratense plants (cultivars
Astred and Redquin) containing WCMV coat protein gene

| | | Antibiotic | |
|---|---|---|---|
| T. pratense | | resistant | PCR screened |
| cultivar | Plasmid | plantlets | (npt2 +ve)¹ |
| Astred | pKYLX71:35S²WCMV4 | 11 | 6 |
| Redquin | pKYLX71:35S²WCMV4 | 8 | 2 |

PCR reactions were performed with primers detecting the npt2 selectable marker gene

### EXAMPLE 2.8

### Characteristaion of Transgenic M. sativa. Lines Carrying the AMV C at Protein Gene

Lucerne was successfully transformed with the coding region of the AMV coat protein gene from two Australian isolates of subgroups I and II AMV, using binary vectors containing either the bar or the nptII resistance marker gene. For the binary vectors pT17 and pTW5 containing the bar gene, independent transformed lucerne plants were successfully identified using the PAT assay to detect the bar gene conferring resistance to the herbicide Basta. For the binary vectors designated pTP5, pBS5 and pBS31, which comprise the nptII selectable marker gene, independent transformants were successfully identified using the nptII enzyme assay. Northern analysis were also performed to detect mRNAs encoding the viral coat proteins as described supra. Whilst several independent transgenic lucerne lines were identified using these assays (Table 7D), non-transformed plants did not possess any detectable signals in these assays.

TABLE 7D
Molecular analysis of lucerne transformed with binary
vector pBS5 containing the AMV coat protein ORF
and the nptll selectable marker gene.

| | | Nptll | Northern | |
|---|---|---|---|---|
| Lucerne | | Enzyme | Signal | |
| Line | Line Code | Assay | (AMV) | Results |
| Non- | NT 7 | − | − | Non transgenic |
| Transgenic | NT 13 | − | − | Non transgenic |
| pBS5 | 1 | − | − | Non transgenic |
| Transformed | 2 | + | ++++ | Transgenic |
| Lucerne | 3 | + | +++ | Transgenic |
| | 4 | + | +++ | Transgenic |
| | 5 | − | − | Non transgenic |
| | 6 | − | − | Non transgenic |
| | 7 | − | − | Non transgenic |
| | 8 | + | ++++ | Transgenic |

### EXAMPLE 2.9

### Characterisation of Transgenic T. subterraneum. Lines Carrying the SCSV Coat Protein Gene

A total of 20 putative transgenic plants that grew in the presence of Basta selection in tissue cultere were obtained and tested for expression of the bar gene by the Basta leaf painting bioassay after transfer into the glasshouse. They were also analysed for the presence of the SCSV CP mRNA transcript by northern blot analysis.

The results presented in Table 8 show that all control non-transgenic subterranean clover plants (normal plants grown from commercial seed and control non-transgenic regenerants) were susceptible to Basta painting and did not contain any RNA molecules that are able to hybridise to the SCSV coat protein-encoding probe. Six plant lines were found to be transgenic by their resistance to Basta painting and also had a readily visible hybridising SCSV coat protein mRNA band. A seventh line (SCSV Line 8) was found to have high levels of hybridising SCSV coat protein mRNA but was susceptible to Basta. The other lines were presumable un-transformed regenerants as they were both susceptible to Basta painting and negative in the northern analysis (Table 8).

TABLE 8
Evaluation of SCSV coat protein transgenic subterranean clover lines

| | Northern | |
|---|---|---|
| | (SCSV CP) | Basta Resistance |
| Subterranean Clover Line | in T₀ Parent | among T₁ Progenies |
| Gosse Non-transgenic line 1 | − | 0/17 |
| Gosse Non-transgenic line 2 | − | 0/13 |
| Gosse Non-transgenic line 3 | − | 0/19 |
| Gosse Control Regenerant line 1 | − | 0/15 |
| Gosse Control Regenerant line 2 | − | 0/17 |
| Gosse Control Regenerant line 3 | − | 0/18 |
| Regenerant line 2 | − | 0/20 |
| Regenerant line 3 | − | 0/19 |
| Regenerant line 5 | − | 0/17 |
| Regenerant line 6 | − | 1/20 |
| Regenerant line 7 | − | 0/20 |
| Regenerant line 10 | − | 0/9 |
| Regenerant line 11 | − | 0/19 |
| Regenerant line 12 | − | 0/12 |
| Regenerant line 13 | − | 0/18 |
| Regenerant line 15 | − | 0/20 |
| Regenerant line 17 | − | 0/11 |
| Regenerant line 18 | − | 0/19 |
| Regenerant line 20 | − | 0/20 |
| SCSV CP transgenic line 1 | + | 10/17 |
| SCSV CP transgenic line 4 | + | 13/19 |
| SCSV CP transgenic line 8 | +++ | 1/20 |
| SCSV CP transgenic line 9 | + | 9/20 |
| SCSV CP transgenic line 14 | ++++ | 15/20 |
| SCSV CP transgenic line 16 | + | 14/19 |
| SCSV CP transgenic line 19 | ++ | 11/17 |

Part III of the Experimental Section:

Evaluation of Transgenic Plants for Virus Resistance Characteristics

### EXAMPLE 3.1

### Methods for Virus Inoculation and Determination of Resistance

Transgenic plants, prepared as described in Part II of the experimental section, were tested for resistance to different isolates of AMV, CYVV, or WCMV, using established protocols. Representative non-transgenic genotypes were used as controls to assess the transgenic plants for virus resistance characteristics, and these control plant lines were selected based on the conclusion that their range of susceptibility to the virus isolates was representative of the particular Trifolium spp. cultivar or M. sativa cultivar being tested. To confirm virus infectivity and symptom development, representative Immune, Resistant, and Susceptible plants were bioassayed using Cowpeas (AMV, WCMV) and Chenopodium amaranticolor (AMV, CYVV). Resistance againsta virus was assessed on the bases of symptom recognition, infectivity, and virus bioassay data on indicator hosts. Both primary regenerant transformed lines (i.e. T₀ lines), and progeny of the primary transformants (i.e. T₁ plants), were screened for virus resistance by mechanical inoculation and assessed for symptom development.

1. Plant Preparation

Transgenic lines were tested with 3 representative non-transgenic control lines representative of the cultivar being tested. Transformed lines (i.e. T₀ or T₁ lines) were maintained and multiplied for virus inoculation by vegetative propagation. Approximately 14 stem cuttings were made for each transformed line. About 4 cuttings taken for each transgenic line were used for each inoculum level, and 2 cutting were retained for use as non-inoculated controls. Cuttings were transferred to a glasshouse mister and allowed to grow roots. Rooted plantlets were transferred to soil and were grown under PC2 glasshouse conditions according to the established procedures of the Institutional Biosafety Committee. The transplanted cuttings were then allowed to grow to a good size, generally for about 4-6 weeks, in the glasshouse, before being inoculated with virus Some cutting-back of plants was needed, such that at least 6 newly-expanded young leaves were available per cutting (2-3 shoots/cutting). The plants were kept in the dark for up to 24 hrs before inoculation

2. Preparation of AMV Inoculum for Mechanical Inoculation

AMV strains YC1.2, YD3.2, WC10 and WC28 were used for mechanical inoculation.

Initially, plants were inoculated using freshly-purified virus preparations isolated from the tobacco cultivars Samson, Xanthi or White Burley. AMV was purified as described by Van Vloten-Doting and Jaspars (1972). The quality of each virus preparation was verified by electron microscopy. To determine the concentration of virus in the inoculum, absorption at 260 nm was measured and the concentration calculated based upon an extinction coefficient of 5 for AMV. Each inoculum was diluted in 10 mM Phosphate buffer pH 7.4, 1% (w/v) carborundum, to a concentration of 50 μg/ml, 100 μg/ml, and 200 μg/ml. For each plant, 0.4 ml of inoculum was used.

In later inoculations, the inoculum was prepared by extracting sap from virus-infected plants using a sap extractor and diluted in 5 volumes of 100 mM Phosphate buffer, pH 7.4 (i.e. 1 g leaf fresh weight to 5 ml buffer).

3. Preparation of CYVV Inoculum for Mechanical Inoculation

CYVV strains WC1, WC16 and WC18, isolated from white clover from various regions of Australia, were used for mechanical inoculation.

The CYVV inoculum was prepared by extracting sap from CYVV-infected white clover plants, using a sap extractor, and diluted in 5 volumes of 100 mM Phosphate buffer, pH 7.4 (i.e. 1 g leaf fresh weight to 5 ml buffer).

4. Preparation of WCMV Inoculum for Mechanical Inoculation

WCMV isolates Ham12, Ham22 and WC16, isolated from white clover grown in Victoria, Australia, and New South Wales, Australia, were used for mechanical inoculation.

The WCMV inoculum was prepared by extracting sap from virus WCMV-infected white clover plants, using a sap extractor, and diluted in 5 volumes of 100 mM Phosphate buffer, pH 7.4 (i.e. 1 g leaf fresh weight to 5 ml buffer).

5. Virus Inoculation

Each plant, prepared as described supra, was inoculated with 0.4 ml of ice-cold virus inoculum, by rubbing the inoculum 5 times onto the upper surface of each leaflet of 6 leaves. After inoculation, leaves were rinsed with water.

6. Aphid Transmission Tests

Aphid transmission tests were carried out in PC2 cold frames using vegetatively-propagated clonal plants, prepared as described above for mechanical inoculation. Aphids were applied to virus-infected white clover plants, and allowed to spread onto the testplants over a period of up to 4 weeks before they were eliminated by insecticide sprays.

7. Evaluation of Inoculated Plants

Infected plants were monitored daily for the development of local lesions, and any lesions detected were counted. Infected plants were also monitored weekly for the development of systemic symptoms, which were assessed on transgenic clover plants at 3 and 6 weeks post-infection, and, on transgenic lucerne plants at 5 and 8 weeks post-infection.

8. Virus Bioassay of Inoculated Plants

Representative Immune, Resistant, and Susceptible plants, were bioassayed, using Cowpeas which test positive for AMV and WCMV, but negative for CYVV (Table 9); and Chenopodium amaranticolor which tests positive for AMV and CYVV, but negative for WCMV (Table 9), to identify the presence of AMV, CYVV or WCMV in the inoculated plants.

Young leaves from inoculated plants were ground in 1 ml of chilled 100 mM Phosphate buffer (pH7.4), using a sap extractor, and collected in Eppendorf tubes. The ice-cold extracts were inoculated onto young indicator plants.

9. ELISA

Virus accumulation in the inoculated plants was estimated by double-sandwich ELISA detection of virus in leaf samples, as described by Clark and Adams (1977). Antibody reagents for AMV, CYVV and WCMV detection were prepared from rabbit polyclonal antisera against the respective viruses.

TABLE 9
Summary of bioassay symptoms for AMV, CYVV and WCMV on diagnostic
susceptible and non-susceptible indicator hosts

| | Maintenance | Indicator host symptoms (Local Lesions; Systemic symptoms) | | | |
|---|---|---|---|---|---|
| Virus | host & symptoms | Broadbean | Ch. Amaranticolor | Cowpeas | Tobacco |
| Potexvirus: | Broad bean: | Large diffused ch | NS | Medium yellow-green | NS |
| WCMV | Sys v ch; ch spot | spot + nec | | ch spot & ring spot | |
| | | ring; | | (5-7 dpi); faint v. ch | |
| Subgroup I | N. glutinosa: | Nec ring, nec spot, | Numerous small yellow ch | Numerous small red | Ch spot + nec spots + |
| AMV: AMV- | Sys fine v nec | ringspot (red | spot + nec spot (2-3 dpi); | nec spot (2-3 dpi); no | Ringspot (3-4 dpi); |
| WC28; | partial ring; | brown); nec spot + | Fine ch lines + nec lines | systemic symptoms | fine v nec; mosaic |
| AMV-YC1.2; | yellow | v ch | parallel to veins; mosaic; no wilting | | |
| | ch patch/spot | | | | |
| Subgroup II | Cowpea: | Nec spot (red | Numerous small yellow ch | Numerous small ch | Faint ch spot + ringspot |
| AMV: AMV- | Sys yellow ch | brown); | spot + nec spots + specks | spot + nec spot + ringspot | (3-4 dpi); |
| YD3.2 | | mild v ch; fine nec | (3-4 dpi); fine ch lines + nec | (3-4 dpi); | mild ch or mosaic |
| | | | lines parallel | bright yellow ch | |
| | | | To veins; mosaic; no wilting | | |
| Potyvirus: | Broad bean: | Large nec spot | Few large white nec spot | NS | Faint ch spot & nec |
| CYVV-300 | Severe sys yellow | (dark brown); ch & | (7-10 dpi) followed by | | ringspot (6-7 dpi); |
| | ch + nec; | nec, vein yellow; | wilting; nec spot, nec + wilting | | No systemic |
| | occasional | occasional shoot | | | symptoms |
| | Wilting of shoots | wilting | | | |
| Potyvirus: | Broad bean: | NS or faint ch spot | Medium ch spot + ringspot + | NS | NS |
| BYMV-S | Sys green | (green); faint ch | Occasional nec spot | | |
| | mosaicing | spot + mosaic | (10-12 dpi); | | |
| | | | No systemic symptoms | | |

Nec, Necrotic;
ch, chlorotic;
v, veinal;
sys, systemic;
NS, diagnostic non-susceptible;
dpi, days post-infection

### EXAMPLE 3.2

### Assessment of White and Red Clover Plants for AMV Resistance by Mechanical Inoculation

A total of 259 confirmed independent transgenic white clover lines, of cultivars Irrigation, Haifa and Waverley, transformed with 10 different constructs were tested for resistance to AMV by mechanical inoculation. About 21% of these lines (57 lines) were found to be immune, as determined by the absence of detectable infection by three Australian isolates of AMV, representing both subgroup I and sub-group II of AMV. Additionally, 27% of the lines tested were determined as being resistant to AMV sub-group I and sub-group II virus isolates, by virtue of their showing less than 50% infection in the form of lesions or systemic symptoms. None of the 19 non-transgenic lines tested showed any degree of immunity or resistance, as all non-transgenic lines exhibited more than 85% infection. The results of a typical inoculation experiment are presented in Table 10. The proportion of AMV-immune transgenic lines obtained with the different binary vector constructs are tabulated in Table 11A.

TABLE 10
Resistance of transgenic white clover lines against AMV strain WC28 at 47 days
post-infection as determined by assessment of symptoms and bioassay

| | | % Infection per Inoculum Level | | | Mean % | WC28 |
|---|---|---|---|---|---|---|
| Construct | Line | 50 ug/ml | 100 ug/ml | 200 ug/ml | Infection* | Results* |
| Non-Transgenic | H1 | 83 | 100 | 100 | 94 | S |
| Haifa | H12 | 83 | 83 | 100 | 89 | S |
| | HNN | 67 | 83 | 100 | 83 | S |
| pTW5 Lines | TWC 122 | 100 | 100 | 100 | 100 | S |
| | TWC 125 | 0 | 0 | 0 | 0 | I |
| | TWC 126 | 0 | 40 | 60 | 33 | R |
| | TWC 129 | 0 | 0 | 0 | 0 | I |
| | TWC 131 | 0 | 0 | 0 | 0 | I |
| PTW12 Lines | TWC 115 | 100 | 100 | 100 | 100 | S |
| (antisense coat | TWC 152 | 100 | 100 | 100 | 100 | S |
| protein) | TWC 111 | 33 | 60 | 83 | 59 | S |
| | TWC 155 | 0 | 53 | 17 | 23 | R |

S, susceptible (more than 50% infected),
R, resistant (less than 50% infected),
I, immune (no detectable infection)

TABLE 11A
Correlation of immunity against AMV in white clover with
gene construct used to transform plants

| Binary | Selectable Marker | Resistance gene | No of Immune Lines | | |
|---|---|---|---|---|---|
| Vector | Gene | insert | Irrigation | Haifa | Waverley |
| pT17 | 35Spro-bar-OCS | 35Spro-CP1-35Sterm | — | 1/13 | — |
| pTW5 | 35Spro-bar-OCS | ASSU-CP1-TobSSU | — | 11/33 | 15/42 |
| pTW12 | 35Spro-bar-OCS | ASSU-a/sCP1- | — | 0/28 | — |
| | | TobSSU | | | |
| pTP5 | nospro-nptll- | ASSU-CP1-TobSSU | 3/17 | 2/8 | 3/12 |
| | nosterm | | | | |
| pTP12 | nospro-nptll- | ASSU-a/sCP1- | — | 0/12 | — |
| | nosterm | TobSSU | | | |
| pTP31 | nospro-nptll- | ASSU-CP2-TobSSU | 4/15 | 2/8 | 7/12 |
| | nosterm | | | | |
| pTP16 | nospro-nptll- | ASSU-a/sCP2- | — | 0/12 | — |
| | nosterm | TobSSU | | | |
| pKYLX35S²: | nospro-nptll- | d35S-CP1-rbcS | 2/15 | 3/11 | — |
| AMV4 | nosterm | | | | |
| pBS5 | SCSV1-nptll- | ASSU-CP1-TobSSU | — | 3/13 | — |
| | SCSV3 | | | | |
| pBSS5 | SCSV1-nptll- | SCSV4-CP1-SCSV5 | — | 1/14 | — |
| | SCSV3 | | | | |
| Total | | | 9/47 | 23/152 | 25/66 |

35Spro, CaMV 35 promoter;
bar, protein coding region of the bar gene;
ocs, octopine synthase gene terminator;
35Sterm, CaMV 35S terminator;
CP, AMV coat protein gene in sense orientation;
nospro, nopaline synthase gene promoter;
nosterm, nopaline synthase gene terminator;
a/sCP, AMV coat protein gene in antisense orientation;
ASSU, A. thaliana SSU gene promoter;
TobSSU, tobacco SSU gene terminator;
nptll, protein-coding region of the neomycin phosphotransferase gene;
d35S, duplicated CaMV 35S promoter;
rbcS, pea rbcS-E9 terminator;
SCSV1, Sub-clover stunt virus region 1 promoter;
SCSV4, Sub-clover stunt virus region 4 promoter;
SCSV3, Sub-clover stunt virus region 3 terminator;
SCSV5, Sub-clover stunt virus region 5 terminator.

With regard to the susceptible non-transgenic or transgenic lines, the inoculated plants showed no or very few local lesions. Systemic symptoms were not first seen until 21 days post-infection (dpi). At about 4-5 weeks post-infection, there were definite mosaic symptoms on all infected plants, which progressed to maximum infection levels by 6-8 weeks post-infection. There were no recovery phenotypes even after 30 weeks post-infection.

With regard to the immune plants, which did not produce local lesions or systemic symptoms, no virus was detectable from any of the immune plants by ELISA or bioassay after inoculation, even when inoculated with 4-fold the virus concentration needed to achieve infection of the non-transgenic susceptible lines. These AMV-immune lines were phenotypically indistinguishable from the non-transgenic control plants and produced similar foliage yields under glasshouse conditions. More importantly, whilst susceptible lines of white clover suffered foliage yield losses of 25% as a consequence of infection, the immune lines did not exhibit any reduction in yield following infection.

Three independent T₀ lines (RA4.2, RA4.3, and RA7.1) of transgenic red clover transformed with the binary vector pKYLX71:35S²AMV4 cp all expressing the AMV coat protein gene and all containing a single TDNA copy were tested for resistance to AMV by mechanical inoculation. The results showed one line (RA4.2) was immune to AMV infection while the other two were susceptible (Table 11B).

TABLE 11B
Results of mechanical inoculation of
transgenic lines of red clover with AMV

| | PCR AMV Coat | 1^{st} assessment | 2^{nd} assessment |
|---|---|---|---|
| Plant code | Protein | (23/10/01) | (31/10/01) |
| Cowpea indicator | na | 310 lesions | 310 lesions |
| Renegade | na | +(3/3) | +(3/3) |
| C1.10(control) | − | +(1/1) | +(1/1) |
| C1.18(control) | − | +(1/1) | +(1/1) |
| RA4.2-T₀ | + | −(3/3) | −(3/3) |
| RA4.3-T₀ | + | −(1/1) | +(1/1) |
| RA7.1-T₀ | + | +(4/4) | +(4/4) |

1) Virus inoculum was derived from white clover infected with AMV isolate WC28
2) C1.10, and C1.18 are seed derived cv. Renegade plants
“+” = positive
“−” = negative

### EXAMPLE 3.3

Assessment of White Clover Plants for AMV Resistance Following Aphid Transmission

A transgenic AMV-resistant line of white clover, and a transgenic immune line of white clover, as determined in the preceding Example by mechanical inoculation of virus, were tested for resistance to AMV transmission by aphid vectors. Results indicated that the immune line remained immune to virus infection by aphid vectors, and that the resistant line exhibited resistance against AMV transmitted by aphid vectors, compared to the non-transgenic control line (Table 12).

TABLE 12
Resistance to AMV following aphid transmission of virus

| Week | | | | | | | | |
|---|---|---|---|---|---|---|---|---|
| after | | | | | | | | |
| releasing% | Aphid incidence | | | | % AMV infection | | | |
| aphids | S | NT | R | I | S | NT | R | I |
| | Experiment I | | | | | | | |
| 0 | 0 | 0 | 0 | 0 | 100 | 0 | 0 | 0 |
| 2 | 60 | 42 | 59 | 47 | — | — | — | — |
| 3 | 69 | 65 | 76 | 73 | — | — | — | — |
| 4 | 64 | 68 | 77 | 68 | 100 | 39 | 4 | 0 |
| 5 | — | — | — | — | 100 | 75 | 7 | 0 |
| 6 | — | — | — | — | 100 | 93 | 68 | 0 |
| | Experiment II | | | | | | | |
| 0 | 0 | 0 | 0 | 0 | 100 | 0 | 0 | 0 |
| 2 | 21 | 5 | 19 | 8 | — | — | — | — |
| 3 | 23 | 20 | 18 | 25 | — | — | — | — |
| 4 | 32 | 23 | 27 | 33 | 100 | 0 | 0 | 0 |
| 5 | — | — | — | — | 100 | 60 | 8 | 0 |
| 6 | — | — | — | — | 100 | 70 | 62 | 0 |

S, Source plants;
NT, Non-transgenic line;
R, resistant transgenic line as determined by mechanical inoculation;
I, Immune transgenic line as determined by mechanical inoculation

### EXAMPLE 3.4

### Assessment of White Clover Plants for CYVV Resistance by Mechanical Inoculation and Bioassay for Virus Infectivity

Transgenic white clover lines carrying the CYVV coat protein gene in the binary vector pBH1 were tested for resistance characteristics against CYVV following mechanical inoculation or aphid transmission of three isolates of CYVV.

Table 13 shows the results of a typical experiment for assessing resistance against CYVV isolate WC1, following mechanical inoculation of plants with the virus. After inoculation, local lesions comprising distinct necrotic spots were observed on the leaves of C. amaranticolor control plants at 7-10 days post-infection (Table 9). In white clover, infection of non-resistant non-transformed lines with CYVV produced systemic faint chlorotic spots on the leaves of non-transgenic plants about three weeks post-infection.

In contrast, some transgenic white clover plants expressing CYVV coat protein were observed to be resistant to CYVV, by virtue of their failure to exhibit detectable symptoms associated with CYVV infection and free from CYVV after bioassays on indicator hosts (Table 13). Those transgenic white clover lines that were immune to CYVV isolate WC1, following mechanical inoculation with virus, were also found to be immune to the CYVV isolates WC16 and WC18, when the latter isolates were transmitted by the aphid vectors, Aphis craccivora or Myzus persicae.

Table 14 summarises the results obtained with all white clover lines tested. In particular, lines BH1-4, BH1-11, BH1-12, and BH1-13 were immune to all isolates tested. No correlation was observed between the immunity of transgenic plants and the level of coat protein gene expression.

TABLE 13
Resistance of transgenic white clover lines carrying plasmid
pBH1 against CYVV strain WC1 at 49 days post-infection

| | Infection rate | Bioassay | % | Assess- |
|---|---|---|---|---|
| Line | and symptoms | results | infection | ment |
| C. amaranticolor | 5/5 | 5/5+ | 100 | S |
| Non-transformed | 6/6 Veinal and | 6/6+ | 100 | S |
| control line NT I5 | interveinal chlorosis | | | |
| BH1-14 | 2/6 very faint ch | 2/6+ | 33 | R |
| | spots | | | |
| BH1-17 | 3/6 Faint ch spots | 3/6+ | 50 | S |
| BH1-13 | 6/6 No symptoms | 0/6+ | 0 | I |
| BH1-19 | 2/6 Faint ch spots | 2/6+ | 30 | R |

S, Susceptible (greater than 50% infection);
R, Resistant (less than 50% infection);
I, Immune (no infection).

TABLE 14
Summary of resistance characteristics of non-transformed
(NT) white clover and transformed lines carrying
the binary vector pBH1

| | Mechanical infection | | | | |
|---|---|---|---|---|---|
| | CYVV WC1 | | Aphid transmission | | |
| | Systemic | Bioassay | CYVV | CYVV | |
| Lines | Symptoms | Result | WC16 | WC18 | Assessment |
| NT I2 | 83% | 83% | 100% | 75% | S |
| NT I4 | 14% | 14% | 50% | 80% | S |
| NT I5 | 100% | 100% | 100% | 100% | S |
| BH1-1 | 100% | 100% | ND | ND | S |
| BH1-2 | 100% | 100% | ND | ND | S |
| BH1-3 | 100% | 100% | ND | ND | S |
| BH1-4 | 0% | 0% | 0% | 0% | I |
| BH1-6 | 100% | 100% | ND | ND | S |
| BH1-9 | 100% | 100% | ND | ND | S |
| BH1-10 | 100% | 100% | ND | ND | S |
| BH1-11 | 0% | 0% | 0% | 0% | I |
| BH1-12 | 0% | 0% | 0% | 0% | I |
| BH1-13 | 0% | 0% | 0% | 0% | I |
| BH1-15 | 40% | 80% | ND | ND | S |

S, Susceptible (greater than 50% infection);
R, Resistant (less than 50% infection);
I, Immune (no infection)
ND, not tested.

### EXAMPLE 3.5

### Assessment of White and Red Clover Plants for WCMV Resistance by Mechanical Inoculation and Bioassay for Virus Infectivity

Transgenic white clover lines carrying the binary vector pKYLX71:35S²wcm4 cp (hereinafter “4S lines”) were tested by mechanical inoculation against three isolates of the virus. At 7-10 days post-infection, numerous local WCMV-induced lesions, comprising distinct yellow-green chlorotic spots, were observed on the cotyledons of the cowpea indicator plants (Table 9). In a typical experiment (Table 15), infected non-resistant non-transformed white clover lines exhibited systemic faint veinal chlorotic lesions on the leaves about two to three weeks post-infection.

In contrast, transgenic plants that were resistant to WCMV have no detectable symptoms. As shown in Table 15, line 4S8 was asymptomatic following mechanical inoculation, however produced a positive bioassay result, indicating that infection had occurred. Accordingly, this line was designated as resistant. Line 4S30 failed to exhibit symptoms following mechanical inoculation with WCMV and did not test positive after bioassays on the Cowpea indicator host in one experiment (Table 15) but in another, like 4S8, produced a positive bioassay result (Table 16), and was also designated as a resistant rather than an immune line.

Independent T₀ lines of red clover transformed with the binary vector pKYLX71:35S²wcm4 cp containing WCMV coat protein gene all expressing the WCMV coat protein gene (Table 7B) were tested for resistance to WCMV by mechanical inoculation. The results showed no immune or resistant plants were detected at 42 days post-infection.

TABLE 15
Resistance of non-transformed (NT) plants and transgenic
white clover lines carrying plasmid pKYLX71:35S²wcm4cp
against WCMV strain WC16

| | Infection rate | Bioassay | % | |
|---|---|---|---|---|
| Lines | and symptoms | results | infection | Assessment |
| Cowpea | 5/5 Local lesions | 5/5+ | 100 | S |
| indicator | | | | |
| NT I2 | 4/6 Faint interveinal | 4/6+ | 67 | S |
| | and veinal chlorosis | | | |
| NT I4 | 6/6 Faint interveinal | 6/6+ | 100 | S |
| | and veinal chlorosis | | | |
| NT I5 | 6/6 Faint interveinal | 6/6+ | 100 | S |
| | and veinal chlorosis | | | |
| 4S2 | 6/6 Faint interveinal | 6/6+ | 100 | S |
| | and veinal chlorosis | | | |
| 4S8 | 6/6 No symptoms or | 6/6+ | 100 | R |
| | very faint chlorosis | | | |
| 4S30 | 6/6 No symptoms | 0/6+ | 0 | I |

S, Susceptible (greater than 50% infection);
R, Resistant (less than 50% infection);
I, Immune (no infection).

Table 16 summarises the characterisation of all the WCMV coat protein transgenic white clover lines tested. No correlation between virus resistance and immunity and level of WCMV coat protein gene expression was observed.

TABLE 16
Summary of resistance characteristics of non-
transformed (NT) white clover and transformed lines
carrying the binary vector pKYLX71:35S²wcmv4cp

| | | | |
|---|---|---|---|
| | | Infection Confirmed | |
| Lines | % with symptoms | by Bioassay | Assessment |

| | | | |
|---|---|---|---|
| NT I2 | 60% | Yes | S |
| NT I4 | 100% | Yes | S |
| NT I5 | 100% | Yes | S |
| NT IC1 | 65% | Yes | S |
| 4S2 | 100% | Yes | S |
| 4S4 | 50% | Yes | S |
| 4S8 | 0% | Yes | R |
| 4S10 | 50% | Yes | S |
| 4S11 | 50% | Yes | S |
| 4S30 | 0% | Yes | R |
| 4S55 | 100% | Yes | S |

S, Susceptible (greater than 50% infection);
R, Resistant (less than 50% infection);

### EXAMPLE 3.6

Assessment of M. sativa (lucerne) Plants for AMV Resistance by Mechanical Inoculation and Bioassay for Virus Infection

A total of 119 confirmed independent transgenic lines of M. sativa cv. Siriver and M. sativa cv. Aquarius, transformed with 10 different binary vector constructs, were tested for resistance to AMV by mechanical inoculation. Data presented in Table 17 indicate that a greater proportion of immune transgenic lines were produced using binary vectors comprising the A. thaliana SSU promoter to regulate coat protein gene expression, compared to the CaMV 35S promoter.

Data for a representative experiment are presented in Table 18. In particular, lines 15, 16, and 30 carrying the binary vector pTP5 were shown to be immune to AMV in the bioassay, whilst lines 8 and 24 were shown to be resistant to AMV infection.

Of the 119 transformed lines generated, 29 lines (approximately 20%) were shown to have immunity against both subgroups I and II AMV isolates (i.e. they were asymptomatic and tested negative in the bioassay), while another 21 lines (about 17%) were shown to be resistant (i.e. they exhibited less than 50% infection as determined by bioassay). None of the 16 non-transgenic lines tested showed any degree of resistance (i.e. all lines produced over 75% infection in standard bioassay).

TABLE 17
Summary of AMV immune transgenic lucerne
lines derived from various constructs

| | | | Proportion of |
|---|---|---|---|
| Binary | Selectable Marker | | immune |
| Vector | Gene | Resistance gene insert | lines |
| NT | None | None | 0/16 |
| pT17 | 35Spro-bar-OCS | 35Spro-CP1-35Ster | 2/26 |
| pTW5 | 35Spro-bar-OCS | ASSU-CP1-TobSSU | 15/24 |
| PTW12 | 35Spro-bar-OCS | ASSU-a/sCP1-TobSSU | 0/24 |
| pTP5 | Nospro-nptll-noster | ASSU-CP1-TobSSU | 3/12 |
| pBS5 | SCSV1-nptll-SCSV3 | ASSU-CP1-TobSSU | 6/20 |
| PBS31 | SCSV1-nptll-SCSV3 | ASSU-CP2-TobSSU | 3/13 |
| Total | | | 29/119 |

35Spro, CaMV 35 promoter;
bar, protein coding region of the bar gene;
ocs, octopine synthase gene terminator;
35Ster, CaMV 35S terminator;
CP, AMV coat protein gene in sense orientation;
nospro, nopaline synthase gene promoter;
noster, nopaline synthase gene terminator;
a/sCP, AMV coat protein gene in antisense orientation;
ASSU, A. thaliana SSU gene promoter;
TobSSU, tobacco SSU gene terminator;
nptll, protein-coding region of the neomycin phosphotransferase gene;
SCSV1, Sub-clover stunt virus region 1 promoter;
SCSV3, Sub-clover stunt virus region 3 terminator.

In general, the lucerne cultivars were generally less susceptible to AMV than white clover. AMV-related symptoms were also more variable on lucerne than on white clover plants, varying as widely between different genotypes of the same cultivar as between cultivars. Frequently, the symptoms of AMV infection on lucerne also exhibited a cyclic amelioration. The inoculated lucerne plants showed no or very few local lesions and systemic symptoms were usually milder and usually took longer to develop than for white clover, not becoming visible until 5-6 weeks post-inoculation. There were no recovery phenotypes even after 30 weeks post-infection.

As with white clover, no virus was detectable from any of the immune plants by ELISA or bioassays after inoculation. These AMV-immune lines were phenotypically indistinguishable from the non-transgenic controls and produced similar foliage yields under glasshouse conditions. The AMV-immune lines were unaffected while susceptible lines suffered foliage yield loss of about 20% after AMV infection.

TABLE 18
Summary of resistance characteristics of non-transformed
(NT) lucerne and transformed lines carrying the binary
vector pTP5 against AMV

| Plant | Infection rates | | | % mean | |
|---|---|---|---|---|---|
| Lines | 50 ug/ml | 100 ug/ml | 200 ug/ml | infection | Assessment |
| NT 2 | 2/6 | 4/6 | 5/6 | 61% | S |
| NT 7 | 3/6 | 4/6 | 6/6 | 72% | S |
| NT 13 | 5/6 | 5/6 | 6/6 | 89% | S |
| NT-R1 | 2/6 | 4/6 | 6/6 | 67% | S |
| NT-R2 | 2/6 | 5/6 | 6/6 | 72% | S |
| pTP5-3 | ¾ | ¾ | ¾ | 75% | S |
| pTP5-4 | 4/4 | 4/4 | ¾ | 92% | S |
| pTP5-8 | 2/4 | ¼ | 0/4 | 25% | R |
| pTP5-9 | ¾ | ¾ | 4/4 | 83% | S |
| pTP5-11 | 4/4 | ¼ | ¼ | 50% | S |
| pTP5-15 | 0/4 | 0/4 | 0/4 | 0% | I |
| pTP5-16 | 0/4 | 0/4 | 0/4 | 0% | I |
| pTP5-19 | 4/4 | ¾ | 4/4 | 92% | S |
| pTP5-24 | ¼ | 0/4 | 0/4 | 8% | R |
| pTP5-30 | 0/4 | 0/4 | 0/4 | 0% | I |
| pTP5-54 | 4/4 | 2/4 | 4/4 | 83% | S |
| pTP5-55 | 2/4 | 4/4 | ¾ | 75% | S |

NT, non-transgenic lines from seedlings (NT2, NT7, NT13) or by regeneration in transformation experiment (NT-R1, NT-R2);
S, Susceptible (greater than 50% infection);
R, Resistant (less than 50% infection);
I, Immune (no infection).

### EXAMPLE 3.7

### Assessment of Lucerne Plants for AMV Resistance Following Aphid Transmission

The transgenic immune lines of M. sativa, as determined in the preceding Example by mechanical inoculation of virus and bioassay, were tested for resistance to AMV transmission by aphid vectors. Results indicated that the immune line remained immune to virus infection by aphid vectors, and that the resistant line exhibited resistance against AMV transmitted by aphid vectors, compared to the non-transgenic control line (Table 18B).

TABLE 18B
Summary of resistance transformed lucerne lines
carrying the binary vector pTP5 against AMV strain
YC1.2 by aphid transmission

| | % Infection by | |
|---|---|---|
| Plant | Mechanical | |
| Lines | Inoculation | % Infection by Aphid transmission |
| NT 2 | 61% | 50% |
| NT 7 | 72% | 45% |
| NT 13 | 89% | 70% |
| pTP5-4 | 92% | 60% |
| pTP5-8 | 25% | 15% |
| pTP5-15 | 0% | 0% |
| PTP5-16 | 0% | 0% |
| pTP5-30 | 0% | 0% |

NT, non-transgenic lines from seedlings (NT2, NT7, NT13)

### EXAMPLE 3.8

Assessment of Subterranean Clover Plants for AMV Resistance by Mechanical Inoculation

Transgenic subterranean clover plants were screened for virus resistance essentially as described for BYMV by Chu et al. (1999). Transformed T₀ lines were self-fertilised and allowed to set seed. T₁ seedlings were screened for transgene expression by spraying with Basta (0.4 g/L PPT) at the two-leaf stage. In this test, all control non-transgenic seedlings were killed. The resultant Basta resistant transgenic T₁ plants were clonally propagated from auxiliary shoots prior to stolon elongation and flowering. Five vegetatively propagated cuttings were made for each line for each inoculum level, plus two non-inoculated controls. The cuttings were allowed to grow to a good size, for about 6-7 weeks, in the glasshouse before being inoculated with virus. Representative non-transgenic lines were also tested as controls.

AMV strain WC28 was used for mechanical inoculation as described above for the transgenic white clover plants. Virus inoculum was prepared by extracting sap from AMV infected white clover plants using a sap extractor at a dilution level of 1:5 of leaf materials to 100 mM phosphate buffer, pH 7.4 (W/V). The virus inoculum contained 1% carborundum. Plants were cut back to one to two shoots with 4-5 fully expanded leaves and were kept in the dark for up to 24 hrs before inoculation with ice-cold virus inoculum. Each plant was inoculated with 0.4 ml of inoculum by rubbing the inoculum onto the upper surface of each leaflet of 4 youngest fully expanded leaves, 5 times. Under these conditions infection of non-transformed control plants reached over 80% of inoculated plants in each trial.

After inoculation, local lesions on co-inoculated local lesion control plants were counted. Symptoms on the subterranean clover plants were assessed at 3, 5 and 8 weeks p.i. AMV immunity was confirmed by bioassay on cowpeas. Virus accumulation in inoculated plants was estimated by double-sandwich ELISA detection of virus in leaf samples, as described by Clark and Adams (1977).

A T₁ progeny plant most resistant to Basta from each of the two AMV coat protein transgenic subterranean clover lines was tested for virus resistance using non-transgenic lines as controls. Typical symptoms of AMV infection appeared on the control non-transgenic plants at about 3 weeks pi and consisted of mild systemic interveinal chlorosis on young and mature leaves. However, none of the two transgenic lines became infected with AMV even at 8 weeks pi as confirmed by bioassays on cowpeas and lack of symptoms (Table 19). These lines were used as the source parents for crossing with the SCSV resistant subclover lines (see below) to produce multiple virus resistant plants.

TABLE 19
Evaluation of AMV resistance in transgenic
subterranean clover lines by mechanical inoculation

| | AMV Infection | |
|---|---|---|
| Subterranean Clover Line | After Challenge | Results |
| Gosse Non-transgenic line 1 | 4/5 | Susceptible |
| Gosse Non-transgenic line 2 | 5/5 | Susceptible |
| Gosse Non-transgenic line 3 | 4/5 | Susceptible |
| Gosse Control Regenerant line 1 | 5/5 | Susceptible |
| Gosse Control Regenerant line 2 | 5/5 | Susceptible |
| Gosse Control Regenerant line 3 | 4/5 | Susceptible |
| AMV CP transgenic line 1 | 0/5 | Immune |
| AMV CP transgenic line 2 | 0/5 | Immune |

### EXAMPLE 3.9

Assessment of Subterranean Clover Plants for SCSV Resistance by Aphid Transmission Tests

Aphid transmission tests were done in PC2 cold frames using vegetatively propagated clonal subterranean clover plants prepared as described above for mechanical inoculation. SCSV-infected subterranean clover plants, prepared with newly isolated SCSV from the field, were used as the virus source. The aphid vector, Aphis craccivora, was applied to the source plants and allowed to spread onto the test plants over a period of 2 weeks after which the plants were sprayed with a pyrethrin insecticide to remove the aphids. Subterranean clover seedlings cv. Mt. Barker, a susceptible variety of subterranean clover, was used as the positive control.

SCSV infection was readily identified by symptom development and confirmed by ELISA. Virus accumulation in inoculated plants was estimated by double-sandwich ELISA detection of virus in leaf samples, as described by Clark and Adams (1977). Antibody reagents for SCSV detection were prepared from rabbit polyclonal antisera against the virus.

Only the T₁ transgenic subterranean clover lines that survived the Basta screen were assessed for SCSV resistance. After aphid transmission, plants were assessed by symptoms at 5 and 8 weeks post-inoculation. The relative levels of resistance were based on percentage of infected plants, severity of symptoms and disease recovery, if present. Typical severe stunting symptoms of SCSV developed in the control subterranean clover plants, cvs Mt. Barker and Gosse about 3 weeks pi. and became fully infected by 5 weeks pi.

Marked differences in infectivity and/or symptom severity were evident between the highly resistant, moderately resistant and susceptible transgenic subclover lines (Table 20A). In the highly resistant lines (SC4 and SC19) up to 80% of the inoculated replicates were completely symptomless. The remaining infected replicates showed either mild symptoms or a delayed development of systemic symptoms compared with the non-transgenic control plants. In the moderately resistant line SC16, the infected replicates developed mainly mild systemic symptoms. The susceptible transgenic lines developed viral symptoms that were indistinguishable from the controls. No recovery from disease symptoms was observed in any of the lines. The best resistant progenies derived from the lines SC19 and SC 4 were used as the source parents for crossing with the AMV resistant subterranean clover lines to produce AMV plus SCSV double virus resistant plants.

TABLE 20A
Summary of SCSV Coat Protein Transgenic Challenge

| Lines | BastaR | Northern | Infection | Severity | Final |
|---|---|---|---|---|---|
| Mt. Barker | No | − | 30/32 (94%) | Severe | Susceptible |
| NT | No | − | 12/13 (92%) | Severe | Susceptible |
| Reg 1 | No | − | 12/16 (75%) | Mod-Severe | Susceptible |
| Reg 2 | No | − | 10/15 (67%) | Mod-Severe | Susceptible |
| Reg 2 | No | − | 17/19 (89%) | Severe | Susceptible |
| SC1 | 0.4 g/L | + | 5/5 (100%) | Severe | Susceptible |
| SC4 | 0.4 g/L | + | 3/9 (33%) | Mild | Resistant |
| SC6 | 0.4 g/L | − | 5/5 (100%) | Moderate | Susceptible |
| SC9 | 0.4 g/L | + | 7/7 (100%) | Moderate | Susceptible |
| SC14 | 0.4 g/L | ++++ | 37/39 (95%) | Mod-Severe | Susceptible |
| SC16 | 0.4 g/L | + | 16/26 (62%) | Mild-Mod | Mod resistant (mild) |
| SC19 | 0.4 g/L | ++ | 4/20 (20%) | Mild-Mod | Resistant |

### EXAMPLE 3.10

White Clover Transformed with the Binary Vector pBH2 Containing a CYVV Coat Protein Gene in a Sense/Antisense Inverted rep at (Hairpin RNA)

This example shows the production of white clover plants transformed with a construct pBH2 containing a CYVV sense/antisense inverted repeat derived from the coat protein gene (FIG. 18B). The inverted repeat is composed of the sense sequence of the CYVV coat protein gene from nucleotides 1-820 and the antisense sequence from nucleotides 1-530 ligated at the sail and EcoRI sites.

Eleven putative transgenic lines were challenged with CYVV WC1 and WC18. The results, shown in Table 20B, produced two CYVV immune lines (pBH2-10 and pBH2-12).

TABLE 20B
Summary of White Clover pBH2 (CYVV
CP inverted repeat + hph) Transformants

| | Lines | CYVV Infection | Final Results |
|---|---|---|---|
| Non-Transgenic | NT I2 | 4/5 | CYVV S |
| Control Lines | NT I5 | 5/5 | CYVV S |
| BH2 | BH2/2a | 6/6 | CYVV S |
| Transformed | BH2/2b | 6/6 | CYVV S |
| Lines | BH2/3 | 6/6 | CYVV S |
| | BH2/4 | 6/6 | CYVV S |
| | BH2/5 | 6/6 | CYVV S |
| | BH2/6 | 6/6 | CYVV S |
| | BH2/7 | 6/6 | CYVV S |
| | BH2/8 | 6/6 | CYVV S |
| | BH2/9 | 6/6 | CYVV S |
| | BH2/10 | 0/6 | CYVV I |
| | BH2/12 | 0/6 | CYVV I |

S, Susceptible (greater than 50% infection);
R, Resistant (less than 50% infection);
I, Immune (no infection).

Part IV of the Experimental Section:

Molecular Analysis of Virus-resistance in Transgenic Plant Lin s

### EXAMPLE 4.1

### Molecular Analysis of AMV Resistance in Transgenic White Clover, Red Clover and Lucerne Plants

Factors affecting the production of AMV-immune transgenic white clover plants were further analysed on the basis of cultivar, gene construct (promoter) used, and source and orientation of the coat protein gene.

There was no significant difference in the proportion of AMV immune lines obtained when different cultivars were transformed.

However, we noted a significant effect of the promoter used to control coat protein gene expression. A significantly higher proportion of immune lines were obtained from a particular transformation experiment when the A. thaliana SSU gene promoter was used, compared to the proportion of immune lines generated using the CaMV 35S promoter or SCSV promoter sequences. Binary vectors comprising the A. thaliana SSU gene promoter produced at least 4-fold more immune white clover lines, and 7-fold more immune lucerne lines, than did those binary vectors comprising the CaMV 35S promoter or the SCSV4 promoter (Table 21).

TABLE 21
Effect of different promoters on the
production of transformed white clover
and lucerne plants that are immune to AMV

Promoter and proportion
of immune lines

| Plant | 35S | ASSU | SCSV4 |
|---|---|---|---|
| White Clover | 1/13 (7%) | 15/49 (31%) | 1/14 (7%) |
| Lucerne | 2/26 (7%) | 18/36 (50%) | |

35S, CaMV 35 promoter;
ASSU, A. thaliana SSU gene promoter;
SCSV4, Sub-clover stunt virus region 4 promoter.

A comparison of the three coat protein genes used from the two AMV subgroups showed that all were equally as effective in producing immune lines of white clover, red clover and lucerne (Table 22). In conjunction with the A. thaliana SSU gene promoter, the AMV coat protein genes derived from isolates H1 (sub-group I) or YD3.2 (sub-group II) routinely produced 20-30% immune plants (Table 22).

TABLE 22
Effect of different sources of AMV coat
protein gene on the production of transformed
pasture legume plants that are immune to AMV
as indicated using lucerne, red and white clover.

| | | | Proportion |
|---|---|---|---|
| | | | immune |
| | AMV isolate for Coat | | lines |
| Binary vector | Protein Gene | Promoter | produced |
| PKYLX71:35S²AMV4 | WC3 (Subgroup I) | d35S | 3/18 |
| | | | (˜16%) |
| pTP5 | H1 (Subgroup I) | ASSU | 10/45 |
| | | | (˜20%) |
| pTP31 | YD3.2 (Subgroup II) | ASSU | 9/43 |
| | | | (˜20%) |
| pBS5 | H1 (Subgroup I) | ASSU | 6/20 |
| | | | (30%) |
| pBS31 | YD3.2 (Subgroup II) | ASSU | 3/13 |
| | | | (˜23%) |

Comparison of the orientation of the same coat protein gene using the same selectable marker also showed that only the virus-sense construct produced AMV immune lines although the anti-sense construct did produce resistant lines (Table 23).

TABLE 23
Effect of sense orientation of AMV coat protein gene on
the production of AMV immune transgenic white clover

| | | | Proportion | |
|---|---|---|---|---|
| | | Orientation | of | Proportion of |
| | | of coat | immune | resistant |
| Construct | Promoter | protein ORF | lines | lines |
| NT white clover | None | None | 0/19 | 0/19 |
| pTW5 white clover | ASSU | Sense | 15/49 | 7/49 |
| pTW12 white | ASSU | Antisense | 0/52 | 7/52 |
| clover | | | | |
| NT lucerne | None | None | 0/16 | 0/16 |
| pTW5 lucerne | ASSU | Sense | 15/24 | 3/24 |
| pTW12 lucerne | ASSU | Antisense | 0/24 | 3/24 |

To determine the molecular basis of AMV resistance (not necessarily immunity) in the transgenic white clover and lucerne, representative immune, resistant and susceptible transgenic lines were analysed by Southern, Northern and Western blots. Our data indicate that, for those gene constructs utilising the A. thaliana SSU promoter to regulate expression of the AMV coat protein gene, those transgenic lines having the highest levels of coat protein expression were more likely to be resistant or immune against AMV, as indicated by the results of northern and western blotting. In contrast, transgene copy number was not a factor in conferring resistance or immunity on plants. (Table 24). On the other hand, testing lines with different level of AMV coat protein mRNA transcript showed that immune white clover lines were mainly obtained from plants expressing high levels of coat protein-encoding mRNA (data not shown). Thus, whilst the primary consideration for enhancing the number of immune plants in a transformation experiment was the use of the A. thaliana SSU promoter or the duplicated CaMV 35S promoter, those transformed lines which expressed the coat protein under control of the A. thaliana SSU promoter, at the highest level were more likely to be resistant or immune against AMV.

TABLE 24
Correlation of AMV Resistance to Coat Protein Transgene
Expression in plants carrying the vectors pTW5 and pBS5.

| | | | | | |
|---|---|---|---|---|---|
| | | | | | Transgene |
| | | Response | Northern | Western | Copies |
| Constructs | Plant Lines | to AMV | Signal | Signal | (Southern) |

Lucerne:

| | | | | | |
|---|---|---|---|---|---|
| Non- | NT 7 | S | − | ND | ND |
| Transgenic | NT13 | S | − | ND | ND |
| PBS5 | 2 | I | ++++ | ND | ND |
| | 3 | I | +++ | ND | ND |
| | 8 | I | ++++ | ND | ND |
| | 13 | I | +++++ | ND | ND |
| | 15 | I | +++++ | ND | ND |
| | 20 | I | +++ | ND | ND |
| | 4 | R | +++ | ND | ND |
| | 14 | R | ++ | ND | ND |
| | 19 | R | ++ | ND | ND |
| | 1 | S | + | ND | ND |
| | 5 | S | − | ND | ND |
| | 6 | S | + | ND | ND |
| | 7 | S | − | ND | ND |
| | 9 | S | − | ND | ND |
| | 10 | S | − | ND | ND |
| | 11 | S | − | ND | ND |
| | 12 | S | + | ND | ND |
| | 16 | S | − | ND | ND |
| | 17 | S | − | ND | ND |
| | 180 | S | − | ND | ND |

White Clover:

| | | | | | |
|---|---|---|---|---|---|
| Non- | H9 | S | − | − | 0 |
| Transgenic | H12 | S | − | − | 0 |
| PTW5 | 208 | I | +++ | +++ | 1 |
| | 148 | I | ++++ | +++ | 4 |
| | 144 | I | +++ | +++ | 1 |
| | 135 | R | +++ | + | 4 |
| | 109 | S | − | + | ND |
| | 130 | S | + | + | 1 |
| | 134 | S | − | + | 8 |
| | 142 | S | − | − | ND |
| | 122 | S | − | − | ND |
| | 123 | S | − | − | ND |

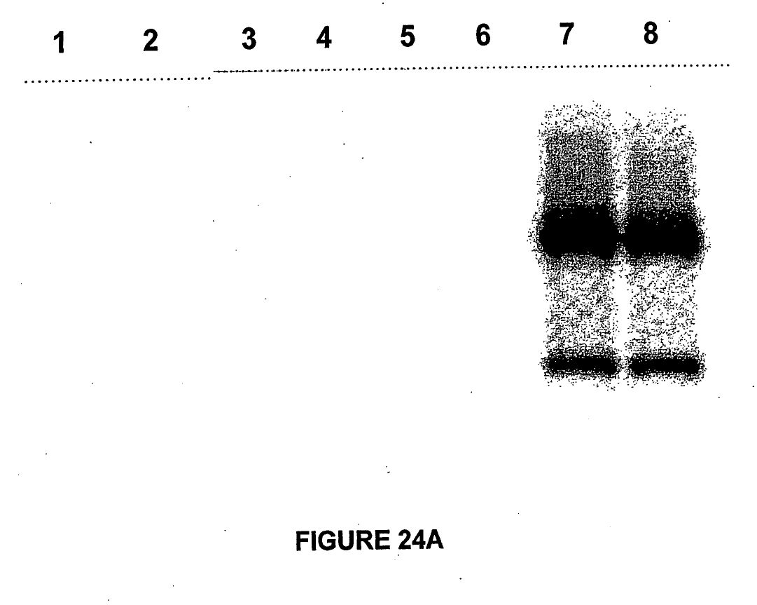

Additional experiments indicated that there was no detectable degradation of mRNA encoding the AMV coat protein in immune lines following inoculation with virus (FIG. 24A, FIG. 24B, and Table 25), and that there was no symptom recovery or induced-resistance after inoculation. These results collectively indicated that resistance against AMV was not conferred by RNA-mediated gene silencing (Lindbro et al., 1993; Dougherty et al., 1994).

TABLE 25
Effect of AMV inoculation on level of plant mRNA encoding
AMV coat protein in susceptible and immune lines
of white clover carrying the binary vector pTP5

| | | MRNA level | mRNA level after |
|---|---|---|---|
| Constructs | Line | before inoculation | inoculation |
| None | H9 | − | − |
| pTP5 | 446 (S) | + | + |
| pTP5 | 447 (I) | ++++ | ++++ |

### EXAMPLE 4.2

### Molecular Analysis of CYVV Resistance in Transgenic White Clover Plants

The CYVV coat protein gene construct pBH1, comprising the A. thaliana SSU gene promoter driving expression of the CYVV coat protein gene, was highly-effective in producing CYVV immune white clover plants. In these plants, there was no correlation between coat protein transgene copy number or gene expression and the acquisition of the virus resistance phenotype, in marked contrast to our observations for the acquisition of AMV coat protein-mediated resistance in white clover.

### EXAMPLE 4.3

### Molecular Analysis of WCMV Resistance in Transgenic White Clover Plants

The WCMV coat protein gene construct pKYLX35S²wcmv4, comprising the duplicated CaMV 35S gene promoter driving expression of the WCMV coat protein gene, was effective in producing WCMV resistant white clover plants. In these plants, there was no correlation between coat protein transgene copy number or gene expression and the acquisition of the virus resistance phenotype, in marked contrast to our observations for the acquisition of AMV coat protein-mediated resistance in white clover.

Jayasena et al (2001) suggested that when the CaMV 35S promoter was used, there was no correlation between the level of protein accumulation and virus resistance and is consistent with both AMV, RNA and protein being involved (Yusibov and Loesch-Fries, 1995). Also, their results showed that the resistance is not stably inherited in all the transgenic progenies. In contrast, in the present invention, when the ASSU promoter was used, there was a direct correlation between coat protein level and virus resistance which was 100% inherited in all transgenic offsprings.

Part V of the Experimental Section:

Field Trials of Virus-Resistant Transgenic Plants

### EXAMPLE 5.1

### Field Trial of Primary Transgenic (T₀) Virus-Resistant White Clover Lines (GMAC Planned Release PR64/67)

It may not be assumed that resistance to virus infection by sap inoculation under glasshouse conditions necessarily leads to resistance to natural infection in the field. Accordingly, application was made to GMAC for permission to carry out field trials of primary transgenic (i.e. T₀ lines) white clover plants at a strategic site over a two year period. Growth, disease incidence and persistence of the transformed plants, and the potential for spread of the recombinant coat protein genes into adjacent white clover trap plants, were assessed.

The five AMV-immune transformed lines and single AMV-resistant transformed line were tested in the field over a two year period, using the two non-transgenic lines as controls.

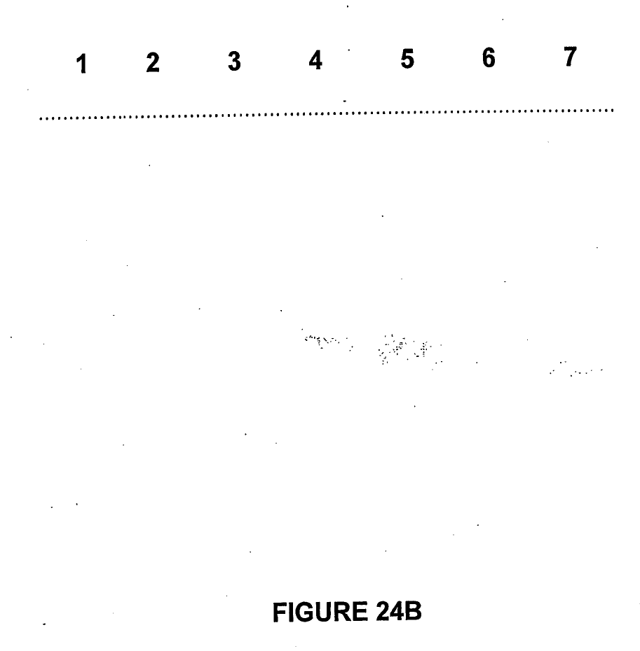

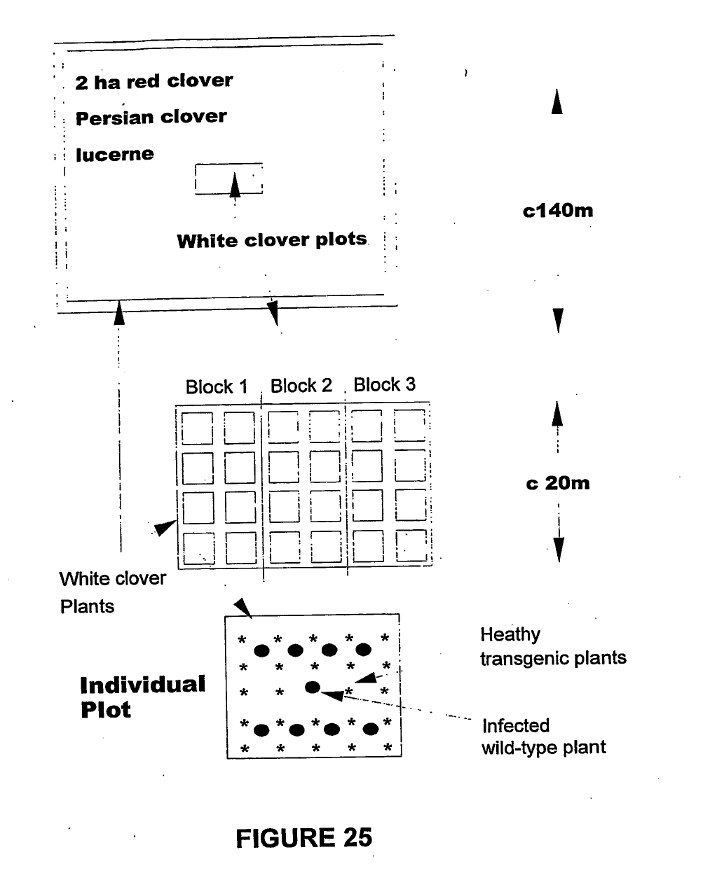

In particular, a randomised block trial of 8 lines of T. repens cv. Haifa and T. repens cv.Irrigation comprising 6 transformed lines carrying the AMV coat protein and exhibiting resistance (1 line) or immunity (5 lines) against AMV under glasshouse conditions, and 2 wild-type lines, was established. The plants used in the trial are shown in Table 26. The plants were grown as 3 replicate blocks of the 8 lines, in the centre of a 2 ha field having a perimeter comprising a mixture of non-transformed red clover (T. pretense), Persian clover (T. resupinatum) and luceme (M. saliva), depicted in FIG. 25. For each transformed or non-transformed line, a total of 72 vegetatively-propagated cuttings were planted within the 3 replicate blocks, spaced 50 cm apart, in 5 m square plots. Each plot consisted of 25 plants in a 5×5 array having 24 clones of each line, surrounding a central cutting of AMV-infected wild-type white clover. An extra 8 AMV-infected source plants were placed between the two top rows and two bottom rows of test plants (FIG. 25). In addition, two rows of non-transgenic white clover were sown, one immediately surrounding the transformed lines, and the other surrounding the entire 2 ha field, about 1 m inside the fence, to facilitate an assessment of the spread of the recombinant AMV coat protein-encoding gene into wild type populations.

To assist the spread of AMV throughout the field trial, two species of aphids (A. craccivora and M. persicae) were released at monthly intervals during the spring growing season (i.e. between August and October).

Plants were evaluated monthly for the occurrence and/or spread of AMV infection, by standard bioassay on indicator host plants as described herein above (i.e by sap inoculation to cowpea and Chenopodium), and by molecular analyses to determine AMV coat protein gene expression. Growth characteristics of individual plants were assessed each season. Seed were also collected from plants grown in the trap rows, and a combination of PCR screening and antibiotic selection on G418 were carried out on the progeny plants, to determine whether or not the recombinant genes had flowed from the central plots into the surrounding non-transformed plants of the perimeter.

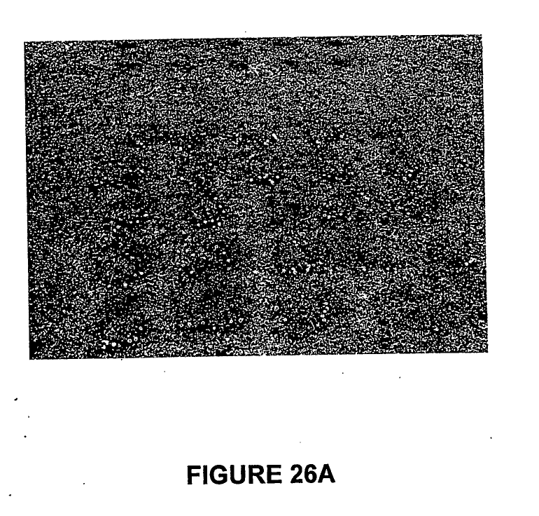

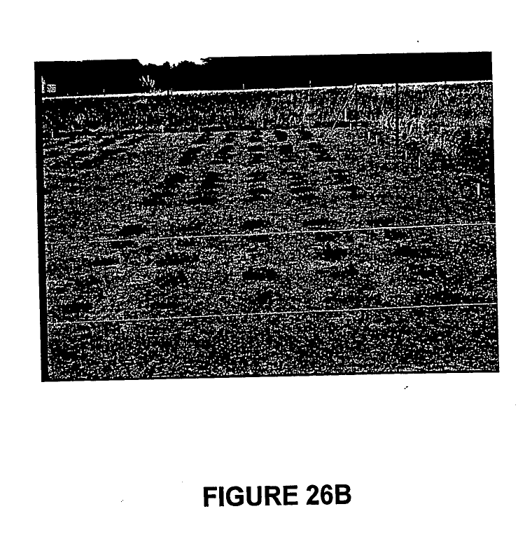

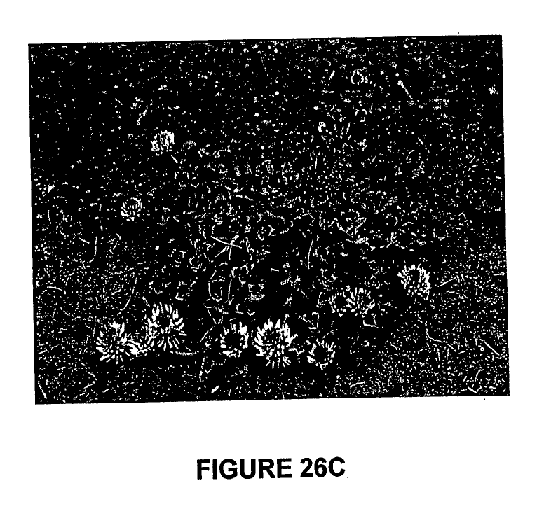

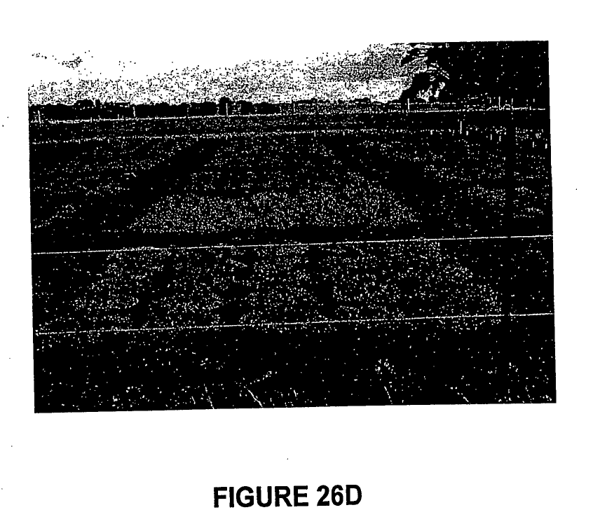

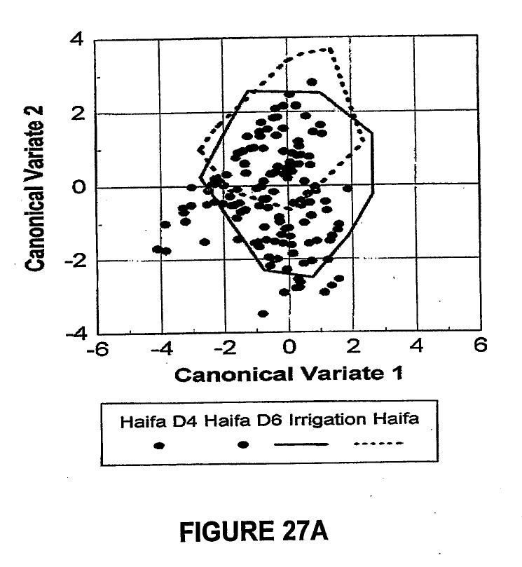

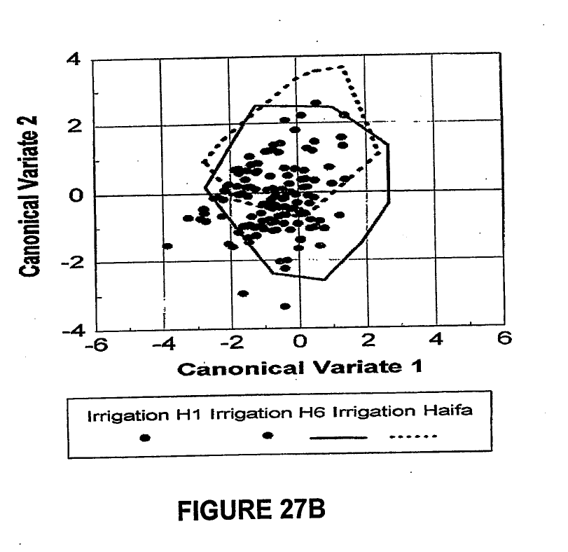

Data shown in FIG. 26 and FIG. 27 show the diameter, height, stolon density at plant center, stolon density at plant edge, leaf colour, form of leaf, and flower number of transformed and non-transformed plants in the trial. These data indicate that there are no detectable phenotypic differences between the transformed and non-transformed lines.

TABLE 26
White clover lines tested in the field trial of T₀ plants
carrying the AMV coat protein-encoding gene

| | | | WC28 |
|---|---|---|---|
| | Independent | % infection in | phenotype |
| Construct | line | glasshouse tests | in glasshouse |
| Non Transgenic | C1 | 83 | Susceptible |
| Non Transgenic | C2 | 92 | Susceptible |
| pTP5 | TWC 451 | 8 | Resistant |
| pTP5 | TWC 447 | 0 | Immune |
| pKYLX71:35S²:AMV4 | H1 | 0 | Immune |
| pKYLX71:35S²:AMV4 | H6 | 0 | Immune |
| pKYLX71:35S²:AMV4 | D2 | 0 | Immune |
| pKYLX71:35S²:AMV4 | D6 | 0 | Immune |

Northern analysis to detect recombinant mRNA encoding the AMV coat protein in both uninfected transformed and non-transformed lines grown in the field trial showed the presence of such mRNA only in the transformed lines. Additionally, in these northern hybridisations, AMV-infected plants and uninfected transformed plants are distinguished by the intensity and pattern of the signal corresponding to the coat protein-encoding mRNA of AMV. In particular, uninfected transgenic plants expressing the mRNA of the transformed coat protein gene produce a much weaker single band than infected plants, and, in contrast to this, infected plants produce two strong viral RNA bands corresponding to the viral RNA3 and RNA4. The recombinant AMV coat protein-encoding mRNA of non-infected transformed plants also has a different mobility on RNA gels to the AMV RNA4 present in virus-infected plants, because of the additional CaMV 35S 5′-UTR sequences present in the recombinant coat protein gene. All plants testing positive for AMV viral RNA3 and RNA4 in northern hybridisations were also scored positive for AMV symptoms and bioassay. Whilst symptoms developed on the non-transformed plants that produced a positive signal in northern hybridisations, no symptoms or positive bioassay result was returned for uninfected plants that showed only the expression of the recombinant AMV RNA4.

Furthermore, seed collected from the transformed lines produced progeny plants of which at least 50% carried the introduced nptII selectable marker gene and were resistant to G418, as expected for the progeny of a primary regenerated transformed plant intercrossing with a mixture of transgenic and non-transgenic lines available at the trial site.

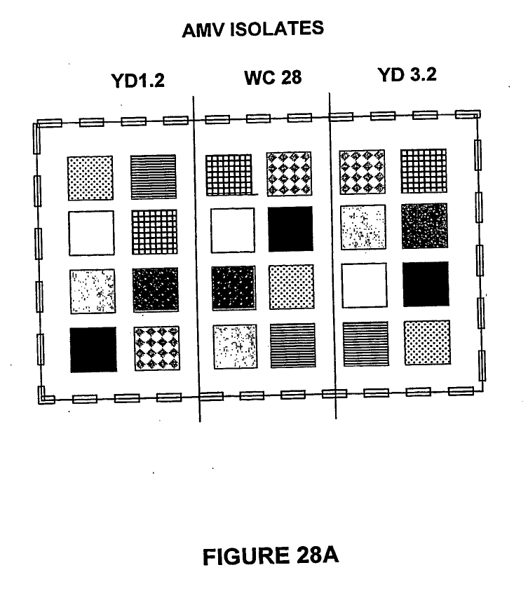

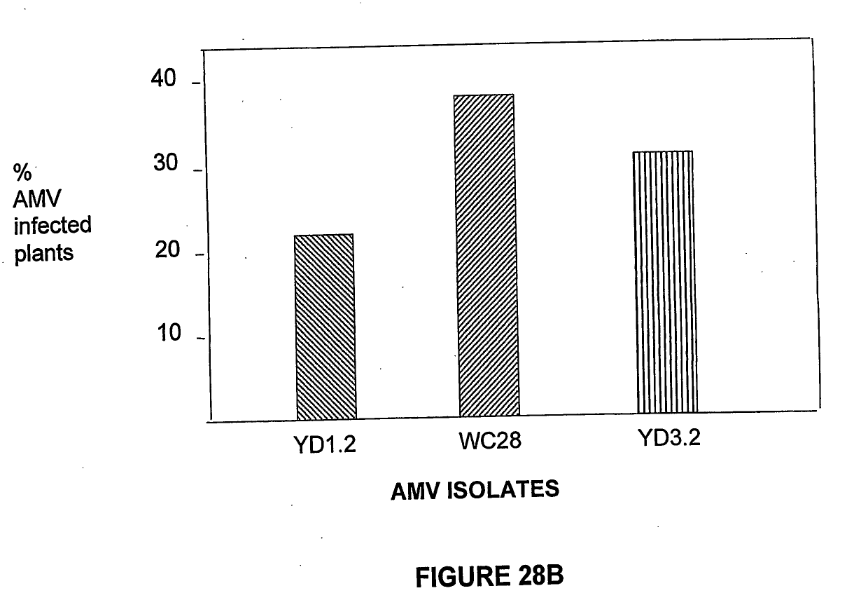

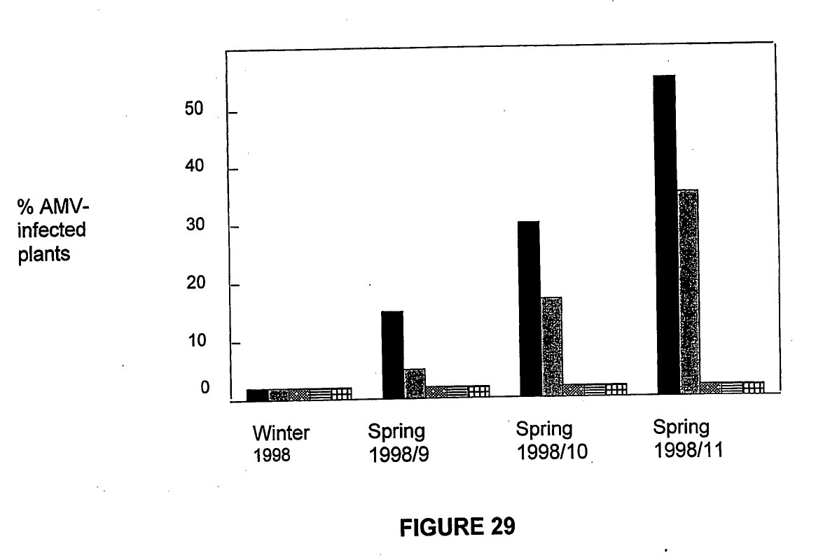

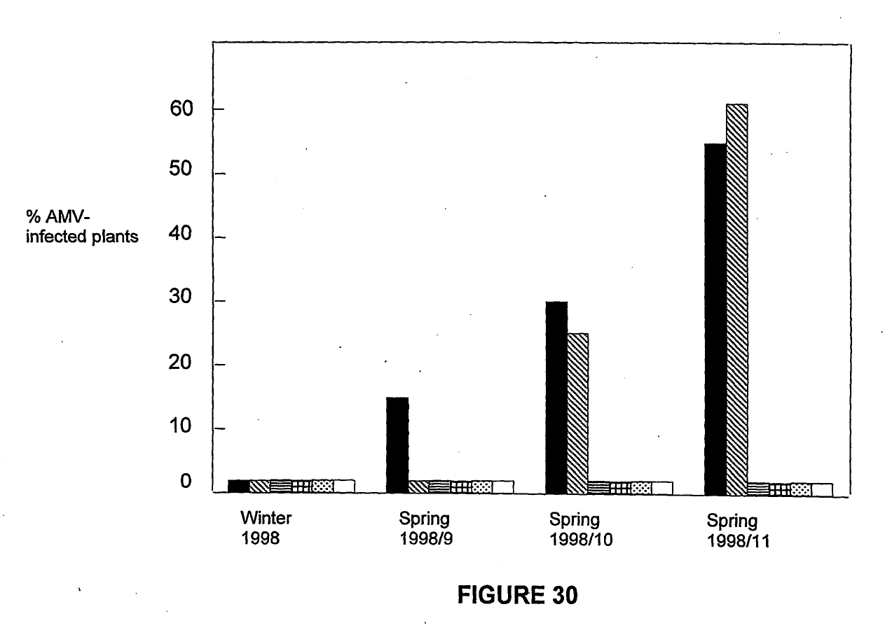

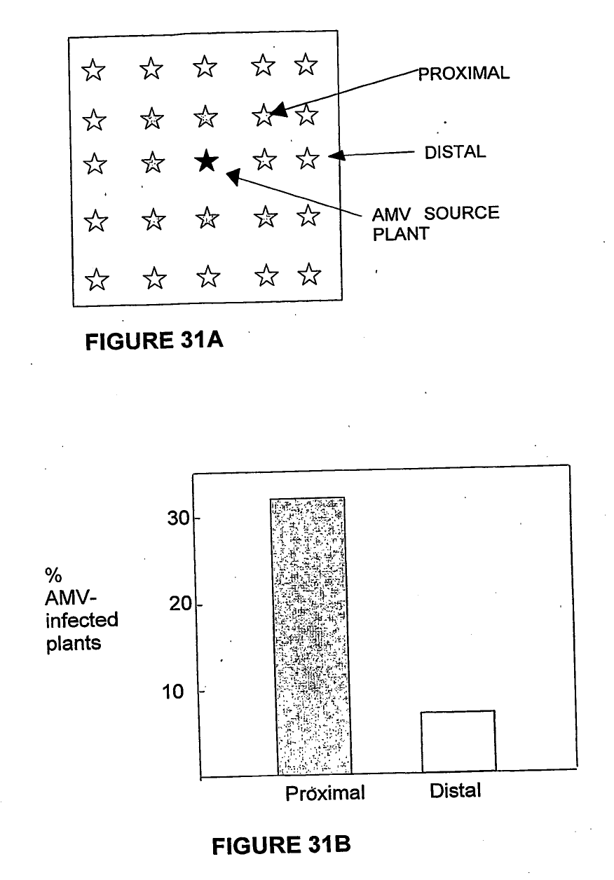

Symptom assessment, northern analysis and bioassays on indicator plants indicated that up to 60% of the non-transgenic lines became infected with all three isolates of AMV, however less than 50% of the resistant line and none of the immune lines became infected with the virus (FIGS. 28-30). The results of these analyses also showed that AMV spread much more efficiently when the test plants were within 1 m from the source plants (FIG. 31). Accordingly, the procedures described herein are suitable for the preparation of transformed plants which exhibit virus-resistance or virus-immunity under both glasshouse and field conditions.

### EXAMPLE 5.2

### Multisite Field Trial of Primary Transgenic (T₁) Virus-Resistant White Clover Lines (GMAC Planned Release 64X)

Field testing of the AMV-immune white clover lines was extended to evaluate the T₁ generation of white clover plants transformed with the AMV coat protein gene under the control of the double CaMV 35S promoter and the nptII gene under the control of the nopaline synthase (nos) promoter. These plants have been derived from crosses between the two original T₀ cv. Irrigation plants carrying the transgenes at the H1 and H6 loci, being used in the PR64/PR67 combined trial, with wild-type white clover plants. The two T₀ plants proved immune to AMV in the previous T₀ field trial. The aim of the T₁ trial was to assess the stability of protection against AMV infection conferred by the transgenes at two loci in different genetic backgrounds and in two different geographical regions. This is a necessary step in the production of germplasm for later cultivar development.

The T₁ lines were evaluated over two years at two geographically diverse sites. The first was at the Pastoral and Veterinary Institute, Agriculture Victoria, Hamilton in Victoria and the second was in a lucerne farm at Howlong, New South Wales.

A. Field Trial Layout:

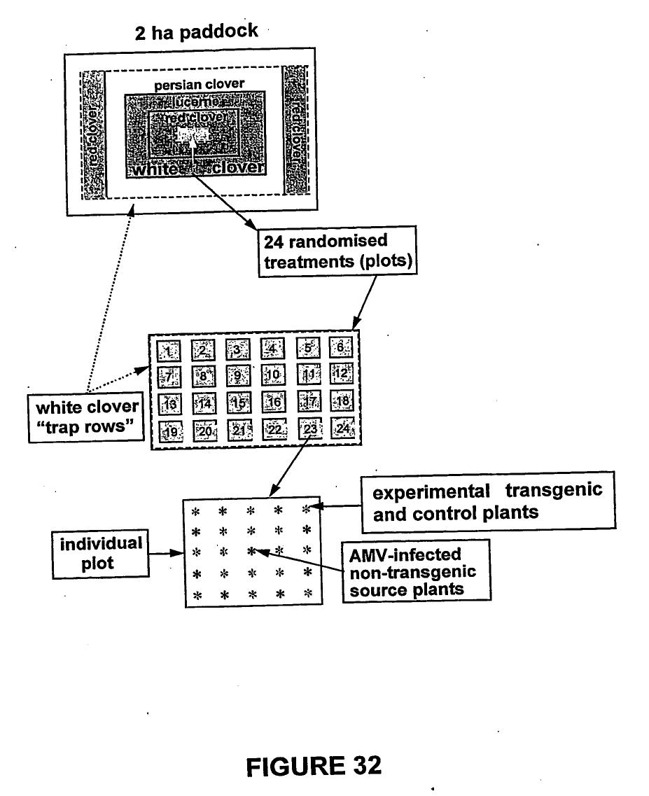

The design for the T₁ trial (FIG. 32) was similar to that used for To trial. The field trial comprised 24 plots distributed in 6 columns of four rows. The 24 plots were at the centre of a 2 hectare paddock planted with red clover, lucerne and Persian clover (long flowering) in 18 m concentric bands around the plots. In addition there was a 1 m wide band of white clover ‘pollen trap’ plants immediately around the experimental plots and just inside the perimeter fence of the 2 hectare paddock.

B. Individual Experimental Plot Design:

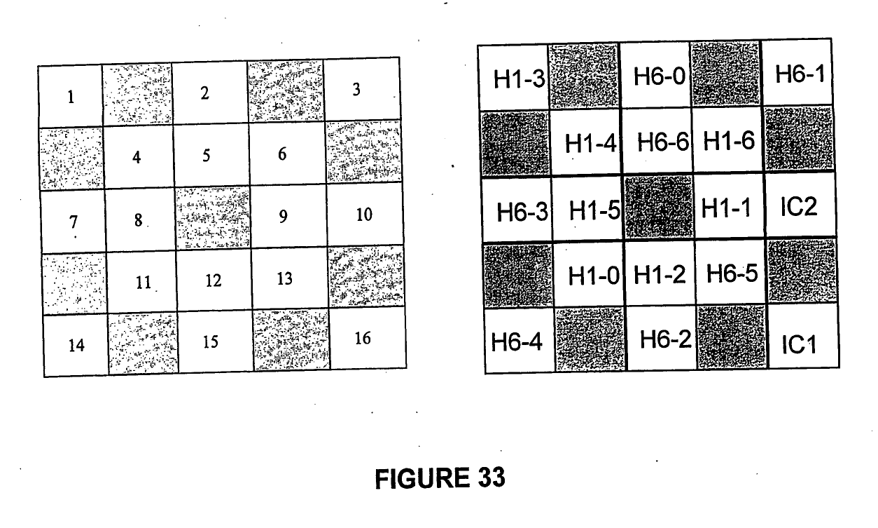

Each of the 24 experimental plot at the centre of the 2 ha paddock had the design described in FIG. 33. Each plot consisted of an array of 5 by 5 plants at 0.6 m spacing of which 9 were AMV isolate WC28-infected wild-type white clover clones (AMV source plants) and 16 were experimental plants planted on positions 1-16 in each plot (FIG. 33). The 16 experimental plants comprised 2 wild-type ‘Irrigation’ controls, 1 T₀ plant of H1 genotype, 1 T₀ plant of H6, 6 AMV resistant T₁ genotypes carrying the H1 transgene, and 6 AMV resistant T₁ genotypes carrying the H6 transgene. The list of plant codes and genotypes of these 16 experimental plants is detailed on Table 27.

Each plot contained one replicate clone of each of the same 16 lines. The replication for the planting of the 16 experimental plants for each of the 24 experimental plots was fully randomised. There were thus 24 replicates of each of the two wild type ‘Irrigation’ plants, 24 replicates of each of the 2 transgenic T₀ genotypes (H1 and H6) and 24 replicates of each of the 12 transgenic T₁ lines (6 genotypes carrying the H1 transgene and 6 genotypes carrying the H6 transgene). The paths between plots were bare ground and 1 m wide. The field trial compared the field resistance of plants to AMV, and general growth form of the 16 experimental lines.

TABLE 27
List of plant material indicating plant code, transgene/genotype,
status/generation and number of plants required for each planned
field release site

| Plant | | Status/ | N° plants |
|---|---|---|---|
| code | Transgene/genotype | generation | required/trial site |
| A | None - wild type Irrigation IC1 | WT | 24 |
| B | None - wild type Irrigation I4 | WT | 24 |
| C | H1-0: H1 Irrigation | T₀ | 24 |
| D | H6-0: H6 Irrigation | T₀ | 24 |
| E | H1-1: H1x IC1 Irrigation cross | T₁ | 24 |
| F | H1-2: H1x IC1 Irrigation cross | T₁ | 24 |
| G | H1-3: H1x IC1 Irrigation cross | T₁ | 24 |
| H | H1-4: H1x IC1 Irrigation cross | T₁ | 24 |
| I | H1-5: H1x IC1 Irrigation cross | T₁ | 24 |
| J | H1-6: H1x IC1 Irrigation cross | T₁ | 24 |
| K | H6-1: H6x I2 Irrigation cross | T₁ | 24 |
| L | H6-2: H6x I2 Irrigation cross | T₁ | 24 |
| M | H6-3: H6x I2 Irrigation cross | T₁ | 24 |
| N | H6-4: H6x I4 Irrigation cross | T₁ | 24 |
| O | H6-5: H6x I4 Irrigation cross | T₁ | 24 |
| P | H6-6: H6x I4 Irrigation cross | T₁ | 24 |

C. Trial Maintenance:

AMV spread was facilitated by releasing the two endemic species of aphids, Myzus persicae and Aphis craccivora, on the AMV-source plants of the experimental plots to aid AMV infection.

D. Field Trial Results

During the first year after trial establishment, plant growth parameters (plant height and width, stolon density and flower numbers) were measured and virus infection was visually assessed at both sites at monthly intervals from July to December. Suspected infected plants were sampled for virus bioassays.

Two batches of aphids were released at the field trial sites from August to October. All plants from replicate block 2 from each site were then sampled twice for bioassays from the end of October to December. The results showed 98% correlation between AMV field symptoms and AMV positive bioassays.

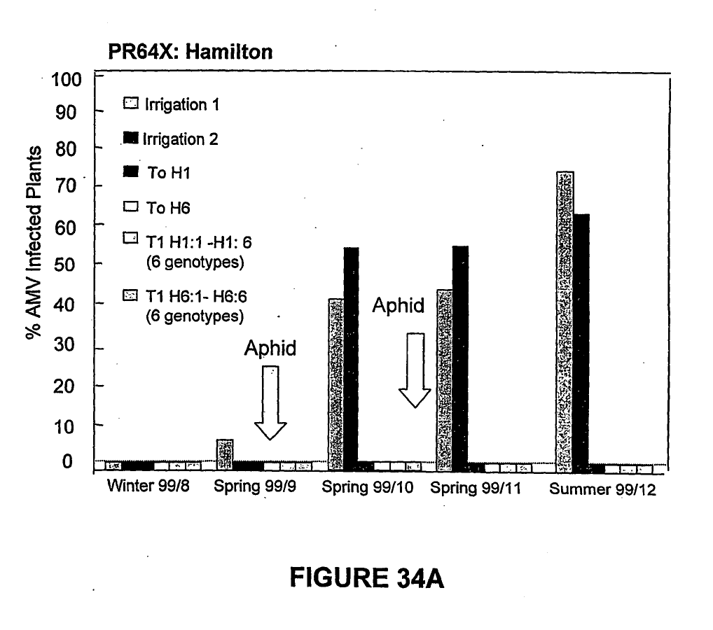

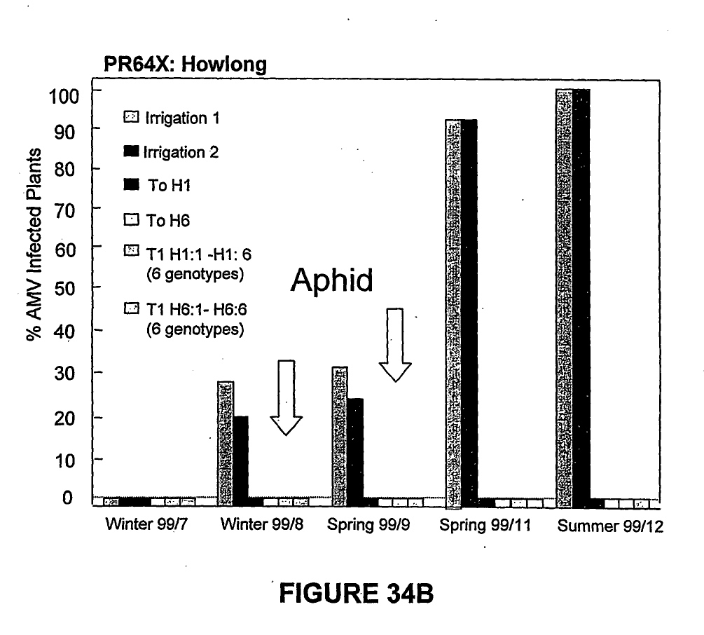

At Hamilton, AMV spread was delayed by cold weather and required artificial aphid release for the virus to spread. Only 2% (1 in 48) of the non-transgenic plants were infected with AMV before aphid release. However, by December after aphid release, up to 85% of the non-transgenic lines were infected with AMV while all the transgenic lines remained uninfected (FIG. 34A). No CYVV or WCMV were detected at the site.

At Howlong, the more favourable conditions at the site permitted natural spread of AMV before aphid was released so that by early September the non-transgenic lines were 29% infected with AMV while the two T₀ and 12 T₁ transgenic immune lines remained uninfected in the field. (FIG. 34B). These infections were presumably due to spread by natural aphids. By November, over 90% of the non-transgenic lines were infected with AMV while all the transgenic lines remained uninfected. CYVV was also found to be wide-spread at Howlong, infecting 10% of the plants at the site. WCMV was not detected.

The presence of AMV in each plant was assessed twice in early winter and early summer in the second year.

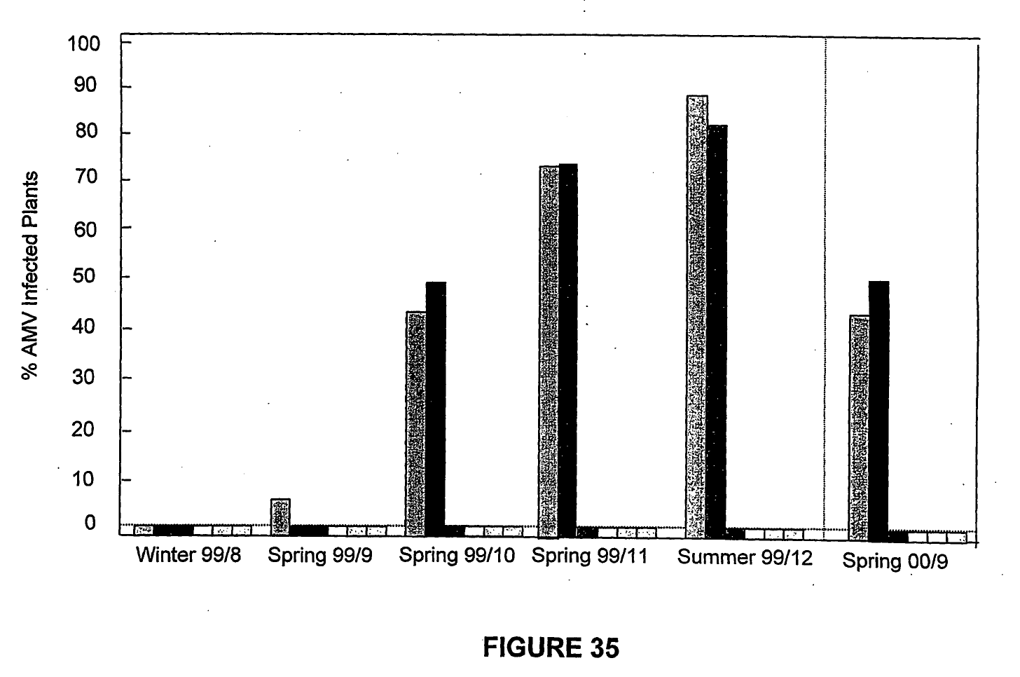

At Hamilton, more than half of the control AMV^{s} non-transgenic plants succumbed to the combined effects of AMV infection and abiotic stress and were replaced. The first assessment of virus infection in September 2000, for the second growth season, showed that between 40-50% of the replanted and surviving control AMV^{s} non-transgenic plants were infected by AMV, while all transgenic plants sampled remained immune to infection by AMV (FIG. 35). Additional aphid releases were made on 5^{th} September and 4th Oct. 2000 to ensure AMV spread for the second year.

At Howlong, AMV spread in control AMV^{s} non-transgenic plants reached 100% in June 2000, while all transgenic plants sampled remained immune to infection by AMV.

Representative clones of each of the 14 transgenic lines were sampled seasonally to evaluate transgene expression level by northern analysis. Transgene expression in all the T₁ lines was found to be stable under field conditions indicating that the transgene is also stably expressed in the transgenic progeny.

All transgenic plants grew well at both sites and set seed. Measurement of agronomic characters defining growth form (diameter, height, stolon density at plant center, stolon density at plant edge, flower numbers) were carried out twice each year. Statistical analysis of these growth parameters showed that the T₁ and T₀ transgenic plants were of the same general form as the non-transgenic plants in the trial. The T1 transgenic plants thus behaved as normal white clover plants.

### EXAMPLE 5.3

### Multisite Field Trial of Transgenic T₂ Virus-Resistant White Clover Lines (GMAC Planned Release 64X(2))

Field testing of the AMV-resistant white clover lines was further extended to evaluate the T₂ generation from the intercross of the T₁ elite AMV immune plants (heterozygous at the single H1 and H6 transgene loci). T₂ generation progeny plants expressing the AMV coat protein gene and carrying the transgene in either a heterozygous or a homozygous state were then identified by PCR and northern analysis of offspring plants. A field trial was then carried out to test whether the immunity to AMV observed in the primary (T₀) and T₁ offspring of the transgenic white clover plants under field conditions also occurs in the T₂ elite offspring (homozygous and heterozygous for the AMV immunity transgene) derived from these plants. In parallel a selection nursery trial with the aim of allowing for the identification of parental lines of white clover with the transgene introgressed in a homozygous state was established.

A total of 336 T₂ generation plants derived from the H1 and H6 genotypes were evaluated at the Howlong site while 1300 T2 generation offspring plants were planted in Hamilton. Similar design of trial. sites with respect to all biosafety features were used in these trials as in the PR64X as described in Example 5.2.

A1. Field Trial at Howlong Site:

The field trial was established in May 2001. The general layout of the field trial at Howlong involves one central experimental site containing 24 plots distributed in an array of 6 columns by four rows. As for the T₁ PR64X field trial, each experimental plot consists of an array of 5 by 5 plants at 0.6 m spacing of which 9 are AMV-infected wild-type white clover clones (AMV source plants) and 16 are experimental plants planted on positions 1-16 in each plot (FIG. 33). The 16 experimental plants comprise 2 wild-type controls, 1 T₀ plant of H1 genotype, 1 T₀ plant of H6 genotype, 6 T₂ genotypes derived from the T₁ elite transgenic H1 plant, and 6 T₂ genotypes derived from the corresponding T₁H6 elite transgenic plant as detailed in table 28B.

Each plot contains one replicate clone of each of the same 16 lines thus 24 of each line was planted. The replication of these 16 experimental plants in each of the 24 experimental plots was fully randomised, as described for Example 5.2.

A2. Field Trial at Hamilton Site:

The field trial at Hamilton evaluated 1,300 transgenic T₂ white clover plants (putatively homozygous for the AMV coat protein transgene, derived from the primary transformation events H1 and H6) in elite ‘Mink’-type genetic background (Table 28A). The design involves one central experimental plot with a spatial distribution of 1500 plants arranged in an array of 30 rows by 50 columns at the centre of a 2 hectare paddock. The 1500 test plants included 200 repeated control plants uniformly distributed within the plot. These controls were clones of 10 non-transgenic white clover parents of the cv. ‘Mink’ and are indicated as A, B, C, D, E, F, G, H, I and J in Table 28C. The remainder of the test plants consisted of 1,300 transgenic T₂ white clover plants (putatively homozygous for the AMV coat protein transgene, derived from the primary transformation events H1 and H6) in elite ‘Mink’-type genetic background.

TABLE 28A
Genotype composition of elite T2 AMV resistant germplasm
putatively homozygous for the AMV coat protein transgene, derived
from the primary transformation events H1 and H6 and included
in the Hamilton trial.
T2 H1 Genotypes for Hamilton:

| | ♂ | | | | | | | | | | | |
|---|---|---|---|---|---|---|---|---|---|---|---|---|
| ♀ | E₁ | E₂ | E₃ | E₆ | E₈ | E₉ | E₁₀ | E₁₁ | E₁₂ | E₁₃ | E₁₄ | E₁₅ |
| E₁ | | | | | | | | | | | | |
| E₂ | 2 | | | | | | | | | | | |
| E₃ | 17 | 24 | | | | | | | | | | |
| E₆ | 1 | 19 | 10 | | | | | | | | | |
| E₈ | 4 | 0 | 6 | 8 | | | | | | | | |
| E₉ | 15 | 27 | 5 | 14 | 2 | | | | | | | |
| E₁₀ | 3 | 4 | 8 | 0 | 15 | 2 | | | | | | |
| E₁₁ | 21 | 0 | 21 | 20 | 11 | 9 | 0 | | | | | |
| E₁₂ | 23 | 14 | 30 | 4 | 4 | 3 | 2 | 13 | | | | |
| E₁₃ | 11 | 12 | 9 | 8 | 0 | 14 | 4 | 6 | 28 | | | |
| E₁₄ | 30 | 24 | 31 | 2 | 5 | 15 | 19 | 11 | 31 | 29 | | |
| E₁₅ | 7 | 5 | 30 | 25 | 31 | 30 | 28 | 11 | 29 | 17 | 29 | |

Total: 894 genotypes present
T2 H6 Genotypes for Hamilton:

| | ♂ | | | | | | | | | | | |
|---|---|---|---|---|---|---|---|---|---|---|---|---|
| ♀ | E₁ | E₂ | E₃ | E₆ | E₈ | E₉ | E₁₀ | E₁₁ | E₁₂ | E₁₃ | E₁₄ | E₁₅ |
| E₁ | | | | | | | | | | | | |
| E₂ | 5 | | | | | | | | | | | |
| E₃ | 11 | 1 | | | | | | | | | | |
| E₇ | 13 | 2 | 3 | | | | | | | | | |
| E₈ | 2 | 4 | 5 | 10 | | | | | | | | |
| E₉ | 12 | 3 | 7 | 12 | 11 | | | | | | | |
| E₁₀ | 10 | 5 | 0 | 15 | 7 | 2 | | | | | | |
| E₁₁ | 5 | 5 | 12 | 2 | 1 | 8 | 4 | | | | | |
| E₁₂ | 0 | 4 | 1 | 10 | 9 | 7 | 3 | 3 | | | | |
| E₁₃ | 6 | 6 | 3 | 3 | 14 | 4 | 8 | 3 | 3 | | | |
| E₁₄ | 9 | 9 | 10 | 12 | 4 | 3 | 6 | 4 | 0 | 7 | | |
| E₁₅ | 9 | 4 | 13 | 5 | 8 | 5 | 12 | 5 | 11 | 2 | 10 | |

Total: 412 genotypes present

B. Results of T2 AMV Field Trials

The Howlong field trial to evaluate 6 T2 homozygous lines of H1 and 6 of H6 for field resistance to AMV was established on 28 May 2001. Aphid was released during August and September.

The field trial was evaluated as for the T1 PR64X field trialin Example 5.2. Initial assessment in August and September showed all the transgenic lines were AMV resistant but about 9% of the non-transgenic control plants was infected with AMV. Further assessments made in October showed that AMV infection in the non-transgenic control lines increased rapidly to 63% while all the transgenic lines were virus free (Table 28B).

The T2 lines were also assessed for transgene expression in late winter (August) in the Howlong trial. Representative transgenic plants from each line were sampled for northern hybridization analysis (3 replicates of the 14 transgenic lines and 3 replicates of the non-transgenic controls) and transgene expression was found to be stable under field conditions. Growth analysis over the first growth season also showed that the T₂ transgenic white clover plants are of the same general form as the non-transgenic plants in the field.

TABLE 28B
Assessment of AMV infection in Howlong T2 transgenic
white clover trial.

| | | | AMV Coat | |
|---|---|---|---|---|
| | | | Protein | |
| | | | mRNA | |
| Plant | | Status/ | northern | % AMV |
| code | Transgene/genotype | generation | signal | infection |
| A | None - wild type | WT | − | 50 |
| | Mink clone 1 | | | |
| B | None - wild type | WT | − | 63 |
| | Mink clone 2 | | | |
| C | H1-0: Irrigation | T₀ | + | 0 |
| D | H6-0: Irrigation | T₀ | + | 0 |
| E | H1-1: Mink cross | T₂ | + | 0 |
| F | H1-2: Mink cross | T₂ | + | 0 |
| G | H1-3: Mink cross | T₂ | + | 0 |
| H | H1-4: Mink cross | T₂ | + | 0 |
| I | H1-5: Mink cross | T₂ | + | 0 |
| J | H1-6: Mink cross | T₂ | + | 0 |
| K | H6-1: Mink cross | T₂ | + | 0 |
| L | H6-2: Mink cross | T₂ | + | 0 |
| M | H6-3: Mink cross | T₂ | + | 0 |
| N | H6-4: Mink cross | T₂ | + | 0 |
| O | H6-5: Mink cross | T₂ | + | 0 |
| P | H6-6: Mink cross | T₂ | + | 0 |

The Hamilton trial containing the 1300 transgenic homozygous T2 AMV resistant plants was established on 22 May 2001. All plants were assessed for virus infections in September 2001. as shown in Table 28C.

The results showed all the 1300 transgenic lines were AMV resistant but many of the non-transgenic control lines were infected with AMV, ranging from 0 to 80% infection rate.

TABLE 28C
Assessment of AMV infection in Hamilton T2 transgenic
white clover trial.

| | | | AMV | |
|---|---|---|---|---|
| | | | Coat | |
| | | | Prot in | |
| | | | mRNA | |
| Plant | | Status/ | northern | % AMV |
| code | Transgene/genotype | generation | signal | infection |
| A | None - wild type Mink clone 1 | WT | − | 10 |
| B | None - wild type Mink clone 2 | WT | − | 25 |
| C | None - wild type Mink clone 1 | WT | − | 0 |
| D | None - wild type Mink clone 2 | WT | − | 15 |
| E | None - wild type Mink clone 1 | WT | − | 80 |
| F | None - wild type Mink clone 2 | WT | − | 0 |
| G | None - wild type Mink clone 1 | WT | − | 35 |
| H | None - wild type Mink clone 2 | WT | − | 15 |
| I | None - wild type Mink clone 1 | WT | − | 0 |
| J | None - wild type Mink clone 2 | WT | − | 35 |
| K | H1 T2 Homozygous Lines | T₂ | + | 0 |
| L | H6 T2 Homozygous Lines | T₂ | + | 0 |

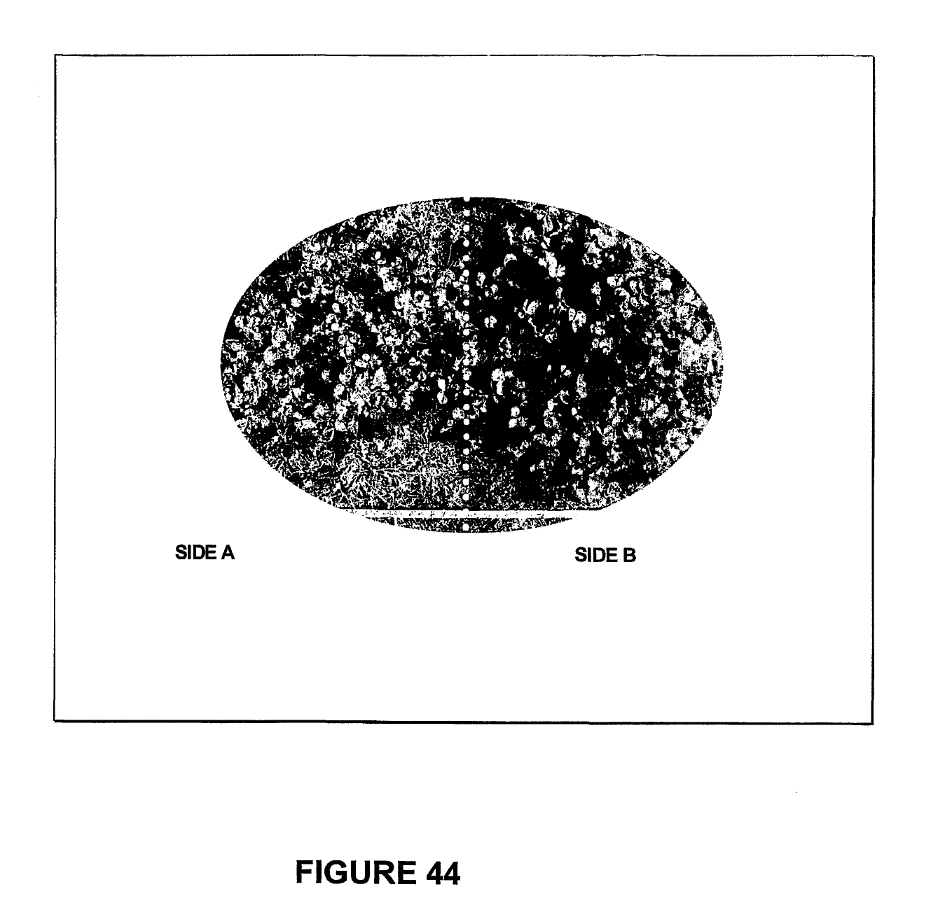

Plants were also assessed for agronomic characters defining growth form (diameter, height, stolon density at plant center, stolon density at plant edge, flower numbers) in late spring and in mid winter (not shown). FIG. 44 shows the difference in growth and potential yields of the susceptible non-transgenic white clover and a corresponding AMV immune transgenic plant. The virus infected susceptible white clover plant cv. ‘Irrigation’ shown on side A displays lower dry matter production, decreased persistence and decreased nutritional value, while the Virus immune transgenic white clover plant cv. ‘Irrigation’ shown on side B displays increased dry matter production and increased persistence.

Timmerman-Vaughan et al (2001) showed that when they used the CaMV 35S promoter in their coat protein gene construct, the resistance to AMV was only partially inherited in the offsprings of most lines and that they were only partially resistant in the field. In contrast, all the ASSU lines tested in the above field trial were immune.

Part VI of the Experimental Section:

Production of Multiple Virus Resistance

### EXAMPLE 6.1

### Production of CYVV-WCMV Double Virus Resistance Using a Single Gene Construct

Attempts were made to produce double CYVV+WCMV resistant white clover plants by transformation using the binary vector pBH3 that contained both the CYN and WCMV coat protein gene (FIG. 17). The results in Table 29. showed that the construct was capable of producing plants with CYVV immunity and WCMV resistance (pBH3-12). In addition, a total of 25 transgenic plants derived from wild-type seed transformed with pBH3 containing the WCMV+CYN coat protein genes were challenged against WCMV and CYVV. The results showed 16 of the 25 lines having immunity/strong resistance against CYN and that the presence of the WCMV gene did not affect the expression of the CYN resistant phenotype.

TABLE 29
Summary of Challenge of White Clover BH3
(Double CYVV CP + WCMV CP + hph) Transformants

| | | Virus Infection Levels | | |
|---|---|---|---|---|
| Lines | PCR hph | CYVV | WCMV | Final Results |

Non-Transgenic Control Lines

| | | | | |
|---|---|---|---|---|
| NT I2 | (−ve) | 5/5, CYVV S | 5/5, WCMV S | All S |
| NT I4 | (−ve) | 5/5, CYVV S | 5/5, WCMV S | All S |
| NT I5 | (−ve) | 4/5, CYVV S | 5/5, WCMV S | All S |

BH3 transformed normal cv. Irrigation white clover
(CYVV CP + WCMV CP + hph)

| | | | | |
|---|---|---|---|---|
| BH3/2 | (+ve/+ve) | 0/5, CYVV I | 4/5, WCMV S | CYVV I |
| BH3/4 | (+ve/+ve) | 5/5, CYVV S | 5/5, WCMV S | All S |
| BH3/5 | (+ve/+ve) | 0/5, CYVV I | 5/5, WCMV S | CYVV I |
| BH3/9 | (+ve/+ve) | 0/5, CYVV I | 5/5, WCMV S | CYVV I |
| BH3/10 | (+ve/+ve) | 2/5, CYVV R | 5/5, WCMV S | CYVV R |
| BH3/11 | (+ve/+ve) | 2/5, CYVV R | 4/5, WCMV S | CYVV R |
| BH3/12 | (+ve/+ve) | 0/5, CYVV I | 3/5, WCMV R | CYVV I, |
| | | | | WCMV R |
| BH3/13 | (+ve/+ve) | 0/5, CYVV I | 5/5, WCMV S | CYVV I |
| BH3/14 | (+ve/+ve) | 0/5, CYVV I | 5/5, WCMV S | CYVV I |
| BH3/15 | (+ve/+ve) | 0/5, CYVV I | 5/5, WCMV S | CYVV I |
| BH3/16 | (+ve/+ve) | 0/5, CYVV I | 5/5, WCMV S | CYVV I |

BH3 lines were challenged with WCMV (Ex Ham 22/18) and CYVV (WC18) viruses simultaneously and infections confirmed by bioassays

### EXAMPLE 6.2

### Production of AMV-CYVV-WCMV Triple Virus Resistant Whit Clover by Supertransformation and Virus Resistance Screening

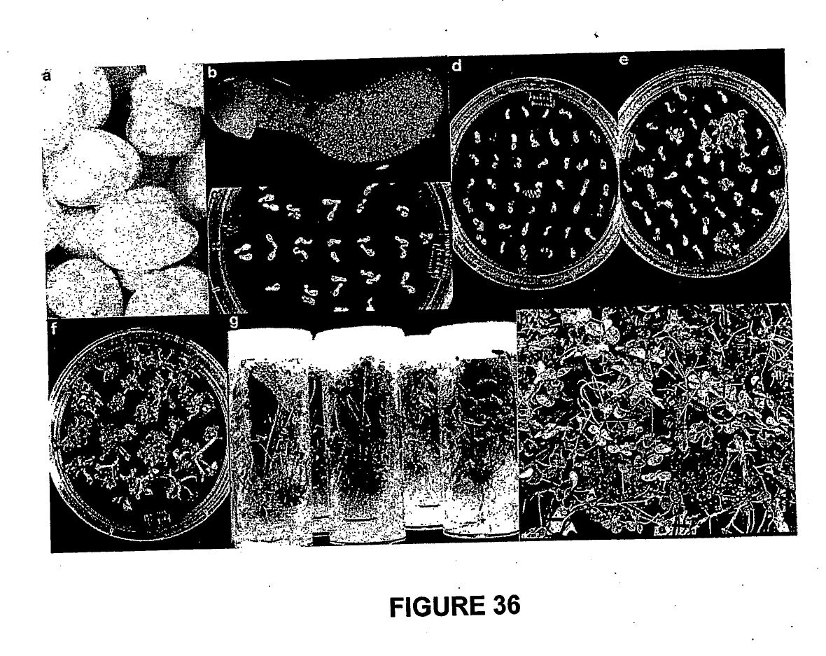

The plasmid pBH3, containing chimeric WCMV and CYVV coat protein genes and the hph selectable marker gene driven by the CaMV 19S promoter, was constructed to assess the suitability of the hph gene coding for hygromycin resistance for use as a second selectable marker gene in the supertransformation of the AMV coat protein transgenic white clover already containing the nptII selectable marker gene. Over ten thousand T₁ seed was obtained by crossing the H1 and H6 AMV-immune transgenic white clover line with wild type white clover. Half of these seed will be transgenic for the AMV coat protein gene. More than 12,000 cotyledonary explants from T₁ transgenic AMV-resistant seed were transformed with the binary vector pBH3. Over 200 putative transgenic white clover plants were produced under hygromycin selection (FIG. 36).

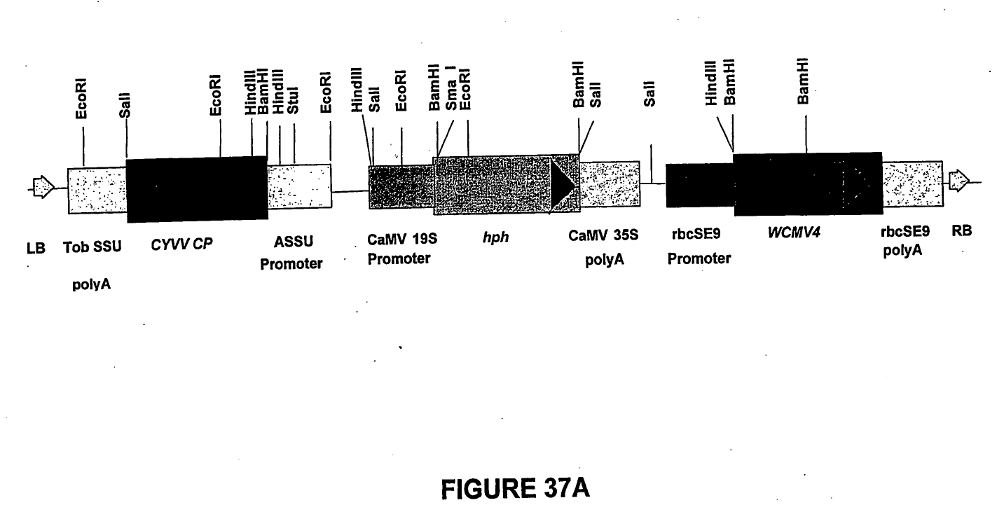

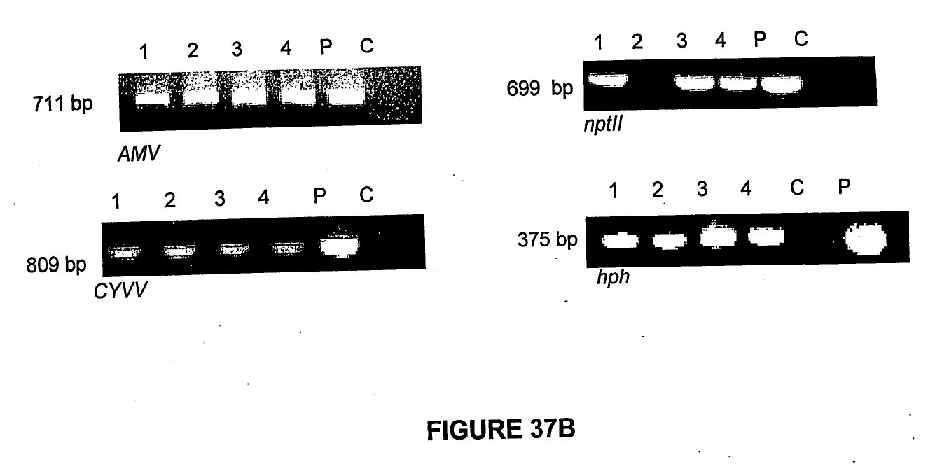

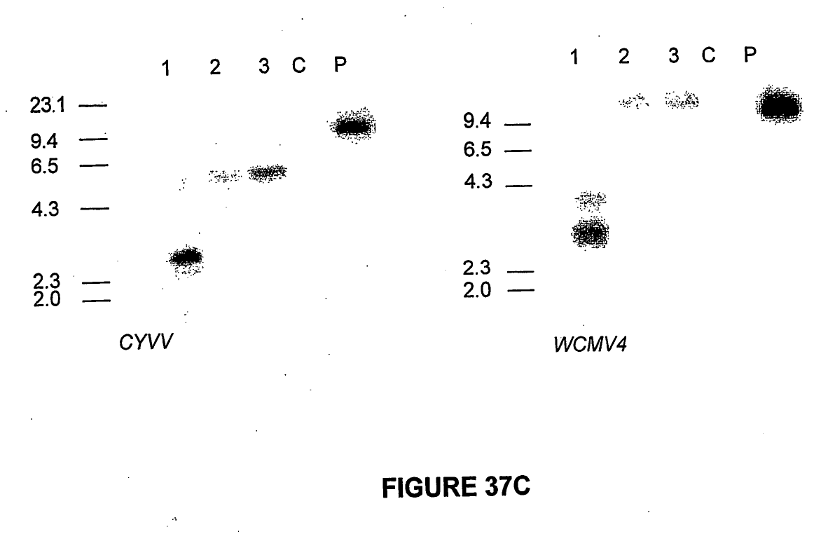

The 200 hygromcyin resistant putative transgenic plantlets were screened by PCR for the presence of the hph gene after supertransforming the AMV coat protein transgenic seed with the WCMV+CYVV double construct, pBH3. Further characterisation of the putative transgenic plants by PCR for presence of AMV and CYVV coat protein (CP) genes and northern hybridisation analysis to detect WCMV CP gene expression, was also performed. Two independent super-transformants carrying all three virus CP transgenes and expressing the WCMV CP gene have been characterised by Southern hybridisation analysis. One supertransformation event containing a single T-DNA insert with the CYVV and WCMV CP genes has been identified. Representative results from these analyses are shown in FIG. 37.

All putative supertransformants were challenged with all three viruses (AMV, WCMV and CYVV) using replicate cuttings of these plants. A representative experiment is shown in Table 30A.

The results showed that a total of 8 confirmed supertransformants were obtained which shows that two plants were immune to both AMV and CYVV (Table 30B).

TABLE 30A
Challenging BH3-H1 & BH3-H6 Supertransformants Against AMV, CYVV and WCMV

| | | Virus Challenge (Number | Final |
|---|---|---|---|
| Northern | PCR hph | infected/Number tested) | Results |

| | | | | | |
|---|---|---|---|---|---|
| Lines | (AMV CP) | (pBH3) | AMV WC28 | CYVV WC18 | WCMV Ex Ham 22/18 |

Non-Transgenic Control Lines

| | | | | | | |
|---|---|---|---|---|---|---|
| NT I2 | (−ve) | (−ve) | 6/6 | 6/6 | 6/6 | All S |
| NT I4 | (−ve) | (−ve) | 13/13 | 13/13 (LL) | 11/11 | All S |
| NT I5 | (−ve) | (−ve) | 6/9 | 10/10 | 9/9 | All S |

BH3-H1 supertransformed white clover (T1H1C Seed)

| | | | | | | |
|---|---|---|---|---|---|---|
| BH3-H1 5.3 | (+ve) | (+ve) | 0/5, I | 5/5 | 5/5 | AMV I |
| BH3-H1 7.1 | (−ve) | (+ve) | 5/5 | 5/5 | 5/5 | All S |
| BH3-H1 | (+ve) | (+ve) | 0/5, I | 5/5 | 5/5 | AMV I |
| 74.2 | | | | | | |
| BH3-H1 20 | (−ve) | (+ve) | 5/5 | 0/5, I | 5/5 | CYVV I |
| BH3-H1 21 | (−ve) | +ve) | 5/5 | 0/5, I | 5/5 | CYVV I |
| BH3-H1 22 | (−ve) | (+ve) | 4/5 | 0/5, I | 5/5 | CYVV I |
| BH3-H1 1 | (+ve) | (−ve) | 0/5, I | 5/5, R, L | 5/5 | AMV I + CYVV R |
| BH3-H1 1 | (+ve) | (−ve) | 0/5, I | 5/5 | 5/5 | AMV I |
| BH3-H1 B | (−ve) | (+ve) | 5/5 | 5/5, R, L | 5/5 | CYVV R |

BH3-H6 supertransformed white clover (H6 × Elite seed)

| | | | | | | |
|---|---|---|---|---|---|---|
| BH3-H6 2 | (+ve) | (−ve) | 0/5, I | 5/5 | 5/5 | AMV I |
| BH3-H6 3 | (+ve) | (−ve) | 0/5, I | 5/5 | 5/5 | AMV I |
| BH3-H6 4 | (−ve) | (−ve) | 3/5 | 5/5, R, L | 5/5 | CYVV R |
| BH3-H6 5 | (−ve) | (+ve) | 2/5 | 0/5, I | 5/5 | CYVV I |
| BH3-H6 1a | (+ve) | (+ve) | 0/5, I | 5/5 | 5/5 | AMV I |
| BH3-H6 1b | (+ve) | (+ve) | 0/5, I | 5/5 | 5/5 | AMV I |
| BH3-H6 1c | (+ve) | (+ve) | 0/5, I | 5/5 | 5/5 | AMV I |
| BH3-H6 2a | (+ve) | (+ve) | 0/5, I | 0/5, I | 5/5 | AMV + CYVV I |
| BH3-H6 2b | (+ve) | (+ve) | 0/5, I | 0/5, I | 5/5 | AMV + CYVV I |
| BH3-H6 3a | (+ve) | (−ve | 0/5, I | 5/5 | 5/5 | AMV I |
| BH3-H6 4 | (−ve) | (+ve) | 5/5 | 5/5 | 5/5 | All S |
| BH3-H6 5 | (+ve) | (−ve) | 0/5, I | 0/5, I | 5/5 | AMV + CYVV I |
| BH3-H6 6 | (−ve) | (−ve) | 5/5 | 5/5 | 5/5 | All S |
| BH3-H6 7 | (+ve) | (−ve) | 0/5, I | 5/5 | 5/5 | AMV I |

Note:
R, L = Plants resistant and showing latent infection, ie. very low virus levels (2-5% of controls and showing no symptoms).
I = Immune, no virus detectable and no symptoms.

TABLE 30B
Virus Resistance/Susceptibility Phenotypes of
AMV CP:WCMV4 CP:CYVV CP
Super-Transformed Transgenic White Clover Plants

| | | AMV | WCMV | CYVV |
|---|---|---|---|---|
| | | Resistance | Resistance | Resistance |
| Cultivar | Plant Code | Phenotype | Phenotype | Phenotype |
| Irrigation | pBH3-5.3 | I | S | S |
| Irrigation | pBH3-74.1 | I | S | S |
| Irrigation | pBH3-74.2 | I | S | S |
| Irrigation | pBH3-H1-4 | I | S | S |
| Irrigation | pBH3-H6-1a | I | S | S |
| Irrigation | pBH3-H6-1b | I | S | S |
| Irrigation | pBH3-H6-1c | I | S | S |
| Irrigation | pBH3-H6-2a | I | S | I |
| Irrigation | pBH3-H6-2b | I | S | I |

S = Susceptible (80-100% infection)
R = Resistant (<50% infection)
I = Immune (0% infection

Part VII of the Experimental Section:

Analysis of Segregation of Coat Pr tein Genes in Transgenic Plants

### EXAMPLE 7.1

### Analysis of Segregation of AMV Coat Protein Gene in AMV-Immune and AMV-Resistant White Clover Plants

The genotypes H1 and H6 transgenic plants each carrying a single copy of the AMV coat protein gene were crossed with three wild-type untransformed white clover plants. The T₁ seed from these crosses was scarified and germinated in soil. The progenies were analysed by northern and Southern hybridisation to check the inheritance and segregation ratios of the transgenes and challenged with AMV WC28 to correlate resistance with gene expression.

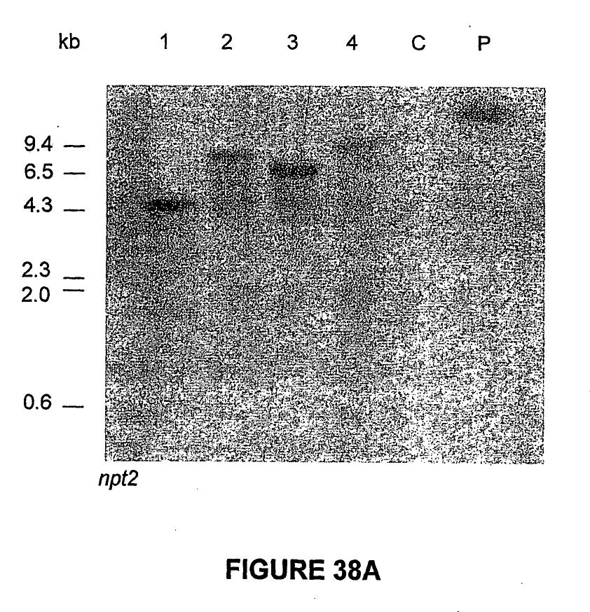

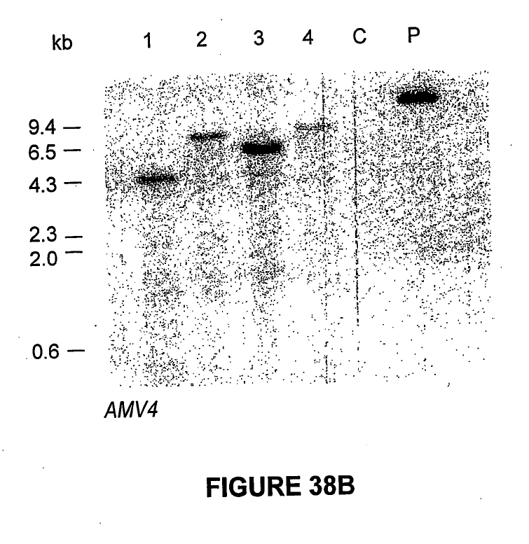

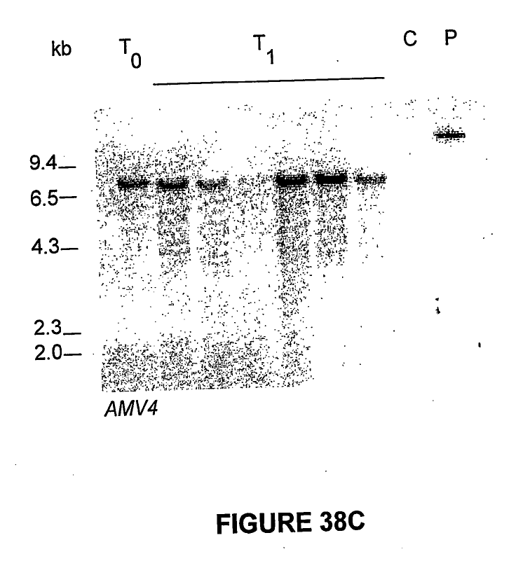

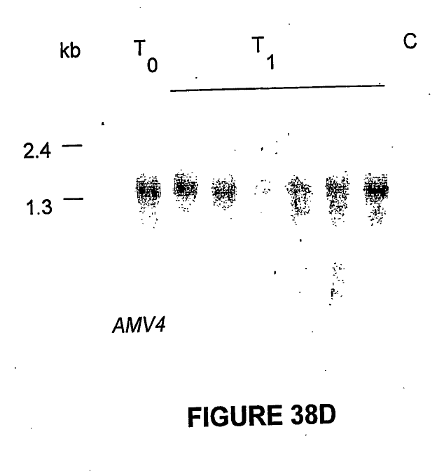

The transgenic T₀ H1 and H6 parents and the transgenic T₁ progenies derived from the H1xWT and H6xWT crosses all expressed the AMV coat protein gene as determined by northern hybridisation analysis (Table 31 and FIG. 38). The transgenic T₁ progenies from both genotypes H1 and H6, identified by northern analysis, were all immune to infection by AMV after mechanical inoculation under containment glasshouse conditions, while all the non-transgenic progenies (determined by a negative northern result) were all susceptible (Table 31). The results showed that the segregation ratio of the AMV coat protein transgene at both loci was about 0.5 as expected.

The result of the Southern analyses with nptII and AMV4 hybridisation probes showed that all the transgenic T₁ progenies carried the same construct (FIG. 38A) containing the AMV4 cDNA including the AMV coat protein gene under the control of a double CaMV 35S promoter together with the neomycin phosphotransferase (nptII) gene under the control of the nopaline synthase (nos) promoter] as their T₀ transgenic parents (FIGS. 38B-D).

TABLE 31
Correlation of AMV resistance with northern hybridisation signals
in T₁ transgenic lines

| Lines | GENOPTYPE | NORTHERN | AMV CHALLENGE |
|---|---|---|---|
| H1 | T0 immune parent | (+)ve | I |
| H6 | T0 Immune parent | (+)ve | I |
| I2 | WT Parent | (−)ve | S |
| I4 | WT Parent | (−)ve | S |
| I5 | WT Parent | (−)ve | S |
| 224.1 | I4 × H6 | (+)ve 3 | I |
| 224.2 | I4 × H6 | (+)ve 5 | I |
| 224.6 | I4 × H6 | (+)ve 5 | I |
| 225.1 | I4 × H6 | (−)ve | S |
| 225.2 | I4 × H6 | (−)ve | S |
| 225.3 | I4 × H6 | (+)ve 5 | I |
| 225.4 | I4 × H6 | (+)ve 5 | I |
| 230.1 | I4 × H6 | (−)ve | S |
| 230.4 | I4 × H6 | (+)ve 4 | I |
| 226.1 | H6 × I4 | (−)ve | S |
| 226.2 | H6 × I4 | (+)ve 5 | I |
| 227.1 | H6 × I4 | (−)ve | S |
| 227.8 | H6 × I4 | (−)ve | S |
| 229.3 | H6 × I4 | (−)ve | S |
| 229.4 | H6 × I4 | (−)ve | S |
| 229.8 | H6 × I4 | (−)ve | S |
| 229.9 | H6 × I4 | (+)ve 5 | I |
| 229.11 | H6 × I4 | (+)ve 5 | I |
| 231.2 | I2 × H6 | (+)ve 5 | I |
| 231.3 | I2 × H6 | (+)ve 5 | I |
| 231.6 | I2 × H6 | (+)ve 5 | I |
| 231.7 | I2 × H6 | (−)ve | S |
| 231.9 | I2 × H6 | (−)ve | S |
| 231.10 | I2 × H6 | (−)ve | S |
| 228.1 | H6 × I2 | (−)ve | S |
| 228.3 | H6 × I2 | (−)ve | S |
| 228.4 | H6 × I2 | (−)ve | S |
| 228.5 | H6 × I2 | (+)ve 3 | I |
| 228.6 | H6 × I2 | (+)ve 2 | I |

Part VIII of the Experimental Section:

Development of Methods f r Improvement of Transgenic Trifolium spp. Germplasm

### EXAMPLE 8.1

### Breeding Scheme for Producing Elite T2 AMV-Resistant White Clover Germplasm Homozygous for the AMV Coat Protein Gene Using Selective Progeny Screening and Test Cross Analysis

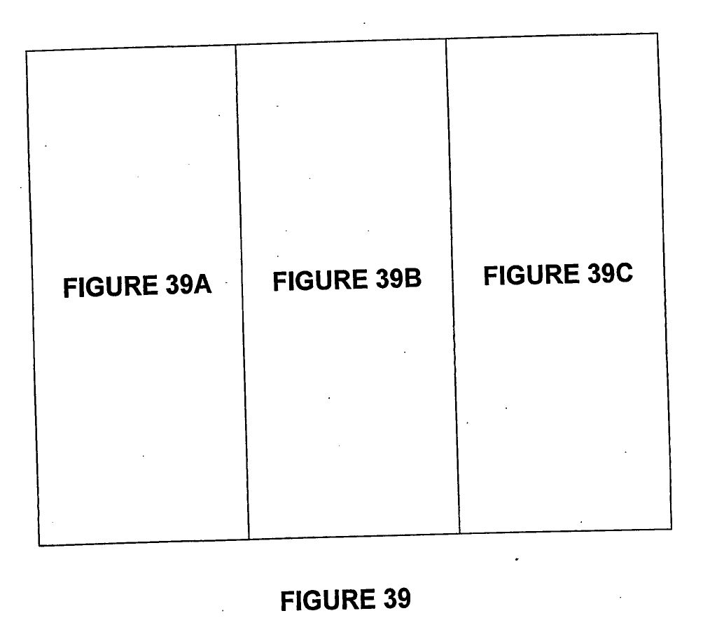

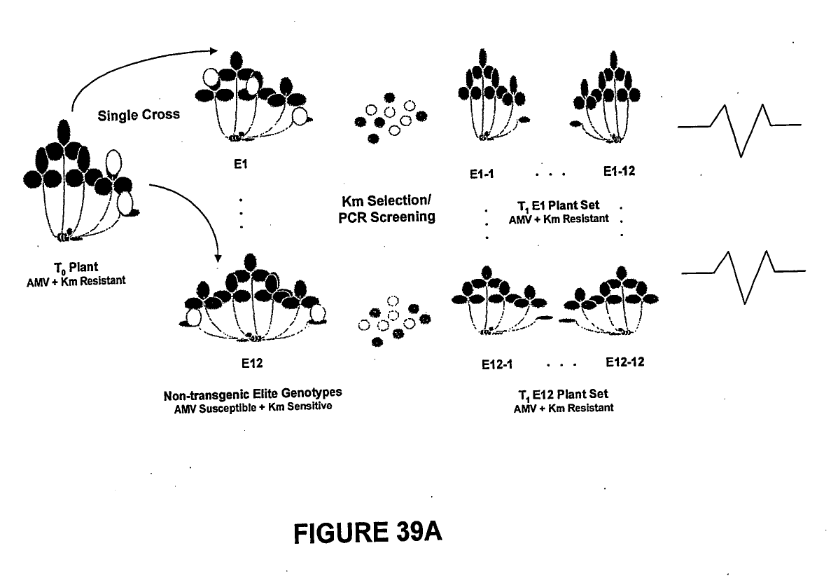

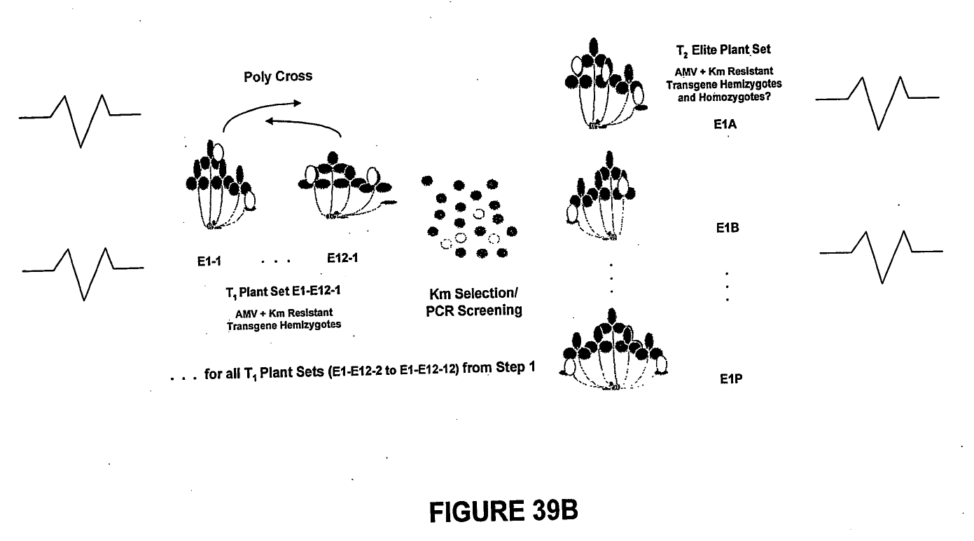

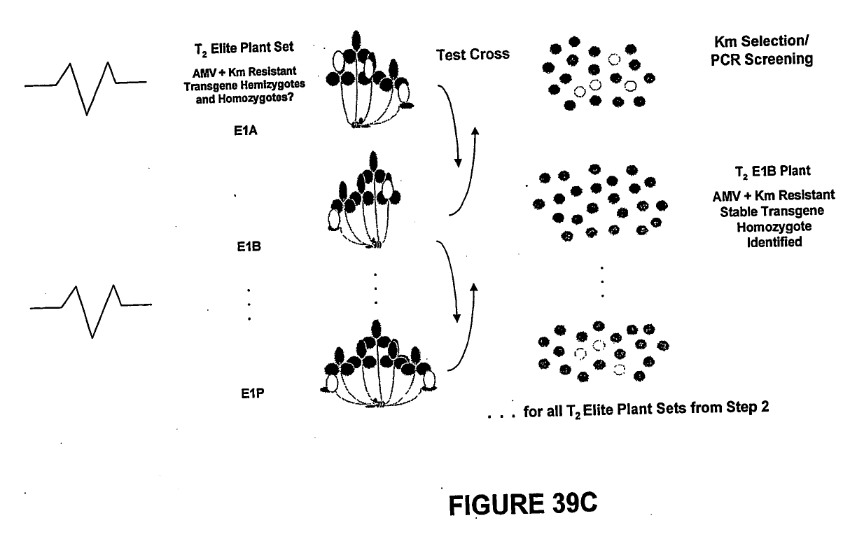

The steps involved in the production of transgenic germplasm are presented in FIG. 39. Briefly, hemizygous T1 plants were produced from a cross of primary transgenic AMV immune H1 or H6 genotypes with 12 elite white clover lines (Table 32A), and these were screened for resistance to kanamycin, and by PCR to confirm the presence of the nptII gene. Northern and virus resistance testing of progeny plants from each elite cross was also conducted. We produced virus resistant transgenic lines from each cross. Putative homozygous AMV-resistant elite lines were then produced by intercrossing sets of hemizygous transgenic T1 plants from the elite crosses, and conducting the appropriate virus screens and PCR tests to confirm virus-resistance and the presence of the nptII gene in each transgenic T2 line. In this way, virus resistant transgenic T2 seedlings from each of the elite sets of intercrosses were identified. To identify homozygous transgenic T2 plants from the elite crosses, the transgenic T2 plants from each of the elite sets of intercrosses were back-crossed onto non-transgenic material and the resulting T3 seedlings were screened by PCR for the presence of the nptII gene. A transgenic T₂ Parent that produces T3 seed that were shown to be 100% transgenic was classified as being homozygous for the transgene. In this way, homozygous virus resistant transgenic plants were identified for each of two genotypes, and from each elite parent line. Finally, polycrosses of the homozygous virus resistant elite lines are conducted for cultivar development.

In the above steps, to facilitate crosses, conditions were established that would allow year-round flowering in the containment glasshouse for all the white clover lines to be crossed. For all these lines, flowering commenced earlier at the longer day length of 16 hours, but more flowers were produced on more plants at the lower day length of 13 hours.

TABLE 32A
List of elite breeders' lines crossed with the
H1 and H6 transgenic plants

| Elite Lines | Flowering | Number of Crosses |
|---|---|---|
| E1 | + | 2 |
| E2 | + | 4 |
| E3 | ++ | 2 |
| E7 | + | 2 |
| E8 | ++ | 2 |
| E9 | ++ | 2 |
| E10 | + | 2 |
| E11 | ++ | 2 |
| E12 | ++ | 2 |
| E13 | + | 2 |
| E14 | ++ | 2 |
| E15 | ++ | 4 |

1. Kanamycin Selection of Transgenic T₁ Seedlings

When screening large numbers of seedlings in a breeding program, growth on 100 mg/l kanamycin is useful selection of transgenic seedlings. In preliminary tests, almost all non-transgenic seedlings produced bleached first leaves when grown in this manner, but 3/27 produced green first leaves. T₁ seed obtained from the elite x transgenic crosses were scarified and germinated on media containing essential salts and 100 mg/l kanamycin. Germination rates of these seed were highly variable, ranging from 17-65%. When germinated under kanamycin, most of the seedlings produced normal roots and green leaves, while a few were bleached or showing reduced root growth and branching. Up to 30 germinated seedlings were transplanted from the kanamycin plates into soil and further tested by challenging with AMV and by PCR to confirm the presence of nptII sequences in the transgenic kanamycin resistant seedlings. For lines that germinated poorly in kanamycin-containing media, the seed that failed to germinate were washed and re-germinated in soil. Many of these germinated normally in soil and were transplanted and screened as described for the kanamycin resistant seedlings.

2. AMV Challenge, nptII PCR Assay and Northern Analysis of T₁ progeny

Up to 30 kanamycin resistant T₁ seedlings from each elite x transgenic cross were characterised by screening for the presence of the nptII gene using PCR or non-radioactive DNA dot blot, and testing for AMV resistance. AMV resistant transgenic elite progeny lines were confirmed by northern blot for the presence of the AMV coat protein mRNA. The results showed 100% correlation between AMV resistance phenotypes and the presence of a northern signal in the progenies tested while the nptII PCR produced some false negatives (Table 32B). The nptII dot blot also produced some false negatives but was more sensitive than the PCR in detecting transgenic progenies. These results again confirmed that the use of northern analysis is the most reliable mean of identifying AMV-immune transgenic lines.

TABLE 32B
Typical results of an experiment screening for transgenic T1
progenies by kanamycin resistance screening, AMV resistance
testing, nptll PCR and dot blot and northern analysis in the white
clover germplasm development program

| | | | | | | Parent |
|---|---|---|---|---|---|---|
| | | | nptll | | | for |
| | Kanamycin | nptll | DNA | AMV | AMV CP | poly- |
| Lines | resistance | PCR | dot blot | status | Northern | cross |
| H6 × E8-1 | Resistant | +? | + | R | + | Yes |
| H6 × E8-2 | Resistant | + | + | R | + | Yes |
| H6 × E8-3 | Resistant | + | + | R | + | Yes |
| H6 × E8-4 | Resistant | − | + | R | + | Yes |
| H6 × E8-5 | Resistant | + | + | R | + | Yes |
| H6 × E8-6 | Resistant | − | +? | R | + | Yes |
| H6 × E8-7 | Resistant | + | + | R | + | Yes |
| H6 × E8-8 | Resistant | + | + | R | + | Yes |
| H6 × E8-9 | Resistant | − | +? | R | + | Yes |
| H6 × E8-10 | Resistant | + | + | R | + | Yes |
| H6 × E8-11 | Resistant | − | +? | R | + | Yes |
| H6 × E8-12 | Resistant | − | − | R | + | Yes |
| H6 × E8-13 | Resistant | + | + | R | + | Yes |
| H6 × E8-14 | Resistant | + | + | R | + | Yes |
| H6 × E8-15 | Resistant | + | + | R | + | Yes |
| H6 × E8-16 | Resistant | + | + | R | + | Yes |
| H6 × E8-17 | Resistant | + | + | R | + | Yes |
| H6 × E8-18 | Resistant | + | + | R | + | Yes |
| H6 × E8-19 | Resistant | + | + | R | + | Yes |
| H6 × E8-20 | Resistant | + | + | R | + | Yes |
| H6 × E8-21 | Resistant | − | − | S | − | No |
| H6 × E8-22 | Resistant | − | − | S | − | No |
| H6 × E8-23 | Resistant | + | + | R | + | Yes |
| H6 × E8-24 | Resistant | + | + | R | + | Yes |
| H6 × E8-25 | Resistant | − | − | S | − | No |
| H6 × E8-26 | Resistant | + | + | R | + | Yes |
| H6 × E8-27 | Resistant | + | + | R | + | Yes |
| H6 × E8-28 | Resistant | + | + | R | + | Yes |
| H6 × E8-29 | Resistant | + | + | R | + | Yes |
| H6 × E8-30 | Resistant | − | − | S | − | No |

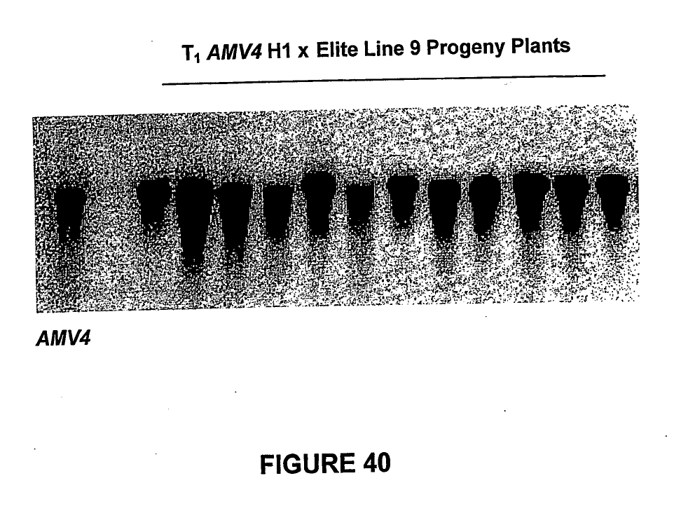

In summary, top crosses of the AMV immune cv. ‘Irrigation’ transgenic H1 and H6 genotypes with 12 elite white clover genotypes has been completed. The T₁ progeny from these single crosses between the 12 different elite white clover breeding lines (parental lines of the cv. ‘Mink’) and the two AMV^{r} T₀ transgenic white clover plants H1 and H6 were screened for the presence of the nptII gene by PCR. The expression of the AMV resistance gene was verified by northern hybridisation analysis using the AMV CP gene probe (FIG. 40). All transgenic T₁ lines from these crosses were confirmed to be AMV resistant by challenge experiments. A total of 12 AMV resistant progeny plants expressing the AMV coat protein gene from each cross with an elite parent were identified and grown to maturity.

3. Identification of Transgenic T₂ Seedlings

Twelve independent transgenic T₁ progeny lines derived from each AMV transgenic x elite parent cross were inter-crossed with 12 progenies from another AMV transgenic x elite parent cross to produce the T₂ seed (see FIG. 32B). Up to 20 T₂ seed from each of the elite transgenic diallel crosses were germinated in soil. Transgenic seedlings were identified by a northern dot blot procedure for detecting the AMV coat protein mRNA and confirmed by challenging with AMV (32C). The results showed 100% correlation between dot blot positive and AMV resistant T2 progenies. Therefore all subsequently all T2 progenies were tested by a RNA dot blot method to identify the transgenic progenies. All RNA dot blot positive lines and a proportion of the dot blot negative lines were subjected to AMV challenge using aphids. The results showed that under high aphid population pressure only 1% of the RNA dot blot positive lines (5/495) was infected and in contrast over 48% of the dot blot negative lines (117/245) were infected. All infected dot blot positive lines were discarded.

TABLE 32C
Molecular and resistance screening f T2 progenies to
identify transgenic lite germplasm material.

| | | T2 | RNA | AMV | |
|---|---|---|---|---|---|
| Progeny | | Progeny | Dot Blot | Resis- | |
| Sets | T1 Crosses | # | Result | tance | Conclusion |
| E1 × E2 | E1.30 × E2.16 | 1 | (−) | S | Non-transgenic |
| | | 2 | (+) | NS | Transgenic |
| | | 3 | (+) | NS | Transgenic |
| | | 4 | (−) | S | Non-transgenic |
| | | 5 | (−) | S | Non-transgenic |
| | | 6 | (+) | NS | Transgenic |
| | | 7 | (+) | NS | Transgenic |
| | | 8 | (+) | NS | Transgenic |
| | | 9 | (+) | NS | Transgenic |
| | | 10 | (+) | NS | Transgenic |
| | | 12 | (−) | S | Non-transgenic |
| | Total | 11 | 7/11 (+) | | |
| | E1.16 × E2.25 | 1 | (−) | S | Non-transgenic |
| | | 2 | (−) | S | Non-transgenic |
| | | 3 | (+) | NS | Transgenic |
| | | 4 | (−) | S | Non-transgenic |
| | | 5 | (−) | S | Non-transgenic |
| | | 6 | (+) | NS | Transgenic |
| | | 7 | (−) | S | Non-transgenic |
| | | 9 | (−) | S | Non-transgenic |
| | | 10 | (+) | NS | Transgenic |
| | | 11 | (+) | NS | Transgenic |
| | | 12 | (+) | NS | Transgenic |
| | | 13 | (+) | NS | Transgenic |
| | | 14 | (+) | NS | Transgenic |
| | | 15 | (+) | NS | Transgenic |
| | | 16 | (−) | S | Non-transgenic |
| | Total | 15 | 8/15 (+) | | |

4. Identification of Homozygous T2 Lines

To identify homozygous lines, T₂ transgenic plants were subjected to progeny testing by back crossing to non-transgenic material. T₃ seed collected from the back-crosses were germinated and up to 16 seedlings from each cross were screened for the presence of the AMV transgene by northern blot, PCR for the nptII gene and AMV resistance (Table 32D). T₂ parents producing 100% transgenic progenies are determined to be homozygous for the AMV resistance gene.

TABLE 32D
Analysis of T2 Germplasm for Homozgosity and Hemizygosity by Test Crosses

| T2 Test Cross Plants | | | | T3 Progeny Tests | | | |
|---|---|---|---|---|---|---|---|
| | AMV | | | | | AMV | |
| | RNA | Transgenic | AMV | nptll | AMV | northern | Inferred |
| T2 Plant Line | signal | Status | Resistance | PCR (+) | Resistance | (+) | Zygosity |
| 10.19 × 11.3 (2) | + | Transgenic | I | 2/11 | 2/11 | 2/11 | Hemi |
| 7.3 × 10.10 (5) | + | Transgenic | I | 13/13 | 13/13 | 13/13 | Homo |
| 10.10 × 13.6 (5) | + | Transgenic | I | 4/6 | 7/10 | 7/10 | Hemi |
| 9.27 × 15.8 (6) | + | Transgenic | I | 4/7 | 4/10 | 4/10 | Hemi |
| 9.20 × 15.20 (5) | + | Transgenic | I | 3/11 | 3/11 | 3/11 | Hemi |
| 3.22 × 14.24 (5) | + | Transgenic | I | 4/5 | 3/6 | 3/6 | Hemi |
| 3.22 × 14.24 (6) | + | Transgenic | I | 3/9 | 3/9 | 3/9 | Hemi |
| 11.5 × 15.7 (7) | + | Transgenic | I | 5/8 | 5/8 | 5/8 | Hemi |
| 7.1 × 10.23 (2) | + | Transgenic | I | 14/14 | 14/14 | 14/14 | Homo |
| 10.19 × 11.3 (4) | + | Transgenic | I | 16/16 | 10/10 | 10/10 | Homo |
| 3.22 × 14.24 (4) | + | Transgenic | I | 16/16 | 16/16 | 16/16 | Homo |
| 8.13 × 14.15 (9) | − | Non- | S | 0/11 | 0/11 | 0/11 | Azygote |
| | | Transgenic | | | | | |
| 3.22 × 14.24 (11) | − | Non- | S | 0/11 | 0/11 | 0/11 | Azygote |
| | | Transgenic | | | | | |

### EXAMPLE 8.2

### Outline of Scheme for Producing AMV-Resistant White Clover Germplasm Based Upon the Identification of Plants that are Homozygous for Transgenes Using High-Throughput Quantitative PCR Transgene Detection

In total 645 controlled diallel cross combinations of T₁ elite plants from the transformation events H1 and H6 were performed and over 20,000 T₂ seeds have been produced for the identification of 1,600 H1 and H6 homozygous transformation events in elite ‘Mink-type’ background for further selection.

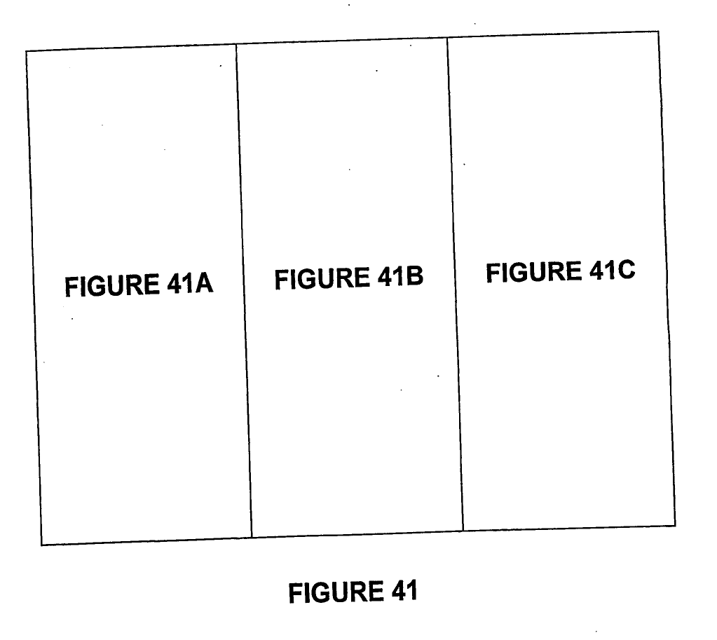

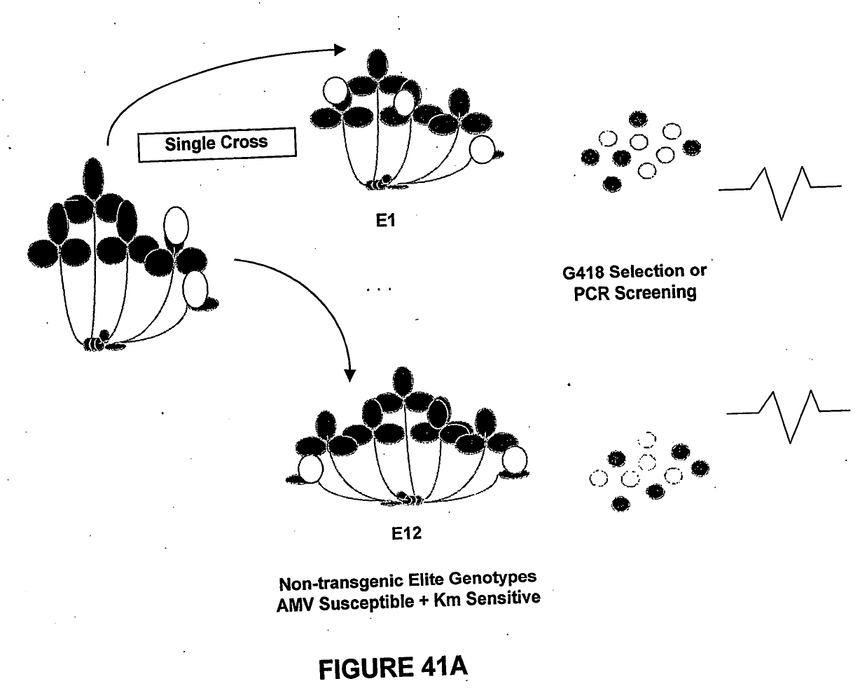

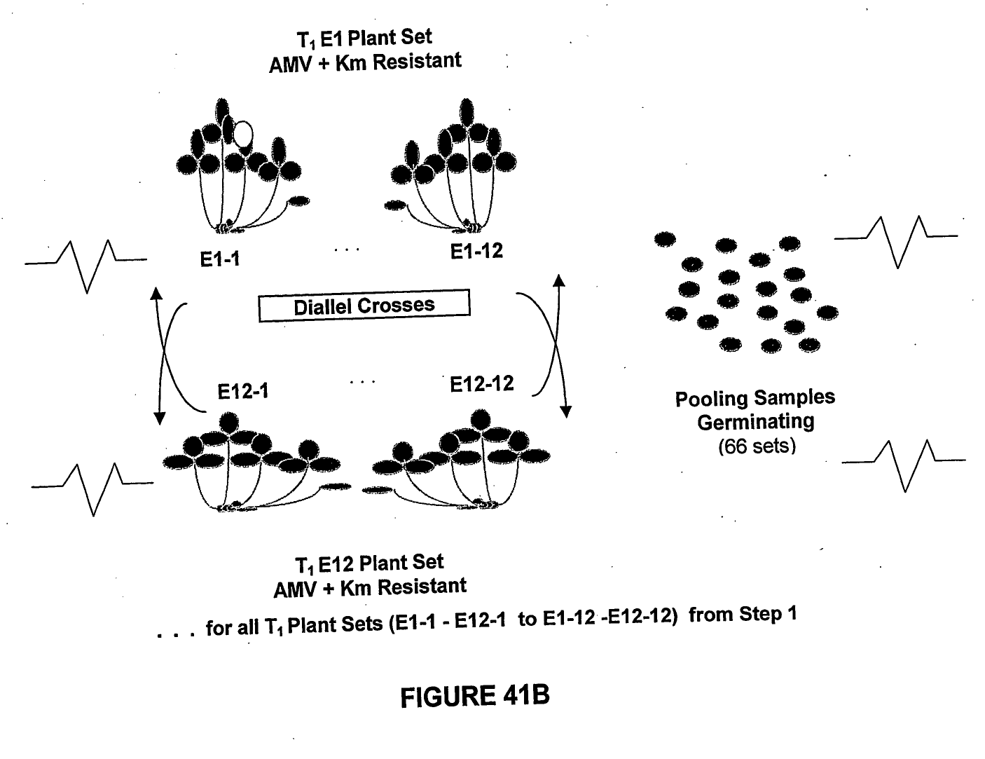

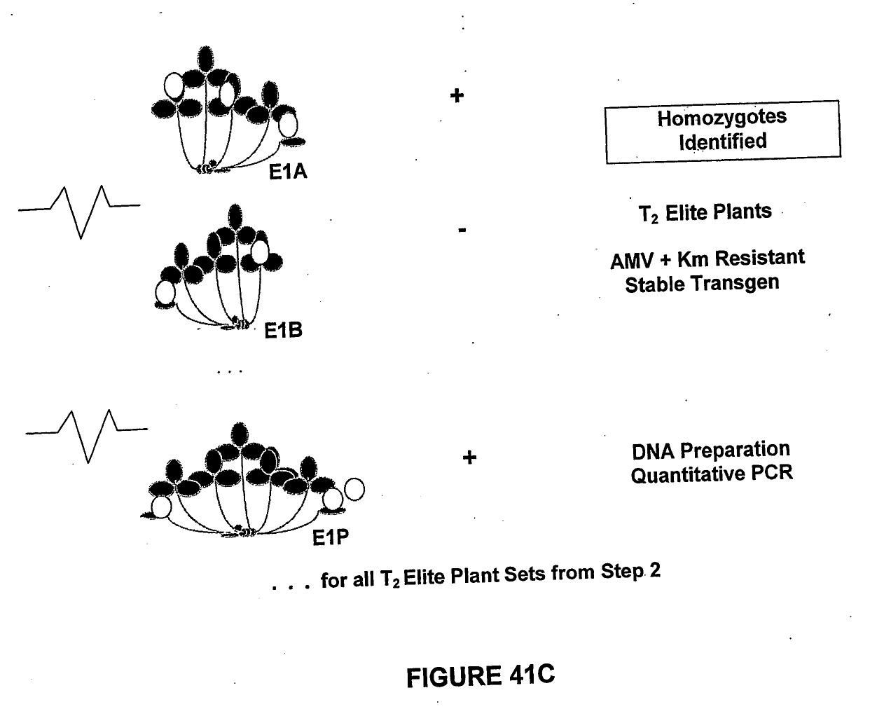

As an alternative to the test-cross approach described above which is highly laborious and time consuming, a new high-throughput direct method for the identification of homozygous T₂ progeny based on quantitative PCR detection has been developed. This method facilitates the identification of the 1,600 T₂ ‘Mink’-type derived AMV immune white clover plants (homozygous for the AMV CP transgene) for the establishment of the breeding nursery in Hamilton. An outline of this procedure is provided in FIG. 41 and the result is shown in the Example below.

### EXAMPLE 8.3

### Evaluation of the Taqman Quantitative PCR Analysis for Discrimination between Heterozygous vs Homozygous T₂ AMV^{r}/KM^{r} White Clover Progeny

1) DNA Extraction:

DNA samples were extracted from one mature trifoliate leaf (c. 50 mg fresh weight) of T₂ progeny from the H1 transformed AMV immune white clover line using the reagents provided in the DNEasy 96 kit (QIAGEN Cat. No. 69181) in combination with a MM300 mixer mill and a QiAGEN 96-well plate centrifugation system according to the protocols provided by the manufacturer. Typical yields of DNA remained constant (10-20 μg of high quality HMW DNA/trifoliate leaf) if due care to harvest similar amount of material was taken. The through-put was 2×96 samples in 2 hours (excluding time required for harvesting material, transferring it into the 192 individual collection tubes and freeze drying it).

2) Primer and Probe Design:

Forward and reverse primers as well as separate probe primer (fluorescently labelled) were designed using the Primer Express Software package (Applied Biosystems). The target sequence was the nptII gene (accession no. V00618, id. ISTN5X). The sequences of the synthesised primers and fluorescently labelled probe were:

Forward Primer:
5′-GGCTATGACTGGGCACAACA-3′;
Reverse Primer:
5′-ACCGGACAGGTCGGTCTTG-3′; and
Probe:
5′-Fam-CTCTGATGCCGCCGTGTTCCG-Tamra-3′.

The forward and reverse primers were used in end point PCR reactions and were found to amplify the expected 155 bp DNA amplicon using the DNA extracted from T₂ progeny containing the nptII and AMV4 transgenes.

3) Quantitative PCR Analysis:

a) Template:

Two positive DNA samples G4 and G5 (independent T₂ genotypes derived from the H1 white clover transformation event), and one negative control sample F2 (non-transformed white clover cv. ‘Irrigation’) which had been isolated using the DNEasy kit and tested by end point PCR were selected for the analysis.

b) PCR Components (per 50 μl reaction):

| | | |
|---|---|---|
| Taqman universal PCR master mix¹ | 25 | μl; |
| primers (fwd/rev) | 300 | nM; |
| nptll probe (FAM labelled) | 200 | nM; |
| 18S rDNA probe +primers | 2.5 | μl; and |
| template DNA in dH₂0 | up to 50 | μl |

(¹Taqman universal master mix (2x) supplied by Applied Biosystems; Cat. No. 4304437).

c) Experimental Design:

To evaluate the feasibility of discriminating between DNA samples originating from T₂ progeny plants with one (heterozygote) vs two (homozygote) nptII transgene copy(ies) per diploid genome equivalent and in the absence of any homozygote plants identified by test crosses an alternative approach was taken. A two-fold difference in absolute number of starting target sequences should theoretically correspond to a displacement of the curves representing the accumulation of PCR products from samples containing one (heterozygote) vs two (homozygote) doses of transgenes by one cycle in the logarithmic amplification phase of the PCR reaction.

A 2-fold dilution series of the independent DNA samples should provide sufficient data for a proper evaluation of the feasibility of the TaqMan system ensuring the reproducibility and sensitivity required for the intended purpose.

d) Serial Dilutions of Template DNA:

DNA samples were diluted 1:2, 1:4, 1:8, . . . 1:128 (in triplicate independent experiments). The starting amounts of total DNA were around 500 ng.

e) Quantitative PCR Reaction:

A master-mix containing Taqman Universal PCR Master Mix, primers, nptII FAM-labelled probe, VIC-labelled probe and primer pair designed from conserved 18 S rDNA sequences (Applied Biosystems Cat. No. 4308329) was dispensed into a MicroAmp Optical 96 well reaction plate (Applied Biosystems Cat. No. N801-0560). The serial dilutions of the three different DNA samples were added to the wells. The wells were sealed with MicroAmp Optical Caps (Applied Biosystems Cat. No. N801-0935) and transferred from the PCR set-up area to the ABI PRISM 7700. The thermocycler profile is as described below:

HOLD for 2 min at 50° C.;

HOLD for 10 min at 95° C.;

40 cycles, each cycle consisting of 15 sec at 95° C., followed by 1 min at 62° C.; and

HOLD at 4° C.

The samples were analysed using the Sequence Detector Software v 1.7 on a Macintosh G3 Power PC.

4) Results:

The C_{T} values (representing the first cycle where fluorescence signal was detected above background) of the 8 no template controls (no DNA) were in the range of 38-40, thus indicating that there is none or negligible contamination of samples with exogenous nptII sequences.

The incremental change in C_{T} values in the 2-fold individual dilution series of the two positive samples G4 and G5 is very close to 1 (Table 33). The average change in C_{T} value for the diluted G4 sample being 1.14 while the average change in C_{T} value for the diluted G5 sample is 1.03. This applies to a 128-fold range in transgene-dosage. When the values are corrected for the internal 18S rDNA control giving ΔC_{T} values the two samples show a consistent difference of on average 2.2 cycles indicating that the G4 sample contains on average 4 times more of the transgene compared to the C5 sample.

TABLE 33
Results From Evaluation of Taqman Quantitativ PCR

| | |
|---|---|
| C_{T} nptll | ΔC_{T} (nptll - 18S) |

| Template | (Mean values of triplicates) | | (Mean values of triplicates) | |
|---|---|---|---|---|
| Dilution | G4 sample | G5 sample | G4 sample | G5 sample |
| 0.5 | 26.97 | 25.23 | 11.39 | 8.96 |
| 0.25 | 27.24 | 26.32 | 11.39 | 8.72 |
| 0.125 | 28.59 | 27.35 | 11.46 | 9.07 |
| 0.0625 | 29.51 | 28.22 | 11.46 | 9.35 |
| 0.0312 | 30.36 | 29.06 | 10.39 | 8.70 |
| 0.0156 | 31.58 | 30.42 | 11.55 | 9.50 |

The high sensitivity of detection in the experiment outlined above (single copy transgene detected in a 100-fold dilution range of DNA template of high complexity) indicates that the amount and quality of the DNA prepared from one single trifoliate leave using the high throughput DNeasy 96 well kit is well suited for the intended purpose.

The consistency in the displacement of the C_{T} values (indicating a stepwise displacement of the curves representing the accumulation of the PCR products by one cycle of amplification) displayed in the 2-fold dilution series of the two positive samples G4 and G5 suggests that this technology can be used for the intended purpose, namely to categorise the DNA samples in groups representing progeny containing the transgene in homozygote or heterozygote state. This was shown to be the case with a trial run of a random number of T2 progenies (Table 34A. and 34B). The results showed that all RNA dot blots and AMV challenge agreed 100% with each other. There was a 16/19 agreement between the Taqman results with the test-cross analysis.

TABLE 34A
Homozygosity Testing using Taqman: Confirmation by Molecular Analysis
and AMV Challenge of T3 Progenies from Test Crosses

| | Back | T3 | NPTll | AMV | RNA Dot | Final |
|---|---|---|---|---|---|---|
| T2 Parents | Cross # | seedling # | PCR | Challenge | Blot Result | Results |
| 7.3 × 10.10 (5)+ | 352 | 1 | + | AMVI (0/4+) | + | Agree |
| Taqman = B1 = | | 2 | + | AMVI (0/4+) | + | Agree |
| 2 copies | | 3 | + | AMVI (0/4+) | + | Agree |
| | | 1 | + | AMVI (0/4+) | + | Agree |
| | | 2 | + | AMVI (0/4+) | + | Agree |
| | | 3 | + | AMVI (0/4+) | + | Agree |
| | | 4 | + | AMVI (0/4+) | + | Agree |
| | 367 | 1 | + | AMVI (0/4+) | + | Agree |
| | | 2 | + | AMVI (0/4+) | + | Agree |
| | | 3 | + | AMVI (0/4+) | + | Agree |
| | | 4 | + | AMVI (0/4+) | + | Agree |
| | | 5 | + | AMVI (0/4+) | + | Agree |
| | | 6 | + | AMVI (0/4+) | + | Agree |
| Taqman | | | 13/13 | 13/13 (I) | 13/13+ | Agree |
| Agreement | | | | | | |
| 10.19 × 11.3 (2)+ | 318 | 1 | + | AMVI (0/4+) | + | Agree |
| Taqman = A1, C3 = | | 2 | − | AMVS (3/4+) | − | Agree |
| 1 copy | | 3 | − | AMVS (2/4+) | − | Agree |
| | 353 | 1 | − | AMVS (2/4+) | − | Agree |
| | | 2 | − | AMVS (2/4+) | − | Agree |
| | | 3 | − | AMVS (2/4+) | − | Agree |
| | | 4 | + | AMVI (0/4+) | + | Agree |
| | 357 | 1 | − | AMVS (3/4+) | − | Agree |
| | | 2 | − | AMVS (3/4+) | − | Agree |
| | | 3 | − | AMVS (1/4+) | − | Agree |
| | | 4 | − | AMVS (2/4+) | − | Agree |
| Taqman | | | 2/11 | 2/11 | 22/11 | Agree |
| Agreement | | | | | | |
| 3.22 × 14.24 (4)+ | 354 | 1 | + | AMVI (0/4+) | + | Agree |
| Taqman = F2 = | | 2 | + | AMVI (0/4+) | + | Agree |
| 2 copies | | 3 | + | AMVI (0/4+) | + | Agree |
| | | 4 | + | AMVI (0/4+) | + | Agree |
| | | 5 | + | AMVI (0/4+) | + | Agree |
| | 363 | 1 | + | AMVI (0/4+) | + | Agree |
| | | 2 | + | AMVI (0/4+) | + | Agree |
| | | 3 | + | AMVI (0/4+) | + | Agree |
| | | 4 | + | AMVI (0/4+) | + | Agree |
| | | 5 | + | AMVI (0/4+) | + | Agree |
| | 370 | 1 | + | AMVI (0/4+) | + | Agree |
| | | 2 | + | AMVI (0/4+) | + | Agree |
| | | 3 | + | AMVI (0/4+) | + | Agree |
| | | 4 | + | AMVI (0/4+) | + | Agree |
| Taqman | | | 16/16 | 16/16 | 16/16 | Agree |
| Agreement | | | | | | |
| 9.20 × 15.20 (5)+ | 358 | 1 | + | AMVI (0/4+) | + | Agree |
| Taqman = E1 = | | 2 | + | AMVI (0/4+) | + | Agree |
| 1 copy | | 3 | + | AMVI (0/4+) | + | Agree |
| | | 4 | − | AMVS (4/4+) | − | Agree |
| | 359 | 1 | − | AMVS (1/4+) | − | Agree |
| | | 2 | − | AMVS (2/4+) | − | Agree |
| | | 3 | − | AMVS (3/4+) | − | Agree |
| | | 4 | − | AMVI (1/4+) | − | Agree |
| | 362 | 1 | − | AMVS (3/4+) | − | Agree |
| | | 2 | − | AMVS (3/4+) | − | Agree |
| | | 3 | − | AMVS (3/4+) | − | Agree |
| Taqman | | | 3/11 | 3/11 | 3/11 | Agree |
| Agreement | | | | | | |

| | TaqMan | NPTll | AMV | RNA Dot | Final |
|---|---|---|---|---|---|
| Controls | Result | PCR | Challenge | Blot Result | Results |
| I2 | 0 Copy | − | AMVS (4/4+) | − | Agree |
| I4 | 0 Copy | − | AMVS (4/4+) | − | Agree |
| I5 | 0 Copy | − | AMVS (4/4+) | − | Agree |
| Taqman | | 0/3 | 0/3 | 0/3 | Agree |
| Agreement | | | | | |

TABL 34B
Confirmati n of Taqman Analysis of T2 Germplasm for Homozgosity and Hemizygosity
by Test Cross s

| T2 Parents being Test-Crossed | | | | | T3 Progeny Tests | | | | |
|---|---|---|---|---|---|---|---|---|---|
| | AMV | Trans- | AMV | | | AMV | | | |
| T2 Plant | RNA | genic | Resist- | TaqMan | nptll | Resist- | AMV | Inferr d | Agree- |
| Lines | signal | Status | ance | Copy # | PCR | ance | RNA | Zygosity | ment |
| 10.19 × | + | T | I | 1 | 2/11 | 2/11 | 2/11 | Hemi | Yes |
| 11.3 (2) | | | | | | | | | |
| 7.3 × | + | T | I | 2 | 13/13 | 13/13 | 13/13 | Homo | Yes |
| 10.10 (5) | | | | | | | | | |
| 10.10 × | + | T | I | 1 | 4/6 | 7/10 | 7/10 | Hemi | Yes |
| 13.6 (5) | | | | | | | | | |
| 9.27 × | + | T | I | 1 | 4/7 | 4/10 | 4/10 | Hemi | Yes |
| 15.8 (6) | | | | | | | | | |
| 9.20 × | + | T | I | 1 | 3/11 | 3/11 | 3/11 | Hemi | Yes |
| 15.20 (5) | | | | | | | | | |
| 3.22 × | + | T | I | 1/2 | 4/5 | 3/6 | 3/6 | Hemi | No |
| 14.24 (5) | | | | | | | | | |
| 7.1 × | − | NT | S | 0 | 0/15 | 0/15 | 0/15 | Azygote | Yes |
| 10.23 (3) | | | | | | | | | |
| 3.22 × | + | T | I | 1 | 3/9 | 3/9 | 3/9 | Hemi | Yes |
| 14.24 (6) | | | | | | | | | |
| 11.5 × | + | T | I | 1 | 5/8 | 5/8 | 5/8 | Hemi | Yes |
| 15.7 (7) | | | | | | | | | |
| 7.1 × | + | T | I | 2 | 14/14 | 14/14 | 14/14 | Homo | Yes |
| 10.23 (2) | | | | | | | | | |
| 8.18 × | − | NT | S | 1 | 0/9 | 0/9 | 0/9 | Azygote | No |
| 15.23 (1) | | | | | | | | | |
| 10.19 × | + | T | I | 1 | 16/16 | 10/10 | 10/10 | Homo | No |
| 11.3 (4) | | | | | | | | | |
| 9.20 × | − | NT | S | 0 | 0/7 | 0/7 | 0/7 | Azygote | Yes |
| 15.20 (2) | | | | | | | | | |
| 3.22 × | + | T | I | 2 | 16/16 | 16/16 | 16/16 | Homo | Yes |
| 14.24 (4) | | | | | | | | | |
| 15 | − | NT | S | 0 | 0 | 0 | 0 | Azygote | Yes |
| 12 | − | NT | S | 0 | 0 | 0 | 0 | Azygote | Yes |
| 8.13 × | − | NT | S | 0 | 0/11 | 0/11 | 0/11 | Azygote | Yes |
| 14.15 (9) | | | | | | | | | |
| 3.22 × | − | NT | S | 0 | 0/11 | 0/11 | 0/11 | Azygote | Yes |
| 14.24 (11) | | | | | | | | | |
| 10.19 × | + | T | I | 1 | 3/8 | 3/8 | 3/8 | Hemi | Yes |
| 11.3 (2) | | | | | | | | | |

T = transgenic
NT = non-transgenic

The use of an internal control gene such as the 18S rDNA gene allows for a more accurate sample comparison minimising the effects of variations inquality and starting concentration of DNA template and avoiding time-consuming quantification of concentrations of each DNA sample to be analysed (Table 34C).

TABLE 34C
Observed dCt values for the npt2 transgene
using 18S rRNA as an internal control.

| Transgene Copy # | 18S rRNA | Npt2 | dCt |
|---|---|---|---|
| 0 | <19 | >35 | >16 |
| 1 | <19 | 29-31 | 10-12 |
| 2 | <19 | <28 | 9-10 |

Based on comprehensive analysis of 840 T2 progenies and 762 T3 progenies from test crosses comparing the results of RNA dot blot, AMV resistance testing and quantitative PCR using the TaqMan transgene detection system, the high-throughput quantitative PCR transgene detection is:

99% correlated with transgene mRNA detection

99% correlation with resistance phenotype

91% correlation with test-cross analysis for the identification of homozygous transgene genotypes

85% reproduciblity with a second taqman quantitative detection for the AMV transgene

Consistent with the expected Mendellian ratio expected from a diallel cross (1 copy=65%, 2 copies=35% out of 762 transgenic T3 progenies)

### EXAMPLE 8.4

### Mass Screening of T2 AMV-Resistant White Clover Germplasm for Genotypes Homozygous for Transgenes Using High-Throughput Quantitative PCR Transgene Detection

The Taqman quantitative PCR analysis has been found to be suitable for use as a high tthrough-put screening system for discrimination between heterozygous vs homozygous T₂ AMV^{r}/KM^{r} white clover progeny. Using this system the inventors have successfully identified

1216 homozygous H1 locus genotypes out of 5294 T2 progenies

449 homozygous H6 genotype out of 2198 progenies

These results show that it is possible to screen over 7000 seedlings over a short period of 4 months by two technicians.

### EXAMPLE 8.5

### High Throughput Screening for npt2, AMV Coat Protein and CYVV Coat Protein Sequences in Transgenic Progenies using Taqman Based PCR

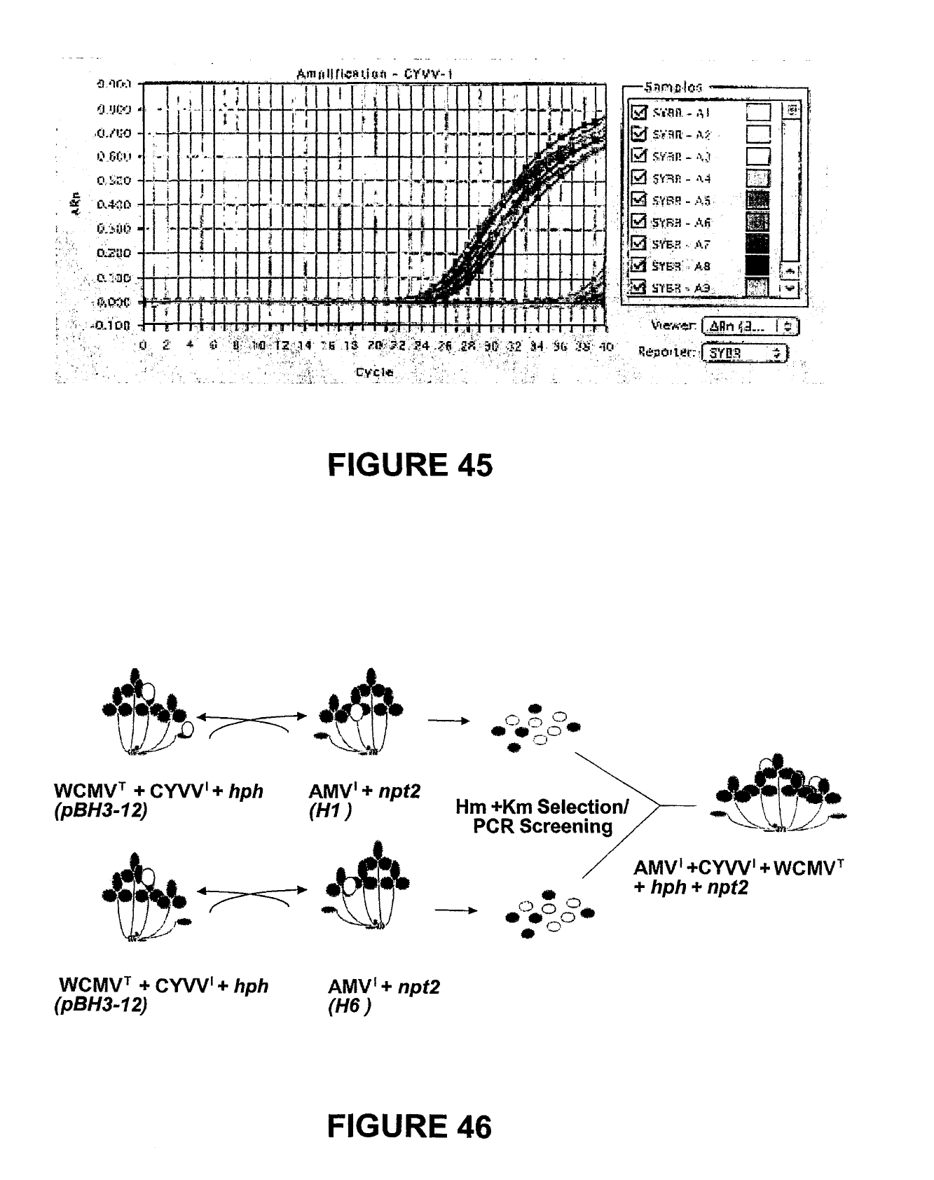

Given the 99% accuracy of the Taqman quantative PCR system for rapid identification of transgenic genotypes from large scale diallel crosses, we have also shown that the system can be used to detect for the presence or absence of specific transgenes during the course of molecular breeding of transgenic plants, such as in the production of dual virus resistant germplasma by crossing plants each with a single virus resistant gene. FIG. 45 shows the level of discrimination achievable and Table 34D shows the 100% accuracy of the Taqman quantitative PCR system for identifying expected transgenic and non-trangenic genotypes of white clover in a molecular breeding program. This system has been applied successfully for the detection of a range of transgenes including npt2, AMV coat protein and CYVV coat protein genes.

TABLE 34D
Typical results of an experiment screening for transgenic
progenies by RNA dot blot and by the Taqman quantitative
PCR system of high throughput screening for npt2 in the
white clover germplasm development program

| | | RNA Dotblot | TaqMan | |
|---|---|---|---|---|
| T2 Progeny # | T3 Progeny # | Result | npt2 | Agreement |
| 1.30 × 2.16/7 | 1 | + | + | Yes |
| Homozygote | 2 | + | + | Yes |
| | 3 | + | + | Yes |
| | 4 | + | + | Yes |
| | 5 | + | + | Yes |
| | 6 | + | + | Yes |
| | 7 | + | + | Yes |
| | 8 | + | + | Yes |
| | 9 | + | + | Yes |
| | 10 | + | + | Yes |
| | 11 | + | + | Yes |
| 1.24 × 7.4/4 | 1 | + | + | Yes |
| Homozygote | 2 | + | + | Yes |
| | 3 | + | + | Yes |
| | 4 | + | + | Yes |
| | 5 | + | + | Yes |
| | 6 | + | + | Yes |
| | 7 | + | + | Yes |
| | 8 | + | + | Yes |
| | 9 | + | + | Yes |
| | 10 | + | + | Yes |
| | 11 | + | + | Yes |
| | 12 | + | + | Yes |
| 1.26 × 9.2/10 | 1 | − | − | Yes |
| Hemizygote | 2 | + | + | Yes |
| | 3 | − | − | Yes |
| | 4 | − | − | Yes |
| | 5 | + | + | Yes |
| | 6 | + | + | Yes |
| | 7 | + | + | Yes |
| | 8 | + | + | Yes |
| | 9 | − | − | Yes |
| | 10 | − | − | Yes |
| | 11 | + | + | Yes |
| | 12 | + | + | Yes |
| | 13 | − | − | Yes |
| | 14 | − | − | Yes |
| 8.13 × 14.15/9 | 1 | − | − | Yes |
| Azygote | 2 | − | − | Yes |
| | 3 | − | − | Yes |
| | 4 | − | − | Yes |
| | 5 | − | − | Yes |
| | 6 | − | − | Yes |
| | 7 | − | − | Yes |
| | 8 | − | − | Yes |
| | 9 | − | − | Yes |
| | 10 | − | − | Yes |
| | 11 | − | − | Yes |

Part IX of the Experimental Section:

Pyramiding Transgenic Virus Resistance Traits by Sexual Crossing

### EXAMPLE 9.1

### Production of AMV Plus CYVV Double Virus Resistant White Clover by Crossing Single Virus Resistant Lines

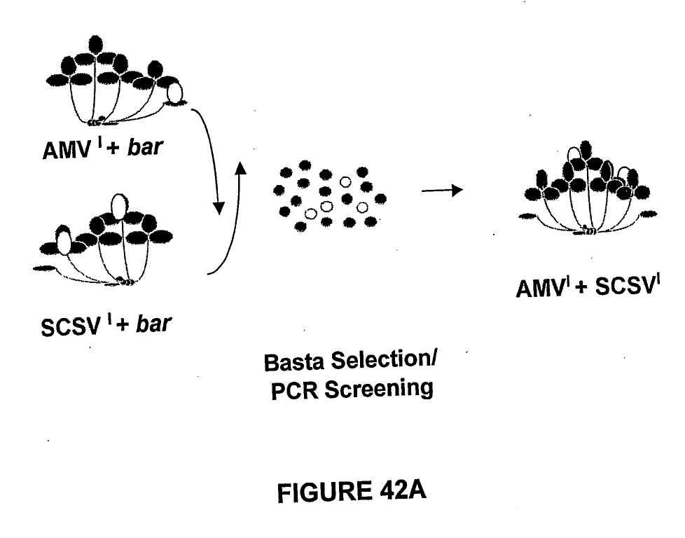

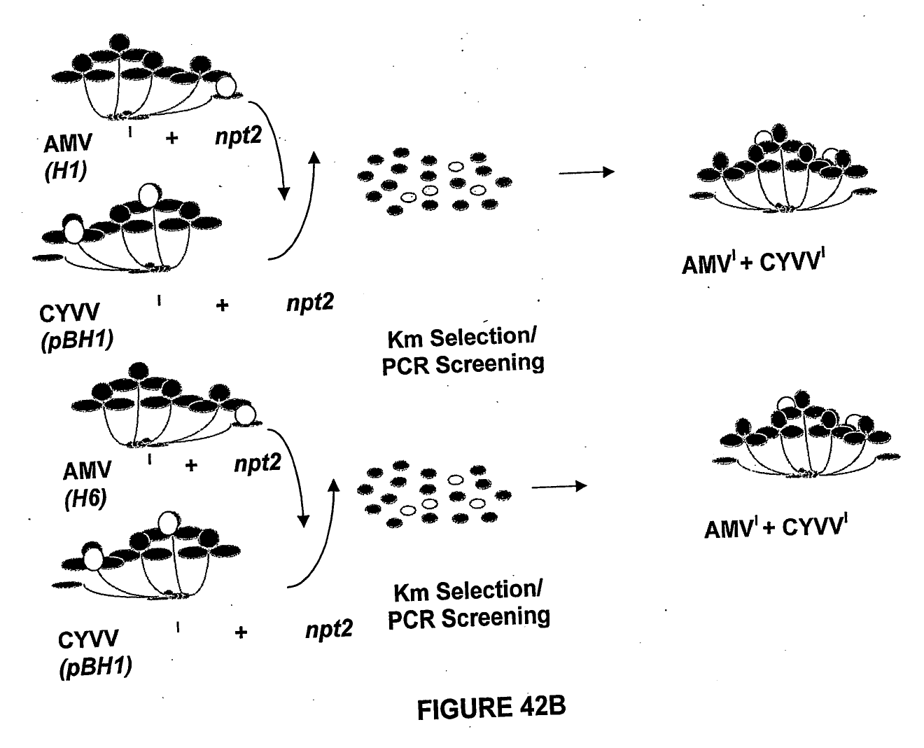

In this Example the inventors have used the best AMV or CYVV resistant transgenic lines as the source parents in sexual crosses, to produce AMV plus CYVV double virus resistant plants. The strategy is graphically described in FIG. 42B and the background of the parents is shown in Table 35A. The inventors conducted the following crosses:

four homozygous H1 derived AMV immune lines (A, B, C, D) crossed with with one transgenic BH1 (CYVV CP) CYVV immune line (BH1-4), two putative transgenic BH2 (CYVV duplex) CYVV immune lines (BH2-10 and BH2-12), one putative non-transgenic but CYVV immune line (BH1-18) and one non-transgenic but CYVV resistant line (14).

four homozygous H6 derived AMV immune lines (B1, F1, B2, F2) crossed with the above BH1, BH2 and non-transgenic CYVV immune/resistant lines.

and as a control, the BH1, BH2, and non-transgenic CYVV immune/resistant lines were test-crossed with a CYVV susceptible (15) non-transgenic plant.

TABLE 35A
AMV and CYVV single virus resistant plants
used for pyramiding double virus resistance

| | | CYVV | AMV, |
|---|---|---|---|
| | | Coat | CYVV |
| | Parental | Protein | Resistance |
| | Lines | Gene PCR | Phenotype |
| AMV Resistant | B1 | − | H6 AMVr |
| Source Parents | B2 | − | H6 AMVr |
| | F2 | − | H6 AMVr |
| | F1 | − | H6 AMVr |
| | A | − | H1 AMVr |
| | B | − | H1 AMVr |
| | C | − | H1 AMVr |
| | D | − | H1 AMVr |
| CYVV Resistant | BH1-4 | + | T CYVVr |
| Source Parents | BH1-18 | − | CYVVr |
| | BH2-10 | − | CYVVr |
| | BH2-12 | − | CYVVr |
| | I4 | − | NT CYVVr |

NT = non-transgenic;
T = transgenic

The progenies from these crosses were screened for the presence or absence of the CYVV coat protein transgene and tested for CYVV resistance. The result of a typical experiment is shown in Table 35B. The Table showed that in the crosses shown, there was a direct correlation between the presence of the CYVV coat protein gene with CYVV immunity in the progenies. The cross between BH1-4 CYVV immune parent and the F1 AMV immune parent produced progenies with both AMV and CYVV resistance. In contrast, all progenies from the 14×15 non-trangenic cross were susceptable to CYVV.

TABLE 35B
CYVV Resistance of CYVV × H6 - AMV Gross Progenies

| Transgenic | | | | | |
|---|---|---|---|---|---|
| CYVV | AMV | | | Progeny | Progeny |
| Resistance | Resistance H6 | Cross | Progeny | CYVV | CYVV |
| Parent | Parent | # | Seedling # | PCR | Resistance |
| BH1 - 4 | F1 | 536 | 1 | + | I |
| | | | 2 | + | I |
| | | | 3 | + | I |
| | | | 4 | − | S |
| | | | 8 | + | I |
| | | | 9 | − | I |
| | | | 10 | + | I |
| | | | 13 | + | I |
| | | | 15 | − | S |
| Non | | | | | |
| transgenic | | | | | |
| CYVV | | | | Progeny | Progeny |
| Resistance | Control Test | Cross | Progeny | CYVV | CYVV |
| Parent | Cross Parent | # | Seedling # | PCR | Resistance |
| I4 | I5 | 565 | 1 | − | S |
| | | | 2 | − | S |
| | | | 3 | − | S |
| | | | 4 | − | S |
| | | | 6 | − | S |
| | | | 7 | − | S |
| | | | 8 | − | S |
| | | | 9 | − | S |

S = Susceptible (>50% infection)
I = Immune (0% infection)

When a total of 117 progeny seedlings from the various combination of crosses between AMV and CYVV immune parents were tested for CYVV resistance screening and molecular analysis, the results produced 67 AMV+CYVV resistant lines (Table 35C.). Analysis of the result showed that there were approximately twice as many CYVV immune lines among the CYVV coat protein PCR positive progenies (87%) than among the PCR negative progenies (46%).

TABLE 35C
Production of AMV plus CYVV resistant plants by
sexual crossing between AMV and CYVV immune parents

| | | | |
|---|---|---|---|
| | Parent 1: | Parent1: | |
| CYVV | Homozygous | Homozygous | |
| Resistant | H1 AMV Immune | H6 AMV Immune | NT Test |
| Parents | Parents | Parents | Parent (I5) |

AMV × Resistance Summary (CYVV PCR+)
Total = 27/31 = 87% CYVVr

| | | | |
|---|---|---|---|
| BH1-4 | 6/9 | 18/19 | 3/3 |

AMV × Resistance Summary (CYVV PCR−)
Total = 40/86 = 46.57% CYVVr

| | | | |
|---|---|---|---|
| BH1-4 | 2/4 | 3/8 | 2/6 |
| BH2-10 | 2/4 | 5/13 | |
| BH2-12 | 3/13 | | |
| BH2-18 | 3/4 | 10/15 | 10/11 |
| I4 | | | 0/8 |

### EXAMPLE 9.2

### Production of AMV Plus WCMV Double Virus Resistant Red Clover by Crossing Single Virus Resistant Lines

Also tested were T₁ offspring plants from crosses between the an AMV immune T₀ red clover line (RA4.2) with two independent To lines (RW9.3 and RW11.2, see Table 7B) of red clover expressing the WCMV coat protein gene. The results (Table 36) showed that AMV immunity can be recovered readily from the resultant progenies but no WCMV resistant lines were obtained as the parents were all susceptible to WCMV. This demonstrates that sexual crosses between AMV and WCMV coat protein transgenic lines have not affected the stability of the AMV immunity in the offsprings.

TABLE 36
AMV resistance in transgenic progeny lines
of red clover obtained by crossing parents
expressing AMV and WCMA coat protein genes.

| | | PCR | | |
|---|---|---|---|---|
| Virus¹ | Plant code | AMV | WCMV | AMV Resistance |
| AMV | Cowpea | na | na | 310 lesions |
| | indicator | | | |
| | Renegade | na | na | S |
| | C1.10(control) | − | − | S |
| | C1.18(control) | − | − | S |
| | RA4.2-T₀ | + | − | I |
| | BF1.24-T₁ | − | + | S |
| | CF1.34-T₁ | + | − | I |
| | FF1.13-T₁ | + | + | I |

¹Virus inoculum was derived from white clover infected with AMV isolate WC28
2) C1.10, and C1.18 are seed derived cv. Renegade plants
S = Susceptible (>50% infection)
I = Immune (0% infection)

The results shown in Examples 9.1 and 9.2 demonstrate that it is possible to produce various pasture legume plants with multiple virus resistance by crossing single and double virus resistant plants expressing the corresponding virus resistance genes. These include:

Production of double AMV plus SCSV virus resistant subterranean clover by crossing SCSV resistant transgenic lines SC19 and SC 4 with AMV resistant subterranean clover lines. The strategy is graphically described in FIG. 42A.

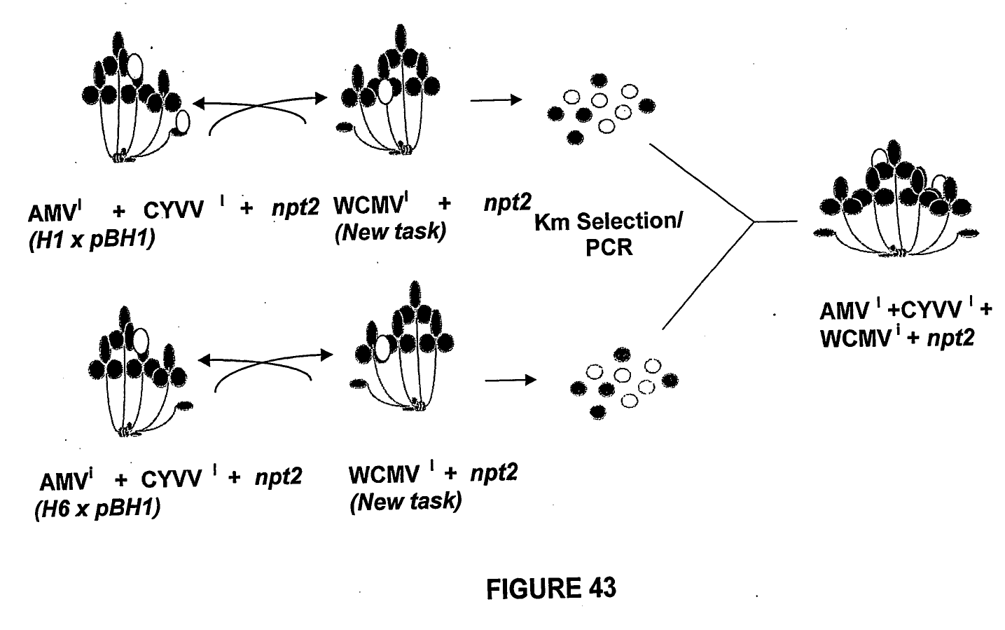

Production of triple virus-resistant lines by crossing AMV plus CYVV double virus resistant white clover with a future WCMV resistant white clover line as described in FIG. 43.

Production of triple virus-resistant lines by crossing an AMV resistant white clover line with a future CYVV plus WCVV double virus resistant white clover line as depicted in FIG. 46.

Part X of the Experimental Section:

### Identification of Suitable Promoters other than CaMV35S

As described herein above, we have shown that both the A. thaliana SSU and the SCSV promoters are effective for driving virus coat protein gene constructs for developing virus resistant plants. We have shown that the A. thaliana SSU promoter is particularly more efficient than the CaMV 35S promoter in conferring immunity against AMV in white clover and lucerne, and when used to drive the CYVV coat protein gene, is very efficient in protection against CYVV in white clover. Thus it should be useful for expressing virus resistance gene other than coat protein, and in legume crops, such as subterranean clover, red clover, Persian clover, lentil and chickpea, etc., that are affected by these viruses other then white clover and lucerne. Similarly, the SCSV promoter constructs described have been demonstrated to be as efficient as the CaMV 35S promoter in conferring immunity against AMV in white clover and lucerne. It should also be useful for expressing virus resistance gene other than the coat protein genes and in other legume crops. Since a number of the SCSV promoters are available for use in the same plants, these promoters are particularly useful where a number of different promoters are required to drive multiple genes in the same plant, such as for developing plants with multiple virus resistant genes. Double-resistant AMV+CYVV plants are crossed with a WCMV resistant white clover line as described in FIG. 46 to produce triple-resistant lines.

Part XI of the Experimental Section:

A N vel Strategy t C nfer R sistanc to Virus on Plants Using a Modif d Viral Replication Protein.

A protein mediated mechanism by which the 1a protein molecules defective in ATP binding could confer resistance is by binding with 2a protein molecules to form dysfunctional viral replication complexes. With a large number of defective 1a molecules present in the cytoplasm of cells, 2a protein molecules synthesized by infecting virus should not be able to form functional replication complexes and thereby stop further viral replication and infection.

All four genomic AMV RNAs (1-4) from strain 425 have been cloned into pUC 9 based vectors with a 35S promoter and a nos terminator (pCa17T, pCa27T, pCa32T and pCa42T, respectively for RNA 1, 2, 3 and 4 obtained from Dr John Bol, Gorlaeus Laboratories Leiden University Netherlands), and have been shown to be infectious when they are all co-inoculated onto Nicotian tabacum cv Samsun NN (Neeleman et al, 1993). To demonstrate that the putative ATP binding site in the AMV 1a protein is presumably involved in ATP hydrolysis and that the mutations proposed to the motif make it dysfunctional, we made mutant derivatives of the AMV RNA 1 infectious clone and then test their infectivity.

### EXAMPLE 11.1

### Cloning Strategy for the Development of AMV RNA 1 Infectious Clone Mutant Deriviatives with Defective ATP Binding

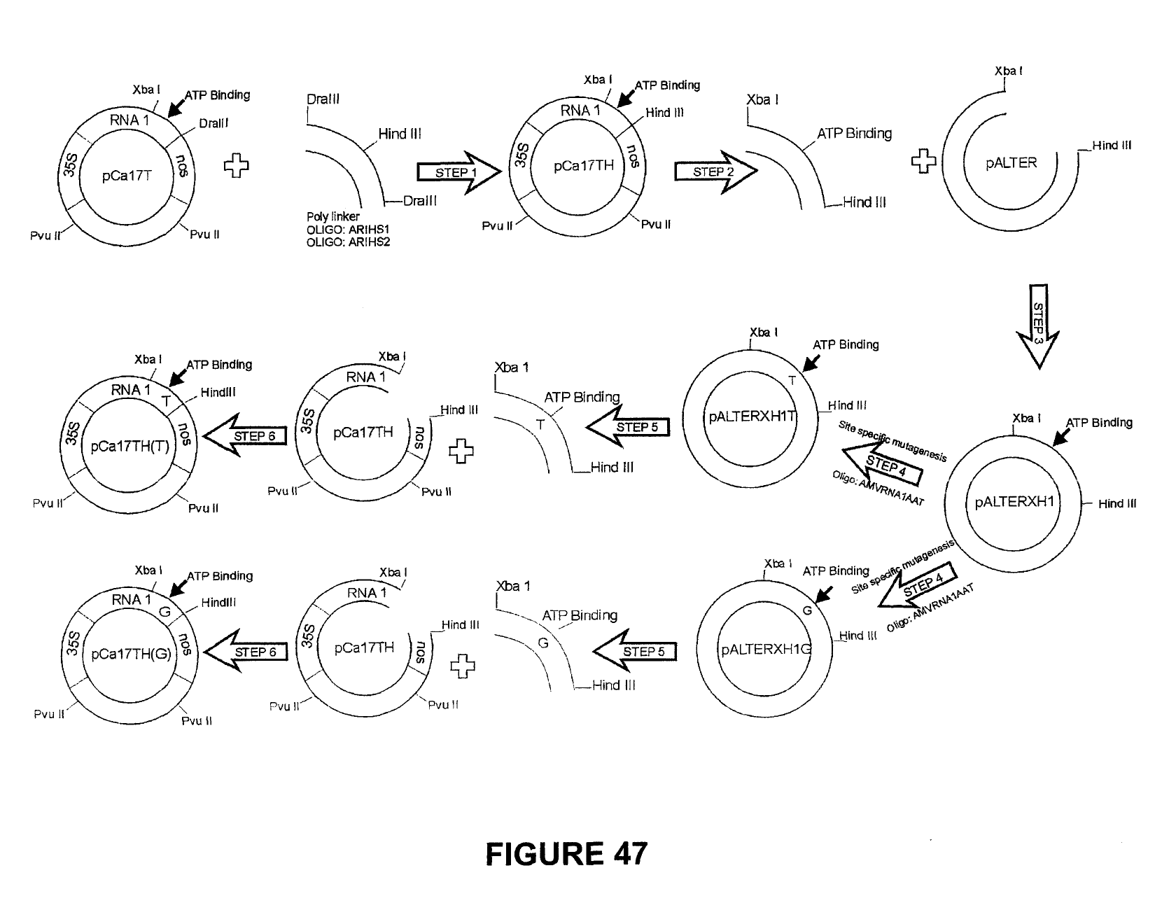

The cloning strategy for the development of the mutant AMV RNA 1 clones with defective ATP binding is summarized in FIG. 47. The AMV RNA 1 infectious clone pCa17T was digested with the DraIII restriction enzyme and a polylinker with oligonucleotides AR1HS1 (nucleotide sequence 5′-GTGAAGCTTCCCGGGCACTGG-3′; SEQ ID NO: 46) and AR1HS2 (nucleotide sequence 5′-ACCCACTTCGAAGGGCCCGTG-3′; SEQ ID NO: 47) were ligated with T4 DNA ligase (Promega). The polylinker introduced one HindIII and one SmaI restriction enzyme site so as to allow the DNA coding sequence for the ATP binding motif to be cloned into the site specific mutagenesis vector p-ALTER-1 (Promega). The plasmid formed is called pCa17TH. pCa17TH plasmid was then digested with XbaI and HindIII restriction enzymes with the resultant fragment containing the DNA sequence coding for the ATP binding motif cloned into XbaI and HindIII digested pALTER-1 to produce the plasmid pALTERXH1.

Oligonucleotides (21 nucleotides) were designed for the site-specific mutagenesis of the ATP binding site. The DNA and protein sequences designated AMVRNA1GAA (for changing a codon from AAA to GAA), and AMVRNA1AAT (for changing a codon from AAA to AAT) are shown below, where the sequence of the oligonucleotides (AMVRNA1GAA and AMVRNA1AAT) used for the site specific mutagenesis is indicated by the line above the DNA sequence. The T and G DNA base changed is indicated by an underline as is the resultant amino adid change. The change in Mutant G is referred as being the ‘G’ series from the base changed, similarly Mutant T is referred to as the ‘T’ series.

```
Wild Type:
              5′GGA GTT GCT GGT TGC GGA AAA ACC ACC AAT A 3′, (SEQ ID NO: 48)
                  G V A G C G K T
Mutant G: AMVRNA1GAA
              5′GGA GTT GCT GGT TGC GGA GAA ACC ACC AAT A 3′, (SEQ ID NO: 49)
    G V A G C G E T
using the oligonucleotide AMVRNA1AAT (5′-GTTGCGGAAATACCACCAATA-3′; SEQ ID NO: 50)
Mutant T: AMVRNA1AAT
              5′GGA GTT GCT GGT TGC GGA AAT ACC ACC AAT A 3′, (SEQ ID NO: 51)
    G V A G C G N T
using the oligonucleotide AMVRNA1GAA (5′-GTTGCGGAGAAACCACCAATA-3′;. SEQ ID NO: 52)
```

Site-specific mutagenesis was undertaken using Promega Altered Sites®II in vitro Mutagenesis System. Two plasmids containing the required changes to the DNA sequence coding for the ATP binding motif were produced, called pALTERXH1G and pALTERXH1T—the last letter in the name of these plasmids refers to the DNA base changed. The mutagenesis was confirmed by sequencing. The pALTERXH1G and pALTERXH1T plasmids were digested with XbaI and HindIII and the respective DNA fragment containing the sequence for the now mutated ATP binding motif were re-cloned back into pCa17TH to produce the plasmids pCa17TH(G) and pCa17TH(T). The plasmids were transformed into E. coli strain ES1301 mutS. Single colonies containing the mutant plasmids were then selected for plasmid purification (miniprep) and sequence analysis to ensure the desired mutation had been incorporated.

DNA sequencing was carried out in plasmid DNA using the ABI Prism Dye Terminator Cycle Sequencing System (part# 402078) manufactured and supplied by Perkin Elmer. Template DNA was prepared by precipitation with PEG 8000 and 5 pmol of primer was used per reaction.

The DNA sequence coding for the ATP binding motif was always confirmed in putative clones in both the forward and reverse directions using the primer AMV1ATPFP 5′ GTCTTTGTTGACCAATCTTGCGTC 3′ (SEQ ID NO: 53), and the primer AMV1ATPRP (5′ AACTTTGTCAACGGTGAACAATCG 3′) (SEQ ID. NO: 54), respectively. The AMV1ATPFP primer binds at a position 80 nucleotides to the 5′ side of the sequence coding for the ATP binding motif and the AMV1ATPRP binds at a position 95 nucleotides to the 3′ side.

### EXAMPLE 11.2

### Analysis of Alfalfa Mosaic Virus RNA 1 with Dysfunctional ATP Binding Motif Mutants by Infectivity Studies on Cowpea

Large Scale Plasmid Preparation

Large quantities of the plasmid DNA of the infectious clones and the RNA1 mutant derivatives required for infectivity studies were prepared using a Maxi Plasmid Preparation Kit (catalogue # 12163) manufactured by Qiagen, using 2L cultures. All of the plasmids were grown in E. coli strain DH5α. The quantity and quality of DNA was determined by spectrophotometer and agarose gel electrophoresis.

Inoculation and Infectivity Evaluation of the Infectious Clones and their Derivatives

The plasmid DNA of each infectious and derivative clone was digested separately with PvuII which cleaves at positions 200 bp upstream of the 35S promoter and 90 bp downstream of the nos terminator (Neeleman et al, 1993; see also FIG. 47). Complete digestion was confirmed by agarose gel electrophoresis and the quantity of DNA was estimated using A₂₆₀. Mixtures of the infectious clones to give the appropriate amount of each digested plasmid were made prior to inoculation and were verified by gel electrophoresis.

Cowpeas (Vigna unguiculata, cultivar Blackeye) were inoculated when the first leaves reached full expansion which ranged from 4 to 6 six days after germination in the glasshouse. Only plants with uniform growth and no emerging shoot tips were used. The selected seedlings were sensitised to virus infection by being placed in the dark for about 16 hours before inoculation. A small sprinkling of 37 μm carborundum was placed onto each half leaf immediately before a water mixture (20 μl) of the plasmids of the infectious clones and derivatives was applied. The leaves were gently rubbed five times. After a period of 5 to 15 minutes, the inoculated leaves were washed with water. Local lesions were assessed and counted between 4 and 7 days after inoculation.

The infectious clones (pCa17T, pCa27T, pCa32T and pCa42T which code for the AMV genomic RNAs 1, 2, 3 and 4 respectively) were inoculated onto the half leaves of cowpeas at three different levels (0.5 μg, 2.0 μg and 10 μg of each construct), with four replicates, and using AMV isolate WC28 viral inocula as a positive control. The results showed that inoculations using 2.0 μg of each infectious clone gave around five times the number of local lesions as the 0.5 μg but a similar number to that where 10 μg of each infectious clone was inoculated. Single lesions from both the infectious clones and AMV isolate WC28 were re-inoculated onto cowpeas to confirm that they were indeed caused by virus infection. The appearance of the lesions in the re-inoculation were the same as for those on the initial inoculated leaves. In the negative control, no lesions were observed where water or 0.1M phosphate buffer (pH 7.4) was inoculated.

Comparison of the Infectivity of Unmodified to Modified AMV RNA 1 Infectious Clones.

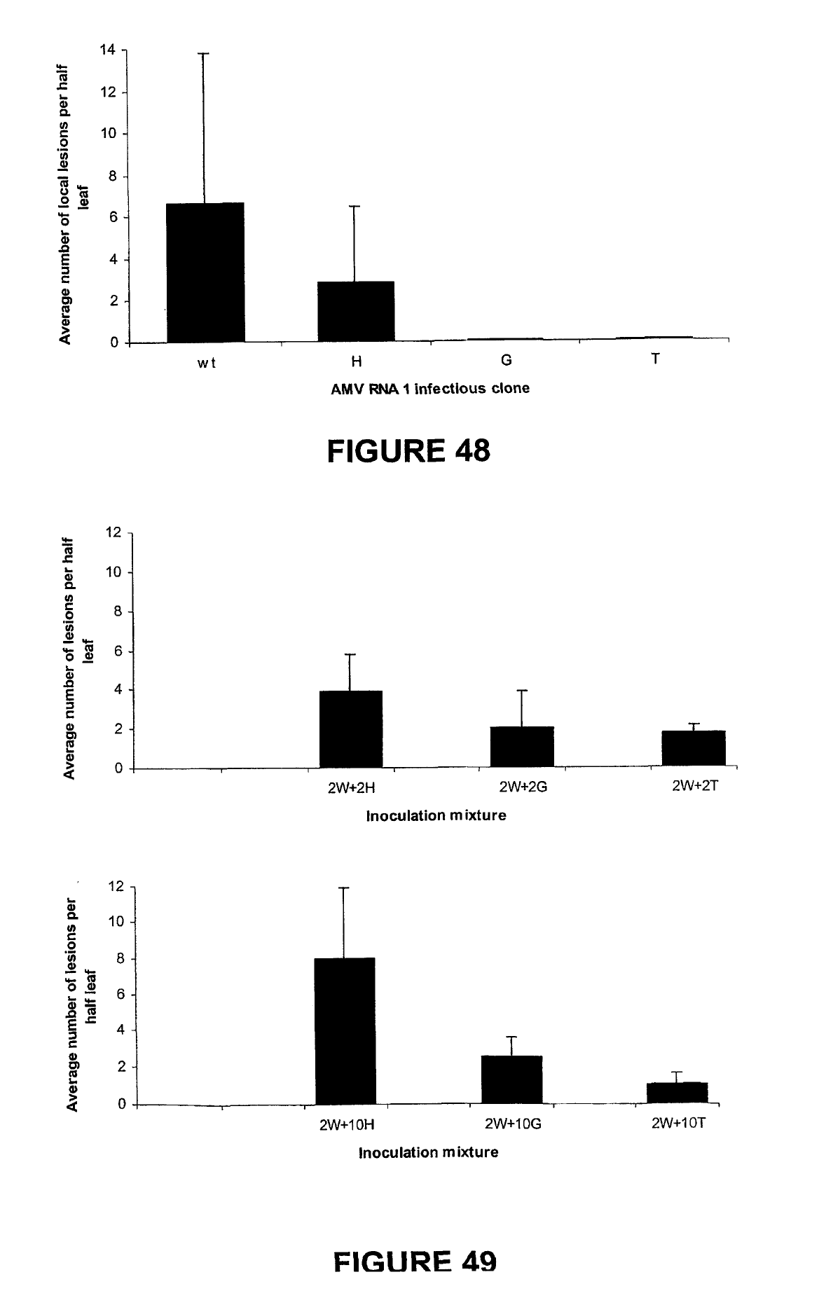

The infectivity of the wild type AMV RNA 1 infectious clone pCa17T was compared with the three made as described above, pCa17TH, pCa17TH(G) and pCa17TH(T), by inoculating separately 2 μg each of the plasmids with 2 μg of each of the other infectious clones required for viral infection (pCa27T, pCa32T and pCa42T) representing AMV RNAs 2-4. All plasmids were digested with the restriction enzyme PVUII prior to inoculation. Four replicate Cowpea half leaves were inoculated in two separate experiments. The results of each experiment were comparable and the results pooled, and are shown in FIG. 48.

The infectious clone with the insertion of the polylinker sequence in the 3′ untranslated region (pCa17TH) had a 50% reduction in infectivity compared to the ‘wild type’ (pCa17T). In contrast, both plasmids which contained the modified ATP binding motif (pCa17TH(G) and pCa17TH(T)) did not give rise to any local lesions and therefore were deemed to be non-infectious.

The constructs with the modified ATP binding sites were not infectious regardless of the amount of DNA inoculated or the amount of the RNA 2-4 plasmid DNA. This confirms the results of the previous experiment that the constructs pCa17TH(G) and pCa17TH(T) were not infectious. It can be concluded that the putative ATP binding motif in the AMV RNA 1 gene is essential for virus infection and that both mutations stop the P-loop from undertaking ATP hydrolysis in vivo.

Competitive Inhibition of Virus Infection by Mutant Forms of the AMV RNA 1 Infectious Clone.

To test if AMV 1a protein derived from the mutant forms of the AMV RNA 1 infectious clones that are presumably defective for ATP binding could inhibit in vivo the infection of AMV, mixtures of the unmodified (2 μg) and modified (2 μg or 10 μg) AMV RNA 1 infectious clones were co-inoculated onto cowpea half leaves with 2 μg of each of the AMV RNAs 2-4 infectious clones. The results are summarized in FIG. 49. The number(s) in the label for each inoculation mixture refers to the amount (μg) of the AMV RNA 1 infectious clone added. The letter ‘W’ refers to the unmodified AMV RNA 1 infectious clone pCa17T, whilst ‘H’ refers to pCa17TH, ‘G’ refers to pCa17TH(G) and ‘T’ to pCa17TH(T).

When the unmodified infectious clone, pCa17T, was co-inoculated with pCa17TH there was no change in the number of lesions compared to when pCa17T alone was inoculated (data not shown).

When pCa17T was co-inoculated with pCa17TH(G) or pCa17TH(T) there was a approximately a 50% decrease in the number of lesions regardless if either 2 μg or 10 μg of the modified clones was inoculated. This is strong evidence that the pCa17TH(G) and pCa17TH(T) clones produce AMV 1a protein defective in ATP binding and which can interfere with the replication and hence replication of AMV whereas the pCa17TH construct produces functional AMV 1a protein. This is the first time that a defective viral protein has been shown to interfere with virus infectivity in vivo.

When 10 μg of the AMV RNA 1 derived plasmid clones were inoculated with 2 μg of the AMV RNA 1 infectious clone there was no statistical difference between the different co-inoculation combinations at the 5% level, however there was marginal significant difference at the 5% level between the inoculation using the ‘H’ plasmid and the ‘T’ plasmid (p=0.056).

### EXAMPLE 11.3

### Production and Evaluation of Transgenic Tobacco and White Clover Containing the Wild Type and Mutant ATP Motif Forms of the AMV RNA 1 Gene for Resistance to AMV Infection.

On the assumption that the mutation of the putative ATP binding site in the AMV RNA 1 infectious clones negate the function of the protein synthesized, and that defective AMV 1a protein is able to compete with the wild type AMV 1a protein to form non-functional replication complexes with AMV 2a protein, the next step was to transform plants to express the mutant forms of AMV RNA 1 gene and to test for resistance to AMV infection. Two plant species, tobacco, as a model plant system, and white clover as a commercial plant with high susceptibility and little natural resistance to AMV infection were chosen to evaluate this proposed mechanism of virus resistance.

Cloning Strategy for the Development of Binary Vectors Containing the Wild Type and Mutant ATP Motif Forms of the AMV RNA 1 Gene

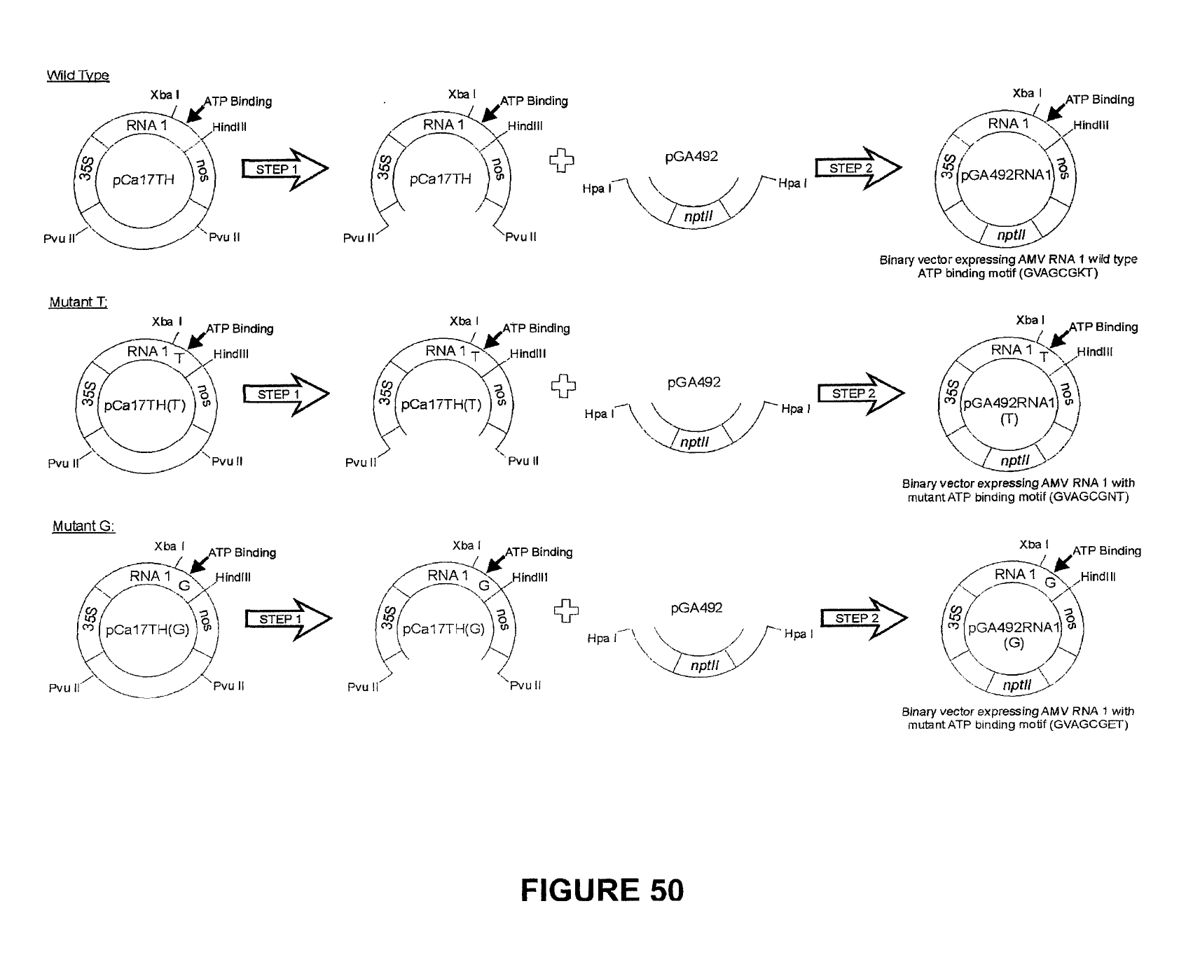

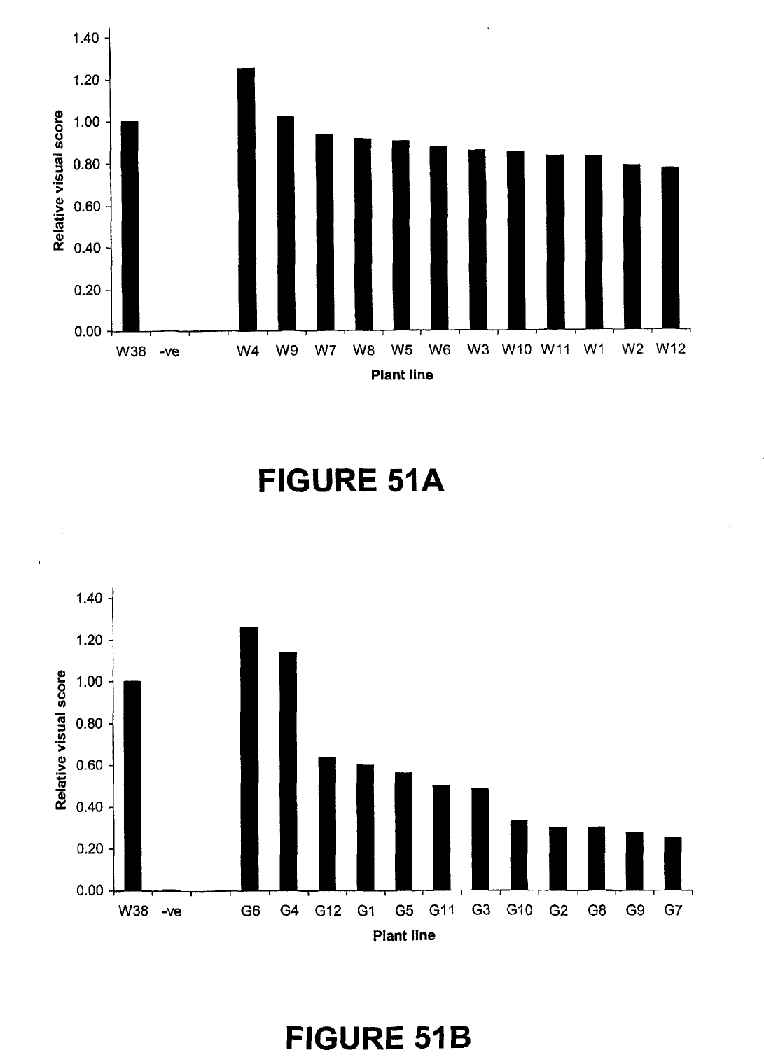

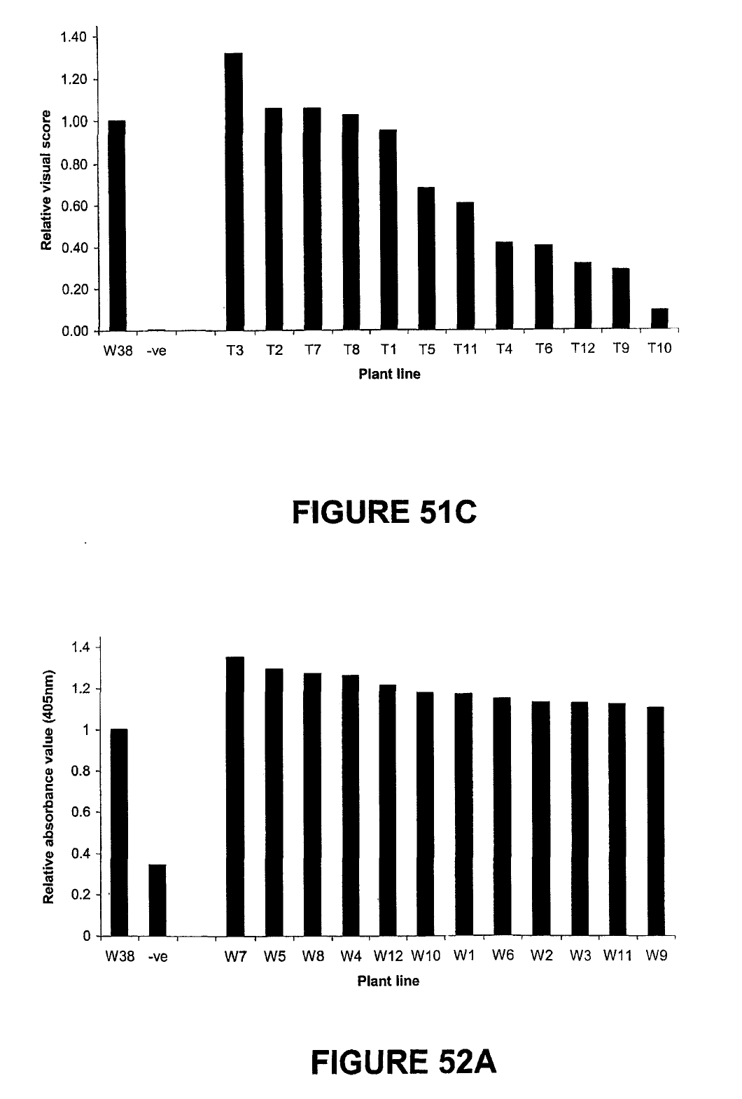

The cloning strategy for the development of the binary vectors containing the wild type or mutant AMV RNA 1 gene is summarized in FIG. 50. The plasmids pCa17TH (AMV RNA 1 infectious clone with a polylinker containing a HindIII restriction enzyme recognition sequence inserted at a DraIII site in the 3′ untranslated region) and the plasmids pCa17TH(G) and pCa17TH(T) (plasmids the same as pCa17TH except that the DNA coding for the ATP binding motif has been mutated) were digested separately with PvuII (step 1, FIG. 50).

The binary vector pGA492 (which has as the selectable marker for plant transformation the nptII gene with the 35S promoter and nos terminator to confer resistance to kanamycin) was digested with HpaI. The PvuII digested fragment of the pCa17TH and related mutant plasmids containing the RNA 1 gene was ligated with the HpaI fragment of pGA492 containing the left and right borders and the nptII gene (step 2, FIG. 50). Since the restriction enzymes PvuII and HpaI cleave DNA to give a blunt end, the orientation of the ligated DNA was confirmed by a number of diagnostic restriction enzyme analysis so that the nptII and AMV RNA 1 genes were cloned in the same direction.

Transformation and Regeneration of Tobacco

The triparental mating of Agrobacterium tumefaciens strain AGL1 with the pGA492 based binary vectors was carried out as described by Ditta et al, 1980. Transformation of tobacco cultivar W38 was carried out essentially as described by Horsh et al (1984).

Transformation and Regeneration of White Clover

The transformation and regeneration of white clover followed the protocol described by Larkin et al., 1996.

Northern Blot Analysis

The extraction of RNA from leaves followed with some modification the protocol of Higgins et al., 1976. The preparation of randomly-primed radioactive probe used the ‘Ready-To-Go’ labelling beads manufactured by Amersham-Pharmacia-Biotech (Cat. #27-9240-01) and followed the suggested protocol. Hybridization was carried out for periods between 6 and 48 hours with labelled probe prepared as in Section 2.6 in modified southern buffer containing 10% w/v dextran sulphate. The blots were washed with 2×SSC at room temperature, then 2×SSC 0.1% SDS 0.1% Sodium pyrophosphate at 42° C. and then 0.1% SSC 2×SSC 0.1% SDS 0.1% Sodium pyrophosphate at 42° C. before the membrane exposed BioMax MS film (Kodak) at −80° C. with a BioMax MS intensifying screen (Kodak).

RT-PCR Reactions

All RT-PCR reactions used the OneStep RT-PCR Kit manufactured by Qiagen (Cat.# 210212) and the suggested protocol was followed. For RT-PCR reactions to detect the nptII transcript, the ‘Q’ solution, which contains betaine, as provided by the manufacturer was used with the primers:

| | |
|---|---|
| amv1F (5′GAATGCTGACGCCCAATC 3′) | SEQ ID NO 55 |
| and | |
| amv1R (5′CCATTTGTCCTTTGACTC 3′). | SEQ ID NO 56 |

The npt177F primer is complimentary to the nptII gene sequence 77 nucleotides from the start of the open reading frame. The npt11922R primer is complimentary to the sequence 922 nucleotides from the start of the open reading frame. The primers are expected to produce a DNA fragment that is 845 base pairs. The temperature sequence used was; 50° C. for 30 minutes, 95° C. for 15 minutes, 94° C. for 40 seconds, 50° C. for 40 seconds, 72° C. for 1 minute, with the last three steps repeated 35 cycles, followed by 72° C. for 10 minutes. For RT-PCR reactions to detect the AMV RNA 1 or related transcript, the primers used were:

npt1177F (5′GCACAACAGACAATCGGCTGCTC 3′) and
npt11922R (5′AGCACGAGGAAGGCGGTCAG 3′).

The amv1F primer is complimentary to the AMV RNA 1 sequence three nucleotides from the start of the open reading frame. The amv1R primer is complimentary to the AMV RNA 1 sequence 1086 nucleotides from the start of the open reading frame. The primers are expected to produce a DNA fragment that is 1000 base pairs.

Mechanical Virus Inoculation of Plants

AMV isolate WC28 virus inoculum was used throughout. Each transgenic line was vegetatively propagated for virus inoculation, using untransformed lines as the negative controls.

Eight uniform clones were selected for each transgenic line. For tobacco, these are plants that had their first three leaves fully formed. Three replicate clones per line were inoculated with 1:50 w/v dilution of AMV isolate WC28 virus inocula and three with 1:100 w/v dilution of the same inocula and two were not inoculated as the negative controls with one for each inoculation. Tobacco plants were inoculated with 50 μl per half leaf of the appropriately diluted (with 0.1 M phosphate buffer pH 7.4) virus inoculum containing 1% w/v carborundum. The virus inoculum was applied to the first three seedling leaves (six half leaves) of tobacco plants that had been kept in the dark for a period of four to six hours and were then gently rubbed by hand across the leaves five times. After inoculation, the plants were washed with water.

White clover plants to be inoculated were kept in the dark overnight and for at least 4 hours the following day. 100 μl of the virus inoculum with 1% carborundum was applied to each of three leaves (three leaflets each—9 leaflets in total per plant) with each leaflet being rubbed by hand five times. After inoculation, the plants were washed with water.

AMV ELISA Assays

Double antibody sandwich ELISA assays were used to estimate the level of AMV in the leaves of tobacco and white clover plants as described by Clark and Adams, 1977.

Visual Scoring of Symptoms on Virus Inoculated Tobacco Plants

Tobacco plants were assessed for the number of lesions on each inoculated leaf. A score from 0 to 5 was given for each leaf on a plant. Score 0 was given when no lesions could be observed, score 1 when 1 to 20 lesions were present, score 2 when 21 to 40 lesions were present, score 3 for 41 to 80 lesions, score 4 for 81 to 160 lesions and a score of 5 if more than 160 lesions were present.

Evaluation of Transgenic Tobacco Containing the Wild Type and Mutant ATP Motif Forms of the AMV RNA 1 Gene for Resistance to AMV Infection

Twelve independent putative transformed tobacco plants were selected for each binary vector—pGA492RNA1 (wild type—coded as ‘W’), pGA492RNA1 (G) (Mutant G coded as ‘G’) and pGA492RNA1 (T) (Mutant T coded as ‘T’)—and were confirmed to be transgenic by PCR analysis for the presence of the nptII gene.

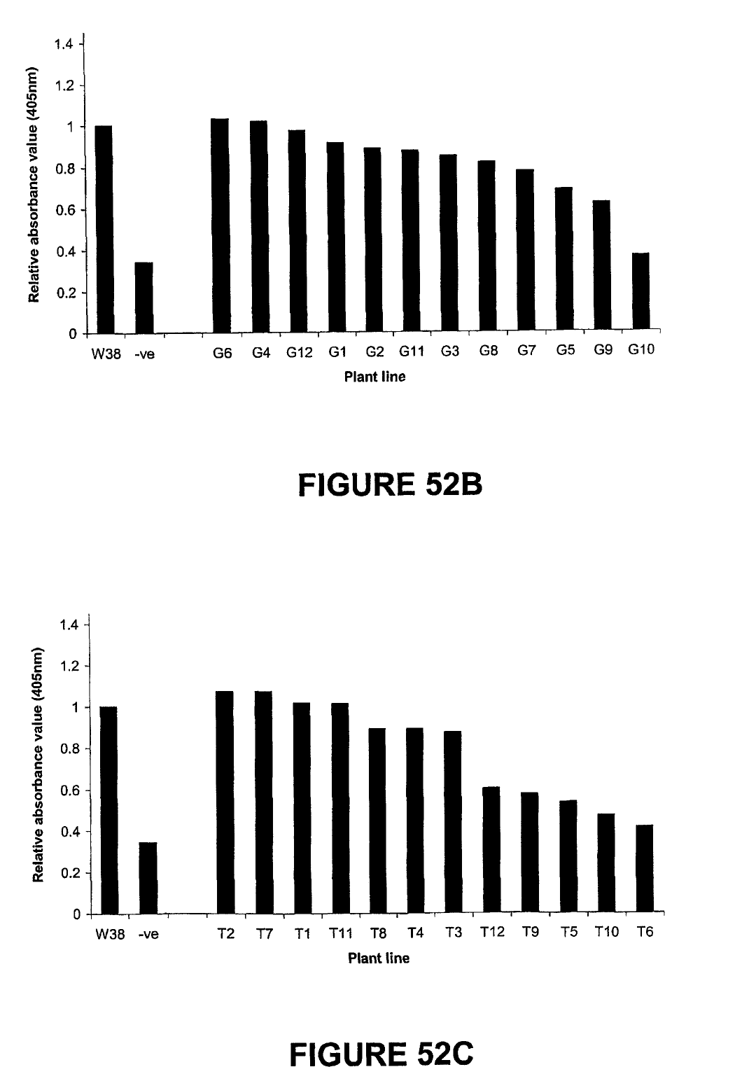

The visual score of symptom severity (number of local lesions) on each inoculated leaf of each plant was assessed six to eight days after inoculation. The results for the three inoculated leaves were combined with those of the other plants of the same transgenic line. Differences were observed between lines transformed with the ‘W’ construct compared to the lines transformed with the ‘G’ and ‘T’ constructs (FIG. 52):. All values were calculated relative to the value obtained from the inoculated untransformed control (WC38). All of the ‘W’ lines had a similar visual score to the untransformed ‘W38’ internal control reference plants (FIG. 52A). The ‘G’ and ‘T’ lines had a range in the visual score with a number of plant lines having a low score with of less than 50% of the ‘W38’ control (FIGS. 52B and 52C), indicating that some virus resistant lines were obtained with these constructs.

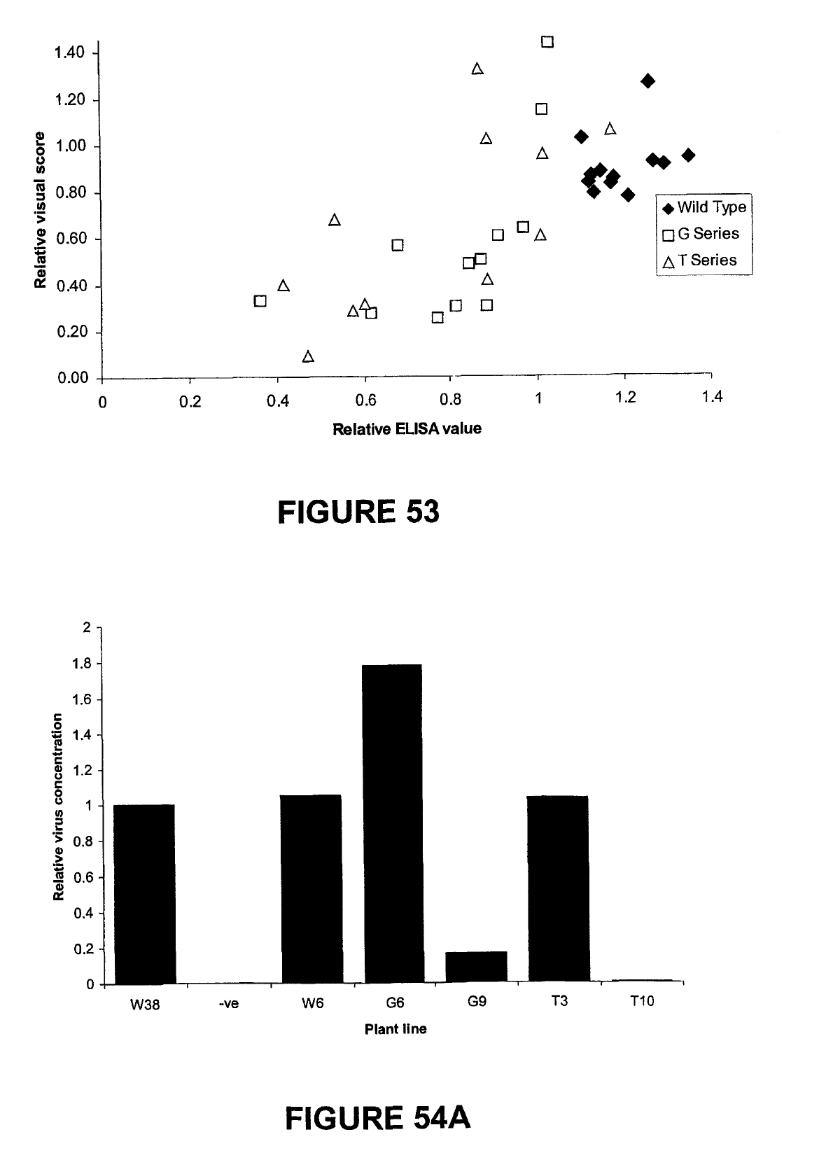

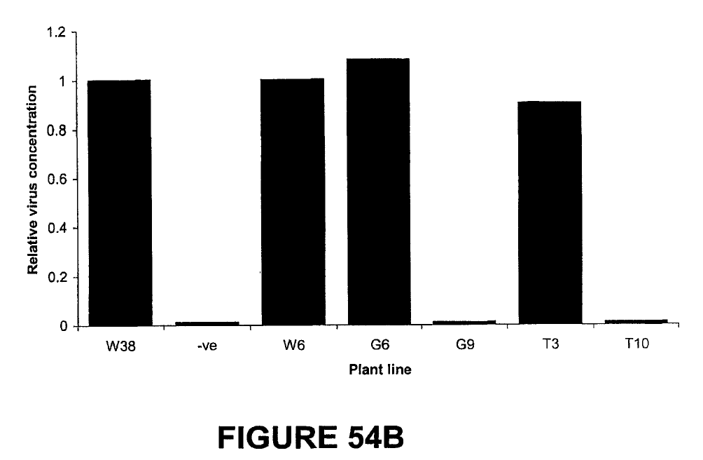

ELISA assays were conducted on 1:1000 v/v diluted sap extracted from leaf discs taken from the three inoculated leaves of each plant six to eight days after inoculation. As for the visual score, the results for the three leaves were combined with that of the other plants of the same transgenic line (FIG. 53). The ‘W’ lines generally had a higher ELISA reading than the ‘W38’ internal control reference plants (FIG. 53). In contrast, the ‘G’ and ‘T’ lines had a range of ELISA readings with some lines being similar to the ‘W38’ control plants but with others similar to that of the un-inoculated control plants (FIG. 53). FIG. 54 showed that there is a direct correlation between visual symptoms and ELISA values for each line. Thus, the ‘G’ and ‘T’ series plants show a range of phenotypes varying from those having both low visual scores and o ELISA values to that where both the visual score and ELISA values are similar to that of the ‘W’ series. It is clear than some of the ‘G’ and ‘T’ series plants have attenuated symptoms of virus infection in contrast with all the ‘W’ series plants which have the same symptoms of infection as the untransformed plants.

To investigate further the level of resistance response of select plants, another inoculation was undertaken and the level of virus accumulation was estimated on both the inoculated and systemic leaves. Serial dilutions were made of the sap extracted from leaf strips of the leaves and used for ELISA analysis. The results for the inoculated leaves are given in FIG. 54A. The susceptible W6, G6 and T3 lines had virus levels similar to or higher than that of the control W38. In contrast, both the resistant G9 and T10 lines had very little virus detectable, with the T10 showing barely above the ‘-ve’ control.

It was evident that plants showing lower levels of virus infectivity in the inoculated leaves also had a delay or a decrease in the symptoms of virus infection in the first systemic leaf. Serial dilution ELISA conducted on leaf strips of the first systemic leaves 10 days after inoculation on the same plants produced results that were consistent with that of the inoculated leaves (FIG. 54B).

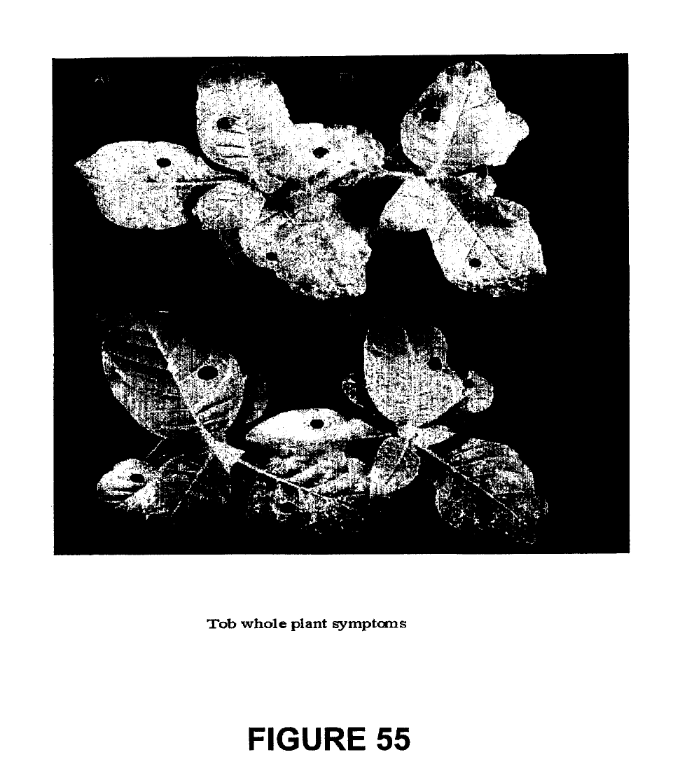

In summary, all transgenic tobacco lines containing the ‘W’ construct had similar severity of symptoms (the number of local lesions and the degree of local and systemic necrosis) of virus infection as the untransformed control line ‘W38’ at the whole plant (FIG. 55), inoculated leaf and the systemic leaf levels. In contrast, AMV resistant transgenic tobacco lines containing the ‘G’ and ‘T’ constructs showed very attenuated symptoms at the whole plant (FIG. 55), inoculated and systemic leaf level.

Evaluation of Transgenic White Clover Containing the Wild Type and Mutant ATP Motif Forms of the AMV RNA 1 Gene for R sistanc to AMV Infection

Independent putative transformed white clover plants were obtained for each binary vector—pGA492RNA1 (wild type—coded as ‘W’), pGA492RNA1 (G) (Mutant G coded as ‘G’) and pGA492RNA1 (T) (Mutant T coded as ‘T’)—and were confirmed to be transgenic by PCR analysis for the presence of the nptII gene. The untransformed control plants selected for comparison to the transformed plants are three genotypes of the white clover of the same cultivar Haifa with a range of susceptibility to AMV infection. The H12 genotype has relatively low susceptibility to AMV infection, following by HNN with the HC genotype being the most susceptible.

Replicate cuttings of a similar size and growth habit with 10 to 15 leaves were selected and inoculated at two different concentrations of AMV isolate WC28 virus.

The results of the first inoculation involving the ‘W’ and ‘T’ lines are given in Table 37. The untransformed white clover plants are strains H12, HC and HNN. The transformed plants have been engineered to express the wild type (W series) and mutant for ATP binding (T series) AMV RNA 1 genes. Symptom severity is rated from very mild (least severe), mild, moderate severe to severe (most severe).

As observed in the tobacco lines, the ‘W’ white clover lines had similar levels of infection as the untransformed controls. Further, the symptoms of AMV infection, clearing between the veins of the leaves and localized necrosis, in the ‘W’ lines were identical to that of the non-transgenic control plants. In the case of the ‘T’ lines, a range of symptoms of virus infections were observed. One line, T7, did not show any virus infection. Both the T6 and T2 lines showed high levels of infection but the symptoms were very much attenuated, especially in the T2 line which was very difficult to detect. Final visual assessment was confirmed by bioassay of representative immune, resistant and susceptible plants, using Chenopodium amaranticolor and cowpeas as indicator hosts. As with coat protein-mediated AMV resistance testing, there was 100% correlation between visual assessment and bioassay results.

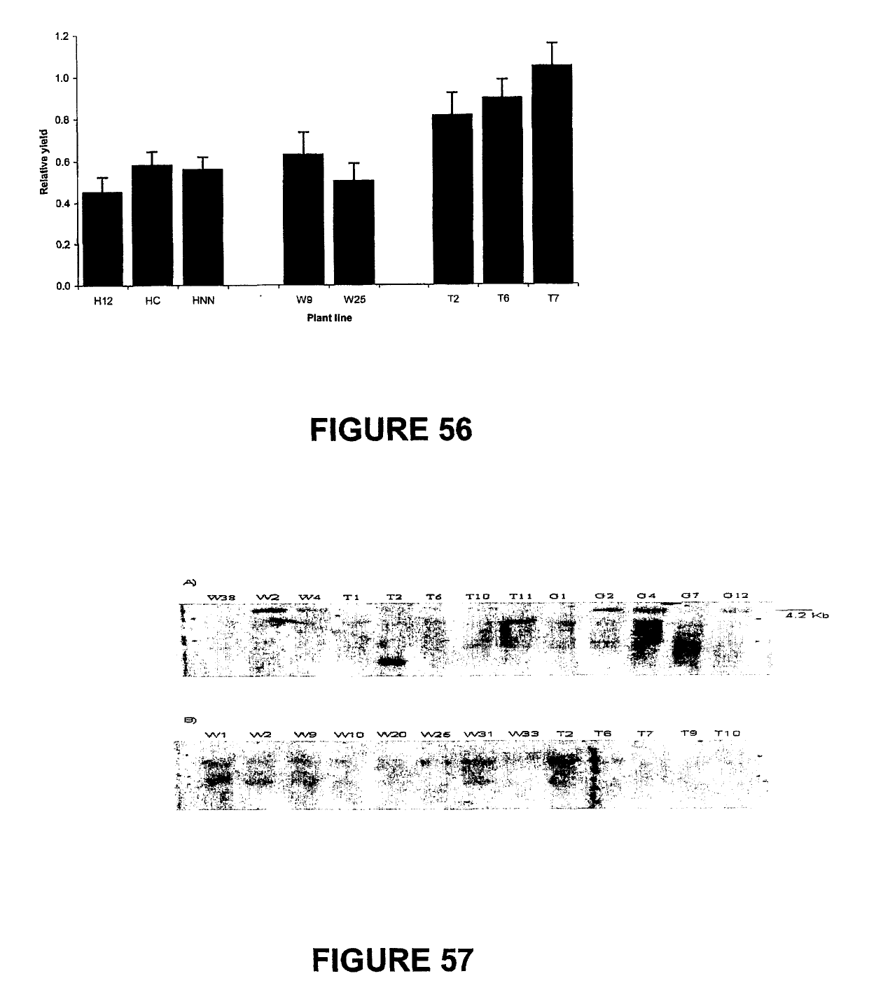

The six inoculated plants of lines H12, HC, HNN, W9, W25, T2, T6 and T7 were grown for a further two months in the glasshouse and were analysed for total biomass production. The results, summarized in FIG. 56, show that the biomass yield of the inoculated untransformed plants (H12,HC, HNN) and the ‘W’ series plants (W9 and W25) was less than that of the ‘T’ series plants. The inoculated T7 line plants had the same yield as the un-inoculated plants.

TABLE 37
Results of the assessment of untransformed and transformed white
clover plants following inoculation with AMV isolate WC28 virus
infected sap at two dilutions (1:10 and 1:5 w/v).

| | Number of plants | | | | |
|---|---|---|---|---|---|
| | infected/inoculated | | | | |
| | Virus | Virus | Plants | | |
| Plant | inoculum | inoculum | infected | Symptom | |
| Line | level, 1:10 | level, 1:5 | (%) | severity | Rating |
| H12 | 2/3 | 2/3 | 67 | Moderate Severe | Susceptible |
| HC | 3/3 | 3/3 | 100 | Moderate Severe | Susceptible |
| HNN | 3/3 | 3/3 | 100 | Moderate Severe | Susceptible |
| W1 | 3/3/ | 3/3 | 100 | Moderate Severe | Susceptible |
| W2 | 3/3 | 3/3 | 100 | Moderate Severe | Susceptible |
| W9 | 3/3 | 3/3 | 100 | Moderate Severe | Susceptible |
| W10 | 3/3 | 2/3 | 83 | Moderate Severe | Susceptible |
| W20 | 3/3 | 3/3 | 100 | Moderate Severe | Susceptible |
| W25 | 2/3 | 3/3 | 83 | Severe | Susceptible |
| W31 | 2/3 | 2/3 | 67 | Moderate Severe | Susceptible |
| W33 | 3/3 | 3/3 | 100 | Moderate Severe | Susceptible |
| T2 | 3/3 | 3/3 | 100 | Very mild | Susceptible |
| T6 | 2/3 | 3/3 | 83 | Mild | Susceptible |
| T7 | 0/3 | 0/3 | 0 | No symptoms | Immune |
| T8 | 1/3 | 3/3 | 67 | Moderate Severe | Susceptible |
| T9 | 0/3 | 2/3 | 33 | Moderate Severe | Resistant |
| T10 | 3/3 | 3/3 | 100 | Moderate Severe | Susceptible |

### EXAMPLE 11.4

### Molecular Analysis of AMV RNA 1 Wild Type and Mutant Transgene Expression in Transgenic Tobacco and White Clover

Northern blot analysis was conducted on the RNA samples to detect both the AMV RNA 1 gene mRNA and the nptII gene mRNA. Great difficulty was encountered in detecting the mRNA of the AMV RNA 1 gene and the mutant derivatives in both tobacco and white clover. Several different RNA extractions were tried along with probes made from the DNA of pCa17TH, the HindIII XbaI fragment containing DNA coding for the ATP binding motif from pCa17TH and from a 1000 bp PCR amplified DNA fragment of the 5′ terminal end of the coding region of the AMV RNA 1 gene.

For some tobacco plants, a band of the approximate size to that expected could be observed (4.2 kb) (FIG. 57). No band of the approximate size expected could be detected in the RNA from the white clover lines (FIG. 57). The northern blot shown in FIG. 57 is from a hybridisation using a probe that had been random primed from a HindIII XbaI fragment from pCa17TH containing the DNA coding for the ATP binding site and which had been exposed to film for 6 days. W38 is non-transgenic control. W lines are plants transformed with the wild-type RNA 1a gene. T and G lines are plants transformed with the T and G mutant derivatives of the AMV RNA 1a gene, respectively

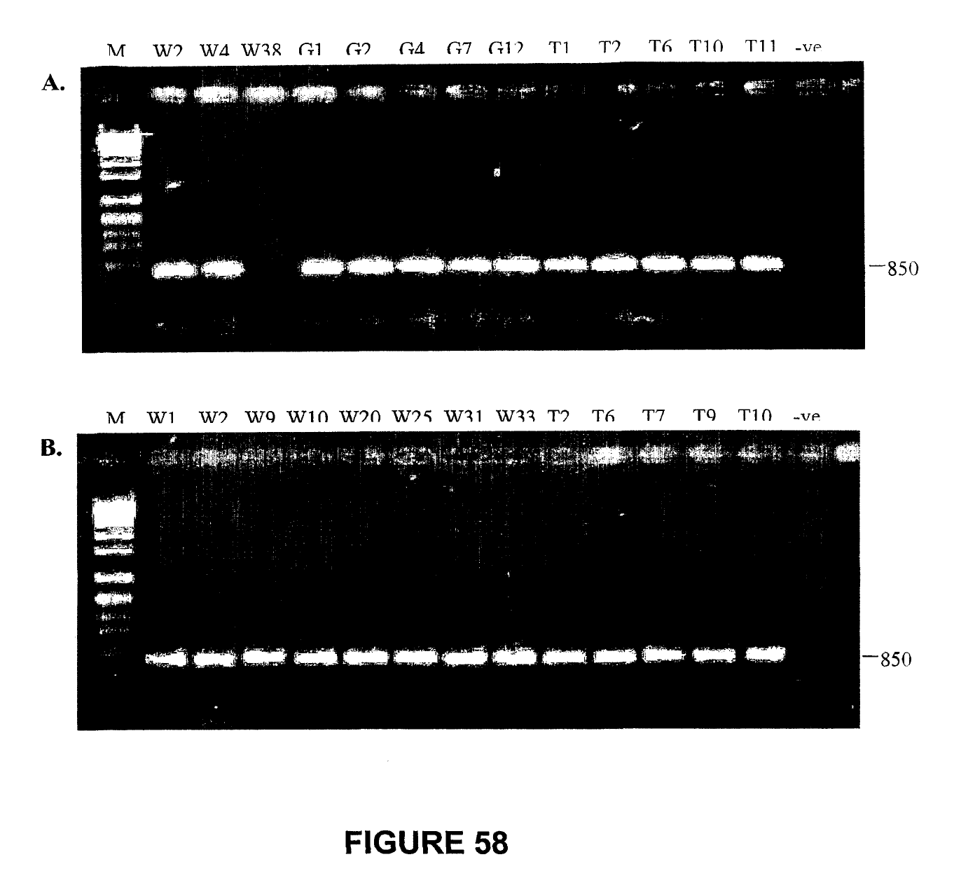

Given the apparent low level of expression of the AMV RNA 1 wild type and mutant derivatives in both tobacco and white clover, RT-PCR for both this gene and the nptII gene was undertaken. Using DNAse I treated RNA samples, it was possible to detect only the nptII mRNA in all samples (FIG. 58). Despite many attempts using different conditions and primers, mRNA corresponding to the AMV RNA 1 gene could not be detected. However in RNA samples that had not been treated with DNAse 1 the AMV RNA 1 gene was detected by PCR but not in the negative controls (data not shown). These results indicated that the nptII gene is being expressed in all of the transgenic plants and although the AMV RNA 1 gene is present in the genome of the plants it is either being expressed at an undetectably low level or gene silencing is taking place.

### REFERENCES

1. An et al, (1985) EMBO J. 4:277-284.

2. Armstrong, C. L., et al (1990). Plant Cell Reports 9: 335-339.

3. Ausubel, F. M., et al (1987). In: Current Protocols in Molecular Biology. Wiley Interscience (ISBN 047150338). Baker and Williams, eds.

4. Bilang et al, (1991) Gene 100, 247-250.

5. Boevink, P., et al (1995). Virology 207, 354-361.

6. Bradford, M. (1976) Anal. Biochem. 72, 248-254.

7. Campbell, C. L. and Moyer, J. W. (1984). Plant Disease 68, 1033-1035.

8. Christou, P., et al (1988). Plant Physiol 87, 671-674.

9. Chu, P. W. G. and Helms, K. (1988). Virology, 167, 38.

10. Chu, P., et al (1995). In: “Pathogenesis and Host Parasite Specificity in Plant Diseases. Histopathological, biochemical, genetic and molecular bases. Vol. III, Viruses and Virolds.” (R. P. Singh, U. S. Singh and K. Kohmoto, eds.). Pergamon Press, pp 311-341.

11. Chu, P. W. G., et al (1999). Ann. Appl. Biol. 135,469-480.

12. Clark, M. F. and Adams, A. N. (1977) J. Gen. Virog. 34, 475-483.

13. Crossway et al, (1986) Mol. Gen. Genet. 202,179-185.

14. Davenport G. F. and Baulcombe D. C. (1997). J. General Virology 78:1247-1251.

15. Ditta G., et al (1980). Proc. Natl. Acad. Sci. (USA) 77:7347-7351.

16. Gorbalenya A. E. and Koonin E. V. (1989). Nucleic Acids Research 17:8413-8440.

17. Horsch R. B., et al (1984). Science 227:1229-1231.

18. Jayasena K. W., et al (2001). Aust. J. Agricultural Research 52:67-72.

19. Konola J. T., et al (1994). J. Molecular Biology 237:20-34.

20. Koonin E. V. (1997). Science 275:1489-1490.

21. De Block, M., et al (1987). EMBO J. 6: 2513-2518.

22. Del Sal, G., et al (1989). Bio/Techniques 7, 514-518.

23. Devereaux, J., et al (1984). Nucleic acids Res. 12, 387-395.

24. Dougherty et al, (1994) Mol. Plant-Microb. Interact. 7, 544-552.

25. Edwardson, J. R. and Christie, R. G. (1986). Viruses Infecting Forage Legumes, Vols I-III. Agricultural Experimental Stations, Institute of Food and Agricultural Sciences, University of Forida, Gainesville.

26. Edwardson, J. R. and Christie. R. (1991). The potyvirus group, Volume 1. Florida Agricultural Experiment Station, Monograph Series No. 16-1.

27. Fromm et al, (1985) Proc. Natl. Acad. Sci. (USA) 82,5824-5828.

28. Garrett, R. G. (1991). Proc. White Clover Conf Department of Agriculture, Victoria, pp. 50-57.

29. Garrett, R. G. (1992). Impact of viruses on pasture legume productivity. Terminating Report. DRDC Project DAV 134. Department of Agriculture, Victoria.

30. Gibson, P. B., et al (1981). Plant Disease 65, 50-51.

31. Gibson, P. B., et al (1989). Crop Sci 29,241-242.

32. Hajdukiewicz et al, (1994) Plant Mol. Biol. 25, 989-994.

33. Helms, K., et al (1993). Aust. J. Agric. Res. 44, 1837-1862.

34. Herrera-Estrella et al (1983a) Nature 303, 209-213.

35. Herrera-Estrella et al., (1983b) EMBO J. 2, 987-995.

36. Herrera-Estrella et al. (1985) In: Plant Genetic Engineering, Cambridge University Press, NY, pp 63-93.

37. Higgins, T. J. V. and Spencer, D. (1991). Plant Sci. 74, 89-98.

38. Hollings, M. and Brunt, A. A. (1981 a). In: Handbook of Plant Virus Infections: Comparative Diagnosis., Elsevier/North Holland, Amsterdam, (Kurstak, E. ed.) pp. 731-807.

39. Hollings, M. and Brunt, A. A. (1981b). Potyvirus group. CMI/AAB. Descriptions of Plant Viruses, No. 245.

40. Jobling S. A. and Gehrke L. (1987). Nature 325:622-625.

41. Johnstone, G, R. and McLean, G. D. (1987). Annals Appl. Biol. 110: 421-440.

42. Jones, R, A, C. (1994). Aust J. Agric. Res. 45:1427-1444.

43. Jones, R. A. C. (1996). In: Pasture and Forage Crop Pathology; Am. Soc. Agron., Crop Sci. Soc. Am., and Soil Sci. Soc. Am., WI, USA, (Eds S Chakraborty, K T Leath, R A Skipp, G A Pederson, R A Bray, G C M Latch, F W Nutter, and W Forest Jr.), pp. 303-322.

44. Jones et al., (1992) Transgenic Res. 1, 285.

45. Khan, M. R. I., et al. (1994). Agrobacterium-mediated transformation of subterranean clover (Trifolium subterraneum L.). Plant Physiol. 114 (In press).

46. Khandjian, (1987) Biotechnology 5, 165-167.

47. Klausner, (1987) Biotechnology 5, 551-556.

48. Klein T M, et al. (1988). Proc Natl Acad Sci (USA) 85,4305-4309.

49. Krens, F. A., et al. (1982). Nature 296, 72-74.

50. Larkin et al, (1996) Transgenic Res. 5, 325-335.

51. Logan K. M. and Knight K. L. (1993). J. Molecular Biology 232:1048-1059

52. Latch, G. C. M. and Skipp, R. A. (1987). Diseases. In: White Clover. Baker, M. J. and Williams, W. M., eds. CAB International, Wallingford, U.K. pp. 421-460.

53. Lazo, G. R., et al. (1991). Biotechnol. 9, 963-967.

54. Laemmli and Farre, (1973) J. Mol. Biol. 80, 575-599.

55. Lindbro et al, (1993) Plant Cell 5, 1749-1759.

56. Mason, W. (1993). In: White Clover. A key to increasing mik yields. Dairy Reseach and Development Coporation. Agmedia, Melbourne.

57. Matthews, R. E. F. (1991). In: Plant Virology, Third edition. Academic Press, New York.

58. Mazur, B. J. and Chui, C. (1985). Nucl. Acids Res. 13:2373-2386.

59. McDonnell, R. E., et al. (1987). Plant Mol. Biol. Rep. 5,380-386.

60. McPherson, M. J., et al. (1991) In: PCR: A Practical Approach. (series editors, D. Rickwood and B. D. Hames) IRL Press Limited, Oxford. pp1-253.

61. Milne, R. G. (1988). In: The Plant Viruses, Vol.4., (Milne, R. G., ed.)Plenum Press, New York, pp. 333-335.

62. Neeleman L., et al (1993). Virology 196:883-887.

63. Nikandrow, A. and Chu, P. W. G. (1991). In: Proc. White Clover Conf. Dept Agriculture, Victoria, pp. 64-67.

64. Paszkowski et al, (1984) EMBO J. 3, 2717-2722.

65. Pearson, C. J., et al. (1997.) Aust. J. Agric. Res. 48:453-465.

66. Pietrzak, M., et al. (1986). Nucl. Acids Res. 14:5857-5868.

67. Randles, J. W., et al., (2000). In: Virus Taxonomy. Vol. 7^{th} Report of the International Committee on Taxonomy of Viruses.

68. Reichmann, J. L., et al. J. Gen. Virol. 73:1-16.

69. Sanger, F., et al., (1977). Proc. Natl. Acad. Sci. USA, 74, 5463-5467.

70. Sambrook, J., et al. (1989). In: Molecular Cloning: a laboratory manual. Cold Spring Harbor Laboratory Press.

71. Saraste M., Sibbald P. R. and Wittinghofer A. (1990). TIBS 15:430434

72. Story R. M., et al (1993). Science 259:1892-1896

73. Schardl, C. L., et al. (1987). Gene 61:1-11.

74. Schroeder, H. E., et al., (1991). Aust. J. Plant. Physiol. 18: 495-505.

75. Shukla, D. D., et al. (1994). In: The Potyviridae., CAB International, Wallingford, Oxon, United Kingdom.

76. Spencer, et al., (1990) Theor Appl. Genet 79, 625-631.

77. Tabe, L. M., et al. (1995) J. Animal Sci. 73:2752-2759.

78. Taylor, N. L., and Ghabrial, S. A. (1986). In: Viruses infecting forage legumes, Vol. III, University of Florida Press, Gainesville, USA (Eds., J. R. Edwardson and R. G. Christie), pp. 609-623.

79. Timmerman-Vaughan, G. M., et al. (2001). Crop Science 41, 846-853.

80. Towbin, H., et al. (1979). Proc. Natl Acad. Sci.(USA) 76, 4350-4354.

81. Van Vloten-Doting and Jaspars (1972). Virol. 48, 699-708.

82. Voisey, C. R., et al. (1994). Plant Cell Rep. 13:309-314.

83. Walker J. E, et al (1982). EMBO 1:945-951.

84. Wang M-B., et al. (2000). Molecular Plant Pathology 1:347-356.

85. Ward, C. W. and Shukla, D. D. (1991). Intervirol. 32, 269-296.

86. Waterhouse, P. M., et al (1998). Proc. Natl. Acad. Sci. (USA) 95:13959-13964.

87. Waterhouse, P. M., et al (2001). Nature 411:834-842.

88. Yoshida, M. and Amano T. (1995). FEBS Letters 359:1-5.

89. Yusibov, V., and Loesch-Fries, L. S. (1995). Proc. Natl. Acad. Sci. (USA) 92: 8980-8984.

1. A method of conferring on a leguminous plant, immunity to a pathogenic plant virus, comprising introducing to said plant an isolated nucleic acid molecule comprising nucleotides encoding a virus-encoded coat protein or a dysfunctional viral replicase or an iRNA comprising a hairpin RNA, wherein the leguminous plant is immune to the plant virus under field conditions by virtue of the presence of the isolated nucleic acid molecule.

2. The method according to claim 1 wherein the plant virus is selected from the group consisting of bromoviruses, potyviruses, potexviruses, and nanoviruses.

3. The method according to claim 1, wherein the plant virus is selected from the group consisting of alfalfa mosaic virus (AMV), clover yellow vein virus (CYVV), sub-clover stunt virus (SCSV), bean yellow mosaic virus (BYMV) and white clover mosaic virus (WCMV).

4. The method according to claim 1 wherein the plant is a pasture or forage legume.

5. The method according to claim 1 wherein the plant is of a species selected from the group consisting of Trifolium spp. and Medicago spp.

6. The method according to claim 5 wherein the plant is of a species selected from the group consisting of T. repens, T. subterraneum, T. pratense, T. michelianum, T. isthmocarphum, and M. sativa.

7. The method according to claim 1, wherein the isolated nucleic acid molecule encodes a dysfunctional viral replicase modified in an NTP binding motif.

8. The method according to claim 7, wherein the dysfunctional viral replicase is modified in an ATP binding motif.

9. The method according to claim 1, wherein the virus-encoded coat protein comprises an amino acid sequence selected from the group consisting of SEQ ID Nos: 2, 4, 6, 8, 10, 12, 14, 16, 18, 26, 31, 33, and 35.

10. The method according to claim 1, wherein the isolated nucleic acid molecule encoding a virus-encoded coat protein comprises nucleotides having a nucleotide sequence selected from the group consisting of SEQ ID Nos: 1, 3, 5, 7, 9, 11, 13, 15, 17, 25, 30, 32, and 34.

11. The method according to claim 1, where the isolated nucleic acid molecule encodes a virus-encoded coat protein or a dysfunctional viral replicase which is produced in the leguminous plant.

12. The method according to claim 1, wherein the isolated nucleic acid molecule encodes a virus-encoded protein or a dysfunctional viral replicase which is not produced in the leguminous plant.

13. The method according to claim 1, wherein the iRNA comprises nucleotides having a nucleotide sequence derived from a nucleotide sequence encoding a virus-encoded coat protein or a dysfunctional viral replicase.

14. The method according to claim 1, wherein the isolated nucleic acid molecule is expressed in the leguminous plant under the control of a strong constitutive promoter.

15. The method according to claim 14, wherein the promoter is selected from the group consisting of (i) a SCSV promoter; (ii) a pea rbcS-E9 promoter; (iii) a CaMV 35S promoter; (iv) a duplicated promoter; (v) a CaMV 19S promoter; and (vi) a A. thaliana SSU promoter.

16. The method according to claim 14, wherein the promoter is selected from the group consisting of (i) a SCSV region 4 (SCSV4) promoter; (ii) a duplicated CaMV 35S promoter; and (iii) a A. thaliana SSU promoter.

17. The method according to claim 1 further comprising introducing to said leguminous plant a second isolated nucleic acid molecule, which second isolated nucleic acid molecule confers enhanced resistance to a second plant virus on said leguminous plant.

18. The method according to claim 17, wherein the first and second isolated nucleic acid molecules are introduced into the leguminous plant by sequential rounds of transformation.

19. The method according to claim 17 wherein the second plant virus is selected from the group consisting of alfalfa mosaic virus (AMV), clover yellow vein virus (CYVV), sub-clover stunt virus (SCSV), bean yellow mosaic virus (BYMV) and white clover mosaic virus (WCMV).

20. The method according to claim 1 wherein said isolated nucleic acid molecule is introduced to the said leguminous plant by a process comprising:
(a) transforming a plant cell with said isolated nucleic acid molecule to produce a transformed plant cell;
(b) regenerating a whole plant from said transformed plant cell; and
(c) obtaining a progeny plant from said whole plant wherein said progeny plant contains one or more gene copies of the isolated nucleic acid molecule.

21. A method of transforming a leguminous plant said method comprising introducing to a leguminous plant cell, tissue or organ, an isolated nucleic acid molecule comprising an ASSU promoter or a d35S promoter operably linked to a nucleotide sequence encoding a virus-encoded coat protein or a dysfunctional viral replicase or an iRNA comprising a hairpin RNA, and regenerating a transformed leguminous plant from the plant cell, tissue or organ, wherein the transformed leguminous plant is immune to a pathogenic plant virus under field conditions by virtue of the presence of the isolated nucleic acid molecule.

22. The method according to claim 21 wherein said isolated nucleic acid molecule is expressed to produce the virus-encoded coat protein or dysfunctional viral replicase in the transformed leguminous plant.

23. A method of producing a leguminous plant with enhanced viral resistance comprising crossing two parent plants each having enhanced viral resistance or immunity against one or more different viruses, wherein at least one parent plant has been produced by the method of claim 1, whereby the progeny leguminous plant has enhanced viral resistance or immunity.

24. An isolated nucleic acid molecule comprising nucleotides encoding the coat protein of a virus, wherein said coat protein has an amino acid sequence selected from the group consisting of SEQ ID Nos: 2, 4, 6, 8, 10, 12, 14, 16, 18, 26, 31, 33, and 35.
(a) 25. The isolated nucleic acid molecule according to claim 24 wherein said nucleotides have a sequence selected from the group consisting of SEQ ID Nos: 1, 3, 5, 25 and 30.

26. A gene construct comprising the isolated nucleic acid molecule of claim 24 and a promoter sequence for regulating expression of said nucleotides.

27. The gene construct according to claim 26 further comprising a terminator sequence.

28. The gene construct according to claim 26 further comprising a selectable marker gene.

29. The gene construct according to claim 26 comprising nucleotides encoding 2 or more coat proteins wherein each coat protein comprises amino acids having a sequence selected from the group consisting of SEQ ID Nos: 2, 4, 6, 8, 10, 12, 14, 16, 18, 26, 31, 33, and 35.

30. The gene construct according to claim 26 further comprising a binary vector.

31. The gene construct according to claim 26 suitable for A. tumefaciens-mediated transformation of a plant cell.

32. A transformed leguminous plant that is immune to a pathogenic plant virus under field conditions, wherein the plant comprises an isolated nucleic acid molecule encoding a virus-encoded coat protein or a dysfunctional viral replicase or an iRNA comprising a hairpin RNA, wherein the leguminous plant is immune to the plant virus under field conditions by virtue of the presence of the isolated nucleic acid molecule.

33. The transformed leguminous plant according to claim 32 wherein the plant virus is selected from the group consisting of bromoviruses, potyviruses, potexviruses, and nanoviruses.

34. The transformed leguminous plant according to claim 32 wherein the plant virus is selected from the group consisting of alfalfa mosaic virus (AMV), clover yellow vein virus (CYVV), sub-clover stunt virus (SCSV), bean yellow mosaic virus (BYMV) and white clover mosaic virus (WCMV).

35. The transformed leguminous plant according to claim 32 wherein the plant is a pasture or forage legume.

36. The transformed leguminous plant according to claim 32 wherein the plant is of a species selected from the group consisting of Trifolium spp. and Medicago spp.

37. The transformed leguminous plant according to claim 32 wherein the plant is of a species selected from the group consisting of T. repens, T. subterraneum, T. pratense, T. michelianum, T. isthmocarphum and M. sativa.

38. The transformed leguminous plant according to claim 32, wherein the isolated nucleic acid molecule encodes a dysfunctional viral replicase modified in an NTP binding motif.

39. The transformed leguminous plant according to claim 38, wherein the dysfunctional viral replicase is modified in an ATP binding motif.

40. The transformed leguminous plant according to claim 32, wherein the virus-encoded coat protein comprises amino acids having an amino acid sequence selected from the group consisting of SEQ ID Nos: 2, 4, 6, 8, 10, 12, 14, 16, 18, 26, 31, 33, and 35.
(a) 41. The transformed leguminous plant according to claim 32, wherein the isolated nucleic acid molecule comprises nucleotides having a nucleotide
(a) sequence selected from the group consisting of SEQ ID Nos: 1, 3, 5, 7, 9, 11, 13, 15, 17, 25, 30, 32, and 34.

42. The transformed leguminous plant according to claim 32, where the isolated nucleic acid molecule encodes a virus-encoded coat protein or a dysfunctional viral replicase which is not produced in the leguminous plant.

43. The transformed leguminous plant according to claim 32, wherein the isolated nucleic acid molecule encodes a virus-encoded coat protein or dysfunctional viral replicase which is produced in the leguminous plant.

44. The transformed leguminous plant according to claim 32, wherein the iRNA comprises nucleotides having a nucleotide sequence derived from a nucleotide sequence encoding a virus-encoded coat protein or a dysfunctional viral replicase.

45. The transformed leguminous plant according to claim 32, wherein the isolated nucleic acid molecule is expressed in the leguminous plant under the control of a strong constitutive promoter.

46. The transformed leguminous plant according to claim 45, wherein the promoter is selected from the group consisting of (i) a SCSV promoter; (ii) a pea rbcS-E9 promoter; (iii) a CaMV 35S promoter; (iv) a duplicated promoter; (v) a CaMV 19S promoter; and (vi) a A. thaliana SSU promoter.

47. The transformed leguminous plant according to claim 45, wherein the promoter is selected from the group consisting of (i) a SCSV region 4 (SCSV4) promoter; (ii) a duplicated CaMV 35S promoter; and (iii) a A. thaliana SSU promoter.

48. The transformed leguminous plant according to claim 32, wherein the plant further comprises a second isolated nucleic acid molecule which enhances resistance to a second plant virus

49. The transformed leguminous plant according to claim 48, wherein the plant has enhanced resistance to at least two viruses selected from the group alfalfa mosaic virus (AMV), clover yellow vein virus (CYVV), sub-clover stunt virus (SCSV), bean yellow mosaic virus (BYMV) and white clover mosaic virus (WCMV).

50. A method of identifying a gene of interest in a primary transformant plant or a progeny plant thereof comprising
(a) conducting a PCR replication cycle on a sample of interest;
(b) detecting a PCR product; and
(c) analysing the presence or absence of a PCR product above background to determine whether a plant is homozygous, heterozygous or azygous for a gene of interest.

51. A method according to claim 50 wherein the PCR replication cycle incorporates a marker and detection of the PCR product is by detection of the marker.

52. A method according to claim 50 wherein the number of PCR replication cycles required to detect the PCR product above background determines whether a plant is homozygous, heterozygous or azygous for a gene of interest.


SEQUENCE LISTING (56 sequences)

SEQ ID NO: 1 - DNA, 666 bp, alfalfa mosaic virus strain H1
  CDS (1)..(663)
atgagttcttcacaaaagaaagctggtgggaaagctggtaaacctactaaacgttctcagaactatgctgccttgcgcaaagctcaactgccgaagcctccggcgttgaaagtcccggttgtaaaaccgacgaatactatactgccacagacgggctgcgtgtggcaaagcctcgggacccctctgagtctgagctcttttaatgggctcggcgtgagattcctctacagttttctgaaggatttcgcgggacctcggatcctcgaagaggatctgatttacaggatggtgttttctataacaccgtcccatgccggcaccttttgtctcactgatgacgtgacgactgaggatggtagggccgttgcgcatggtaatcccatgcaagtatttcctcaaggcccgtttcacgttaatgggaagttcggggttgagttggtcttcacagctcctacccatgcgggaatgcaaaaccaaaattttaagcattcctatgccgtagccctctgtctggacttcgatgcgcagcctgaggggtctaaaaacccctcataccgattcaacgaagtttgggtcgagaaaaaggcgttcccgcgagcagggcccctccgcagtttgattactgtggggctgcttgacagaagtgacgatcttgatcgtcattga

SEQ ID NO: 2 - protein, 221 aa, alfalfa mosaic virus strain H1
MSSSQKKAGGKAGKPTKRSQNYAALRKAQLPKPPALKVPVVKPTNTILPQTGCVWQSLGTPLSLSSFNGLGVRFLYSFLKDFAGPRILEEDLIYRMVFSITPSHAGTFCLTDDVTTEDGRAVAHGNPMQVFPQGPFHVNGKFGVELVFTAPTHAGMQNQNFKHSYAVALCLDFDAQPEGSKNPSYRFNEVWVEKKAFPRAGPLRSLITVGLLDRSDDLDRH

SEQ ID NO: 3 - DNA, 666 bp, alfalfa mosaic virus strain WC3
  CDS (1)..(663)
atgagttcttcacaaaagaaagctggtgggaaagctggtaaacctactaaacgttctcagaactatgctgccttgcgcaaagctcaactgccgaaacctccggcgttgaaagtcccggttgtaaaaccgacgaatactatactgccacagacgggctgcgtgtggcaaagcctcgggacccctctgagtctgagctcttttaatgggcttggcgtgagattcctctacagttttctgaaggatttcgcgggacctcggatcctcgaagaggatctgatttacaggatggtgttttccataacaccgtcccatgccggcaccttttgtctcactgatgacgtgacgactgaggatggtagggccgttgcgcatggtaatcccatgcaggaatttcctcaaggcgtgtttcacgctaatgagaagttcgggtttgagttggtcttcacagctcctacccatgcgggaatgcaaaatcaaaatttcaagcattcctatgccgtagccctctgtctggacttcgatgcgcagcctgagggatctaaaaatccctcataccgattcaacgaagtttgggtcgagagaaaagcgttcccgcgagcagggcccctccgcagtttgattactgtggggctgctcgacgaagctgacgatcttgatcgtcattga

SEQ ID NO: 4 - protein, 221 aa, alfalfa mosaic virus strain WC3
MSSSQKKAGGKAGKPTKRSQNYAALRKAQLPKPPALKVPVVKPTNTILPQTGCVWQSLGTPLSLSSFNGLGVRFLYSFLKDFAGPRILEEDLIYRMVFSITPSHAGTFCLTDDVTTEDGRAVAHGNPMQEFPQGVFHANEKFGFELVFTAPTHAGMQNQNFKHSYAVALCLDFDAQPEGSKNPSYRFNEVWVERKAFPRAGPLRSLITVGLLDEADDLDRH

SEQ ID NO: 5 - DNA, 657 bp, alfalfa mosaic virus strain 425S
  CDS (1)..(654)
atgagttcttcacaaaagaaagctggtgggaaagctggtaaacctactaaacgttcccagaattatgctgctttacgcaaagctcaactgccgaaacctccggcgttgaaagtcccggttgcaaaaccgacgaacactatactgccacagacgggctgcgtgtggcaaagcctcgggacccctctgagtctgagctcttttaacgggctcggcgtgagattcctctacagttttctgaaggatttcacgggacctcggatcctcgaagaggatctgatttacaggatggtgttttccataacaccgtcccatgccggcaccttttgtctcactgatgacgtgacgactgaggatggtagggccgttgcgcatggtaatcccatgcaagaatttcctcatggcgcgtttcacgctaatgagaagttcgggtttgagttggtcttcacagctcctacccatgcgggaatgcaaaatcaaaatttcaagcattcctatgccgtagccctctgtctggacttcgacgcgcagcctgagggatctaaaaatccctcataccgattcaacgaagtttgggttgagagaaaggcgttcccgcgagcagggcccctccgcagtttgattactgtggggttgctcgacgaagctgacgatctttga

SEQ ID NO: 6 - protein, 218 aa, alfalfa mosaic virus strain 425S
MSSSQKKAGGKAGKPTKRSQNYAALRKAQLPKPPALKVPVAKPTNTILPQTGCVWQSLGTPLSLSSFNGLGVRFLYSFLKDFTGPRILEEDLIYRMVFSITPSHAGTFCLTDDVTTEDGRAVAHGNPMQEFPHGAFHANEKFGFELVFTAPTHAGMQNQNFKHSYAVALCLDFDAQPEGSKNPSYRFNEVWVERKAFPRAGPLRSLITVGLLDEADDL

SEQ ID NO: 7 - DNA, 666 bp, alfalfa mosaic virus strain 425M
  CDS (1)..(663)
atgagttcttcacaaaagaaagctggtgggaaagctggtaaacctactaaacgttctcagaactatgctgccttacgcaaagctcaactgccgaagcctccggcgttgaaagtcccggttgtaaaaccgacgaatactatactgccacagacgggctgcgtgtggcaaagcctcgggacccctctgagtctgagctcttttaatgggctcggcgtgagattcctctacagttttctgaaggatttcgcgggacctcggatcctcgaagaggatctgatttacaggatggtgttttccataacaccgtcctatgccggcaccttttgtctcactgatgacgtgacgactgaggatggtagggccgttgcgcatggtaatcccatgcaagaatttcctcatggcgcgtttcacgctaatgagaagttcgggtttgagttggtcttcacagctcctacccatgcgggaatgcaaaaccaaaatttcaagcattcctatgccgtagccctctgtctggacttcgacgcgcagcctgagggatctaaaaatccctcataccgattcaacgaagtttgggtcgagagaaaggcgttcccgcgagcagggcccctccgcagtttgattactgtggggctgctcgacgaagctgacgatcttgatcgtcattga

SEQ ID NO: 8 - protein, 221 aa, alfalfa mosaic virus strain 425M
MSSSQKKAGGKAGKPTKRSQNYAALRKAQLPKPPALKVPVVKPTNTILPQTGCVWQSLGTPLSLSSFNGLGVRFLYSFLKDFAGPRILEEDLIYRMVFSITPSYAGTFCLTDDVTTEDGRAVAHGNPMQEFPHGAFHANEKFGFELVFTAPTHAGMQNQNFKHSYAVALCLDFDAQPEGSKNPSYRFNEVWVERKAFPRAGPLRSLITVGLLDEADDLDRH

SEQ ID NO: 9 - DNA, 666 bp, alfalfa mosaic virus strain 425L
  CDS (1)..(663)
atgagttcttcacaaaagaaagctggtgggaaagctggtaaacctactaaacgttctcagaactatgctgctttacgcaaagctcaactgccgaagcctccggcgttgaaagtcccggttgtaaaaccgacgaatactatactgccacagacgggctgcgtgtggcaaagcctcgggacccctctgagtctgagctcttttaatgggctcggcgcgagattcctctacagttttctgaaggatttcgtgggacctcggatcctcgaagaggatctgatttacaggatggtgttttccataacaccgtcccatgccggcaccttttgtctcactgatgacgtgacgactgaggatggtagggccgtcgcgcatggtaatcccatgcaagaatttcctcatggcgcgtttcacgccaatgagaagttcgggtttgagttggtcttcacagctcctacccatgcgggaatgcaaaatcaaaatttcaagcattcctatgccgtagccctctgtctggacttcgatgcgcagcctgagggatctaaaaatccctcattccgattcaacgaagtttgggtcgagagaaaggcgttcccgcgagcagggcccctccgcagtttgattactgtggggctgttcgacgaagctgacgatcttgatcgtcattga

SEQ ID NO: 10 - protein, 221 aa, alfalfa mosaic virus strain 425L
MSSSQKKAGGKAGKPTKRSQNYAALRKAQLPKPPALKVPVVKPTNTILPQTGCVWQSLGTPLSLSSFNGLGARFLYSFLKDFVGPRILEEDLIYRMVFSITPSHAGTFCLTDDVTTEDGRAVAHGNPMQEFPHGAFHANEKFGFELVFTAPTHAGMQNQNFKHSYAVALCLDFDAQPEGSKNPSFRFNEVWVERKAFPRAGPLRSLITVGLFDEADDLDRH

SEQ ID NO: 11 - DNA, 666 bp, alfalfa mosaic virus strain YSMV
  CDS (1)..(663)
atgagttcttcacaaaagaaagctggtgggaaagctggtaaacctactaaacgttctcagaactatgctgctttacgcaaagctcgactgccgaagcctccggcgttgaaagtcccggttgcaaaaccgacgaataccatactgccacagacgggctgcgtgtggcaaagcctcgggacccctctgagtctgagctcttttaacgggctcggcgtgagattcctctacagttttctgaaggatttcgcgggacctcggatcctcgaagaggatctgatttacaggatggtgttttctataacaccgtcccatgccggtaccttttgtctcactgatgacgtgacgactgaggatggtagggccgttgcgcatggtaatcccatgcaagaatttcctcatggcgcgtttcacgccaatgagaagttcgggtttgagttggtcttcacagctcctacccatgcgggaatgcaaaatcaaaatttcaagcattcctatgccgtagccctttgtttggacttcgatgcgcagcctgagggatctaaaaatccctcataccgattcaacgaagtttgggtcgagagaaaggcgttcccgcgagcagggcccctccgcagtttgattactgtggggctgctcgacgaagctgacgatcttgatcgtcattga

SEQ ID NO: 12 - protein, 221 aa, alfalfa mosaic virus strain YSMV
MSSSQKKAGGKAGKPTKRSQNYAALRKARLPKPPALKVPVAKPTNTILPQTGCVWQSLGTPLSLSSFNGLGVRFLYSFLKDFAGPRILEEDLIYRMVFSITPSHAGTFCLTDDVTTEDGRAVAHGNPMQEFPHGAFHANEKFGFELVFTAPTHAGMQNQNFKHSYAVALCLDFDAQPEGSKNPSYRFNEVWVERKAFPRAGPLRSLITVGLLDEADDLDRH

SEQ ID NO: 13 - DNA, 666 bp, alfalfa mosaic virus strain AMV12509
  CDS (1)..(663)
atgagttcttcacaaaagaaagctggtgggaaagctggtaaatctactaaacgttctcagaactatgctgctttacgcaaagctcaactgccgaagcctccggcgttgaaagtcccggttgcaaaaccgacgaatactatactgccacagacgggctgtgtgtggcaaagcctcgggacccctctgagtctgagctcttttaatgggctcggcgtgagattcctctacagttttctgaaggatttcgcgggacctcggatcctcgaagaggatctgatttacaggatggtgttttctataacaccgtcccatgccggcaccttttgtctcactgatgacgtgacgactgaggatggtagggccgttgcgcatggtaatcccatgcaagaatttcctcatggcgtgtttcacgctaatgagaagttcgggtttagattggtcttcacagctcctacccatgcgggaatgcaaaatcaaaatttcaagcattcctatgccgtagccctctgtttggacttcgatgcgcagcctgagggatctaaaaatccctcataccgattcaacgaagtttgggtcgagagaaaggcgttcccgcgagcagggcccctccgcagtttgattactgtgggtctgttcgacgaagctgactatcttgatcgtcattga

SEQ ID NO: 14 - protein, 221 aa, alfalfa mosaic virus strain AMV12509
MSSSQKKAGGKAGKSTKRSQNYAALRKAQLPKPPALKVPVAKPTNTILPQTGCVWQSLGTPLSLSSFNGLGVRFLYSFLKDFAGPRILEEDLIYRMVFSITPSHAGTFCLTDDVTTEDGRAVAHGNPMQEFPHGVFHANEKFGFRLVFTAPTHAGMQNQNFKHSYAVALCLDFDAQPEGSKNPSYRFNEVWVERKAFPRAGPLRSLITVGLFDEADYLDRH

SEQ ID NO: 15 - DNA, 666 bp, alfalfa mosaic virus strain AMV12510
  CDS (1)..(663)
atgagttcttcacaaaagaaagctggtgggaaagctggtaaatctactaaacgttctcagaactatgctgctttacgcaaagctcaactgccgaagcctccggcgttgaaagtcccggttgcaaaaccgacgaatactatactgccacagacgggctgtgtgtggcaaagccacgggacccctctgagtctgagcttttttaatgggctcggcgtgagattcctctacagttttctgaaggatttcgcgggacctcggatcctcgaagaggatctgatttacaggatggtgttttctataacaccgtcccatgccggcaccttttgtctcactgatgacgtgatgactgaggatggtagggccgttgcgcatggtaatcccatgcaagaatttcctcatggcgtgtttcacgctaatgagaagttcgggtttgagttggtcttcacagctcctacccatgcgggaatgcaaaatcaaaatttcaagcattcctatgccgtagccctctgtttggacttcgatgcgcagcctgagggatctaaaaatccctcatcaagattcaacgaagtttgggtcgagagaaaggcgttcccgcgagcagggcccctccgcagtttgattactgtggggctgctcgacgaagctgacgatcttgatcgtcaatga

SEQ ID NO: 16 - protein, 221 aa, alfalfa mosaic virus strain AMV12510
MSSSQKKAGGKAGKSTKRSQNYAALRKAQLPKPPALKVPVAKPTNTILPQTGCVWQSHGTPLSLSFFNGLGVRFLYSFLKDFAGPRILEEDLIYRMVFSITPSHAGTFCLTDDVMTEDGRAVAHGNPMQEFPHGVFHANEKFGFELVFTAPTHAGMQNQNFKHSYAVALCLDFDAQPEGSKNPSSRFNEVWVERKAFPRAGPLRSLITVGLLDEADDLDRQ

SEQ ID NO: 17 - DNA, 666 bp, alfalfa mosaic virus strain YD3.2
  CDS (1)..(543)
atgagttcttcacaaaagaaagctggtgggaaagctggtaaacctactaaacgttctcagaactatgctgctttacgcaaagctcaactgccgaagcctccggcgttgaaagtcccggttgcaaaaccgacgaatactatactgccacagacgggctgcgtgtggcaaagcctcgggacccctctgagtctgagctctttcaatgggctcggcgtgagattcctctacagttttctgaaggatttcgcgggacctcggatcctcgaagaggatctgatttacaggatggtgttttctataacaccgtgccatgccggcaccttttgtctcactgatgacgtgacgactggggatggtagggccgttgcgcatggtaatcccatgcaagtatttcctcaaggcccgtttcacgccaatgggagttcggggtttgtgttggtcttcacagctcctacccatgcaggaatggaaaatcaaaatttcaagcattcctatgccgtagccctctgtctggacttcgatgcgcagcctgaggggtcttaaaatccctcataccgattcaacgaagtttgggtcgagagaaaggcgttcccgcgagcagggcccctccgcagtttgattactgtggggctgctcgacgaagcttacgatcttgatcgtcattca

SEQ ID NO: 18 - protein, 180 aa, alfalfa mosaic virus strain YD3.2
MSSSQKKAGGKAGKPTKRSQNYAALRKAQLPKPPALKVPVAKPTNTILPQTGCVWQSLGTPLSLSSFNGLGVRFLYSFLKDFAGPRILEEDLIYRMVFSITPCHAGTFCLTDDVTTGDGRAVAHGNPMQVFPQGPFHANGSSGFVLVFTAPTHAGMENQNFKHSYAVALCLDFDAQPEGS

SEQ ID NO: 19 - DNA, 22 bp, Artificial Sequence
  Description of Artificial Sequence primer
ccagatcttccatcatgagttc

SEQ ID NO: 20 - DNA, 26 bp, Artificial Sequence
  Description of Artificial Sequence primer
ccagatcttcaatgacgatcaagatc

SEQ ID NO: 21 - DNA, 41 bp, Artificial Sequence
  Description of Artificial Sequence primer
ggatccggatccacaatggacaaagagaagttgaatgctgg

SEQ ID NO: 22 - DNA, 41 bp, Artificial Sequence
  Description of Artificial Sequence primer
ggatccggatccacaatgggcaaagagcagttaaatgctgg

SEQ ID NO: 23 - DNA, 36 bp, Artificial Sequence
  Description of Artificial Sequence primer
gtcgacgtcgacctagaagcgagcaccagcaatatg

SEQ ID NO: 24 - DNA, 34 bp, Artificial Sequence
  Description of Artificial Sequence primer
gtcgacgtcgacctagaatcgtgctccagcaatg

SEQ ID NO: 25 - DNA, 876 bp, clover yellow vein virus strain CYVV300
  CDS (1)..(873)
tccggcaaagagcagttaaatgctggtgagcaacaaaagtcaaaggacaaggaaccaagacaaagagatcaagagggtgaaaactcaaacagacaaatcattccggacagagacatcaatgcaggtacaactgggactttctcagtacccaaattgaagaaaatatcaggaaagctttcattgcccaaaatcaaaggaaaaggactgctaaatctggaccatttgttagtttatgttccaaaccaagatgacatctctaacaacatagcaactcaagagcagctggaagcgtggcatgaaggagtcaagaatgcgtatgaggtggatgatcagcaaatggaaatcatatgcaatggattgatggtgtggtgcatagagaatggcacgtcaggtgatcttcaaggtgagtggacaatgatgtaccacaccacgtatctcttaccgtgcagtccactagaagttccactcacctgttactacgatggagagaaacaggtaacattcccacttaagccaattcttgactttgccaaaccaacattgaggcaaattatggctcacttttcccaggcagctgagtcatacatagaattcagaaactcaacagagagatatatgcctaggtatgggctgcagagaaatctcacagattatggattggccagatatgcatttgacttctacaggttgacctcaaagacaccagcaagagctcgagaagcacatatgcaaatgaaagcagcagcaataaaaggaaagtcaaaccacatgtttggactggatggcaatgttggaacagacgaggagaacacagaaaggcacacagcaaatgatgttaacaggaacatgcatcatattgctggtgctcgattctag

SEQ ID NO: 26 - protein, 291 aa, clover yellow vein virus strain CYVV300
SGKEQLNAGEQQKSKDKEPRQRDQEGENSNRQIIPDRDINAGTTGTFSVPKLKKISGKLSLPKIKGKGLLNLDHLLVYVPNQDDISNNIATQEQLEAWHEGVKNAYEVDDQQMEIICNGLMVWCIENGTSGDLQGEWTMMYHTTYLLPCSPLEVPLTCYYDGEKQVTFPLKPILDFAKPTLRQIMAHFSQAAESYIEFRNSTERYMPRYGLQRNLTDYGLARYAFDFYRLTSKTPARAREAHMQMKAAAIKGKSNHMFGLDGNVGTDEENTERHTANDVNRNMHHIAGARF

SEQ ID NO: 27 - DNA, 28 bp, Artificial Sequence
  Description of Artificial Sequence primer
aaactcgagcatggacttcactacttta

SEQ ID NO: 28 - DNA, 29 bp, Artificial Sequence
  Description of Artificial Sequence primer
aaactcgagatggcaaccaccacagcaac

SEQ ID NO: 29 - DNA, 38 bp, Artificial Sequence
  Description of Artificial Sequence primer
caggtaccctgaaattttattaaacagaaagcacacac

SEQ ID NO: 30 - DNA, 627 bp, WCMV strain Bundoora
  CDS (1)..(624)
atggcaaccaccacagcaaccactcctccatccttgacagacatccgagccctaaaatacacttcctccaccgtctcagtcgcctcacctgccgaaattgaagccatcaccaaaacctgggctgaaacattcaaaattccaaatgacgtcttgcctctcgcttgctgggatctggctcgtgctttcgctgatgttggcgcttcttctaagtctgaacttactggtgactctgctgctcttgcgggtgtttcacgtaaacaacttgcccaagccatcaaaatccattgcaccattcgccagttctgcatgtacttcgccaatgttgtgtggaacattatgttagataccaaaacaccaccagcatcctggtctaaacttggctacaaagaagagagcaaatttgctggctttgacttctttgatggtgtcaatcatcctgctgcactcatgcctgcagacggcctcatccgtggtccctccgaagctgaactcttagcccatcaaaccgcgaaacaagttgccctccatcgcgacgcaaaacgccgtggcactaacgttgtcaactctgttgaaatcactaacggtcgctccgaccctattggtccccttattacctatccccagtaa

SEQ ID NO: 31 - protein, 208 aa, WCMV strain Bundoora
MATTTATTPPSLTDIRALKYTSSTVSVASPAEIEAITKTWAETFKIPNDVLPLACWDLARAFADVGASSKSELTGDSAALAGVSRKQLAQAIKIHCTIRQFCMYFANVVWNIMLDTKTPPASWSKLGYKEESKFAGFDFFDGVNHPAALMPADGLIRGPSEAELLAHQTAKQVALHRDAKRRGTNVVNSVEITNGRSDPIGPLITYPQ

SEQ ID NO: 32 - DNA, 573 bp, WCMV strain M
  CDS (1)..(570)
atggcaaccaccacagcaaccactccaccatctttgaccgacatccgtgctctgaaatacacctcctccaccgtttctgttgcttcacccgctgagattgaagctatcaccaaaacctgggctgaaaccttcaaaattccgaacgatgtcctgcctctcgcttgttgggatctggctcgtgctttcgctgatgttggcgcttcttctaagtctgaacttactggtgactctgctgctcttgcgggtgtttcaaggaaacaacttgcccaagccatcaaaatccattgcaccattcgccaattctgcatgtacttcgccaatatcgtatggaacattatgctagacaccaaaacaccaccagcatcctggtctaagctaggctacaaagaagagagcaaattcgccggcttcgacttcttcgatggcgtcaaccatcccgctgcccttatgcccgctgacggcctcattcgtggtccttccgacgcggaaatcctagcacaccaaactgccaagcaagtagccctccaccgtgacgcaaaaccgacgtggcacaaacgttgtcaactctgttga

SEQ ID NO: 33 - protein, 190 aa, WCMV strain M
MATTTATTPPSLTDIRALKYTSSTVSVASPAEIEAITKTWAETFKIPNDVLPLACWDLARAFADVGASSKSELTGDSAALAGVSRKQLAQAIKIHCTIRQFCMYFANIVWNIMLDTKTPPASWSKLGYKEESKFAGFDFFDGVNHPAALMPADGLIRGPSDAEILAHQTAKQVALHRDAKPTWHKRCQLC

SEQ ID NO: 34 - DNA, 627 bp, WCMV strain O
  CDS (1)..(624)
atggcaaccaccacagcaaccactcctccatccttgacagacatccgagccctaaaatacacttcctccaccgtctcagtcgcctcacctgctgaaattgaagctatcactaaaacctgggcagaaacattcaaaattccaaatgacgtcttgcctctcgcttgttgggatctggctcgtgctttcgctgatgttggcgcttcttctaagtctgaacttactggtgactctgctgctcttgcgggtgtttcacggaaacaactggctcaagctatcaaaatccattgcaccattcgccagttctgcatgtacttcgccaatgttgtgtggaacatcatgttagataccaaaacaccgccagcatcctggtctaaactcggctataaagaagagagcaaattcgctggctttgacttctttgatggtgtcaatcatcctgctgcactcatgcctgcagacggcctcatccgtggtccttccgaagctgaactcttagcccatcaaaccgcaaaacaagtagccctccatcgcgacgcaaaacgccgtggcaccaacgttgtcaactctgttgaaatcactaacggtcgctccgaccctattggtccccttattacctatccccagtaa

SEQ ID NO: 35 - protein, 208 aa, WCMV strain O; identical to SEQ ID NO: 31

SEQ ID NO: 36 - DNA, 21 bp, Artificial Sequence
  Description of Artificial Sequence primer/probe
gaggctattcggctatgactg

SEQ ID NO: 37 - DNA, 21 bp, Artificial Sequence
  Description of Artificial Sequence primer/probe
atcgggagcggcgataccgta

SEQ ID NO: 38 - DNA, 26 bp, Artificial Sequence
  Description of Artificial Sequence primer/probe
gctggggcgtcggtttccactatcgg

SEQ ID NO: 39 - DNA, 27 bp, Artificial Sequence
  Description of Artificial Sequence primer/probe
cgcataacagcgctcattgactggagc

SEQ ID NO: 40 - DNA, 29 bp, Artificial Sequence
  Description of Artificial Sequence primer/probe
ccagatctcaacgaagatggttgctgttc

SEQ ID NO: 41 - DNA, 24 bp, Artificial Sequence
  Description of Artificial Sequence primer/probe
ccagatctttatacatcaatatac

SEQ ID NO: 42 - DNA, 20 bp, Artificial Sequence
  Description of Artificial Sequence primer/probe
ggctatgactgggcacaaca

SEQ ID NO: 43 - DNA, 19 bp, Artificial Sequence
  Description of Artificial Sequence primer/probe
accggacaggtcggtcttg

SEQ ID NO: 44 - DNA, 21 bp, Artificial Sequence
  Description of Artificial Sequence probe
ctctgatgccgccgtgttccg

SEQ ID NO: 45 - DNA, 654 bp, Artificial Sequence
  Description of Artificial Sequence duplicated CaMV 35S promoter
aacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgat

SEQ ID NO: 46 - DNA, 21 bp, Artificial Sequence
  Description of Artificial Sequence probe or primer
gtgaagcttcccgggcactgg

SEQ ID NO: 47 - DNA, 21 bp, Artificial Sequence
  Description of Artificial Sequence probe or primer
acccacttcgaagggcccgtg

SEQ ID NO: 48 - DNA, 30 bp, Artificial Sequence
  Description of Artificial Sequence probe or primer
ggagttgctggttgcggaaaaaccaccaat

SEQ ID NO: 49 - DNA, 30 bp, Artificial Sequence
  Description of Artificial Sequence probe or primer
ggagttgctggttgcggaaataccaccaat

SEQ ID NO: 50 - DNA, 21 bp, Artificial Sequence
  Description of Artificial Sequence probe or primer
gttgcggaaataccaccaata

SEQ ID NO: 51 - DNA, 31 bp, alfalfa mosaic virus
ggagttgctggttgcggaaataccaccaata

SEQ ID NO: 52 - DNA, 21 bp, alfalfa mosaic virus
gttgcggagaaaccaccaata

SEQ ID NO: 53 - DNA, 24 bp, alfalfa mosaic virus
gtctttgttgaccaatcttgcgtc

SEQ ID NO: 54 - DNA, 24 bp, alfalfa mosaic virus
aactttgtcaacggtgaacaatcg

SEQ ID NO: 55 - DNA, 18 bp, alfalfa mosaic virus
gaatgctgacgcccaatc

SEQ ID NO: 56 - DNA, 18 bp, alfalfa mosaic virus
ccatttgtcctttgactc